SEQUENCE LISTING (77 sequences)

SEQ ID NO: 1 - DNA, 1521 bp, Artificial Sequence
  Description of Artificial Sequence Synthetic polynucleotide
atgtcacggggctccgacctgggcaaaaagctgctggaggccgctagggccgggcaggacgatgaagtgagaatcctgatggccaacggggctgacgtgaatgctaaggatgagtacggcctgacccccctgtatctggctacagcacacggccatctggagatcgtggaagtcctgctgaaaaacggagccgacgtgaatgcagtcgatgccattgggttcactcctctgcacctggcagcctttatcggacatctggagattgcagaagtgctgctgaagcacggcgctgacgtgaacgcacaggataagttcggaaaaaccgcttttgacatcagcattggcaacggaaatgaagacctggctgaaatcctgcagaaactgaatgaacagaaactgattagcgaagaagacctgaaccccgggggaggaggagggagcgggggaggaggcagcggcgggggaggctctggaggaggagggagcggatccatggacatccagatgactcagaccacaagctccctgtctgcaagtctgggcgaccgggtgacaatctcctgcagagcctctcaggatattaggaactacctgaattggtatcagcagaaacctgatggcacagtcaagctgctgatctactataccagccggctgcactcaggcgtgccaagcaaattctcaggaagcggctccgggactgactactccctgaccatctctaacctggagcaggaagatattgctacctatttctgccagcagggcaatacactgccctggacttttgccggaggcaccaaactggagatcaaggggggaggcgggagtggaggcgggggatcaggaggaggaggcagcggaggaggagggtccgaggtccagctgcagcagagcggaccagaactggtgaagcccggagcaagtatgaaaatctcctgtaaggcctcaggatacagcttcaccggctatacaatgaactgggtgaaacagtcccatggcaagaacctggaatggatggggctgattaatccttacaaaggcgtcagcacctataatcagaagtttaaagacaaggccacactgactgtggataagtctagttcaaccgcttacatggagctgctgtccctgacatctgaagacagtgccgtgtactattgtgctcggtctggctactatggggacagtgattggtacttcgatgtctggggacagggcactaccctgaccgtgttttctactagtggcggaggaggatcactcgagagcggacaggtgctgctggaatccaatatcaaagtcctgcccacttggtctacccccgtgcagcctatggctctgattgtgctgggaggagtcgcaggactgctgctgtttatcgggctgggaattttcttttgcgtgcgctgccggcaccggagaaggcaggccgagcgcatgagccagatcaagcgactgctgagcgagaagaaaacctgtcagtgtccccatagattccagaagacctgttcacccatt

SEQ ID NO: 2 - protein, 525 aa, Artificial Sequence
  Description of Artificial Sequence Synthetic polypeptide
MDFQVQIFSFLLISASVIMSRGSDLGKKLLEAARAGQDDEVRILMANGADVNAKDEYGLTPLYLATAHGHLEIVEVLLKNGADVNAVDAIGFTPLHLAAFIGHLEIAEVLLKHGADVNAQDKFGKTAFDISIGNGNEDLAEILQKLNEQKLISEEDLNPGGGGGSGGGGSGGGGSGGGGSGSMDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSGGGGSGGGGSGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFSTSGGGGSLESGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 3 - DNA, 1647 bp, Artificial Sequence
  Description of Artificial Sequence Synthetic polynucleotide
atggactttcaggtgcagattttctcttttctgctgatttccgcaagcgtcatcgagctcggggggggggggtcaggatccatggacatccagatgactcagaccacaagctccctgagcgcatccctgggcgaccgagtgacaatctcatgcagagccagccaggatattaggaactacctgaattggtatcagcagaaacctgacggcacagtcaagctgctgatctactatacttcccggctgcactctggcgtgccaagtaaattctctgggagtggatcaggcactgactactcactgaccatcagcaacctggagcaggaagatattgctacctatttctgccagcagggcaatacactgccctggacttttgcaggcgggaccaaactggagatcaagggcggcggcggaagtggaggaggaggctcaggcggaggagggagcggcggaggaggcagcgaggtccagctgcagcagagcggaccagaactggtgaagcctggcgcatccatgaaaatctcttgtaaggcctctgggtacagtttcaccggatatacaatgaactgggtgaaacagtctcatggcaagaacctggaatggatgggcctgattaatccttacaaaggcgtcagcacctataatcagaagtttaaagacaaggccacactgactgtggataagtctagttcaaccgcttacatggagctgctgtcactgacaagcgaagactccgccgtgtactattgcgctaggagcggatactatggcgactccgattggtacttcgatgtctgggggcagggaactaccctgaccgtgtttagcactagtggaggaggaggctctggaggaggagggagtggaggcgggggatcaggaggaggaggcagcgatatcatgtcacggggctccgacctgggcaaaaagctgctggaggccgctagggccgggcaggacgatgaagtgagaatcctgatggccaacggggctgacgtgaatgctaaggatgagtacggcctgacccccctgtatctggctacagcacacggccatctggagatcgtggaagtcctgctgaaaaacggagccgacgtgaatgcagtcgatgccattgggttcactcctctgcacctggcagcctttatcggacatctggagattgcagaagtgctgctgaagcacggcgctgacgtgaacgcacaggataagttcggaaaaaccgcttttgacatcagcattggcaacggaaatgaagacctggctgaaatcctgcagaaactgaatgaacagaaactgattagcgaagaagacctgaacgtcgacggaggaggagggtctggaggagggggaagtggcgggggaggcagcgggggaggcgggtctctcgagagtggccaggtgctgctggaaagcaatatcaaggtcctgccaacttggtccaccccagtgcagcctatggctctgattgtgctgggaggagtcgcaggactgctgctgtttatcggcctggggattttcttttgcgtgcgctgccggcaccggagaaggcaggctgagcgcatgtctcagattaagcgactgctgagcgagaagaagacctgtcagtgcccccatagattccagaaaacctgttcacccatt

SEQ ID NO: 4 - protein, 547 aa, Artificial Sequence
  Description of Artificial Sequence Synthetic polypeptide
MDFQVQIFSFLLISASVIELGGGGSGSMDIQMTQTTSSLSASLGDRVTISCRASQDIRNYLNWYQQKPDGTVKLLIYYTSRLHSGVPSKFSGSGSGTDYSLTISNLEQEDIATYFCQNTLPWTFAGGTKLEIKGGGGSGGGGSGGGGSGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFSTSGGGGSGGGGSGGGGSGGGGSDIMSRGSDLGKKLLEAARAGQDDEVRILMANGADVNAKDEYGLTPLYLATAHGHLEIVEVLLKNGADVNAVDAIGFTPLHLAAFIGHLEIAEVLLKHGADVNAQDKFGKTAFDISIGNGNEDLAEILQKLNEQKLISEEDLNVDGGGGSGGGGSGGGGSGGGGSLESGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 5 - DNA, 54 bp, Artificial Sequence
  Description of Artificial Sequence Synthetic oligonucleotide
atggatttccaggtccagattttctccttcctgctgatttccgcaagcgtcatt

SEQ ID NO: 6 - protein, 18 aa, Artificial Sequence
  Description of Artificial Sequence Synthetic peptide
MDFQVQIFSFLLISASVI

SEQ ID NO: 7 - DNA, 387 bp, Artificial Sequence
  Description of Artificial Sequence Synthetic polynucleotide
atgtcacggggctccgacctgggcaaaaagctgctggaggccgctagggccgggcaggacgatgaagtgagaatcctgatggccaacggggctgacgtgaatgctaaggatgagtacggcctgacccccctgtatctggctacagcacacggccatctggagatcgtggaagtcctgctgaaaaacggagccgacgtgaatgcagtcgatgccattgggttcactcctctgcacctggcagcctttatcggacatctggagattgcagaagtgctgctgaagcacggcgctgacgtgaacgcacaggataagttcggaaaaaccgcttttgacatcagcattggcaacggaaatgaagacctggctgaaatcctgcagaaactgaat

SEQ ID NO: 8 - protein, 129 aa, Artificial Sequence
  Description of Artificial Sequence Synthetic polypeptide
MSRGSDLGKKLLEAARAGQDDEVRILMANGADVNAKDEYGLTPLYLATAHGHLEIVEVLLKNGADVNAVDAIGFTPLHLAAFIGHLEIAEVLLKHGADVNAQDKFGKTAFDISIGNGNEDLAEILQKLN

SEQ ID NO: 9 - DNA, 30 bp, Artificial Sequence
  Description of Artificial Sequence Synthetic oligonucleotide
gaacagaaactgattagcgaagaagacctg

SEQ ID NO: 10 - protein, 10 aa, Artificial Sequence
  Description of Artificial Sequence Synthetic peptide
EQKLISEEDL

SEQ ID NO: 11 - DNA, 75 bp, Artificial Sequence
  Description of Artificial Sequence Synthetic oligonucleotide
aaccccgggggaggaggagggagcgggggaggaggcagcggcgggggaggctctggaggaggagggagcggatcc

SEQ ID NO: 12 - protein, 25 aa, Artificial Sequence
  Description of Artificial Sequence Synthetic peptide
NPGGGGGSGGGGSGGGGSGGGGSGS

SEQ ID NO: 13 - DNA, 750 bp, Artificial Sequence
  Description of Artificial Sequence Synthetic polynucleotide
atggacatccagatgactcagaccacaagctccctgtctgcaagtctgggcgaccgggtgacaatctcctgcagagcctctcaggatattaggaactacctgaattggtatcagcagaaacctgatggcacagtcaagctgctgatctactataccagccggctgcactcaggcgtgccaagcaaattctcaggaagcggctccgggactgactactccctgaccatctctaacctggagcaggaagatattgctacctatttctgccagcagggcaatacactgccctggacttttgccggaggcaccaaactggagatcaaggggggaggcgggagtggaggcgggggatcaggaggaggaggcagcggaggaggagggtccgaggtccagctgcagcagagcggaccagaactggtgaagcccggagcaagtatgaaaatctcctgtaaggcctcaggatacagcttcaccggctatacaatgaactgggtgaaacagtcccatggcaagaacctggaatggatggggctgattaatccttacaaaggcgtcagcacctataatcagaagtttaaagacaaggccacactgactgtggataagtctagttcaaccgcttacatggagctgctgtccctgacatctgaagacagtgccgtgtactattgtgctcggtctggctactatggggacagtgattggtacttcgatgtctggggacagggcactaccctgaccgtgttttct

SEQ ID NO: 14 - protein, 250 aa, Artificial Sequence
  Description of Artificial Sequence Synthetic polypeptide
MDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSGGGGSGGGGSGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFS

SEQ ID NO: 15 - DNA, 27 bp, Artificial Sequence
  Description of Artificial Sequence Synthetic oligonucleotide
actagtggcggaggaggatcactcgag

SEQ ID NO: 16 - protein, 9 aa, Artificial Sequence
  Description of Artificial Sequence Synthetic peptide
TSGGGGSLE

SEQ ID NO: 17 - DNA, 252 bp, Artificial Sequence
  Description of Artificial Sequence Synthetic polynucleotide
agcggacaggtgctgctggaatccaatatcaaagtcctgcccacttggtctacccccgtgcagcctatggctctgattgtgctgggaggagtcgcaggactgctgctgtttatcgggctgggaattttcttttgcgtgcgctgccggcaccggagaaggcaggccgagcgcatgagccagatcaagcgactgctgagcgagaagaaaacctgtcagtgtccccatagattccagaagacctgttcacccatt

SEQ ID NO: 18 - protein, 84 aa, Artificial Sequence
  Description of Artificial Sequence Synthetic polypeptide
SGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 19 - DNA, 66 bp, Artificial Sequence
  Description of Artificial Sequence Synthetic oligonucleotide
agcggacaggtgctgctggaatccaatatcaaagtcctgcccacttggtctacccccgtgcagcct

SEQ ID NO: 20 - protein, 22 aa, Artificial Sequence
  Description of Artificial Sequence Synthetic peptide
SGQVLLESNIKVLPTWSTPVQP

SEQ ID NO: 21 - DNA, 769 bp, Artificial Sequence
  Description of Artificial Sequence Synthetic polynucleotide
atggccgacatcgtgctgacacagagccccgccatcatgtctgccagccctggcgagaaagtgaccatgacctgtagcgccagcagcagcgtgtcctacatgaactggtatcagcagaagtccggcaccagccccaagcggtggatctacgacacaagcaagctggcctctggcgtgcccgcccactttagaggctctggcagcggcacaagctacagcctgaccatcagcggcatggaagccgaggatgccgccacctactactgccagcagtggtccagcaaccccttcacctttggctccggcacaaagctggaaatcaaccgggccgacaccgcccctacaggcggcggaggatctggcggaggcggatctgggggcggaggaagtggggggggaggatctatggctcaggtgcagctgcagcagtctggcgccgaactggctagacctggcgcctccgtgaagatgagctgcaaggccagcggctacaccttcacccggtacaccatgcactgggtcaagcagaggcctggacagggcctggaatggatcggctacatcaaccccagccggggctacaccaactacaaccagaagttcaaggacaaggccaccctgaccaccgacaagagcagcagcaccgcctacatgcagctgtcctccctgaccagcgaggacagcgccgtgtactactgcgcccggtactacgacgaccactactccctggactactggggccagggcaccacactgaccgtgtctagta

SEQ ID NO: 22 - protein, 256 aa, Artificial Sequence
  Description of Artificial Sequence Synthetic polypeptide
MADIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRADTAPTGGGGSGGGGSGGGGSGGGGSMAQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSS

SEQ ID NO: 23 - DNA, 747 bp, Artificial Sequence
  Description of Artificial Sequence Synthetic polynucleotide
cagaccgtggtgacccaggagcccagcctgaccgtgagccccggcggcaccgtgaccctgacctgcggcagcagcaccggcgccgtgaccagcggctactaccccaactgggtgcagcagaagcccggccaggcccccaggggcctgatcggcggcaccaagttcctggcccccggcacccccgccaggttcagcggcagcctgctgggcggcaaggccgccctgaccctgagcggcgtgcagcccgaggacgaggccgagtactactgcgccctgtggtacagcaacaggtgggtgttcggcggcggcaccaagctgaccgtgctgggcggcggcggcagcggcggcggcggcagcggcggcggcggcagcgaggtgcagctgctggagagcggcggcggcctggtgcagcccggcggcagcctgaagctgagctgcgccgccagcggcttcaccttcaacatctacgccatgaactgggtgaggcaggcccccggcaagggcctggagtgggtggccaggatcaggagcaagtacaacaactacgccacctactacgccgacagcgtgaagagcaggttcaccatcagcagggacgacagcaagaacaccgcctacctgcagatgaacaacctgaagaccgaggacaccgccgtgtactactgcgtgaggcacggcaacttcggcaacagctacgtgagcttcttcgcctactggggccagggcaccctggtgaccgtgagcagc

SEQ ID NO: 24 - protein, 249 aa, Artificial Sequence
  Description of Artificial Sequence Synthetic polypeptide
QTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVLGGGGSGGGGSGGGGSEVQLLESGGGLVQPGGSLKLSCAASGFTFNIYAMNWVRQAPGKGLEWVARIRSKYNNYATYYADSVKSRFTISRDDSKNTAYLQMNNLKTEDTAVYYCVRHGNFGNSYVSFFAYWGQGTLVTVSS

SEQ ID NO: 25 - DNA, 720 bp, Artificial Sequence
  Description of Artificial Sequence Synthetic polynucleotide
gacatccagctgacccagagccccgccatcatgagcgccagccccggcgagaaggtgaccatgacctgcagggccagcagcagcgtgagctacatgaactggtaccagcagaagagcggcaccagccccaagaggtggatctacgacaccagcaaggtggccagcggcgtgccctacaggttcagcggcagcggcagcggcaccagctacagcctgaccatcagcagcatggaggccgaggacgccgccacctactactgccagcagtggagcagcaaccccctgaccttcggcgccggcaccaagctggagctgaagggcggcggcggcagcggcggcggcggcagcggcggcggcggcagcgacatcaagctgcagcagagcggcgccgagctggccaggcccggcgccagcgtgaagatgagctgcaagaccagcggctacaccttcaccaggtacaccatgcactgggtgaagcagaggcccggccagggcctggagtggatcggctacatcaaccccagcaggggctacaccaactacaaccagaagttcaaggacaaggccaccctgaccaccgacaagagcagcagcaccgcctacatgcagctgagcagcctgaccagcgaggacagcgccgtgtactactgcgccaggtactacgacgaccactactgcctggactactggggccagggcaccaccctgaccgtgagcagc

SEQ ID NO: 26 - protein, 240 aa, Artificial Sequence
  Description of Artificial Sequence Synthetic polypeptide
DIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKGGGGSGGGGSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 27 - DNA, 51 bp, Artificial Sequence
  Description of Artificial Sequence Synthetic oligonucleotide
gccgaagcagcagcaaaggaggccgcagcgaaggaagcagctgcgaaggcc

SEQ ID NO: 28 - protein, 17 aa, Artificial Sequence
  Description of Artificial Sequence Synthetic peptide
AEAAAKEAAAKEAAAKA

SEQ ID NO: 29 - DNA, 81 bp, Artificial Sequence
  Description of Artificial Sequence Synthetic oligonucleotide
gccgaggcagctgcaaaggaagctgcggcgaaggaggccgcagcgaaagaagcagcggcaaaagaagcagccgccaaagcc

SEQ ID NO: 30 - protein, 27 aa, Artificial Sequence
  Description of Artificial Sequence Synthetic peptide
AEAAAKEAAAKEAAAKEAAAKEAAAKA

SEQ ID NO: 31 - DNA, 576 bp, Artificial Sequence
  Description of Artificial Sequence Synthetic polynucleotide
atcgtagtgttggcatttcaaaaagcgtctagcatcgtctataagaaggaaggtgaacaagtcgagttttctttcccccttgcatttacggtggaaaagcttacgggtagcggcgagctgtggtggcaagctgaacgggcttcaagctcaaaatcttggattacttttgacttgaagaacaaagaggtgagtgtcaaaagagttactcaggacccaaagcttcaaatggggaagaaacttccgctgcacctgacgttgcctcaggccctgcctcaatatgccggctcaggcaatctgaccctcgcgctggaagctaagaccggaaaattgcaccaggaagtcaatttggttgtgatgcgcgccactcagctccaaaaaaatctcacttgcgaggtatgggggcctacgagcccaaaacttatgctgtctttgaagcttgaaaacaaggaagcgaaagtttctaagcgcgagaaagcggtatgggttttgaatcctgaggctggaatgtggcaatgcctcctgagcgatagcgggcaggtgctgttggagagcaacatcaaggttttgccagcagcc

SEQ ID NO: 32 - protein, 192 aa, Artificial Sequence
  Description of Artificial Sequence Synthetic polypeptide
IVVLAFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPAA

SEQ ID NO: 33 - DNA, 801 bp, Artificial Sequence
  Description of Artificial Sequence Synthetic polynucleotide
atggattttcaggtgcagattttcagcttcctgctaatcagtgcctcagtcataatgtctagagacatcgtgctgacccagagcccccccagcctggccatgtctctgggcaagagagccaccatcagctgccgggccagcgagagcgtgaccatcctgggcagccacctgatccactggtatcagcagaagcccggccagccccccaccctgctgatccagctcgccagcaatgtgcagaccggcgtgcccgccagattcagcggcagcggcagcagaaccgacttcaccctgaccatcgaccccgtggaagaggacgacgtggccgtgtactactgcctgcagagccggaccatcccccggacctttggcggaggcaccaaactggaaatcaagggcagcaccagcggctccggcaagcctggctctggcgagggcagcacaaagggacagattcagctggtgcagagcggccctgagctgaagaaacccggcgagacagtgaagatcagctgcaaggcctccggctacaccttcaccgactacagcatcaactgggtgaaaagagcccctggcaagggcctgaagtggatgggctggatcaacaccgagacaagagagcccgcctacgcctacgacttccggggcagattcgccttcagcctggaaaccagcgccagcaccgcctacctgcagatcaacaacctgaagtacgaggacaccgccacctacttttgcgccctggactacagctacgccatggactactggggccagggcaccagcgtgaccgtgtccagc

SEQ ID NO: 34 - protein, 267 aa, Artificial Sequence
  Description of Artificial Sequence Synthetic polypeptide
MDFQVQIFSFLLISASVIMSRDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 35 - DNA, 735 bp, Artificial Sequence
  Description of Artificial Sequence Synthetic polynucleotide
gatatccagatgactcagacgacctcatcattgtccgccagtttgggggacagggttacaatatcctgccgggcgagccaagacatcagtaaatatcttaattggtaccagcagaaaccagatggtacagtaaaacttcttatctaccacacctctcggctccactctggggttccctctaggttcagtggtagtgggtcaggcaccgactacagccttacgataagcaacttggaacaggaggatatcgcaacttacttctgccaacagggaaataccctgccttacacgttcggtggaggcactaaactggagatcactgggtcaacctctggtagcggtaagcctggctccggcgaaggctccacaaagggtgaggtgaaactccaagagtcaggtcccggtttggtagccccctcacaaagtttgtcagttacttgtaccgtaagcggcgtttccctgcccgattacggtgtgagctggataaggcagccaccgagaaaaggtcttgaatggctgggagtgatctgggggtctgagacaacgtattacaactcagctcttaagagcaggcttacgatcattaaagataacagcaaatctcaagtgttcctcaaaatgaatagccttcaaactgatgatactgccatctattattgtgctaagcattattactatggcggcagttacgcaatggattattgggggcaaggtacctcagtcactgtaagcagc

SEQ ID NO: 36 - protein, 245 aa, Artificial Sequence
  Description of Artificial Sequence Synthetic polypeptide
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 37 - DNA, 237 bp, Artificial Sequence
  Description of Artificial Sequence Synthetic polynucleotide
ctcgagctgaggcccgaggcttctagacctgctgccggcggagccgtgcacaccagaggcctggacttcgccagcgacatctacatctgggcccctctggccggcacctgtggcgtgctgctgctgagcctggtcatcaccctgtactgcaaccaccggaaccggcggagagtgtgcaagtgccccagacccgtggtcaagagcggcgacaagcccagcctgagcgccagatacgtg

SEQ ID NO: 38 - protein, 79 aa, Artificial Sequence
  Description of Artificial Sequence Synthetic polypeptide
LELRPEASRPAAGGAVHTRGLDFASDIYIWAPLAGTCGVLLLSLVITLYCNHRNRRRVCKCPRPVVKSGDKPSLSARYV

SEQ ID NO: 39 - DNA, 234 bp, Artificial Sequence
  Description of Artificial Sequence Synthetic polynucleotide
ctcgagctgaggcccgaggcttctagacctgctgccggcggagccgtgcacaccagaggcctggacttcgccagcgacatctacatctgggcccctctggccggcacctgtggcgtgctgctgctgagcctggtcatcaccctgtacctgtgctgcagacggcggagagtgtgcaagtgccccagacccgtggtcaagagcggcgacaagcccagcctgagcgccagatacgtg

SEQ ID NO: 40 - protein, 78 aa, Artificial Sequence
  Description of Artificial Sequence Synthetic polypeptide
LELRPEASRPAAGGAVHTRGLDFASDIYIWAPLAGTCGVLLLSLVITLYLCCRRRRVCKCPRPVVKSGDKPSLSARYV

SEQ ID NO: 41 - DNA, 213 bp, Artificial Sequence
  Description of Artificial Sequence Synthetic polynucleotide
ctcgagaagaagtccaccctgaagaaacgggtgtcccggctgcccagacccgagacacagaagggccccctgagcagccctatcaccctgggactgctggtggccggcgtgctggtgctgctggtgtctctgggagtggccatccacctgtgctgccggcggagaagggcctgcaagtgccccagactgcggttcatgaagcagttctacaag

SEQ ID NO: 42 - protein, 71 aa, Artificial Sequence
  Description of Artificial Sequence Synthetic polypeptide
LEKKSTLKKRVSRLPRPETQKGPLSSPITLGLLVAGVLVLLVSLGVAIHLCCRRRRACKCPRLRFMKQFYK

SEQ ID NO: 43 - DNA, 735 bp, Artificial Sequence
  Description of Artificial Sequence Synthetic polynucleotide
atggatatccagatgacccagtccccgagctccctgtccgcctctgtgggcgatagggtcaccatcacctgccgtgccagtcaggacatccgtaattatctgaactggtatcaacagaaaccaggaaaagctccgaaactactgatttactatacctcccgcctggagtctggagtcccttctcgcttctctggttctggttctgggacggattacactctgaccatcagcagtctgcaaccggaagacttcgcaacttattactgtcagcaaggtaatactctgccgtggacgttcggacagggcaccaaggtggagatcaaaggcggcggcggaagtggaggaggaggctcaggcggaggagggagcgaggttcagctggtggagtctggcggtggcctggtgcagccagggggctcactccgtttgtcctgtgcagcttctggctactcctttaccggctacactatgaactgggtgcgtcaggccccaggtaagggcctggaatgggttgcactgattaatccttataaaggtgttagtacctacaaccagaagttcaaggaccgtttcactataagcgtagataaatccaaaaacacagcctacctgcaaatgaacagcctgcgtgctgaggacactgccgtctattattgtgctagaagcggatactacggcgatagtgactggtattttgacgtgtggggtcaaggaaccctggtcaccgtctcctcg

SEQ ID NO: 44 - protein, 245 aa, Artificial Sequence
  Description of Artificial Sequence Synthetic polypeptide
MDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSS

SEQ ID NO: 45 - DNA, 735 bp, Artificial Sequence
  Description of Artificial Sequence Synthetic polynucleotide
atggatatccagatgacccagtccccgagctccctgtccgcctctgtgggcgatagggtcaccatcacctgccgtgccagtcaggacatccgtaattatctgaactggtatcaacagaaaccaggaaaagctccgaaactactgatttactatacctcccgcctggagtctggagtcccttctcgcttctctggttctggttctgggacggattacactctgaccatcagcagtctgcaaccggaagacttcgcaacttattactgtcagcaaggtaatactctgccgtggacgttcggacagggcaccaaggtggagatcaaaggcggcggcggaagtggaggaggaggctcaggcggaggagggagcgaggttcagctggtggagtctggcggtggcctggtgcagccagggggctcactccgtttgtcctgtgcagcttctggctactcctttaccggctacactatgaactgggtgcgtcaggccccaggtaagggcctggaatgggttgcactgattaatcctaccaaaggtgttagtacctacaaccagaagttcaaggaccgtttcactataagcgtagataaatccaaaaacacagcctacctgcaaatgaacagcctgcgtgctgaggacactgccgtctattattgtgctagaagcggatactacggcgatagtgactggtattttgacgtgtggggtcaaggaaccctggtcaccgtctcctcg

SEQ ID NO: 46 - protein, 245 aa, Artificial Sequence
  Description of Artificial Sequence Synthetic polypeptide
MDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSEVQLVESGGGLVQPGGSLRLSCAASGYSFTGYTMNWVRQAPGKGLEWVALINPTKGVSTYNQKFKDRFTISVDKSKNTAYLQMNSLRAEDTAVYYCARSGYYGDSDWYFDVWGQGTLVTVSS

SEQ ID NO: 47 - DNA, 60 bp, Artificial Sequence
  Description of Artificial Sequence Synthetic oligonucleotide
atggagacccccgcccagctgctgttcctgctgctgctgtggctgcccgacaccaccggc

SEQ ID NO: 48 - protein, 20 aa, Artificial Sequence
  Description of Artificial Sequence Synthetic peptide
METPAQLLFLLLLWLPDTTG

SEQ ID NO: 49 - DNA, 63 bp, Artificial Sequence
  Description of Artificial Sequence Synthetic oligonucleotide
atggctttgcctgtcacggctcttctgctccctctggccctgcttctgcacgcggcgcgaccc

SEQ ID NO: 50 - protein, 21 aa, Artificial Sequence
  Description of Artificial Sequence Synthetic peptide
MALPVTALLLPLALLLHAARP

SEQ ID NO: 51 - DNA, 732 bp, Artificial Sequence
  Description of Artificial Sequence Synthetic polynucleotide
gaggtgcagctggtggagtctggaggaggcctggtgcagcctggcggctccctgaggctgtcttgcgcagcaagcggcttcaacatctactatagctacatgcactgggtgcgccaggcccctggcaagggcctggagtgggtggcctccatctctccatactatggctacacctcctatgccgactctgtgaagggccggtttacaatcagcgccgatacctccaagaacacagcctatctgcagatgaattccctgagggcagaggacaccgccgtgtactattgcgccagacacggctacgccctggattattggggccagggcaccctggtgacagtgagctccggcagcacatccggatctggcaagccaggctctggagagggaagcaccaagggcgacatccagatgacacagtccccatctagcctgagcgcctccgtgggcgatagggtgaccatcacatgtcgcgcctctcagagcgtgtcctctgccgtggcatggtaccagcagaagcccggcaaggcccctaagctgctgatctacagcgccagctccctgtattccggcgtgccttctcggttctccggctctagaagcggcaccgactttaccctgacaatctctagcctgcagcccgaggatttcgccacatactattgtcagcagagcgtgtgggtgggctactccctgatcacctttggccagggcacaaaggtggagatcaag

SEQ ID NO: 52 - protein, 244 aa, Artificial Sequence
  Description of Artificial Sequence Synthetic polypeptide
EVQLVESGGGLVQPGGSLRLSCAASGFNIYYSYMHWVRQAPGKGLEWVASISPYYGYTSYADSVKGRFTISADTSKNTAYLQMNSLRAEDTAVYYCARHGYALDYWGQGTLVTVSSGSTSGSGKPGSGEGSTKGDIQMTQSPSSLSASVGDRVTITCRASQSVSSAVAWYQQKPGKAPKLLIYSASSLYSGVPSRFSGSRSGTDFTLTISSLQPEDFATYYCQQSVWVGYSLITFGQGTKVEIK

SEQ ID NO: 53 - DNA, 732 bp, Artificial Sequence
  Description of Artificial Sequence Synthetic polynucleotide
gacatccagatgacacagtccccaagctccctgtccgcctctgtgggcgatagggtgaccatcacatgcagggcaagccagtccgtgtctagcgccgtggcatggtaccagcagaagcccggcaaggcccctaagctgctgatctacagcgcctcctctctgtattccggcgtgccatctcggttctctggcagcagatccggcaccgactttaccctgacaatcagctccctgcagcccgaggatttcgccacatactattgccagcagagcgtgtgggtgggctactccctgatcacctttggccagggcacaaaggtggagatcaagggatctaccagcggatccggcaagcctggcagcggagagggatccacaaagggagaggtgcagctggtggagtctggaggaggcctggtgcagcctggcggctctctgaggctgagctgtgcagcatccggcttcaacatctactatagctacatgcactgggtgcgccaggcccccggcaagggcctggagtgggtggcctctatcagcccttactatggctacacctcttatgccgacagcgtgaagggccggtttacaatctccgccgatacctctaagaacacagcctatctgcagatgaattccctgagggcagaggacaccgccgtgtactattgtgccagacacggctacgccctggattattggggccagggcaccctggtgacagtgtctagc

SEQ ID NO: 54 - protein, 244 aa, Artificial Sequence
  Description of Artificial Sequence Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQSVWVGYSLITFGQGTKVEIKGSTSGSGKPGSGEGSTKGEVQLVESGGGLVQPGGSLRLSCAASGFNIYYSYMHWVRQAPGKGLEWVASISPYYGYTSYADSVKGRFTISADTSKNTAYLQMNSLRAEDTAVYYCARHGYALDYWGQGTLVTVSS

SEQ ID NO: 55 - DNA, 1905 bp, Artificial Sequence
  Description of Artificial Sequence Synthetic polynucleotide
atggccctgccagtgaccgccctgctgctgccactggccctgctgctgcacgccgccagacccgaggtgcagctggtggagtctggaggaggcctggtgcagcctggcggctccctgaggctgtcttgcgcagcaagcggcttcaacatctactatagctacatgcactgggtgcgccaggcccctggcaagggcctggagtgggtggcctccatctctccatactatggctacacctcctatgccgactctgtgaagggccggtttacaatcagcgccgatacctccaagaacacagcctatctgcagatgaattccctgagggcagaggacaccgccgtgtactattgcgccagacacggctacgccctggattattggggccagggcaccctggtgacagtgagctccggcagcacatccggatctggcaagccaggctctggagagggaagcaccaagggcgacatccagatgacacagtccccatctagcctgagcgcctccgtgggcgatagggtgaccatcacatgtcgcgcctctcagagcgtgtcctctgccgtggcatggtaccagcagaagcccggcaaggcccctaagctgctgatctacagcgccagctccctgtattccggcgtgccttctcggttctccggctctagaagcggcaccgactttaccctgacaatctctagcctgcagcccgaggatttcgccacatactattgtcagcagagcgtgtgggtgggctactccctgatcacctttggccagggcacaaaggtggagatcaaggagcagaagctgatcagcgaggaggacctgaatcccggggccgaagcagcagcaaaggaggccgcagcgaaggaagcagctgcgaaggccggatccatggatatccagatgacccagtccccgagctccctgtccgcctctgtgggcgatagggtcaccatcacctgccgtgccagtcaggacatccgtaattatctgaactggtatcaacagaaaccaggaaaagctccgaaactactgatttactatacctcccgcctggagtctggagtcccttctcgcttctctggttctggttctgggacggattacactctgaccatcagcagtctgcaaccggaagacttcgcaacttattactgtcagcaaggtaatactctgccgtggacgttcggacagggcaccaaggtggagatcaaaggcggcggcggaagtggaggaggaggctcaggcggaggagggagcgaggttcagctggtggagtctggcggtggcctggtgcagccagggggctcactccgtttgtcctgtgcagcttctggctactcctttaccggctacactatgaactgggtgcgtcaggccccaggtaagggcctggaatgggttgcactgattaatccttataaaggtgttagtacctacaaccagaagttcaaggaccgtttcactataagcgtagataaatccaaaaacacagcctacctgcaaatgaacagcctgcgtgctgaggacactgccgtctattattgtgctagaagcggatactacggcgatagtgactggtattttgacgtgtggggtcaaggaaccctggtcaccgtctcctcgactagtggcggaggaggatcactcgagagcggacaggtgctgctggaatccaatatcaaagtcctgcccacttggtctacccccgtgcagcctatggctctgattgtgctgggaggagtcgcaggactgctgctgtttatcgggctgggaattttcttttgcgtgcgctgccggcaccggagaaggcaggccgagcgcatgagccagatcaagcgactgctgagcgagaagaaaacctgtcagtgtccccatagattccagaagacctgttcacccatt

SEQ ID NO: 56 - protein, 635 aa, Artificial Sequence
  Description of Artificial Sequence Synthetic polypeptide
MALPVTALLLPLALLLHAARPEVQLVESGGGLVQPGGSLRLSCAASGFNIYYSYMHWVRQAPGKGLEWVASISPYYGYTSYADSVKGRFTISADTSKNTAYLQMNSLRAEDTAVYYCARHGYALDYWGQGTLVTVSSGSTSGSGKPGSGEGSTKGDIQMTQSPSSLSASVGDRVTITCRASQSVSSAVAWYQQKPGKAPKLLIYSASSLYSGVPSRFSGSRSGTDFTLTISSLQPEDFATYYCQQSVWVGYSLITFGQGTKVEIKEQKLISEEDLNPGAEAAAKEAAAKEAAAKAGSMDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSTSGGGGSLESGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 57 - DNA, 1905 bp, Artificial Sequence
  Description of Artificial Sequence Synthetic polynucleotide
atggccctgccagtgaccgccctgctgctgccactggccctgctgctgcacgccgcccggcctgacatccagatgacacagtccccaagctccctgtccgcctctgtgggcgatagggtgaccatcacatgcagggcaagccagtccgtgtctagcgccgtggcatggtaccagcagaagcccggcaaggcccctaagctgctgatctacagcgcctcctctctgtattccggcgtgccatctcggttctctggcagcagatccggcaccgactttaccctgacaatcagctccctgcagcccgaggatttcgccacatactattgccagcagagcgtgtgggtgggctactccctgatcacctttggccagggcacaaaggtggagatcaagggatctaccagcggatccggcaagcctggcagcggagagggatccacaaagggagaggtgcagctggtggagtctggaggaggcctggtgcagcctggcggctctctgaggctgagctgtgcagcatccggcttcaacatctactatagctacatgcactgggtgcgccaggcccccggcaagggcctggagtgggtggcctctatcagcccttactatggctacacctcttatgccgacagcgtgaagggccggtttacaatctccgccgatacctctaagaacacagcctatctgcagatgaattccctgagggcagaggacaccgccgtgtactattgtgccagacacggctacgccctggattattggggccagggcaccctggtgacagtgtctagcgagcagaagctgatcagcgaggaggacctgaatcccggggccgaagcagcagcaaaggaggccgcagcgaaggaagcagctgcgaaggccggatccatggatatccagatgacccagtccccgagctccctgtccgcctctgtgggcgatagggtcaccatcacctgccgtgccagtcaggacatccgtaattatctgaactggtatcaacagaaaccaggaaaagctccgaaactactgatttactatacctcccgcctggagtctggagtcccttctcgcttctctggttctggttctgggacggattacactctgaccatcagcagtctgcaaccggaagacttcgcaacttattactgtcagcaaggtaatactctgccgtggacgttcggacagggcaccaaggtggagatcaaaggcggcggcggaagtggaggaggaggctcaggcggaggagggagcgaggttcagctggtggagtctggcggtggcctggtgcagccagggggctcactccgtttgtcctgtgcagcttctggctactcctttaccggctacactatgaactgggtgcgtcaggccccaggtaagggcctggaatgggttgcactgattaatccttataaaggtgttagtacctacaaccagaagttcaaggaccgtttcactataagcgtagataaatccaaaaacacagcctacctgcaaatgaacagcctgcgtgctgaggacactgccgtctattattgtgctagaagcggatactacggcgatagtgactggtattttgacgtgtggggtcaaggaaccctggtcaccgtctcctcgactagtggcggaggaggatcactcgagagcggacaggtgctgctggaatccaatatcaaagtcctgcccacttggtctacccccgtgcagcctatggctctgattgtgctgggaggagtcgcaggactgctgctgtttatcgggctgggaattttcttttgcgtgcgctgccggcaccggagaaggcaggccgagcgcatgagccagatcaagcgactgctgagcgagaagaaaacctgtcagtgtccccatagattccagaagacctgttcacccatt

SEQ ID NO: 58 - protein, 635 aa, Artificial Sequence
  Description of Artificial Sequence Synthetic polypeptide
MALPVTALLLPLALLLHAARPDIQMTQSPSSLSASVGDRVTITCRASQSVSSAVAWYQQKPGKAPKLLIYSASSLYSGVPSRFSGSRSGTDFTLTISSLQPEDFATYYCQQSVWVGYSLITFGQGTKVEIKGSTSGSGKPGSGEGSTKGEVQLVESGGGLVQPGGSLRLSCAASGFNIYYSYMHWVRQAPGKGLEWVASISPYYGYTSYADSVKGRFTISADTSKNTAYLQMNSLRAEDTAVYYCARHGYALDYWGQGTLVTVSSEQKLISEEDLNPGAEAAAKEAAAKEAAAKAGSMDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSTSGGGGSLESGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 59 - DNA, 1914 bp, Artificial Sequence
  Description of Artificial Sequence Synthetic polynucleotide
atggccctgccagtgaccgccctgctgctgccactggccctgctgctgcacgccgccagacccgaggtgcagctggtggagtctggaggaggcctggtgcagcctggcggctccctgaggctgtcttgcgcagcaagcggcttcaacatctactatagctacatgcactgggtgcgccaggcccctggcaagggcctggagtgggtggcctccatctctccatactatggctacacctcctatgccgactctgtgaagggccggtttacaatcagcgccgatacctccaagaacacagcctatctgcagatgaattccctgagggcagaggacaccgccgtgtactattgcgccagacacggctacgccctggattattggggccagggcaccctggtgacagtgagctccggcagcacatccggatctggcaagccaggctctggagagggaagcaccaagggcgacatccagatgacacagtccccatctagcctgagcgcctccgtgggcgatagggtgaccatcacatgtcgcgcctctcagagcgtgtcctctgccgtggcatggtaccagcagaagcccggcaaggcccctaagctgctgatctacagcgccagctccctgtattccggcgtgccttctcggttctccggctctagaagcggcaccgactttaccctgacaatctctagcctgcagcccgaggatttcgccacatactattgtcagcagagcgtgtgggtgggctactccctgatcacctttggccagggcacaaaggtggagatcaaggagcagaagctgatcagcgaggaggacctgaatcccgggggaggaggagggagcgggggaggaggcagcggcgggggaggctctggaggaggagggagcggatccatggatatccagatgacccagtccccgagctccctgtccgcctctgtgggcgatagggtcaccatcacctgccgtgccagtcaggacatccgtaattatctgaactggtatcaacagaaaccaggaaaagctccgaaactactgatttactatacctcccgcctggagtctggagtcccttctcgcttctctggttctggttctgggacggattacactctgaccatcagcagtctgcaaccggaagacttcgcaacttattactgtcagcaaggtaatactctgccgtggacgttcggacagggcaccaaggtggagatcaaaggcggcggcggaagtggaggaggaggctcaggcggaggagggagcgaggttcagctggtggagtctggcggtggcctggtgcagccagggggctcactccgtttgtcctgtgcagcttctggctactcctttaccggctacactatgaactgggtgcgtcaggccccaggtaagggcctggaatgggttgcactgattaatccttataaaggtgttagtacctacaaccagaagttcaaggaccgtttcactataagcgtagataaatccaaaaacacagcctacctgcaaatgaacagcctgcgtgctgaggacactgccgtctattattgtgctagaagcggatactacggcgatagtgactggtattttgacgtgtggggtcaaggaaccctggtcaccgtctcctcgactagtggcggaggaggatcactcgagagcggacaggtgctgctggaatccaatatcaaagtcctgcccacttggtctacccccgtgcagcctatggctctgattgtgctgggaggagtcgcaggactgctgctgtttatcgggctgggaattttcttttgcgtgcgctgccggcaccggagaaggcaggccgagcgcatgagccagatcaagcgactgctgagcgagaagaaaacctgtcagtgtccccatagattccagaagacctgttcacccatt

SEQ ID NO: 60 - protein, 638 aa, Artificial Sequence
  Description of Artificial Sequence Synthetic polypeptide
MALPVTALLLPLALLLHAARPEVQLVESGGGLVQPGGSLRLSCAASGFNIYYSYMHWVRQAPGKGLEWVASISPYYGYTSYADSVKGRFTISADTSKNTAYLQMNSLRAEDTAVYYCARHGYALDYWGQGTLVTVSSGSTSGSGKPGSGEGSTKGDIQMTQSPSSLSASVGDRVTITCRASQSVSSAVAWYQQKPGKAPKLLIYSASSLYSGVPSRFSGSRSGTDFTLTISSLQPEDFATYYCQQSVWVGYSLITFGQGTKVEIKEQKLISEEDLNPGGGGGSGGGGSGGGGSGGGGSGSMDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSTSGGGGSLESGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 61 - DNA, 1914 bp, Artificial Sequence
  Description of Artificial Sequence Synthetic polynucleotide
atggccctgccagtgaccgccctgctgctgccactggccctgctgctgcacgccgcccggcctgacatccagatgacacagtccccaagctccctgtccgcctctgtgggcgatagggtgaccatcacatgcagggcaagccagtccgtgtctagcgccgtggcatggtaccagcagaagcccggcaaggcccctaagctgctgatctacagcgcctcctctctgtattccggcgtgccatctcggttctctggcagcagatccggcaccgactttaccctgacaatcagctccctgcagcccgaggatttcgccacatactattgccagcagagcgtgtgggtgggctactccctgatcacctttggccagggcacaaaggtggagatcaagggatctaccagcggatccggcaagcctggcagcggagagggatccacaaagggagaggtgcagctggtggagtctggaggaggcctggtgcagcctggcggctctctgaggctgagctgtgcagcatccggcttcaacatctactatagctacatgcactgggtgcgccaggcccccggcaagggcctggagtgggtggcctctatcagcccttactatggctacacctcttatgccgacagcgtgaagggccggtttacaatctccgccgatacctctaagaacacagcctatctgcagatgaattccctgagggcagaggacaccgccgtgtactattgtgccagacacggctacgccctggattattggggccagggcaccctggtgacagtgtctagcgagcagaagctgatcagcgaggaggacctgaatcccgggggaggaggagggagcgggggaggaggcagcggcgggggaggctctggaggaggagggagcggatccatggatatccagatgacccagtccccgagctccctgtccgcctctgtgggcgatagggtcaccatcacctgccgtgccagtcaggacatccgtaattatctgaactggtatcaacagaaaccaggaaaagctccgaaactactgatttactatacctcccgcctggagtctggagtcccttctcgcttctctggttctggttctgggacggattacactctgaccatcagcagtctgcaaccggaagacttcgcaacttattactgtcagcaaggtaatactctgccgtggacgttcggacagggcaccaaggtggagatcaaaggcggcggcggaagtggaggaggaggctcaggcggaggagggagcgaggttcagctggtggagtctggcggtggcctggtgcagccagggggctcactccgtttgtcctgtgcagcttctggctactcctttaccggctacactatgaactgggtgcgtcaggccccaggtaagggcctggaatgggttgcactgattaatccttataaaggtgttagtacctacaaccagaagttcaaggaccgtttcactataagcgtagataaatccaaaaacacagcctacctgcaaatgaacagcctgcgtgctgaggacactgccgtctattattgtgctagaagcggatactacggcgatagtgactggtattttgacgtgtggggtcaaggaaccctggtcaccgtctcctcgactagtggcggaggaggatcactcgagagcggacaggtgctgctggaatccaatatcaaagtcctgcccacttggtctacccccgtgcagcctatggctctgattgtgctgggaggagtcgcaggactgctgctgtttatcgggctgggaattttcttttgcgtgcgctgccggcaccggagaaggcaggccgagcgcatgagccagatcaagcgactgctgagcgagaagaaaacctgtcagtgtccccatagattccagaagacctgttcacccatt

SEQ ID NO: 62 - protein, 638 aa, Artificial Sequence
  Description of Artificial Sequence Synthetic polypeptide
MALPVTALLLPLALLLHAARPDIQMTQSPSSLSASVGDRVTITCRASQSVSSAVAWYQQKPGKAPKLLIYSASSLYSGVPSRFSGSRSGTDFTLTISSLQPEDFATYYCQQSVWVGYSLITFGQGTKVEIKGSTSGSGKPGSGEGSTKGEVQLVESGGGLVQPGGSLRLSCAASGFNIYYSYMHWVRQAPGKGLEWVASISPYYGYTSYADSVKGRFTISADTSKNTAYLQMNSLRAEDTAVYYCARHGYALDYWGQGTLVTVSSEQKLISEEDLNPGGGGGSGGGGSGGGGSGGGGSGSMDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSTSGGGGSLESGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 63 - DNA, 1917 bp, Artificial Sequence
  Description of Artificial Sequence Synthetic polynucleotide
atggctttgcctgtcacggctcttctgctccctctggccctgcttctgcacgcggcgcgacccgatatccagatgactcagacgacctcatcattgtccgccagtttgggggacagggttacaatatcctgccgggcgagccaagacatcagtaaatatcttaattggtaccagcagaaaccagatggtacagtaaaacttcttatctaccacacctctcggctccactctggggttccctctaggttcagtggtagtgggtcaggcaccgactacagccttacgataagcaacttggaacaggaggatatcgcaacttacttctgccaacagggaaataccctgccttacacgttcggtggaggcactaaactggagatcactgggtcaacctctggtagcggtaagcctggctccggcgaaggctccacaaagggtgaggtgaaactccaagagtcaggtcccggtttggtagccccctcacaaagtttgtcagttacttgtaccgtaagcggcgtttccctgcccgattacggtgtgagctggataaggcagccaccgagaaaaggtcttgaatggctgggagtgatctgggggtctgagacaacgtattacaactcagctcttaagagcaggcttacgatcattaaagataacagcaaatctcaagtgttcctcaaaatgaatagccttcaaactgatgatactgccatctattattgtgctaagcattattactatggcggcagttacgcaatggattattgggggcaaggtacctcagtcactgtaagcagcgaacagaagctcatttctgaagaagacctcaaccccggagggggagggggaagtgggggagggggtagtggtggcggaggatcaggcggggggggatcaggatccatggatatccagatgacccagtccccgagctccctgtccgcctctgtgggcgatagggtcaccatcacctgccgtgccagtcaggacatccgtaattatctgaactggtatcaacagaaaccaggaaaagctccgaaactactgatttactatacctcccgcctggagtctggagtcccttctcgcttctctggttctggttctgggacggattacactctgaccatcagcagtctgcaaccggaagacttcgcaacttattactgtcagcaaggtaatactctgccgtggacgttcggacagggcaccaaggtggagatcaaaggcggcggcggaagtggaggaggaggctcaggcggaggagggagcgaggttcagctggtggagtctggcggtggcctggtgcagccagggggctcactccgtttgtcctgtgcagcttctggctactcctttaccggctacactatgaactgggtgcgtcaggccccaggtaagggcctggaatgggttgcactgattaatcctaccaaaggtgttagtacctacaaccagaagttcaaggaccgtttcactataagcgtagataaatccaaaaacacagcctacctgcaaatgaacagcctgcgtgctgaggacactgccgtctattattgtgctagaagcggatactacggcgatagtgactggtattttgacgtgtggggtcaaggaaccctggtcaccgtctcctcgactagtggcggaggaggatcactcgagagcggacaggtgctgctggaatccaatatcaaagtcctgcccacttggtctacccccgtgcagcctatggctctgattgtgctgggaggagtcgcaggactgctgctgtttatcgggctgggaattttcttttgcgtgcgctgccggcaccggagaaggcaggccgagcgcatgagccagatcaagcgactgctgagcgagaagaaaacctgtcagtgtccccatagattccagaagacctgttcacccatt

SEQ ID NO: 64 - protein, 639 aa, Artificial Sequence
  Description of Artificial Sequence Synthetic polypeptide
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSEQKLISEEDLNPGGGGGSGGGGSGGGGSGGGGSGSMDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSEVQLVESGGGLVQPGGSLRLSCAASGYSFTGYTMNWVRQAPGKGLEWVALINPTKGVSTYNQKFKDRFTISVDKSKNTAYLQMNSLRAEDTAVYYCARSGYYGDSDWYFDVWGQGTLVTVSSTSGGGGSLESGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 65 - DNA, 1566 bp, Artificial Sequence
  Description of Artificial Sequence Synthetic polynucleotide
atggagacccccgcccagctgctgttcctgctgctgctgtggctgcccgacaccaccggcatgtcacggggctccgacctgggcaaaaagctgctggaggccgctagggccgggcaggacgatgaagtgagaatcctgatggccaacggggctgacgtgaatgctaaggatgagtacggcctgacccccctgtatctggctacagcacacggccatctggagatcgtggaagtcctgctgaaaaacggagccgacgtgaatgcagtcgatgccattgggttcactcctctgcacctggcagcctttatcggacatctggagattgcagaagtgctgctgaagcacggcgctgacgtgaacgcacaggataagttcggaaaaaccgcttttgacatcagcattggcaacggaaatgaagacctggctgaaatcctgcagaaactgaatgaacagaaactgattagcgaagaagacctgaaccccgggggaggaggagggagcgggggaggaggcagcggcgggggaggctctggaggaggagggagcggatccatggatatccagatgacccagtccccgagctccctgtccgcctctgtgggcgatagggtcaccatcacctgccgtgccagtcaggacatccgtaattatctgaactggtatcaacagaaaccaggaaaagctccgaaactactgatttactatacctcccgcctggagtctggagtcccttctcgcttctctggttctggttctgggacggattacactctgaccatcagcagtctgcaaccggaagacttcgcaacttattactgtcagcaaggtaatactctgccgtggacgttcggacagggcaccaaggtggagatcaaaggcggcggcggaagtggaggaggaggctcaggcggaggagggagcgaggttcagctggtggagtctggcggtggcctggtgcagccagggggctcactccgtttgtcctgtgcagcttctggctactcctttaccggctacactatgaactgggtgcgtcaggccccaggtaagggcctggaatgggttgcactgattaatccttataaaggtgttagtacctacaaccagaagttcaaggaccgtttcactataagcgtagataaatccaaaaacacagcctacctgcaaatgaacagcctgcgtgctgaggacactgccgtctattattgtgctagaagcggatactacggcgatagtgactggtattttgacgtgtggggtcaaggaaccctggtcaccgtctcctcgactagtggcggaggaggatcactcgagagcggacaggtgctgctggaatccaatatcaaagtcctgcccacttggtctacccccgtgcagcctatggctctgattgtgctgggaggagtcgcaggactgctgctgtttatcgggctgggaattttcttttgcgtgcgctgccggcaccggagaaggcaggccgagcgcatgagccagatcaagcgactgctgagcgagaagaaaacctgtcagtgtccccatagattccagaagacctgttcacccatt

SEQ ID NO: 66 - protein, 522 aa, Artificial Sequence
  Description of Artificial Sequence Synthetic polypeptide
METPAQLLFLLLLWLPDTTGMSRGSDLGKKLLEAARAGQDDEVRILMANGADVNAKDEYGLTPLYLATAHGHLEIVEVLLKNGADVNAVDAIGFTPLHLAAFIGHLEIAEVLLKHGADVNAQDKFGKTAFDISIGNGNEDLAEILQKLNEQKLISEEDLNPGGGGGSGGGGSGGGGSGGGGSGSMDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSTSGGGGSLESGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 67 - DNA, 1569 bp, Artificial Sequence
  Description of Artificial Sequence Synthetic polynucleotide
atggccttaccagtgaccgccttgctcctgccgctggccttgctgctccacgccgccaggccgatgtcacggggctccgacctgggcaaaaagctgctggaggccgctagggccgggcaggacgatgaagtgagaatcctgatggccaacggggctgacgtgaatgctaaggatgagtacggcctgacccccctgtatctggctacagcacacggccatctggagatcgtggaagtcctgctgaaaaacggagccgacgtgaatgcagtcgatgccattgggttcactcctctgcacctggcagcctttatcggacatctggagattgcagaagtgctgctgaagcacggcgctgacgtgaacgcacaggataagttcggaaaaaccgcttttgacatcagcattggcaacggaaatgaagacctggctgaaatcctgcagaaactgaatgaacagaaactgattagcgaagaagacctgaaccccgggggaggaggagggagcgggggaggaggcagcggcgggggaggctctggaggaggagggagcggatccatggatatccagatgacccagtccccgagctccctgtccgcctctgtgggcgatagggtcaccatcacctgccgtgccagtcaggacatccgtaattatctgaactggtatcaacagaaaccaggaaaagctccgaaactactgatttactatacctcccgcctggagtctggagtcccttctcgcttctctggttctggttctgggacggattacactctgaccatcagcagtctgcaaccggaagacttcgcaacttattactgtcagcaaggtaatactctgccgtggacgttcggacagggcaccaaggtggagatcaaaggcggcggcggaagtggaggaggaggctcaggcggaggagggagcgaggttcagctggtggagtctggcggtggcctggtgcagccagggggctcactccgtttgtcctgtgcagcttctggctactcctttaccggctacactatgaactgggtgcgtcaggccccaggtaagggcctggaatgggttgcactgattaatccttataaaggtgttagtacctacaaccagaagttcaaggaccgtttcactataagcgtagataaatccaaaaacacagcctacctgcaaatgaacagcctgcgtgctgaggacactgccgtctattattgtgctagaagcggatactacggcgatagtgactggtattttgacgtgtggggtcaaggaaccctggtcaccgtctcctcgactagtggcggaggaggatcactcgagagcggacaggtgctgctggaatccaatatcaaagtcctgcccacttggtctacccccgtgcagcctatggctctgattgtgctgggaggagtcgcaggactgctgctgtttatcgggctgggaattttcttttgcgtgcgctgccggcaccggagaaggcaggccgagcgcatgagccagatcaagcgactgctgagcgagaagaaaacctgtcagtgtccccatagattccagaagacctgttcacccatt

SEQ ID NO: 68 - protein, 523 aa, Artificial Sequence
  Description of Artificial Sequence Synthetic polypeptide
MALPVTALLLPLALLLHAARPMSRGSDLGKKLLEAARAGQDDEVRILMANGADVNAKDEYGLTPLYLATAHGHLEIVEVLLKNGADVNAVDAIGFTPLHLAAFIGHLEIAEVLLKHGADVNAQDKFGKTAFDISIGNGNEDLAEILQKLNEQKLISEEDLNPGGGGGSGGGGSGGGGSGGGGSGSMDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSTSGGGGSLESGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 69 - protein, 20 aa, Artificial Sequence
  Description of Artificial Sequence Synthetic peptide
GGGGSGGGGSGGGGSGGGGS

SEQ ID NO: 70 - DNA, 60 bp, Artificial Sequence
  Description of Artificial Sequence Synthetic oligonucleotide
ggaggaggagggagcgggggaggaggcagcggcgggggaggctctggaggaggagggagc

SEQ ID NO: 71 - DNA, 750 bp, Artificial Sequence
  Description of Artificial Sequence Synthetic polynucleotide
atggacatccagatgactcagaccacaagctccctgtctgcaagtctgggcgaccgggtgacaatctcctgcagagcctctcaggatattaggaactacctgaattggtatcagcagaaacctgatggcacagtcaagctgctgatctactataccagccggctgcactcaggcgtgccaagcaaattctcaggaagcggctccgggactgactactccctgaccatctctaacctggagcaggaagatattgctacctatttctgccagcagggcaatacactgccctggacttttgccggaggcaccaaactggagatcaaggggggaggcgggagtggaggcgggggatcaggaggaggaggcagcggaggaggagggtccgaggtccagctgcagcagagcggaccagaactggtgaagcccggagcaagtatgaaaatctcctgtaaggcctcaggatacagcttcaccggctatacaatgaactgggtgaaacagtcccatggcaagaacctggaatggatggggctgattaatcctaccaaaggcgtcagcacctataatcagaagtttaaagacaaggccacactgactgtggataagtctagttcaaccgcttacatggagctgctgtccctgacatctgaagacagtgccgtgtactattgtgctcggtctggctactatggggacagtgattggtacttcgatgtctggggacagggcactaccctgaccgtgttttct

SEQ ID NO: 72 - protein, 250 aa, Artificial Sequence
  Description of Artificial Sequence Synthetic polypeptide
MDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSGGGGSGGGGSGGGGSEVQLQQSGPELVKPGASMKISCKASGYSFTGYTMNWVKQSHGKNLEWMGLINPTKGVSTYNQKFKDKATLTVDKSSSTAYMELLSLTSEDSAVYYCARSGYYGDSDWYFDVWGQGTTLTVFS

SEQ ID NO: 73 - protein, 5 aa, Artificial Sequence
  Description of Artificial Sequence Synthetic peptide
GGGGS

SEQ ID NO: 74 - protein, 15 aa, Artificial Sequence
  Description of Artificial Sequence Synthetic peptide
GGGGSGGGGSGGGGS

SEQ ID NO: 75 - DNA, 1560 bp, Artificial Sequence
  Description of Artificial Sequence Synthetic polynucleotide
atggatttccaggtccagattttctccttcctgctgatttccgcaagcgtcattatgtcacggggctccgacctgggcaaaaagctgctggaggccgctagggccgggcaggacgatgaagtgagaatcctgatggccaacggggctgacgtgaatgctaaggatgagtacggcctgacccccctgtatctggctacagcacacggccatctggagatcgtggaagtcctgctgaaaaacggagccgacgtgaatgcagtcgatgccattgggttcactcctctgcacctggcagcctttatcggacatctggagattgcagaagtgctgctgaagcacggcgctgacgtgaacgcacaggataagttcggaaaaaccgcttttgacatcagcattggcaacggaaatgaagacctggctgaaatcctgcagaaactgaatgaacagaaactgattagcgaagaagacctgaaccccgggggaggaggagggagcgggggaggaggcagcggcgggggaggctctggaggaggagggagcggatccatggatatccagatgacccagtccccgagctccctgtccgcctctgtgggcgatagggtcaccatcacctgccgtgccagtcaggacatccgtaattatctgaactggtatcaacagaaaccaggaaaagctccgaaactactgatttactatacctcccgcctggagtctggagtcccttctcgcttctctggttctggttctgggacggattacactctgaccatcagcagtctgcaaccggaagacttcgcaacttattactgtcagcaaggtaatactctgccgtggacgttcggacagggcaccaaggtggagatcaaaggcggcggcggaagtggaggaggaggctcaggcggaggagggagcgaggttcagctggtggagtctggcggtggcctggtgcagccagggggctcactccgtttgtcctgtgcagcttctggctactcctttaccggctacactatgaactgggtgcgtcaggccccaggtaagggcctggaatgggttgcactgattaatccttataaaggtgttagtacctacaaccagaagttcaaggaccgtttcactataagcgtagataaatccaaaaacacagcctacctgcaaatgaacagcctgcgtgctgaggacactgccgtctattattgtgctagaagcggatactacggcgatagtgactggtattttgacgtgtggggtcaaggaaccctggtcaccgtctcctcgactagtggcggaggaggatcactcgagagcggacaggtgctgctggaatccaatatcaaagtcctgcccacttggtctacccccgtgcagcctatggctctgattgtgctgggaggagtcgcaggactgctgctgtttatcgggctgggaattttcttttgcgtgcgctgccggcaccggagaaggcaggccgagcgcatgagccagatcaagcgactgctgagcgagaagaaaacctgtcagtgtccccatagattccagaagacctgttcacccatt

SEQ ID NO: 76 - protein, 520 aa, Artificial Sequence
  Description of Artificial Sequence Synthetic polypeptide
MDFQVQIFSFLLISASVIMSRGSDLGKKLLEAARAGQDDEVRILMANGADVNAKDEYGLTPLYLATAHGHLEIVEVLLKNGADVNAVDAIGFTPLHLAAFIGHLEIAEVLLKHGADVNAQDKFGKTAFDISIGNGNEDLAEILQKLNEQKLISEEDLNPGGGGGSGGGGSGGGGSGGGGSGSMDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSTSGGGGSLESGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 77 - DNA, 45 bp, Artificial Sequence
  Description of Artificial Sequence Synthetic oligonucleotide
ggcggcggcggaagtggaggaggaggctcaggcggaggagggagc

(19) United States
(12) Patent Application Publication
(10) Pub. No.: US 2024/0016841 A1
(43) Pub. Date: Jan. 18, 2024

(54) CELLS COMPRISING T CELL-ANTIGEN COUPLERS AND USES THEREOF

(71) Applicants: Triumvira Immunologics USA, Inc., Austin, TX (US); McMaster University, Hamilton (CA)

(72) Inventors: Jonathan Lorne Bramson, Oakville (CA); SeungMi Yoo, Hamilton (CA); Sarah Asbury, Hamilton (CA); Allyson Moore, Hamilton (CA); Ling Wang, Mississauga (CA); Brian Philip, Port Elgin (CA)

(21) Appl. No.: 18/251,992

(22) PCT Filed: Nov. 5, 2021
(86) PCT No.: PCT/US2021/058339

§ 371 Date: May 5, 2023

Related U.S. Application Data
(60) Provisional application No. 63/110,902, filed on Nov. 6, 2020.

(51) Int. Cl. A61K 35/17; A61K 31/675; C07K 16/28; C07K 16/32; A61K 39/00; A61K 38/20; A61P 35/00

(52) U.S. Cl. CPC A61K 35/17; A61K 31/675; C07K 16/2803; C07K 16/32; C07K 16/2878; A61K 39/4611; A61K 39/464406; A61K 39/464412; A61K 38/2013; A61P 35/00; A61K 2239/13; A61K 2239/21

(57) ABSTRACT

γδ T cells comprising a molecule comprising (i) a target-specific antigen-binding domain, (ii) an antigen-binding domain that binds a protein associated with a TCR complex, and (iii) a T cell receptor signaling domain polypeptide are provided.

### CROSS-REFERENCE TO RELATED APPLICATIONS

This application claims the benefit of and priority to U.S. Provisional Patent Application No. 63/110,902, filed Nov. 6, 2020, which is incorporated herein by reference in its entirety for all purposes.

### SEQUENCE LISTING

The instant application contains a Sequence Listing which has been submitted electronically in ASCII format and is hereby incorporated by reference in its entirety. Said ASCII copy, created on Nov. 3, 2021, is named TMV-005WO_SL.txt and is 130,056 bytes in size.

### SUMMARY

Disclosed herein, in certain embodiments, are methods of treating a cancer in an individual in need thereof. In some embodiments, the method comprises administering to the individual a therapeutically effective amount of: (i) a γδ T cell comprising a T cell-antigen coupler (TAC) polypeptide; and (ii) zoledronate. In some embodiments, the zoledronate is administered before, after, or simultaneously with the γδ T cell. In some embodiments, the TAC polypeptide comprises: (a) a target-specific antigen-binding domain; (b) an antigen-binding domain that binds a protein associated with a TCR complex; and (c) a TCR co-receptor cytosolic domain and transmembrane domain. In some embodiments, the TAC polypeptide comprises, in order (e.g., from N- to C-terminus): (a) a target-specific antigen-binding domain; (b) an antigen-binding domain that binds a protein associated with a TCR complex; and (c) a TCR co-receptor cytosolic domain and transmembrane domain. In some embodiments, the TAC polypeptide is encoded by a nucleic acid comprising, in order (e.g., from 5′ to 3′): (a) a first polynucleotide encoding a target-specific antigen-binding domain; (b) a second polynucleotide encoding an antigen-binding domain that binds a protein associated with a TCR complex; and (c) a third polynucleotide encoding a TCR co-receptor cytosolic domain and transmembrane domain. In some embodiments, the target specific antigen-binding domain is an antigen-binding domain that binds (e.g., selectively binds) a CD19 antigen, a HER2 antigen, or a BCMA antigen. In some embodiments, the target specific antigen-binding domain is a designed ankyrin repeat (DARPin) polypeptide, single chain variable fragment (scFv), single domain antibody, diabody, affibody, adnectin, affilin, phylomer, fynomer, affimer, peptide aptamer, knottin, centyrin, anticalin, or nanobody. In some embodiments, the target specific antigen-binding domain is a designed ankyrin repeat (DARPin) polypeptide or a single chain variable fragment (scFv). In some embodiments, the protein associated with the TCR complex is a CD3 protein, for example, a CD3 protein of a TCR complex on the γδ T cell. In some embodiments, the CD3 protein is a CD3γ protein, CD3δ protein and/or CD3ε protein. In some embodiments, the CD3 protein is a CD3ε protein. In some embodiments, binding of the CD3 protein induces activation of the γδ T cell. In some embodiments, the antigen-binding domain that binds the protein associated with the TCR complex is derived from an antibody selected from UCHT1, OKT3, F6A, and L2K. In some embodiments, the antigen-binding domain that binds the protein associated with the TCR complex is a UCHT1 antigen-binding domain, for example, an scFv derived from UCHT1. In some embodiments, the UCHT1 antigen-binding domain comprises a Y to T mutation at a position corresponding to amino acid 182 of SEQ ID NO: 14 (Y182T). In some embodiments, the UCHT1 antigen-binding domain is a humanized variant of UCHT1 (huUCHT1), for example, a humanized variant of UCHT1 comprising a Y to T mutation at a position corresponding to amino acid 177 of SEQ ID NO: 44 (huUCHT1 (Y177T)). In some embodiments, the UCHT1 antigen-binding domain comprises an amino acid sequence having at least 80%, at least 85%, at least 90%, at least 95%, at least 96%, at least 97%, at least 98%, at least 99%, or 100% sequence identity with SEQ ID NO: 14 (UCHT1), SEQ ID NO: 72 (UCHT1 (Y182T)), SEQ ID NO: 44 (huUCHT1), or SEQ ID NO: 46 (huUCHT1 (Y177T)). In some embodiments, the antigen-binding domain that binds the protein associated with the TCR complex is OKT3, for example, the antigen-binding domain that binds a protein associated with the TCR complex comprises an amino acid sequence having at least 80%, at least 85%, at least 90%, at least 95%, at least 96%, at least 97%, at least 98%, at least 99%, or 100% sequence identity with SEQ ID NO: 22 (OKT3). In some embodiments, the antigen-binding domain that binds the protein associated with the TCR complex is F6A, for example, the antigen-binding domain that binds the protein associated with the TCR complex comprises an amino acid sequence having at least 80%, at least 85%, at least 90%, at least 95%, at least 96%, at least 97%, at least 98%, at least 99%, or 100% sequence identity with SEQ ID NO: 24 (F6A). In some embodiments, the antigen-binding domain that binds the protein associated with the TCR complex is L2K, for example, the antigen-binding domain that binds the protein associated with the TCR complex comprises an amino acid sequence having at least 80%, at least 85%, at least 90%, at least 95%, at least 96%, at least 97%, at least 98%, at least 99%, or 100% sequence identity with SEQ ID NO: 26 (L2K). In some embodiments, the cytosolic domain is a CD4 cytosolic domain and the transmembrane domain is a CD4 transmembrane domain, or wherein the cytosolic domain is a CD8 cytosolic domain and the transmembrane domain is a CD8 transmembrane domain. In some embodiments, the TCR co-receptor cytosolic domain and transmembrane domain comprises an amino acid sequence having at least 80%, at least 85%, at least 90%, at least 95%, at least 96%, at least 97%, at least 98%, at least 99%, or 100% sequence identity with SEQ ID NO: 18 (CD4 transmembrane and cytosolic domain). In some embodiments, components (a), (b), and/or (c) are connected in any suitable manner, such as in any suitable order and/or comprising any suitable linker(s). In some embodiments, (a), (b), and (c) are fused directly to each other, or joined by at least one linker. In some embodiments, (a) and (c) are fused to (b). In some embodiments, (b) and (c) are fused to (a). In some embodiments, at least one linker joins (a) to (b). In some embodiments, the at least one linker is a G₄S flexible linker, a large protein domain, a long helix structure, or a short helix structure. In some embodiments, the at least one linker comprises an amino acid sequence having at least 80%, at least 85%, at least 90%, at least 95%, at least 96%, at least 97%, at least 98%, at least 99%, or 100% sequence identity with SEQ ID NO: 12 ((G4S)4-based linker), SEQ ID NO: 16 (G4S-based linker), SEQ ID NO: 20 (CD4 based linker), SEQ ID NO: 28 (short helix connector), SEQ ID NO: 30 (long helix connector), SEQ ID NO: 32 (large domain connector), SEQ ID NO: 69 (flexible connector), SEQ ID NO: 73 (G4S flexible linker), or SEQ ID NO: 74 (G4S3 flexible linker). In some embodiments, the TAC polypeptide does not comprise a co-stimulatory domain and/or an activation domain. In some embodiments, the TAC polypeptide further comprises a leader sequence, for example, a leader sequence comprising an amino acid sequence having at least 80%, at least 85%, at least 90%, at least 95%, at least 96%, at least 97%, at least 98%, at least 99%, or 100% sequence identity with SEQ ID NO: 6 (muIgG leader), SEQ ID NO: 48 (huIgG leader), or SEQ ID NO: 50 (huCD8a leader). In some embodiments, the TAC polypeptide comprises an antigen-binding domain that binds (e.g., selectively binds) a CD19 antigen, for example, an antigen-binding domain comprising an amino acid sequence having at least 80%, at least 85%, at least 90%, at least 95%, at least 96%, at least 97%, at least 98%, at least 99%, or 100% sequence identity with SEQ ID NO: 36 (CD19 scFv). In some embodiments, the TAC polypeptide comprises an amino acid sequence having at least 80%, at least 85%, at least 90%, at least 95%, at least 96%, at least 97%, at least 98%, at least 99%, or 100% sequence identity with SEQ ID NO: 64 (CD19 TAC). In some embodiments, the TAC polypeptide is encoded by a nucleic acid comprising a nucleotide sequence having at least 80%, at least 85%, at least 90%, at least 95%, at least 96%, at least 97%, at least 98%, at least 99%, or 100% sequence identity with SEQ ID NO: 63 (CD19 TAC). In some embodiments, the TAC polypeptide comprises an antigen-binding domain that binds (e.g., selectively binds) a HER2 antigen. In some embodiments, the antigen-binding domain that binds a HER2 antigen comprises an antigen binding domain of an antibody selected from Trastuzumab, Pertuzumab, Lapatinib, Neratinib, Ado-trastuzumab Emtansine, Gancotamab, Margetuximab, Timigutuzumab, and Ertumaxomab. In some embodiments the antigen-binding domain that binds a HER2 antigen comprises an amino acid sequence having at least 80%, at least 85%, at least 90%, at least 95%, at least 96%, at least 97%, at least 98%, at least 99%, or 100% sequence identity with SEQ ID NO: 8 (HER2 DARPin). In some embodiments, the TAC polypeptide comprises an amino acid sequence having at least 80%, at least 85%, at least 90%, at least 95%, at least 96%, at least 97%, at least 98%, at least 99%, or 100% sequence identity with SEQ ID NO: 66 (HER2 TAC: huIgG leader sequence; huUCHT1 CD3-binding domain), SEQ ID NO: 68 (HER2 TAC: CD8a leader; huUCHT1 CD3-binding domain), or SEQ ID NO: 76 (HER2 TAC: muIgG leader; huUCHT1 CD3-binding domain). In some embodiments, the TAC polypeptide is encoded by a nucleic acid comprising a nucleotide sequence having at least 80%, at least 85%, at least 90%, at least 95%, at least 96%, at least 97%, at least 98%, at least 99%, or 100% sequence identity with SEQ ID NO: 65 (HER2 TAC: huIgG leader; huUCHT1 CD3-binding domain), SEQ ID NO: 67 (HER2 TAC: CD8a leader; huUCHT1 CD3-binding domain), or SEQ ID NO: 75 (HER2 TAC: muIgG leader; huUCHT1 CD3-binding domain). In some embodiments, the TAC polypeptide comprises an antigen-binding domain that binds (e.g., selectively binds) a BCMA antigen. In some embodiments, the antigen-binding domain that binds a BCMA antigen comprises an antigen binding domain of an antibody selected from Belantamab mafodotin and GSK2857916. In some embodiments, the antigen-binding domain that binds a BCMA antigen comprises an amino acid sequence having at least 80%, at least 85%, at least 90%, at least 95%, at least 96%, at least 97%, at least 98%, at least 99%, or 100% sequence identity with SEQ ID NO: 34 (BCMA scFv), SEQ ID NO: 52 (3625 BCMA scFv, Vh-Vl), or SEQ ID NO: 54 (3625 BCMA scFv, Vl-Vh). In some embodiments, the TAC polypeptide comprises an amino acid sequence having at least 80%, at least 85%, at least 90%, at least 95%, at least 96%, at least 97%, at least 98%, at least 99%, or 100% sequence identity with SEQ ID NO: 56 (BCMA TAC: 3625 BCMA scFv, Vh-Vl, helix linker; huUCHT1 CD3-binding domain), SEQ ID NO: 58 (BCMA TAC: 3625 BCMA scFv, Vl-Vh, helix linker; huUCHT1 CD3-binding domain), SEQ ID NO: 60 (BCMA TAC: 3625 BCMA scFv, Vh-Vl, G4S linker; huUCHT1 CD3-binding domain), or SEQ ID NO: 62 (BCMA TAC: 3625 BCMA scFv, Vl-Vh, G4S linker; huUCHT1 CD3-binding domain). In some embodiments, the TAC polypeptide is encoded by a nucleic acid comprising a nucleotide sequence having at least 80%, at least 85%, at least 90%, at least 95%, at least 96%, at least 97%, at least 98%, at least 99%, or 100% sequence identity with SEQ ID NO: 55 (BCMA TAC: 3625 BCMA scFv, Vh-Vl, helix linker; huUCHT1 CD3-binding domain), SEQ ID NO: 57 (BCMA TAC: 3625 BCMA scFv, Vl-Vh, helix linker; huUCHT1 CD3-binding domain), SEQ ID NO: 59 (BCMA TAC: 3625 BCMA scFv, Vh-Vl, G4S linker; huUCHT1 CD3-binding domain), or SEQ ID NO: 61 (BCMA TAC: 3625 BCMA scFv, Vl-Vh, G4S linker; huUCHT1 CD3-binding domain).

Disclosed herein, in certain embodiments, are methods of making γδ T cells, for example, γδ T cells comprising or expressing a TAC polypeptide. In some embodiments, the method comprises one or more of the following steps: (a) contacting γδ T cells isolated from an individual with zoledronate, a cytokine (e.g., IL-2 and/or IL-15), and/or a CD16 agonist, (b) contacting the γδ T cells with an expression vector comprising a nucleic acid encoding the TAC polypeptide, (c) culturing and/or expanding the cells (for example, for 10-14 days), and (d) removing αβ T cells from the culture. In some embodiments, the γδ T cells are cultured and/or expanded for 10, 11, 12, 13, or 14 days, or for at least 10, 11, 12, 13, or 14 days. In some embodiments, αβ T cells are removed by negative selection of cells including CD4 and/or CD8. In some embodiments, the method results in a culture or composition that is substantially free of αβ T cells (e.g., at least 90%, 95%, 96%, 97%, 98%, or 99%, or 100% of the T cells present in the resulting culture or composition are γδ T cells). In some embodiments, the method results in a culture or composition that is substantially free of cells other than γδ T cells (e.g., at least 90%, 95%, 96%, 97%, 98%, or 99%, or 100% of the cells present in the resulting culture or composition are γδ T cells). In some embodiments, the expression vector is a lentiviral vector, for example, a VSV-G pseudotyped lentiviral vector. In some embodiments, the expression vector is a γ retroviral vector, for example, a GALV pseudotyped γ-retroviral vector. In some embodiments, the TAC polypeptide comprises: (a) a target-specific antigen-binding domain; (b) an antigen-binding domain that binds a protein associated with a TCR complex; and (c) a TCR co-receptor cytosolic domain and transmembrane domain. In some embodiments, the TAC polypeptide comprises, in order (e.g., from N- to C-terminus): (a) a target-specific antigen-binding domain; (b) an antigen-binding domain that binds a protein associated with a TCR complex; and (c) a TCR co-receptor cytosolic domain and transmembrane domain. In some embodiments, the TAC polypeptide is encoded by a nucleic acid comprising, in order (e.g., from 5′ to 3′): (a) a first polynucleotide encoding a target-specific antigen-binding domain; (b) a second polynucleotide encoding an antigen-binding domain that binds a protein associated with a TCR complex; and (c) a third polynucleotide encoding a TCR co-receptor cytosolic domain and transmembrane domain. In some embodiments, the target specific antigen-binding domain is an antigen-binding domain that binds (e.g., selectively binds) a CD19 antigen, a HER2 antigen, or a BCMA antigen. In some embodiments, the target specific antigen-binding domain is a designed ankyrin repeat (DARPin) polypeptide, single chain variable fragment (scFv), single domain antibody, diabody, affibody, adnectin, affilin, phylomer, fynomer, affimer, peptide aptamer, knottin, centyrin, anticalin, or nanobody. In some embodiments, the target specific antigen-binding domain is a designed ankyrin repeat (DARPin) polypeptide or a single chain variable fragment (scFv). In some embodiments, the protein associated with the TCR complex is a CD3 protein, for example, a CD3 protein of a TCR complex on the γδ T cell. In some embodiments, the CD3 protein is a CD3γ protein, CD3δ protein and/or CD3ε protein. In some embodiments, the CD3 protein is a CD3ε protein. In some embodiments, binding of the CD3 protein induces activation of the γδ T cell. In some embodiments, the antigen-binding domain that binds the protein associated with the TCR complex is derived from an antibody selected from UCHT1, OKT3, F6A, and L2K. In some embodiments, the antigen-binding domain that binds the protein associated with the TCR complex is a UCHT1 antigen-binding domain, for example, an scFv derived from UCHT1. In some embodiments, the UCHT1 antigen-binding domain comprises a Y to T mutation at a position corresponding to amino acid 182 of SEQ ID NO: 14 (Y182T). In some embodiments, the UCHT1 antigen-binding domain is a humanized variant of UCHT1 (huUCHT1), for example, a humanized variant of UCHT1 comprising a Y to T mutation at a position corresponding to amino acid 177 of SEQ ID NO: 44 (huUCHT1 (Y177T)). In some embodiments, the UCHT1 antigen-binding domain comprises an amino acid sequence having at least 80%, at least 85%, at least 90%, at least 95%, at least 96%, at least 97%, at least 98%, at least 99%, or 100% sequence identity with SEQ ID NO: 14 (UCHT1), SEQ ID NO: 72 (UCHT1 (Y182T)), SEQ ID NO: 44 (huUCHT1), or SEQ ID NO: 46 (huUCHT1 (Y177T)). In some embodiments, the antigen-binding domain that binds the protein associated with the TCR complex is OKT3, for example, the antigen-binding domain that binds a protein associated with the TCR complex comprises an amino acid sequence having at least 80%, at least 85%, at least 90%, at least 95%, at least 96%, at least 97%, at least 98%, at least 99%, or 100% sequence identity with SEQ ID NO: 22 (OKT3). In some embodiments, the antigen-binding domain that binds the protein associated with the TCR complex is F6A, for example, the antigen-binding domain that binds the protein associated with the TCR complex comprises an amino acid sequence having at least 80%, at least 85%, at least 90%, at least 95%, at least 96%, at least 97%, at least 98%, at least 99%, or 100% sequence identity with SEQ ID NO: 24 (F6A). In some embodiments, the antigen-binding domain that binds the protein associated with the TCR complex is L2K, for example, the antigen-binding domain that binds the protein associated with the TCR complex comprises an amino acid sequence having at least 80%, at least 85%, at least 90%, at least 95%, at least 96%, at least 97%, at least 98%, at least 99%, or 100% sequence identity with SEQ ID NO: 26 (L2K). In some embodiments, the cytosolic domain is a CD4 cytosolic domain and the transmembrane domain is a CD4 transmembrane domain, or wherein the cytosolic domain is a CD8 cytosolic domain and the transmembrane domain is a CD8 transmembrane domain. In some embodiments, the TCR co-receptor cytosolic domain and transmembrane domain comprises an amino acid sequence having at least 80%, at least 85%, at least 90%, at least 95%, at least 96%, at least 97%, at least 98%, at least 99%, or 100% sequence identity with SEQ ID NO: 18 (CD4 transmembrane and cytosolic domain). In some embodiments, components (a), (b), and/or (c) are connected in any suitable manner, such as in any suitable order and/or comprising any suitable linker(s). In some embodiments, (a), (b), and (c) are fused directly to each other, or joined by at least one linker. In some embodiments, (a) and (c) are fused to (b). In some embodiments, (b) and (c) are fused to (a). In some embodiments, at least one linker joins (a) to (b). In some embodiments, the at least one linker is a G₄S flexible linker, a large protein domain, a long helix structure, or a short helix structure. In some embodiments, the at least one linker comprises an amino acid sequence having at least 80%, at least 85%, at least 90%, at least 95%, at least 96%, at least 97%, at least 98%, at least 99%, or 100% sequence identity with SEQ ID NO: 12 ((G4S)4-based linker), SEQ ID NO: 16 (G4S-based linker), SEQ ID NO: 20 (CD4 based linker), SEQ ID NO: 28 (short helix connector), SEQ ID NO: 30 (long helix connector), SEQ ID NO: 32 (large domain connector), SEQ ID NO: 69 (flexible connector), SEQ ID NO: 73 (G4S flexible linker), or SEQ ID NO: 74 (G4S3 flexible linker). In some embodiments, the TAC polypeptide does not comprise a co-stimulatory domain and/or an activation domain. In some embodiments, the TAC polypeptide further comprises a leader sequence, for example, a leader sequence comprising an amino acid sequence having at least 80%, at least 85%, at least 90%, at least 95%, at least 96%, at least 97%, at least 98%, at least 99%, or 100% sequence identity with SEQ ID NO: 6 (muIgG leader), SEQ ID NO: 48 (huIgG leader), or SEQ ID NO: 50 (huCD8a leader). In some embodiments, the TAC polypeptide comprises an antigen-binding domain that binds (e.g., selectively binds) a CD19 antigen, for example, an antigen-binding domain comprising an amino acid sequence having at least 80%, at least 85%, at least 90%, at least 95%, at least 96%, at least 97%, at least 98%, at least 99%, or 100% sequence identity with SEQ ID NO: 36 (CD19 scFv). In some embodiments, the TAC polypeptide comprises an amino acid sequence having at least 80%, at least 85%, at least 90%, at least 95%, at least 96%, at least 97%, at least 98%, at least 99%, or 100% sequence identity with SEQ ID NO: 64 (CD19 TAC). In some embodiments, the TAC polypeptide is encoded by a nucleic acid comprising a nucleotide sequence having at least 80%, at least 85%, at least 90%, at least 95%, at least 96%, at least 97%, at least 98%, at least 99%, or 100% sequence identity with SEQ ID NO: 63 (CD19 TAC). In some embodiments, the TAC polypeptide comprises an antigen-binding domain that binds (e.g., selectively binds) a HER2 antigen. In some embodiments, the antigen-binding domain that binds a HER2 antigen comprises an antigen binding domain of an antibody selected from Trastuzumab, Pertuzumab, Lapatinib, Neratinib, Ado-trastuzumab Emtansine, Gancotamab, Margetuximab, Timigutuzumab, and Ertumaxomab. In some embodiments the antigen-binding domain that binds a HER2 antigen comprises an amino acid sequence having at least 80%, at least 85%, at least 90%, at least 95%, at least 96%, at least 97%, at least 98%, at least 99%, or 100% sequence identity with SEQ ID NO: 8 (HER2 DARPin). In some embodiments, the TAC polypeptide comprises an amino acid sequence having at least 80%, at least 85%, at least 90%, at least 95%, at least 96%, at least 97%, at least 98%, at least 99%, or 100% sequence identity with SEQ ID NO: 66 (HER2 TAC: huIgG leader sequence; huUCHT1 CD3-binding domain), SEQ ID NO: 68 (HER2 TAC: CD8a leader; huUCHT1 CD3-binding domain), or SEQ ID NO: 76 (HER2 TAC: muIgG leader; huUCHT1 CD3-binding domain). In some embodiments, the TAC polypeptide is encoded by a nucleic acid comprising a nucleotide sequence having at least 80%, at least 85%, at least 90%, at least 95%, at least 96%, at least 97%, at least 98%, at least 99%, or 100% sequence identity with SEQ ID NO: 65 (HER2 TAC: huIgG leader; huUCHT1 CD3-binding domain), SEQ ID NO: 67 (HER2 TAC: CD8a leader; huUCHT1 CD3-binding domain), or SEQ ID NO: 75 (HER2 TAC: muIgG leader; huUCHT1 CD3-binding domain). In some embodiments, the TAC polypeptide comprises an antigen-binding domain that binds (e.g., selectively binds) a BCMA antigen. In some embodiments, the antigen-binding domain that binds a BCMA antigen comprises an antigen binding domain of an antibody selected from Belantamab mafodotin and GSK2857916. In some embodiments, the antigen-binding domain that binds a BCMA antigen comprises an amino acid sequence having at least 80%, at least 85%, at least 90%, at least 95%, at least 96%, at least 97%, at least 98%, at least 99%, or 100% sequence identity with SEQ ID NO: 34 (BCMA scFv), SEQ ID NO: 52 (3625 BCMA scFv, Vh-Vl), or SEQ ID NO: 54 (3625 BCMA scFv, Vl-Vh). In some embodiments, the TAC polypeptide comprises an amino acid sequence having at least 80%, at least 85%, at least 90%, at least 95%, at least 96%, at least 97%, at least 98%, at least 99%, or 100% sequence identity with SEQ ID NO: 56 (BCMA TAC: 3625 BCMA scFv, Vh-Vl, helix linker; huUCHT1 CD3-binding domain), SEQ ID NO: 58 (BCMA TAC: 3625 BCMA scFv, Vl-Vh, helix linker; huUCHT1 CD3-binding domain), SEQ ID NO: 60 (BCMA TAC: 3625 BCMA scFv, Vh-Vl, G4S linker; huUCHT1 CD3-binding domain), or SEQ ID NO: 62 (BCMA TAC: 3625 BCMA scFv, Vl-Vh, G4S linker; huUCHT1 CD3-binding domain). In some embodiments, the TAC polypeptide is encoded by a nucleic acid comprising a nucleotide sequence having at least 80%, at least 85%, at least 90%, at least 95%, at least 96%, at least 97%, at least 98%, at least 99%, or 100% sequence identity with SEQ ID NO: 55 (BCMA TAC: 3625 BCMA scFv, Vh-Vl, helix linker; huUCHT1 CD3-binding domain), SEQ ID NO: 57 (BCMA TAC: 3625 BCMA scFv, Vl-Vh, helix linker; huUCHT1 CD3-binding domain), SEQ ID NO: 59 (BCMA TAC: 3625 BCMA scFv, Vh-Vl, G4S linker; huUCHT1 CD3-binding domain), or SEQ ID NO: 61 (BCMA TAC: 3625 BCMA scFv, Vl-Vh, G4S linker; huUCHT1 CD3-binding domain).

Disclosed herein, in certain embodiments, are γδ T cells made by any of the foregoing methods.

Disclosed herein, in certain embodiments, are γδ T cells comprising a T cell-antigen coupler (TAC) polypeptide. In some embodiments, the TAC polypeptide comprises: (a) a target-specific antigen-binding domain; (b) an antigen-binding domain that binds a protein associated with a TCR complex; and (c) a TCR co-receptor cytosolic domain and transmembrane domain. In some embodiments, the TAC polypeptide comprises, in order (e.g., from N- to C-terminus): (a) a target-specific antigen-binding domain; (b) an antigen-binding domain that binds a protein associated with a TCR complex; and (c) a TCR co-receptor cytosolic domain and transmembrane domain. In some embodiments, the TAC polypeptide is encoded by a nucleic acid comprising, in order (e.g., from 5′ to 3′): (a) a first polynucleotide encoding a target-specific antigen-binding domain; (b) a second polynucleotide encoding an antigen-binding domain that binds a protein associated with a TCR complex; and (c) a third polynucleotide encoding a TCR co-receptor cytosolic domain and transmembrane domain. In some embodiments, the target specific antigen-binding domain is an antigen-binding domain that binds (e.g., selectively binds) a CD19 antigen, a HER2 antigen, or a BCMA antigen. In some embodiments, the target specific antigen-binding domain is a designed ankyrin repeat (DARPin) polypeptide, single chain variable fragment (scFv), single domain antibody, diabody, affibody, adnectin, affilin, phylomer, fynomer, affimer, peptide aptamer, knottin, centyrin, anticalin, or nanobody. In some embodiments, the target specific antigen-binding domain is a designed ankyrin repeat (DARPin) polypeptide or a single chain variable fragment (scFv). In some embodiments, the protein associated with the TCR complex is a CD3 protein, for example, a CD3 protein of a TCR complex on the γδ T cell. In some embodiments, the CD3 protein is a CD3γ protein, CD3δ protein and/or CD3ε protein. In some embodiments, the CD3 protein is a CD3ε protein. In some embodiments, binding of the CD3 protein induces activation of the γδ T cell. In some embodiments, the antigen-binding domain that binds the protein associated with the TCR complex is derived from an antibody selected from UCHT1, OKT3, F6A, and L2K. In some embodiments, the antigen-binding domain that binds the protein associated with the TCR complex is a UCHT1 antigen-binding domain, for example, an scFv derived from UCHT1. In some embodiments, the UCHT1 antigen-binding domain comprises a Y to T mutation at a position corresponding to amino acid 182 of SEQ ID NO: 14 (Y182T). In some embodiments, the UCHT1 antigen-binding domain is a humanized variant of UCHT1 (huUCHT1), for example, a humanized variant of UCHT1 comprising a Y to T mutation at a position corresponding to amino acid 177 of SEQ ID NO: 44 (huUCHT1 (Y177T)). In some embodiments, the UCHT1 antigen-binding domain comprises an amino acid sequence having at least 80%, at least 85%, at least 90%, at least 95%, at least 96%, at least 97%, at least 98%, at least 99%, or 100% sequence identity with SEQ ID NO: 14 (UCHT1), SEQ ID NO: 72 (UCHT1 (Y182T)), SEQ ID NO: 44 (huUCHT1), or SEQ ID NO: 46 (huUCHT1 (Y177T)). In some embodiments, the antigen-binding domain that binds the protein associated with the TCR complex is OKT3, for example, the antigen-binding domain that binds a protein associated with the TCR complex comprises an amino acid sequence having at least 80%, at least 85%, at least 90%, at least 95%, at least 96%, at least 97%, at least 98%, at least 99%, or 100% sequence identity with SEQ ID NO: 22 (OKT3). In some embodiments, the antigen-binding domain that binds the protein associated with the TCR complex is F6A, for example, the antigen-binding domain that binds the protein associated with the TCR complex comprises an amino acid sequence having at least 80%, at least 85%, at least 90%, at least 95%, at least 96%, at least 97%, at least 98%, at least 99%, or 100% sequence identity with SEQ ID NO: 24 (F6A). In some embodiments, the antigen-binding domain that binds the protein associated with the TCR complex is L2K, for example, the antigen-binding domain that binds the protein associated with the TCR complex comprises an amino acid sequence having at least 80%, at least 85%, at least 90%, at least 95%, at least 96%, at least 97%, at least 98%, at least 99%, or 100% sequence identity with SEQ ID NO: 26 (L2K). In some embodiments, the cytosolic domain is a CD4 cytosolic domain and the transmembrane domain is a CD4 transmembrane domain, or wherein the cytosolic domain is a CD8 cytosolic domain and the transmembrane domain is a CD8 transmembrane domain. In some embodiments, the TCR co-receptor cytosolic domain and transmembrane domain comprises an amino acid sequence having at least 80%, at least 85%, at least 90%, at least 95%, at least 96%, at least 97%, at least 98%, at least 99%, or 100% sequence identity with SEQ ID NO: 18 (CD4 transmembrane and cytosolic domain). In some embodiments, components (a), (b), and/or (c) are connected in any suitable manner, such as in any suitable order and/or comprising any suitable linker(s). In some embodiments, (a), (b), and (c) are fused directly to each other, or joined by at least one linker. In some embodiments, (a) and (c) are fused to (b). In some embodiments, (b) and (c) are fused to (a). In some embodiments, at least one linker joins (a) to (b). In some embodiments, the at least one linker is a G₄S flexible linker, a large protein domain, a long helix structure, or a short helix structure. In some embodiments, the at least one linker comprises an amino acid sequence having at least 80%, at least 85%, at least 90%, at least 95%, at least 96%, at least 97%, at least 98%, at least 99%, or 100% sequence identity with SEQ ID NO: 12 ((G4S)4-based linker), SEQ ID NO: 16 (G4S-based linker), SEQ ID NO: 20 (CD4 based linker), SEQ ID NO: 28 (short helix connector), SEQ ID NO: 30 (long helix connector), SEQ ID NO: 32 (large domain connector), SEQ ID NO: 69 (flexible connector), SEQ ID NO: 73 (G4S flexible linker), or SEQ ID NO: 74 (G4S3 flexible linker). In some embodiments, the TAC polypeptide does not comprise a co-stimulatory domain and/or an activation domain. In some embodiments, the TAC polypeptide further comprises a leader sequence, for example, a leader sequence comprising an amino acid sequence having at least 80%, at least 85%, at least 90%, at least 95%, at least 96%, at least 97%, at least 98%, at least 99%, or 100% sequence identity with SEQ ID NO: 6 (muIgG leader), SEQ ID NO: 48 (huIgG leader), or SEQ ID NO: 50 (huCD8a leader). In some embodiments, the TAC polypeptide comprises an antigen-binding domain that binds (e.g., selectively binds) a CD19 antigen, for example, an antigen-binding domain comprising an amino acid sequence having at least 80%, at least 85%, at least 90%, at least 95%, at least 96%, at least 97%, at least 98%, at least 99%, or 100% sequence identity with SEQ ID NO: 36 (CD19 scFv). In some embodiments, the TAC polypeptide comprises an amino acid sequence having at least 80%, at least 85%, at least 90%, at least 95%, at least 96%, at least 97%, at least 98%, at least 99%, or 100% sequence identity with SEQ ID NO: 64 (CD19 TAC). In some embodiments, the TAC polypeptide is encoded by a nucleic acid comprising a nucleotide sequence having at least 80%, at least 85%, at least 90%, at least 95%, at least 96%, at least 97%, at least 98%, at least 99%, or 100% sequence identity with SEQ ID NO: 63 (CD19 TAC). In some embodiments, the TAC polypeptide comprises an antigen-binding domain that binds (e.g., selectively binds) a HER2 antigen. In some embodiments, the antigen-binding domain that binds a HER2 antigen comprises an antigen binding domain of an antibody selected from Trastuzumab, Pertuzumab, Lapatinib, Neratinib, Ado-trastuzumab Emtansine, Gancotamab, Margetuximab, Timigutuzumab, and Ertumaxomab. In some embodiments the antigen-binding domain that binds a HER2 antigen comprises an amino acid sequence having at least 80%, at least 85%, at least 90%, at least 95%, at least 96%, at least 97%, at least 98%, at least 99%, or 100% sequence identity with SEQ ID NO: 8 (HER2 DARPin). In some embodiments, the TAC polypeptide comprises an amino acid sequence having at least 80%, at least 85%, at least 90%, at least 95%, at least 96%, at least 97%, at least 98%, at least 99%, or 100% sequence identity with SEQ ID NO: 66 (HER2 TAC: huIgG leader sequence; huUCHT1 CD3-binding domain), SEQ ID NO: 68 (HER2 TAC: CD8a leader; huUCHT1 CD3-binding domain), or SEQ ID NO: 76 (HER2 TAC: muIgG leader; huUCHT1 CD3-binding domain). In some embodiments, the TAC polypeptide is encoded by a nucleic acid comprising a nucleotide sequence having at least 80%, at least 85%, at least 90%, at least 95%, at least 96%, at least 97%, at least 98%, at least 99%, or 100% sequence identity with SEQ ID NO: 65 (HER2 TAC: huIgG leader; huUCHT1 CD3-binding domain), SEQ ID NO: 67 (HER2 TAC: CD8a leader; huUCHT1 CD3-binding domain), or SEQ ID NO: 75 (HER2 TAC: muIgG leader; huUCHT1 CD3-binding domain). In some embodiments, the TAC polypeptide comprises an antigen-binding domain that binds (e.g., selectively binds) a BCMA antigen. In some embodiments, the antigen-binding domain that binds a BCMA antigen comprises an antigen binding domain of an antibody selected from Belantamab mafodotin and GSK2857916. In some embodiments, the antigen-binding domain that binds a BCMA antigen comprises an amino acid sequence having at least 80%, at least 85%, at least 90%, at least 95%, at least 96%, at least 97%, at least 98%, at least 99%, or 100% sequence identity with SEQ ID NO: 34 (BCMA scFv), SEQ ID NO: 52 (3625 BCMA scFv, Vh-Vl), or SEQ ID NO: 54 (3625 BCMA scFv, Vl-Vh). In some embodiments, the TAC polypeptide comprises an amino acid sequence having at least 80%, at least 85%, at least 90%, at least 95%, at least 96%, at least 97%, at least 98%, at least 99%, or 100% sequence identity with SEQ ID NO: 56 (BCMA TAC: 3625 BCMA scFv, Vh-Vl, helix linker; huUCHT1 CD3-binding domain), SEQ ID NO: 58 (BCMA TAC: 3625 BCMA scFv, Vl-Vh, helix linker; huUCHT1 CD3-binding domain), SEQ ID NO: 60 (BCMA TAC: 3625 BCMA scFv, Vh-Vl, G4S linker; huUCHT1 CD3-binding domain), or SEQ ID NO: 62 (BCMA TAC: 3625 BCMA scFv, Vl-Vh, G4S linker; huUCHT1 CD3-binding domain). In some embodiments, the TAC polypeptide is encoded by a nucleic acid comprising a nucleotide sequence having at least 80%, at least 85%, at least 90%, at least 95%, at least 96%, at least 97%, at least 98%, at least 99%, or 100% sequence identity with SEQ ID NO: 55 (BCMA TAC: 3625 BCMA scFv, Vh-Vl, helix linker; huUCHT1 CD3-binding domain), SEQ ID NO: 57 (BCMA TAC: 3625 BCMA scFv, Vl-Vh, helix linker; huUCHT1 CD3-binding domain), SEQ ID NO: 59 (BCMA TAC: 3625 BCMA scFv, Vh-Vl, G4S linker; huUCHT1 CD3-binding domain), or SEQ ID NO: 61 (BCMA TAC: 3625 BCMA scFv, Vl-Vh, G4S linker; huUCHT1 CD3-binding domain).

Disclosed herein, in certain embodiments, are γδ T cells comprising a nucleic acid sequence encoding a T cell-antigen coupler (TAC) polypeptide or an expression vector comprising a nucleic acid sequence encoding a T cell-antigen coupler (TAC) polypeptide. In some embodiments, the expression vector is a lentiviral vector, for example, a VSV-G pseudotyped lentiviral vector. In some embodiments, the expression vector is a γ retroviral vector, for example, a GALV pseudotyped γ-retroviral vector. In some embodiments, the nucleic acid sequence encoding the TAC comprises: (a) a first polynucleotide encoding a target-specific antigen-binding domain; (b) a second polynucleotide encoding an antigen-binding domain that binds a protein associated with a TCR complex; and (c) a third polynucleotide encoding a TCR co-receptor cytosolic domain and transmembrane domain. In some embodiments, the nucleic acid sequence encoding the TAC comprises, in order (e.g., from 5′ to 3′): (a) a first polynucleotide encoding a target-specific antigen-binding domain; (b) a second polynucleotide encoding an antigen-binding domain that binds a protein associated with a TCR complex; and (c) a third polynucleotide encoding a TCR co-receptor cytosolic domain and transmembrane domain. In some embodiments, the target specific antigen-binding domain is an antigen-binding domain that binds (e.g., selectively binds) a CD19 antigen, a HER2 antigen, or a BCMA antigen. In some embodiments, the target specific antigen-binding domain is a designed ankyrin repeat (DARPin) polypeptide, single chain variable fragment (scFv), single domain antibody, diabody, affibody, adnectin, affilin, phylomer, fynomer, affimer, peptide aptamer, knottin, centyrin, anticalin, or nanobody. In some embodiments, the target specific antigen-binding domain is a designed ankyrin repeat (DARPin) polypeptide or a single chain variable fragment (scFv). In some embodiments, the protein associated with the TCR complex is a CD3 protein, for example, a CD3 protein of a TCR complex on the γδ T cell. In some embodiments, the CD3 protein is a CD3γ protein, CD3δ protein and/or CD3ε protein. In some embodiments, the CD3 protein is a CD3ε protein. In some embodiments, binding of the CD3 protein induces activation of the γδ T cell. In some embodiments, the antigen-binding domain that binds the protein associated with the TCR complex is derived from an antibody selected from UCHT1, OKT3, F6A, and L2K. In some embodiments, the antigen-binding domain that binds the protein associated with the TCR complex is a UCHT1 antigen-binding domain, for example, an scFv derived from UCHT1. In some embodiments, the UCHT1 antigen-binding domain comprises a Y to T mutation at a position corresponding to amino acid 182 of SEQ ID NO: 14 (Y182T). In some embodiments, the UCHT1 antigen-binding domain is a humanized variant of UCHT1 (huUCHT1), for example, a humanized variant of UCHT1 comprising a Y to T mutation at a position corresponding to amino acid 177 of SEQ ID NO: 44 (huUCHT1 (Y177T)). In some embodiments, the UCHT1 antigen-binding domain comprises an amino acid sequence having at least 80%, at least 85%, at least 90%, at least 95%, at least 96%, at least 97%, at least 98%, at least 99%, or 100% sequence identity with SEQ ID NO: 14 (UCHT1), SEQ ID NO: 72 (UCHT1 (Y182T)), SEQ ID NO: 44 (huUCHT1), or SEQ ID NO: 46 (huUCHT1 (Y177T)). In some embodiments, the antigen-binding domain that binds the protein associated with the TCR complex is OKT3, for example, the antigen-binding domain that binds a protein associated with the TCR complex comprises an amino acid sequence having at least 80%, at least 85%, at least 90%, at least 95%, at least 96%, at least 97%, at least 98%, at least 99%, or 100% sequence identity with SEQ ID NO: 22 (OKT3). In some embodiments, the antigen-binding domain that binds the protein associated with the TCR complex is F6A, for example, the antigen-binding domain that binds the protein associated with the TCR complex comprises an amino acid sequence having at least 80%, at least 85%, at least 90%, at least 95%, at least 96%, at least 97%, at least 98%, at least 99%, or 100% sequence identity with SEQ ID NO: 24 (F6A). In some embodiments, the antigen-binding domain that binds the protein associated with the TCR complex is L2K, for example, the antigen-binding domain that binds the protein associated with the TCR complex comprises an amino acid sequence having at least 80%, at least 85%, at least 90%, at least 95%, at least 96%, at least 97%, at least 98%, at least 99%, or 100% sequence identity with SEQ ID NO: 26 (L2K). In some embodiments, the cytosolic domain is a CD4 cytosolic domain and the transmembrane domain is a CD4 transmembrane domain, or wherein the cytosolic domain is a CD8 cytosolic domain and the transmembrane domain is a CD8 transmembrane domain. In some embodiments, the third polynucleotide encodes a polypeptide comprising an amino acid sequence having at least 80%, at least 85%, at least 90%, at least 95%, at least 96%, at least 97%, at least 98%, at least 99%, or 100% sequence identity with SEQ ID NO: 18 (CD4 transmembrane and cytosolic domain). In some embodiments, the components encoded by the first, second, and/or third polynucleotides are connected in any suitable manner, such as in any suitable order and/or comprising any suitable linker(s). In some embodiments, the components encoded by (a), components encoded by (b), and components encoded by (c) are fused directly to each other, or joined by at least one linker. In some embodiments, the components encoded by (a) and the components encoded by (c) are fused to the components encoded by (b). In some embodiments, the components encoded by (b) and the components encoded by (c) are fused to the components encoded by (a). In some embodiments, at least one linker joins the components encoded by (a) to the components encoded by (b). In some embodiments, the at least one linker is a G₄S flexible linker, a large protein domain, a long helix structure, or a short helix structure. In some embodiments, the at least one linker comprises an amino acid sequence having at least 80%, at least 85%, at least 90%, at least 95%, at least 96%, at least 97%, at least 98%, at least 99%, or 100% sequence identity with SEQ ID NO: 12 ((G4S)4-based linker), SEQ ID NO: 16 (G4S-based linker), SEQ ID NO: 20 (CD4 based linker), SEQ ID NO: 28 (short helix connector), SEQ ID NO: 30 (long helix connector), SEQ ID NO: 32 (large domain connector), SEQ ID NO: 69 (flexible connector), SEQ ID NO: 73 (G4S flexible linker), or SEQ ID NO: 74 (G4S3 flexible linker). In some embodiments, the nucleic acid sequence does not encode a co-stimulatory domain and/or an activation domain. In some embodiments, the nucleic acid sequence further comprises a leader sequence, for example, a leader sequence comprising an amino acid sequence having at least 80%, at least 85%, at least 90%, at least 95%, at least 96%, at least 97%, at least 98%, at least 99%, or 100% sequence identity with SEQ ID NO: 6 (muIgG leader), SEQ ID NO: 48 (huIgG leader), or SEQ ID NO: 50 (huCD8a leader). In some embodiments, the first polynucleotide encodes an antigen-binding domain that binds (e.g., selectively binds) a CD19 antigen, for example, an antigen-binding domain comprising an amino acid sequence having at least 80%, at least 85%, at least 90%, at least 95%, at least 96%, at least 97%, at least 98%, at least 99%, or 100% sequence identity with SEQ ID NO: 36 (CD19 scFv). In some embodiments, the nucleic acid comprises a nucleotide sequence having at least 80%, at least 85%, at least 90%, at least 95%, at least 96%, at least 97%, at least 98%, at least 99%, or 100% sequence identity with SEQ ID NO: 63 (CD19 TAC). In some embodiments, the nucleic acid encodes an amino acid sequence having at least 80%, at least 85%, at least 90%, at least 95%, at least 96%, at least 97%, at least 98%, at least 99%, or 100% sequence identity with SEQ ID NO: 64 (CD19 TAC). In some embodiments, the first polynucleotide encodes an antigen-binding domain that binds (e.g., selectively binds) a HER2 antigen. In some embodiments, the antigen-binding domain that binds a HER2 antigen comprises an antigen binding domain of an antibody selected from Trastuzumab, Pertuzumab, Lapatinib, Neratinib, Ado-trastuzumab Emtansine, Gancotamab, Margetuximab, Timigutuzumab, and Ertumaxomab. In some embodiments the antigen-binding domain that binds a HER2 antigen comprises an amino acid sequence having at least 80%, at least 85%, at least 90%, at least 95%, at least 96%, at least 97%, at least 98%, at least 99%, or 100% sequence identity with SEQ ID NO: 8 (HER2 DARPin). In some embodiments, the nucleic acid comprises a nucleotide sequence having at least 80%, at least 85%, at least 90%, at least 95%, at least 96%, at least 97%, at least 98%, at least 99%, or 100% sequence identity with SEQ ID NO: 65 (HER2 TAC: huIgG leader; huUCHT1 CD3-binding domain), SEQ ID NO: 67 (HER2 TAC: CD8a leader; huUCHT1 CD3-binding domain), or SEQ ID NO: 75 (HER2 TAC: muIgG leader; huUCHT1 CD3-binding domain). In some embodiments, the nucleic acid encodes an amino acid sequence having at least 80%, at least 85%, at least 90%, at least 95%, at least 96%, at least 97%, at least 98%, at least 99%, or 100% sequence identity with SEQ ID NO: 66 (HER2 TAC: huIgG leader sequence; huUCHT1 CD3-binding domain), SEQ ID NO: 68 (HER2 TAC: CD8a leader; huUCHT1 CD3-binding domain), or SEQ ID NO: 76 (HER2 TAC: muIgG leader; huUCHT1 CD3-binding domain). In some embodiments, the first polynucleotide encodes an antigen-binding domain that binds (e.g., selectively binds) a BCMA antigen. In some embodiments, the antigen-binding domain that binds a BCMA antigen comprises an antigen binding domain of an antibody selected from Belantamab mafodotin and GSK2857916. In some embodiments, the antigen-binding domain that binds a BCMA antigen comprises an amino acid sequence having at least 80%, at least 85%, at least 90%, at least 95%, at least 96%, at least 97%, at least 98%, at least 99%, or 100% sequence identity with SEQ ID NO: 34 (BCMA scFv), SEQ ID NO: 52 (3625 BCMA scFv, Vh-Vl), or SEQ ID NO: 54 (3625 BCMA scFv, Vl-Vh). In some embodiments, the nucleic acid comprises a nucleotide sequence having at least 80%, at least 85%, at least 90%, at least 95%, at least 96%, at least 97%, at least 98%, at least 99%, or 100% sequence identity with SEQ ID NO: 55 (BCMA TAC: 3625 BCMA scFv, Vh-Vl, helix linker; huUCHT1 CD3-binding domain), SEQ ID NO: 57 (BCMA TAC: 3625 BCMA scFv, Vl-Vh, helix linker; huUCHT1 CD3-binding domain), SEQ ID NO: 59 (BCMA TAC: 3625 BCMA scFv, Vh-Vl, G4S linker; huUCHT1 CD3-binding domain), or SEQ ID NO: 61 (BCMA TAC: 3625 BCMA scFv, Vl-Vh, G4S linker; huUCHT1 CD3-binding domain). In some embodiments, the nucleic acid encodes an amino acid sequence having at least 80%, at least 85%, at least 90%, at least 95%, at least 96%, at least 97%, at least 98%, at least 99%, or 100% sequence identity with SEQ ID NO: 56 (BCMA TAC: 3625 BCMA scFv, Vh-Vl, helix linker; huUCHT1 CD3-binding domain), SEQ ID NO: 58 (BCMA TAC: 3625 BCMA scFv, Vl-Vh, helix linker; huUCHT1 CD3-binding domain), SEQ ID NO: 60 (BCMA TAC: 3625 BCMA scFv, Vh-Vl, G4S linker; huUCHT1 CD3-binding domain), or SEQ ID NO: 62 (BCMA TAC: 3625 BCMA scFv, Vl-Vh, G4S linker; huUCHT1 CD3-binding domain).

Disclosed herein, in certain embodiments, are pharmaceutical compositions comprising a γδ T cell disclosed herein, and a pharmaceutically acceptable excipient.

Disclosed herein, in certain embodiments, are methods of treating cancer in an individual in need thereof, comprising administering to the individual a γδ T cell or a pharmaceutical composition disclosed herein. In some embodiments, the cancer is a solid cancer or a liquid cancer. In some embodiments, the cancer is a lung cancer, a breast cancer, multiple myeloma, glioblastoma, gastric cancer, ovarian cancer, stomach cancer, colorectal cancer, urothelial cancer, endometrial cancer, or a colon cancer. In some embodiments, the pharmaceutical composition is administered in combination with zoledronate, IL-2, and/or a CD16 agonist (e.g., an anti-CD16 antibody). In some embodiments, the zoledronate, IL-2, and/or CD16 agonist is administered before, after, or simultaneously with the γδ T cell.

Disclosed herein, in certain embodiments, are methods of treating a cancer comprising a CD19-expressing cancer cell in an individual in need thereof, comprising administering to the individual to the individual a γδ T cell or a pharmaceutical composition disclosed herein (e.g., a pharmaceutical composition comprising a γδ T cell comprising a CD19-TAC or a nucleic acid encoding the CD19-TAC). In some embodiments, the cancer is a B cell malignancy. In some embodiments, the cancer is B cell lymphoma, acute lymphoblastic leukemia (ALL), chronic lymphocytic leukemia (CLL), or Non-Hodgkins Lymphoma. In some embodiments, the pharmaceutical composition is administered in combination with zoledronate, IL-2, and/or a CD16 agonist (e.g., an anti-CD16 antibody). In some embodiments, the zoledronate, IL-2, and/or CD16 agonist is administered before, after, or simultaneously with the γδ T cell.

Disclosed herein, in certain embodiments, are methods of treating a cancer comprising a HER2-expressing cancer cell in an individual in need thereof, comprising administering to the individual to the individual a γδ T cell or a pharmaceutical composition disclosed herein (e.g., a pharmaceutical composition comprising a γδ T cell comprising a HER2-TAC or a nucleic acid encoding the HER2-TAC). In some embodiments, the cancer is a breast cancer, bladder cancer, pancreatic cancer, ovarian cancer, or stomach cancer. In some embodiments, the pharmaceutical composition is administered in combination with zoledronate, IL-2, and/or a CD16 agonist (e.g., an anti-CD16 antibody). In some embodiments, the zoledronate, IL-2, and/or CD16 agonist is administered before, after, or simultaneously with the γδ T cell.

Disclosed herein, in certain embodiments, are methods of treating a cancer comprising a BCMA-expressing cancer cell in an individual in need thereof, comprising administering to the individual to the individual a γδ T cell or a pharmaceutical composition disclosed herein (e.g., a pharmaceutical composition comprising a γδ T cell comprising a BCMA-TAC or a nucleic acid encoding the BCMA-TAC). In some embodiments, the cancer is a leukemia, lymphoma, or multiple myeloma. In some embodiments, the pharmaceutical composition is administered in combination with zoledronate, IL-2, and/or a CD16 agonist (e.g., an anti-CD16 antibody). In some embodiments, the zoledronate, IL-2, and/or CD16 agonist is administered before, after, or simultaneously with the γδ T cell.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A is a schematic of natural T cell activation.

FIG. 1B is a schematic of CAR based T cell activation.

FIG. 1C is a schematic of a T cell-antigen coupler (TAC) based T cell activation.

FIG. 1D is a schematic of natural T cell activation.

FIG. 1E is a schematic of CAR based T cell activation.

FIG. 1F is a schematic of TAC based T cell activation.

FIG. 2A is a schematic of a TAC configuration with a UCHT1 domain being centered between a transmembrane domain (TM) and an antigen binding domain.

FIG. 2B is a schematic of a TAC configuration in which a UCHT1 domain is N-terminal, followed by an antigen binding domain and a transmembrane domain.

FIG. 2C is a schematic of a TAC molecule with a generic antigen binding domain and a UCHT1 domain.

FIG. 3A is a schematic of a TAC molecule with a generic antigen binding domain.

FIG. 3B is a schematic of a TAC with an anti-HER2 DARPin antigen binding domain.

FIG. 3C is a schematic of a TAC with an anti-CD19 scFv antigen binding domain.

FIG. 3D is a schematic of a TAC with an anti-BCMA scFv antigen binding domain.

FIG. 3E is a schematic of a TAC molecule with an anti-HER2 DARPin antigen binding domain.

FIG. 3F is a schematic of a TAC molecule with an anti-BCMA scFv antigen binding domain.

FIG. 4 is a schematic depiction of a γ9δ2 TCR interacting with a target cell. The ligand for the γ9δ2 TCR is BTN3A1, which undergoes conformational changes. Cells that are sensitive to killing by γ9δ2 T cells display an active form of BTN3A1 that can bind the γ9δ2 TCR (right hand image). Cells that are insensitive to γ9δ2 T cells display an inactive form of BTN3A1 that cannot bind the γ9δ2 TCR (left hand image).

FIG. 5 is a schematic depiction of a TAC-expressing γ9δ2 T cell interacting with a target cell. Cells that are sensitive to killing by γ9δ2 T cells display an active form of BTN3A1 that can bind the γ9δ2 TCR (right hand image). Cells that are insensitive to γ9δ2 T cells display an inactive form of BTN3A1 that cannot bind the γ9δ2 TCR (left hand image). The TAC receptor can co-opt the γ9δ2 TCR, similar to a conventional αβ TCR, and direct γ9δ2 T cells to attack otherwise insensitive targets (left hand image).

FIG. 6 is a schematic depiction of a method for generating TAC-expressing γδ T cells.

FIG. 7 depicts the results of a process for engineering γ9δ2 T cells to express TAC receptors. Day 0 shows the frequency of γδ T cells in the starting product. The γ9δ2 T cells were activated with zoledronate and cultured in the presence of IL-2. Following activation, the γ9δ2 T cells were infected with lentivirus encoding the TAC receptor and allowed to expand for 10-14 days at which point they become the dominant population in the culture. Contaminating αβ T cells were removed from the cell product by negative selection of CD4 and CD8 receptors yielding a final product that is >98% γ9δ2 T cells.

FIG. 8 depicts the sensitivity of CD19+ cells (Raji, JeKo-1 and NALM-6 cells) to in vitro killing by γ9δ2 T cells that do not express a TAC.

FIG. 9 depicts the sensitivity of CD19+ cells (Raji, JeKo-1 and NALM-6 cells) to in vitro killing by (i) CD19-TAC-expressing γ9δ2 T cells, (ii) HER2-TAC-expressing γ9δ2 T cells, or (iii) γ9δ2 T cells that do not express a TAC.

FIG. 10 depicts the sensitivity of HER2+ cells (A549 and OVCAR-3 cells) to in vitro killing by (i) CD19-TAC-expressing γ9δ2 T cells, (ii) HER2-TAC-expressing γ9δ2 T cells, or (iii) γ9δ2 T cells that do not express a TAC.

FIG. 11 depicts sensitivity of BCMA+ cells (KMS-11 and MM.1S cells) or BCMA− cells (K562 cells) to in vitro killing by (i) BCMA-TAC-expressing γ9δ2 T cells, or (ii) γ9δ2 T cells that do not express a TAC.

FIG. 12 depicts therapeutic activity of TAC-expressing γ9δ2 T cells in mice bearing CD19-positive/HER2-negative JeKo-1 xenografts. Mice were treated with: (i) γ9δ2 T cells engineered with a TAC specific for CD19 (CD19-TAC γδ T cells; open circle, solid lines), (ii) γ9δ2 T cells engineered with a TAC specific for HER2 (HER2-TAC γδ T cells; solid circles, solid lines), or (iii) carrier medium (Cryostor10™) alone (solid circles, dashed lines).

FIG. 13 depicts in vitro cytotoxicity of HER2-TAC γδ T cells (closed circles) or non-engineered (NTD) γδ T cells (open circles) towards HT1080 or NCI-N87 cells at the indicated effector to target (E:T) ratios

FIG. 14 depicts in vitro cytotoxicity of HER2-TAC γδ T cells (closed triangles) or non-engineered (NTD) γδ T cells (open triangles) towards HT1080 or NCI-N87 cells at the indicated effector to target (E:T) ratios. Target cells were pre-treated with zoledronate prior to co-culture.

FIG. 15 depicts cytokine production for HER2-TAC or non-engineered (NTD) γδ T cells following co-culture with NCI-N87 cells.

FIG. 16 depicts cytokine production for HER2-TAC or non-engineered (NTD) γδ T cells following co-culture with NCI-N87 cells. Target cells were pre-treated with zoledronate prior to co-culture.

FIG. 17 depicts in vitro cytotoxicity of HER2-TAC γδ T cells, non-engineered (NTD) γδ T cells, HER2-TAC αβ T cells, and non-engineered (NTD) up T cells towards NCI-N87 cells at the indicated effector to target (E:T) ratio.

FIG. 18 depicts in vitro cytotoxicity of HER2-TAC γδ T cells, non-engineered (NTD) γδ T cells, HER2-TAC αβ T cells, and non-engineered (NTD) up T cells towards NCI-N87 cells at the indicated effector to target (E:T) ratio. Target cells were pre-treated with zoledronate prior to co-culture.

FIG. 19 depicts expression of CD16 in γδ and αβ T cells.

FIG. 20A depicts γδ T cell CD107a or TNFα expression in response to CD16 stimulation with a plate-bound agonistic CD16 mAb bound at 10, 25, 50, or 100 ng/μL concentrations. FIG. 20B depicts CD16 expression in the unstimulated, non-transduced γδ T cell culture.

FIG. 21 depicts CD107a and TNFα expression (MFI) in BCMA-TAC γδ T cell culture following stimulation with BCMA-Fc and/or CD16 at the indicated concentrations. Mean CD107a and TNFα expression were calculated from experiments conducted with BCMA-TAC γδ T cells derived from 6 donors.

### DETAILED DESCRIPTION

Cancer is a major health challenge, with over 150,000 cases of cancer expected to be diagnosed in Canada alone. While patients with early stage disease are sometimes treated effectively by conventional therapies (surgery, radiation, chemotherapy), few options are available to patients with advanced disease, and those options are typically palliative in nature.

Active immunotherapy seeks to employ the patient's immune system to clear tumors and offers an option to patients who have failed conventional therapies. Generally, this treatment involves infusing patients with large numbers of tumor-specific T cells. This approach has proven to be successful in early phase clinical trials for a number of diseases, including melanoma, myeloma, leukemia, lymphoma and synovial sarcoma. As a specific example, several clinical studies have demonstrated that immunotherapy with T cells are curative in patients with advanced melanoma, confirming the utility of this approach. Additionally, patients suffering from chronic lymphocytic leukemia (CLL) and acute lymphoblastic leukemia (ALL) have also been effectively treated and cured with T cell immunotherapy.

A key challenge facing the clinical application of adoptive T cell therapy is the source of the T cells. Typically, T cells isolated from a tumor-bearing patient are grown to large numbers ex vivo and are administered back into the patient to induce a robust anti-tumor immune response. Tumor specificity is achieved by either: (i) isolating naturally-occurring tumor-specific T cells from the patient; or (ii) engineering bulk T cells from the peripheral blood to express tumor-specific receptors. Naturally occurring tumor-specific T cells are rare and isolating such cells in therapeutic quantities from cancer patients is a laborious and costly procedure. In contrast, it is becoming more efficient to engineer readily available peripheral T cells with tumor-specific receptors through genetic manipulation. Techniques have been developed for this engineering process, which are clinically viable, and several clinical trials have demonstrated the feasibility and efficacy of genetically-engineered T cells for the treatment of cancer.

To this point, most engineered T cell therapies involving genetic modification of the T cells yield: (i) forced expression of T cell receptor (TCR); or (ii) a chimeric antigen receptor (CAR) specific for antigen targets on the tumor. To date, the chimeric antigen receptors used for engineering T cells consist of: (i) a targeting domain, usually a single-chain fragment variable (scFv); (ii) a transmembrane domain; and (iii) a cytosolic domain that contains signaling elements from the T cell receptor and associated proteins. Such chimeric antigen receptors have also been referred to as “T-body” or “Chimeric Immune Receptor” (CIR), but currently, most researchers use the term “CAR”. One advantage of the CAR approach is that it allows any patient's immune cells to be targeted against any desirable target in a major histocompatibility complex (MHC) independent manner. This is appealing as MHC presentation is often defective in tumor cells.

CARs are considered in modular terms and scientists have spent considerable time investigating the influence of different cytoplasmic signaling domains on CAR function. Conventional CARs generally share two main components: (i) the CD3 zeta cytoplasmic domain, which contains immunotyrosine activation motifs (ITAMs) critical for T cell activation; and (ii) components of costimulatory receptors that trigger important survival pathways such as the Akt pathway.

The first-generation CARs employed a single signaling domain from either CD3ζ or FcεRIγ. Second-generation CARs combined the signaling domain of CD3ζ with the cytoplasmic domain of costimulatory receptors from either the CD28 or TNFR family of receptors. Most CAR-engineered T cells that are currently being tested in the clinic employ second-generation CARs where CD3ζ is coupled to the cytoplasmic domain of either CD28 or CD137. These second generation CARs have demonstrated anti-tumor activity in CD19-positive tumors. Third-generation CARs combined multiple co-stimulatory domains, but there is concern that third-generation CARs may lose antigen-specificity.

While CAR-engineered T cells have shown considerable promise in clinical application, they rely on a synthetic method for replacing the native activation signal that is provided by the T cell receptor (TCR). Since this synthetic receptor does not deliver all of the signaling components associated with the TCR (ex. ITAMs on CD3γ, CD3δ, CD3ε), it remains unclear whether the T cells are optimally activated by the CAR or how the CAR activation affects T cell differentiation (ex. progression to memory). Furthermore, since the CAR signaling domains are disconnected from their natural regulatory partners by the very nature of the CAR structure, there is an inherent risk that CARs may lead to a low-level of constitutive activation, which could result in off-target toxicities. Therefore, the synthetic nature of the prototypic CAR may disrupt canonical mechanisms that limit TCR activation, and may underpin the severe toxicity often associated with therapeutic doses of conventional CAR T cells.

Given these limitations, it is preferable to re-direct T cells to attack tumors via their natural TCR. An alternate chimeric receptor, termed a T cell Antigen Coupler (TAC or TAC) receptor, has been developed which employs a distinct biology to direct the T cell to attack tumors. While the CAR is a fully synthetic receptor that stitches together components of T cell receptor (TCR) signaling complex, the TAC receptor re-directs the TCR towards tumor targets and recapitulates the native TCR signaling structure. For example, in some embodiments, the TACs disclosed herein activate natural Major Histocompatibility complex (MHC) signaling through the T cell receptor (TCR), while retaining MHC-unrestricted targeting. Further, the TACs disclosed herein recruit the T Cell Receptor (TCR) in combination with co-receptor stimulation. Moreover, in some embodiments, TACs disclosed herein show enhanced activity and safety.

Currently, engineered T cells are produced using semi-automated workflows that require highly trained technical staff, expensive and sophisticated infrastructure, and a single course of therapy is priced on the order of USD$350,000. For these therapies to become broadly available, it is necessary to reduce the cost of the cell product. Additionally, the current generation of engineered T cells are autologous, which means that each product is individualized and that the patient to be treated must have T cells of sufficient quality and sufficient numbers to generate a therapeutic dose, which cannot be guaranteed. Further, the patient's disease must be stable enough to allow for the manufacturing period to be completed, which typically requires 2-3 weeks; even longer should there be a manufacturing fail that requires the process to be repeated. Finally, logistical challenges will necessitate regional manufacturing centers to ensure expedient delivery of patient material to/from the tertiary care centers where they are treated.

Centralized collection and engineering of T cells from healthy donors can overcome these challenges by providing an off-the-shelf product that can be manufactured in large scale and accessed on demand. The main challenge with implementing such a strategy is graft versus host disease (GVHD). The T cell receptor (TCR) is highly selected for host MHC. When T cells are transferred to a foreign host, the TCR reacts to the foreign MHC and triggers a robust reaction that culminates in GVHD. A non-canonical T cell population, known as γδ T cells, displays innate anti-tumor activity and can be used in non-MHC matched donors without causing GVHD. However, while γδ T cell treatment has been tolerated in clinical settings, therapeutic activity has been modest.

### Certain Terminology

The term “T cell” as used herein refers to a type of lymphocyte that plays a central role in cell-mediated immunity. T cells, also referred to as T lymphocytes, are distinguished from other lymphocytes, such as B cells and natural killer cells, by the presence of a T-cell receptor (TCR) on the cell surface. There are several subsets of T cells with distinct functions, including but not limited to, T helper cells, cytotoxic T cells, memory T cells, regulatory T cells, natural killer T cells, and γδ T cells.

The term “γδ T cell” or “gamma delta T cell” or “gd T cell” as used herein refers to any lymphocyte having a γδ T cell receptor (TCR) on its surface, including one γ-chain and one δ-chain. Most T cells are αβ (alpha beta) T cells which have on their surface a TCR including glycoprotein chains called α (alpha) and β (beta). In contrast, gamma delta (γδ) T cells have a TCR that is made up of one γ (gamma) chain and one δ (delta) chain. Unlike conventional αβ TCR-expressing cells, γδ TCR-expressing cells recognize their targets independent of classical MHC I and II. γδ T cells are also typically characterized by production of abundant pro inflammatory cytokines such as IFN-gamma. γδ T cell marker characteristics typically include, for example, CD3, CD4, CD8, CD69, CD56, CD27 CD45RA, CD45, TCR-Vγ9, TCR-Vδ2, TCR-Vδ1, TCR-Vδ3, NKG2D, CCR5, CCR7, CXCR3, and CXCR5, or combinations thereof. The term γδ T cells includes all subsets of γδ T cells, including, for example, Vδ1, Vδ2, and Vδ3 γδ T cells, as well as naïve, effector memory, central memory, and terminally differentiated γδ T cells. As a further example, the term γδ T cells includes Vδ4, Vδ5, Vδ7, and Vδ8 γδ T cells, as well as Vγ2, Vγ3, Vγ5, Vγ8, Vγ9, Vγ10, and Vγ11 γδ T cells. The present application further contemplates T cells that express one γ-chain or one δ-chain, optionally in combination with a second polypeptide to form a functional TCR.

The term “antigen-binding domain,” refers to any substance or molecule that binds, directly or indirectly, to a target (e.g., BCMA, HER2, or CD19). Antigen-binding domains include antibodies or fragments thereof, peptides, peptidomimetics, proteins, glycoproteins, proteoglycans, carbohydrates, lipids, nucleic acids, or small molecules that bind to a target.

As used herein, unless otherwise indicated, the term “antibody” is understood to mean an intact antibody (e.g., an intact monoclonal antibody), or a fragment thereof, such as a Fc fragment of an antibody (e.g., an Fc fragment of a monoclonal antibody), or an antigen-binding fragment of an antibody (e.g., an antigen-binding fragment of a monoclonal antibody), including an intact antibody, antigen-binding fragment, or Fc fragment that has been modified, engineered, or chemically conjugated. In some embodiments, antibodies are multimeric proteins that contain four polypeptide chains. Two of the polypeptide chains are called immunoglobulin heavy chains (H chains), and two of the polypeptide chains are called immunoglobulin light chains (L chains). The immunoglobulin heavy and light chains are connected by an interchain disulfide bond. The immunoglobulin heavy chains are connected by interchain disulfide bonds. A light chain consists of one variable region (V_{L}) and one constant region (C_{L}). The heavy chain consists of one variable region (V_{H}) and at least three constant regions (CH₁, CH₂ and CH₃). In other embodiments, e.g., camelid-derived antibodies or nanobodies, an antibodies contain a heavy chain and two constant regions (CH₂ and CH₃). The variable regions determine the binding specificity of the antibody. Each variable region contains three hypervariable regions known as complementarity determining regions (CDRs) flanked by four relatively conserved regions known as framework regions (FRs). The extent of the FRs and CDRs has been defined (Kabat, E. A., et al. (1991) SEQUENCES OF PROTEINS OF IMMUNOLOGICAL INTEREST, FIFTH EDITION, U.S. Department of Health and Human Services, NIH Publication No. 91-3242; and Chothia, C. et al. (1987) J. MOL. BIOL. 196:901-917). The three CDRs, referred to as CDR₁, CDR₂, and CDR₃, contribute to the antibody binding specificity. Naturally occurring antibodies have been used as starting material for engineered antibodies, such as chimeric antibodies and humanized antibodies. Examples of antibody-based antigen-binding fragments include Fab, Fab′, (Fab′)₂, Fv, single chain antibodies (e.g., scFv), single domain antibodies (e.g., nanobodies), minibodies, and diabodies. Examples of antibodies that have been modified or engineered include chimeric antibodies, humanized antibodies, and multispecific antibodies (e.g., bispecific antibodies). An example of a chemically conjugated antibody is an antibody conjugated to a toxin moiety.

The term “T cell antigen coupler” or TAC is used interchangeably with “trifunctional T cell antigen coupler” or Tri-TAC and refers to an engineered nucleic acid construct or polypeptide comprising (a) a target-specific ligand or antigen-binding domain, (b) a ligand or antigen-binding domain that binds a protein associated with a T cell receptor (TCR) complex, and (c) a T cell receptor signaling domain.

The term “polynucleotide” and/or “nucleic acid sequence” and/or “nucleic acid” as used herein refers to a sequence of nucleoside or nucleotide monomers consisting of bases, sugars and intersugar (backbone) linkages. The term also includes modified or substituted sequences comprising non-naturally occurring monomers or portions thereof. The nucleic acid sequences of the present application may be deoxyribonucleic acid sequences (DNA) or ribonucleic acid sequences (RNA) and may include naturally occurring bases including adenine, guanine, cytosine, thymidine and uracil. The sequences may also contain modified bases. Examples of such modified bases include aza and deaza adenine, guanine, cytosine, thymidine and uracil; and xanthine and hypoxanthine. The nucleic acids of the present disclosure may be isolated from biological organisms, formed by laboratory methods of genetic recombination or obtained by chemical synthesis or other known protocols for creating nucleic acids.

The term “isolated polynucleotide” or “isolated nucleic acid sequence” as used herein refers to a nucleic acid substantially free of cellular material or culture medium when produced by recombinant DNA techniques, or chemical precursors, or other chemicals when chemically synthesized. An isolated nucleic acid is also substantially free of sequences which naturally flank the nucleic acid (i.e. sequences located at the 5′ and 3′ ends of the nucleic acid) from which the nucleic acid is derived. The term “nucleic acid” is intended to include DNA and RNA and is either double stranded or single stranded, and represents the sense or antisense strand. Further, the term “nucleic acid” includes the complementary nucleic acid sequences.

The term “recombinant nucleic acid” or “engineered nucleic acid” as used herein refers to a nucleic acid or polynucleotide that is not found in a biological organism. For example, recombinant nucleic acids may be formed by laboratory methods of genetic recombination (such as molecular cloning) to create sequences that would not otherwise be found in nature. Recombinant nucleic acids may also be created by chemical synthesis or other known protocols for creating nucleic acids.

The term “polypeptide” or “protein” as used herein describes a chain of amino acids. A polypeptide or protein of this disclosure is a peptide, which usually describes a chain of amino acids. The term protein as used herein also describes a large molecule comprising one or more chains of amino acids and, in some embodiments, is a fragment or domain of a protein or a full length protein. Furthermore, as used herein, the term protein either refers to a linear chain of amino acids or to a chain of amino acids that has been processed and folded into a functional protein. The protein structure is divided into four distinct levels: (1) primary structure—referring to the sequence of amino acids in the polypeptide chain, (2) secondary structure—referring to the regular local sub-structures on the polypeptide backbone chain, such as α-helix and β-sheets, (3) tertiary structure—referring to the three-dimensional structure if monomeric and multimeric protein molecules, and (4) quaternary structure—referring to the three-dimensional structure comprising the aggregation of two or more individual polypeptide chains that operate as a single functional unit. The proteins of the present disclosure, in some embodiments, are obtained by isolation and purification of the proteins from cells where they are produced naturally, by enzymatic (e.g., proteolytic) cleavage, and/or recombinantly by expression of nucleic acid encoding the proteins or fragments of this disclosure. The proteins and/or fragments of this disclosure, in some embodiments, is obtained by chemical synthesis or other known protocols for producing proteins and fragments.

The term “isolated polypeptide” refers to a polypeptide substantially free of cellular material or culture medium when produced by recombinant DNA techniques, or chemical precursors or other chemicals when chemically synthesized.

The term “vector” as used herein refers to a polynucleotide that is used to deliver a nucleic acid to the inside of a cell. In some embodiments, a vector is an expression vector comprising expression control sequences (for example, a promoter) operatively linked to a nucleic acid to be expressed in a cell. Vectors known in the art include, but are not limited to, plasmids, phages, cosmids and viruses.

The term “tumor antigen” or “tumor associated antigen” as used herein refers to an antigenic substance produced in tumor cells that triggers an immune response in a host (e.g. which is presented by MHC complexes). In some embodiments, a tumor antigen is on the surface of a tumor cell.

As used herein, the term “transmembrane and cytosolic domain” refers to a polypeptide that comprises a transmembrane domain and a cytosolic domain of a protein associated with the T cell receptor (TCR) complex. In some embodiments, such transmembrane and cytosolic domain may include, but is not limited to, protein domains that (a) associate with the lipid raft and/or (b) bind Lck.

A “TCR co-receptor” as used herein, refers to a molecule that assists the T cell receptor (TCR) in communicating with an antigen-presenting cell and may be considered part of the first signal that leads to the activation of the TCR. Examples of TCR co-receptors include, but are not limited to, CD4, LAG3, and CD8.

A “TCR co-stimulator” or “co-stimulatory domain” as used herein, refers to a molecule that enhances the response of a T cell to an antigen and may be considered as the second signal that leads to the activation of the TCR. Examples of TCR co-stimulators include, but are not limited to, ICOS, CD27, CD28, 4-1BB (CD 137), OX40 (CD134), CD30, CD40, lymphocyte fiction-associated antigen 1 (LFA-1), CD2, CD7, LIGHT, NKG2C, B7-H3, and a ligand that specifically binds CD83.

The terms “recipient”, “individual”, “subject”, “host”, and “patient”, are used interchangeably herein and in some embodiments, refer to any mammalian subject for whom diagnosis, treatment, or therapy is desired, particularly humans. “Mammal” for purposes of treatment refers to any animal classified as a mammal, including humans, domestic and farm animals, and laboratory, zoo, sports, or pet animals, such as dogs, horses, cats, cows, sheep, goats, pigs, mice, rats, rabbits, guinea pigs, monkeys etc. In some embodiments, the mammal is human. None of these terms require the supervision of medical personnel.

As used herein, the terms “treatment,” “treating,” and the like, in some embodiments, refer to administering an agent, or carrying out a procedure, for the purposes of obtaining an effect. The effect may be prophylactic in terms of completely or partially preventing a disease or symptom thereof and/or may be therapeutic in terms of affecting a partial or complete cure for a disease and/or symptoms of the disease. “Treatment,” as used herein, may include treatment of a disease or disorder (e.g. cancer) in a mammal, particularly in a human, and includes: (a) preventing the disease or a symptom of a disease from occurring in a subject which may be predisposed to the disease but has not yet been diagnosed as having it (e.g., including diseases that may be associated with or caused by a primary disease; (b) inhibiting the disease, i.e., arresting its development; and (c) relieving the disease, i.e., causing regression of the disease. Treating may refer to any indicia of success in the treatment or amelioration or prevention of a cancer, including any objective or subjective parameter such as abatement; remission; diminishing of symptoms; or making the disease condition more tolerable to the patient; slowing in the rate of degeneration or decline; or making the final point of degeneration less debilitating. The treatment or amelioration of symptoms is based on one or more objective or subjective parameters; including the results of an examination by a physician. Accordingly, the term “treating” includes the administration of the compounds or agents of the present invention to prevent, delay, alleviate, arrest or inhibit development of the symptoms or conditions associated with diseases (e.g. cancer). The term “therapeutic effect” refers to the reduction, elimination, or prevention of the disease, symptoms of the disease, or side effects of the disease in the subject.

As used herein, singular forms “a”, “and,” and “the” include plural referents unless the context clearly indicates otherwise. Thus, for example, reference to “an antibody” includes a plurality of antibodies and reference to “an antibody” in some embodiments includes multiple antibodies, and so forth.

As used herein, all numerical values or numerical ranges include whole integers within or encompassing such ranges and fractions of the values or the integers within or encompassing ranges unless the context clearly indicates otherwise. Thus, for example, reference to a range of 90-100%, includes 91%, 92%, 93%, 94%, 95%, 95%, 96%, 97%, etc., as well as 91.1%, 91.2%, 91.3%, 91.4%, 91.5%, etc., 92.1%, 92.2%, 92.3%, 92.4%, 92.5%, etc., and so forth. In another example, reference to a range of 1-5,000 fold includes 1, 2, 3, 4, 5, 6, 7, 8, 9, 10, 11, 12, 13, 14, 15, 16, 17, 18, 19, 20, fold, etc., as well as 1.1, 1.2, 1.3, 1.4, 1.5, fold, etc., 2.1, 2.2, 2.3, 2.4, 2.5, fold, etc., and so forth.

“About” a number, as used herein, refers to range including the number and ranging from 10% below that number to 10% above that number. “About” a range refers to 10% below the lower limit of the range, spanning to 10% above the upper limit of the range.

“Percent (%) identity” refers to the extent to which two sequences (nucleotide or amino acid) have the same residue at the same positions in an alignment. For example, “an amino acid sequence is X % identical to SEQ ID NO: Y” refers to % identity of the amino acid sequence to SEQ ID NO: Y and is elaborated as X % of residues in the amino acid sequence are identical to the residues of sequence disclosed in SEQ ID NO: Y. Generally, computer programs are employed for such calculations. Exemplary programs that compare and align pairs of sequences, include ALIGN (Myers and Miller, 1988), FASTA (Pearson and Lipman, 1988; Pearson, 1990) and gapped BLAST (Altschul et al., 1997), BLASTP, BLASTN, or GCG (Devereux et al., 1984).

As used herein, the term “selective binding” refers to the higher affinity with which a molecule (e.g., a protein such as a target-binding ligand or antigen-binding domain of a TAC) binds its target molecule (e.g., a target antigen such as HER2, BCMA, or CD19) over other molecules. Unless indicated otherwise, the terms “selective binding” and “specific binding” are used interchangeably herein.

### Gamma Delta Cells Expressing a T Cell Antigen Coupler (TAC)

Disclosed herein, in certain embodiments, are gamma delta T cells comprising or expressing a T cell-antigen coupler (TAC) polypeptide. In some embodiments, the TAC polypeptide comprises: (a) a target-specific ligand or antigen-binding domain; (b) a ligand or antigen-binding domain that binds a TCR complex; and (c) a transmembrane domain and cytosolic domain. In some embodiments, the TAC polypeptide does not comprise a co-stimulatory domain. In some embodiments, the TAC polypeptide does not comprise a co-activation domain.

Further disclosed herein, in certain embodiments, are gamma delta T cells comprising a nucleic acid encoding a T cell-antigen coupler (TAC) polypeptide. In some embodiments, the nucleic acid encoding a TAC polypeptide comprises: (a) a first polynucleotide encoding a target-specific ligand or antigen-binding domain; (b) a second polynucleotide encoding a ligand or antigen-binding domain that binds a TCR complex; and (c) a third polynucleotide encoding a transmembrane domain and cytosolic domain. In some embodiments, the nucleic acid encoding a TAC polypeptide does not encode a co-stimulatory domain. In some embodiments, the nucleic acid encoding a TAC polypeptide does not encode a co-activation domain.

Further disclosed herein, in certain embodiments, are methods of treating a cancer in an individual in need thereof, comprising administering to the individual gamma delta T cells comprising or expressing a T cell-antigen coupler (TAC) polypeptide. In some embodiments, the TAC polypeptide comprises: (a) a target-specific ligand or antigen-binding domain; (b) a ligand or antigen-binding domain that binds a TCR complex; and (c) a transmembrane domain and cytosolic domain. In some embodiments, the TAC polypeptide does not comprise a co-stimulatory domain. In some embodiments, the TAC polypeptide does not comprise a co-activation domain.

Further disclosed herein, in certain embodiments, are methods of treating a cancer in an individual in need thereof, comprising administering to the individual gamma delta T cells comprising a nucleic acid encoding a T cell-antigen coupler (TAC) polypeptide. In some embodiments, the nucleic acid encoding a TAC polypeptide comprises: (a) a first polynucleotide encoding a target-specific ligand or antigen-binding domain; (b) a second polynucleotide encoding a ligand or antigen-binding domain that binds a TCR complex; and (c) a third polynucleotide encoding a transmembrane domain and cytosolic domain. In some embodiments, the nucleic acid encoding a TAC polypeptide does not encode a co-stimulatory domain. In some embodiments, the nucleic acid encoding a TAC polypeptide does not encode a co-activation domain.

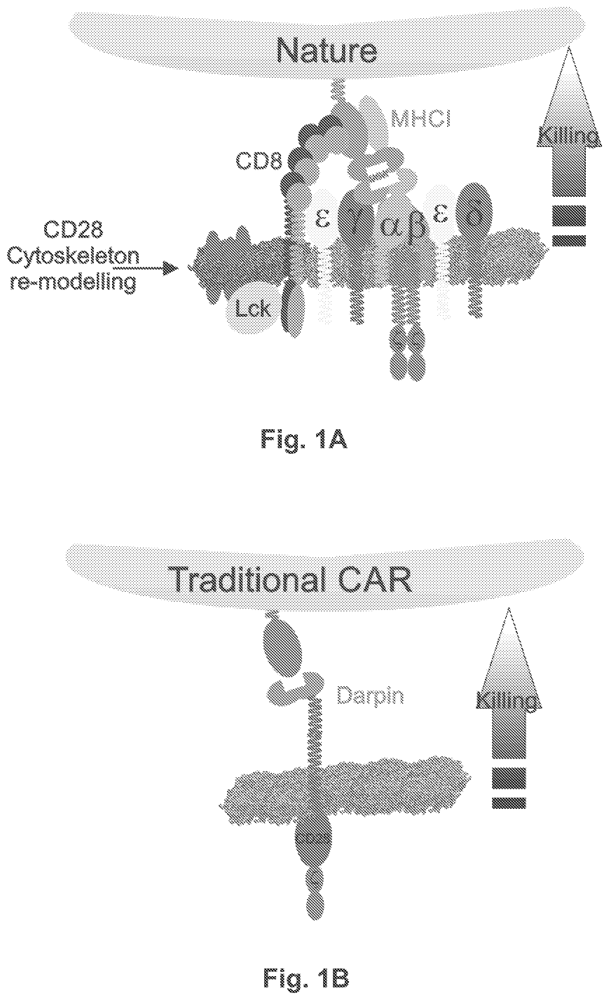

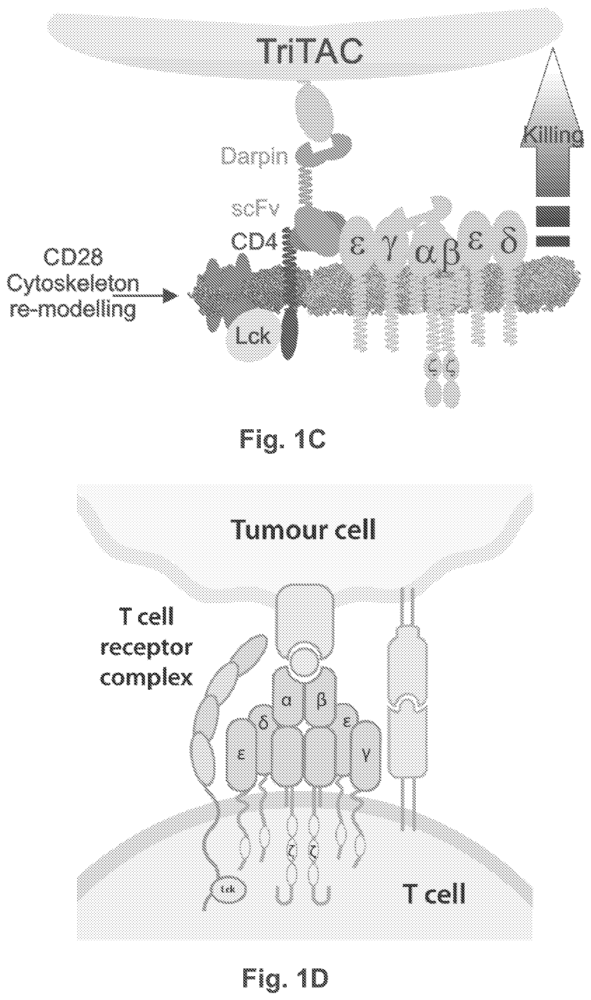

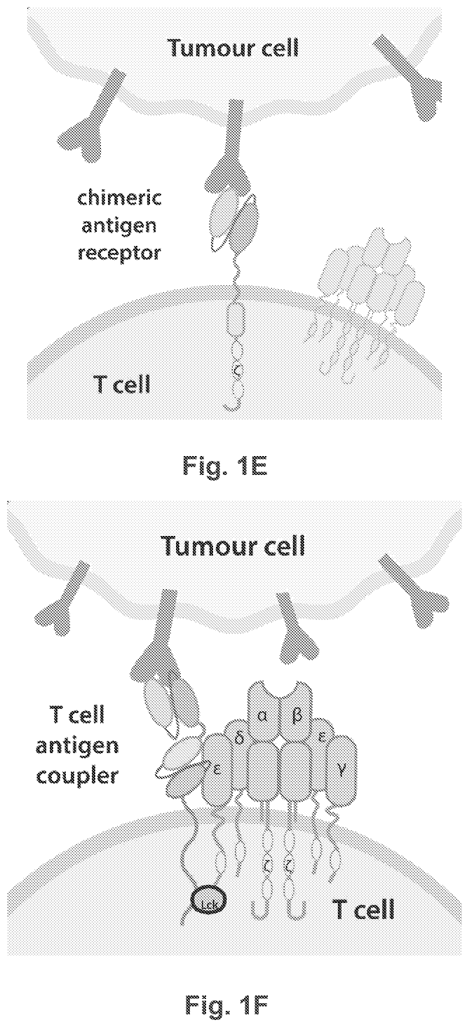

An depiction of T cell activation by a TAC receptor is provided in FIG. 1A-FIG. 1F.

FIG. 1A shows an example of CD8 T cell activation based on the co-assembly of different receptors and their associated protein partners. Initially, the major histocompatibility complex I is presenting an antigen (helix). This is recognized by a T cell receptor (TCR) complex capable of binding the antigen. The TCR complex contains several individual subunits. The α/β domains are able to interact directly with the antigen presented on MHC-I. The α/β domains then interact with several other domains (ε, γ, δ, and ζ), all of which participate in T cell activation via various intracellular activation domains. The TCR complex interacts with MHC-I concurrently with the CD8 co-receptor. The CD8 co-receptor binds to the MHC-I in an antigen independent manner. CD8 directly interacts with Lck, a protein kinase important for activating the TCR receptor complex. The CD8 and Lck interaction also ensures their association with lipid rafts (membrane portion) microdomains, which are hypothesized to organize and encapsulate other relevant signaling moieties (dark spheres). Later stages of activation then lead to CD28 recruitment. If this interaction cascade occurs several times in parallel, T cells become activated and are able to exert their cytotoxic effects.

FIG. 1B provides an overview of Chimeric Antigen Receptors (CARs). CARs seek to reproduce the complex mechanism of T cell activation by combining several key activation domains, such as CD3ζ and CD28 in a single synthetically engineered molecule. The CAR then directly interacts with an antigen of choice using specific binding domains. Depicted here is an ankyrin repeat protein (DARPin). It is believed that several such interactions occurring in parallel lead to T cell activation.

FIG. 1C is an overview of the TAC technology mimicking a natural activation process. T cell activation occurs following ligation of MHC by the TCR and T cell co-receptor (either CD4 or CD8), which simultaneously bind to conserved regions within the MHC molecule. These co-receptors also bind directly to Lck, a protein kinase that is crucial for T cell activation. None of the traditional chimeric receptors or bi-functional proteins engage the co-receptor molecules or Lck. An exemplary TAC includes the transmembrane and intracellular regions of the CD4 co-receptor, which localize to lipid rafts and bind Lck, respectively, fused to a single-chain antibody that binds CD3 (UCHT1; SEQ ID NO: 13, 14 and homologs thereof). This construct is designed to draw the CD3 molecule and the TCR into regions of lipid rafts and bring Lck into the proximity of the TCR, similar to natural MHC binding. To target this receptor, the TAC may include, for example, a designed ankyrin repeat (DARPin) linked to the CD4-UCHT1 chimera.

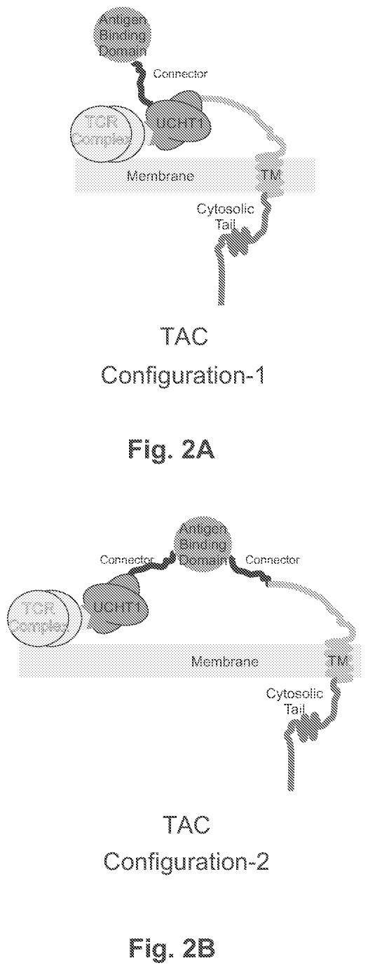

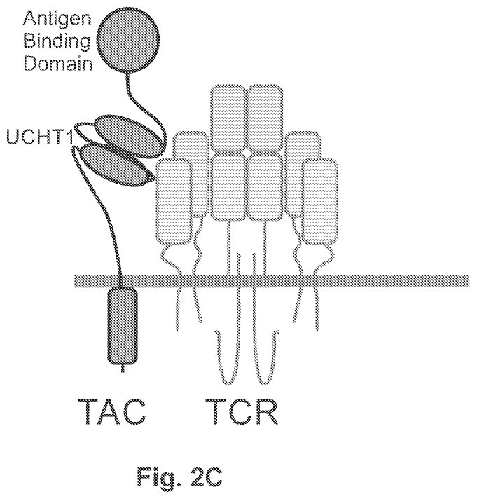

Multiple TAC configurations are possible (e.g., FIG. 2A and FIG. 2B). In an exemplary configuration, configuration 1 (FIG. 2A), the antigen binding domain is located at the N-terminus of the receptor, connected to the CD3 ligand binding domain and then the co-receptor domain. In another exemplary configuration, configuration 2 (FIG. 2B), the CD3 ligand binding domain is located at the N-terminus of the receptor, connected to the antigen binding domain which in turn connects to the co-receptor domain.

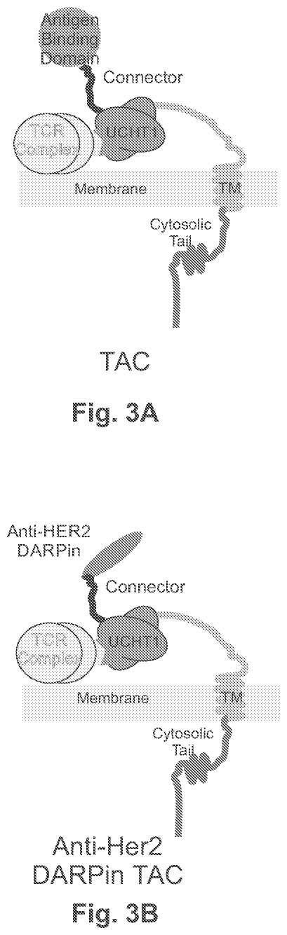

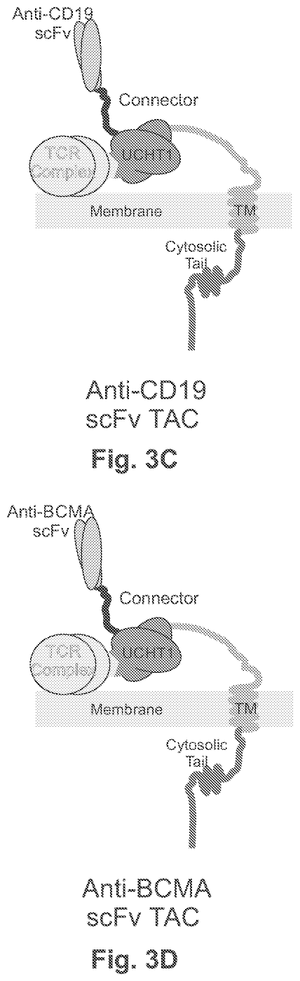

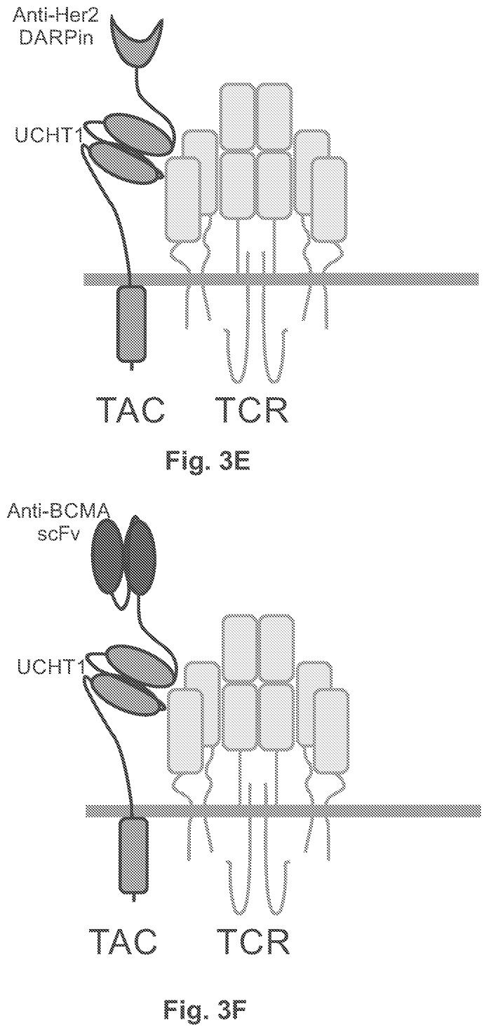

Multiple classes of ligand binding domains can be incorporated into a TAC molecule. FIG. 3A-FIG. 3D depict a general schematic of a configuration 1 TAC (FIG. 3A), a TAC bearing a HER2-specific DARPin (FIG. 3B), a TAC bearing a CD19-specific scFv (FIG. 3C), and a TAC bearing a BCMA-specific scFv (FIG. 3D).

### Target-Specific Antigen Binding Domain

The target-specific antigen binding domain, also referred to as a ligand binds to an antigen on the target cell. In some embodiments, a target cell is a cell associated with a disease state, including, but not limited to, cancer, hematologic malignancy, large B-cell lymphoma, diffuse large B-cell lymphoma, primary mediastinal B cell lymphoma, high grade B-cell lymphoma, or large B cell lymphoma arising from follicular lymphoma. In some embodiments, a target cell is a tumor cell. In some embodiments, a target-specific antigen-binding domain binds to a tumor antigen or tumor associated antigen on a tumor cell. In some embodiments, the target antigen is a tumor antigen. In some embodiments, the tumor antigen when proteinaceous is a sequence of 8 or more amino acids up to the full protein. In some embodiments, the tumor antigen is any number of amino acids in between 8 and the full length protein which comprises at least one antigenic fragment of the full length protein that is presented in a Major Histocompatibility Complex (MHC). Examples of tumor antigens include, but are not limited to, CD19, HER2 (erbB-2), B-cell maturation antigen (BCMA), alphafetoprotein (AFP), carcinoembryonic antigen (CEA), CA-125, MUC-1, epithelial tumor antigen (ETA), tyrosinase, melanoma-associated antigen (MAGE), prostate-specific antigen (PSA), glioma-associated antigen, β-human chorionic gonadotropin, thyroglobulin, RAGE-1, MN-CA IX, human telomerase reverse transcriptase, RU1, RU2 (AS), intestinal carboxyl esterase, mut hsp70-2, M-CSF, prostase, PAP, NY-ESO-1, LAGE-1a, p53, prostein, PSMA, survivin and telomerase, prostate-carcinoma tumor antigen-1 (PCTA-1), ELF2M, neutrophil elastase, CD22, insulin growth factor (IGF)-I, IGF-II, IGF-I receptor and mesothelin.

In some embodiments, the target-specific antigen-binding domain is an antibody or a fragment thereof. In some embodiments, the target-specific antigen-binding domain is selected from single chain antibodies (e.g., single-chain fragment variable antibodies (scFvs)), single domain antibodies (e.g., heavy-chain-only antibodies (VHH), shark heavy-chain-only antibodies (VNAR)), nanobodies, diabodies, minibodies, Fab fragments, Fab′ fragments, F(ab′)₂ fragments, or Fv fragments.

In some embodiments, the target-specific antigen-binding domain is selected from ankyrin repeat proteins (DARPins), affibodies, adnectins, affilins, phylomers, fynomers, affimers, peptide aptamers, lectins, knottins, centyrins, anticalins, peptides, peptidomimetics, proteins, glycoproteins, or proteoglycans that bind to a target, or naturally occurring ligands for a target. In some embodiments, the target-specific antigen-binding domain is a non-protein compound that binds to a target, including but not limited to carbohydrates, lipids, nucleic acids, or small molecules.

In some embodiments, the target-specific antigen-binding domain is a designed ankyrin repeat (DARPin). In some embodiments, the target-specific antigen-binding domain is a single-chain variable fragment (ScFv). In some embodiments, the target-specific antigen-binding domain is a nanobody.

In some embodiments, the tumor antigen is a HER2 antigen. In some embodiments, the HER2 specific antigen-binding domain comprises an antigen binding domain of an antibody selected from Trastuzumab, Pertuzumab, Lapatinib, Neratinib, Ado-trastuzumab Emtansine, Gancotamab, Margetuximab, Timigutuzumab, and Ertumaxomab. In some embodiments, the target-specific antigen-binding domain is a DARPin that binds a HER2 (erbB-2) antigen. In some embodiments, the target-specific antigen-binding domain is a DARPin that specifically binds a HER2 (erbB-2) antigen. In some embodiments, the DARPin targeted to HER2 (erb-2) is encoded by the nucleotide sequence of SEQ ID NO: 7 or comprises the amino acid sequence of SEQ ID NO: 8 (HER2 DARPin).

In some embodiments, the HER2-binding domain comprises an amino acid sequence having at least 70% sequence identity with the amino acid sequence of SEQ ID NO: 8 (HER2 DARPin). In some embodiments, the HER2-binding domain comprises an amino acid sequence having at least 75% sequence identity with the amino acid sequence of SEQ ID NO: 8 (HER2 DARPin). In some embodiments, the HER2-binding domain comprises an amino acid sequence having at least 80% sequence identity with the amino acid sequence of SEQ ID NO: 8 (HER2 DARPin). In some embodiments, the HER2-binding domain comprises an amino acid sequence having at least 85% sequence identity with the amino acid sequence of SEQ ID NO: 8 (HER2 DARPin). In some embodiments, the HER2-binding domain comprises an amino acid sequence having at least 90% sequence identity with the amino acid sequence of SEQ ID NO: 8 (HER2 DARPin). In some embodiments, the HER2-binding domain comprises an amino acid sequence having at least 95% sequence identity with the amino acid sequence of SEQ ID NO: 8 (HER2 DARPin). In some embodiments, the HER2-binding domain comprises an amino acid sequence having at least 96% sequence identity with the amino acid sequence of SEQ ID NO: 8 (HER2 DARPin). In some embodiments, the HER2-binding domain comprises an amino acid sequence having at least 97% sequence identity with the amino acid sequence of SEQ ID NO: 8 (HER2 DARPin). In some embodiments, the HER2-binding domain comprises an amino acid sequence having at least 98% sequence identity with the amino acid sequence of SEQ ID NO: 8 (HER2 DARPin). In some embodiments, the HER2-binding domain comprises an amino acid sequence having at least 99% sequence identity with the amino acid sequence of SEQ ID NO: 8 (HER2 DARPin). In some embodiments, the HER2-binding domain comprises the amino acid sequence of SEQ ID NO: 8 (HER2 DARPin).

In some embodiments, the tumor antigen is a BCMA antigen. In some embodiments, the BCMA specific antigen-binding domain comprises an antigen binding domain of an antibody selected from Belantamab mafodotin, and GSK2857916. In some embodiments, the target-specific antigen-binding domain is a scFv that binds BCMA. In some embodiments, the target-specific antigen-binding domain is a scFv that specifically binds BCMA. In some embodiments, the scFv that binds BCMA is encoded by the nucleotide sequence of SEQ ID NO: 33 or comprises the amino acid sequence of SEQ ID NO: 34.

In some embodiments, the polynucleotide encoding the BCMA-binding domain comprises a nucleotide sequence having at least 70% sequence identity with the nucleotide sequence of SEQ ID NO: 33 (BCMA scFv). In some embodiments, the polynucleotide encoding the BCMA-binding domain comprises a nucleotide sequence having at least 75% sequence identity with the nucleotide sequence of SEQ ID NO: 33 (BCMA scFv). In some embodiments, the polynucleotide encoding the BCMA-binding domain comprises a nucleotide sequence having at least 80% sequence identity with the nucleotide sequence of SEQ ID NO: 33 (BCMA scFv). In some embodiments, the polynucleotide encoding the BCMA-binding domain comprises a nucleotide sequence having at least 85% sequence identity with the nucleotide sequence of SEQ ID NO: 33 (BCMA scFv). In some embodiments, the polynucleotide encoding the BCMA-binding domain comprises a nucleotide sequence having at least 90% sequence identity with the nucleotide sequence of SEQ ID NO: 33 (BCMA scFv). In some embodiments, the polynucleotide encoding the BCMA-binding domain comprises a nucleotide sequence having at least 95% sequence identity with the nucleotide sequence of SEQ ID NO: 33 (BCMA scFv). In some embodiments, the polynucleotide encoding the BCMA-binding domain comprises a nucleotide sequence having at least 96% sequence identity with the nucleotide sequence of SEQ ID NO: 33 (BCMA scFv). In some embodiments, the polynucleotide encoding the BCMA-binding domain comprises a nucleotide sequence having at least 97% sequence identity with the nucleotide sequence of SEQ ID NO: 33 (BCMA scFv). In some embodiments, the polynucleotide encoding the BCMA-binding domain comprises a nucleotide sequence having at least 98% sequence identity with the nucleotide sequence of SEQ ID NO: 33 (BCMA scFv). In some embodiments, the polynucleotide encoding the BCMA-binding domain comprises a nucleotide sequence having at least 99% sequence identity with the nucleotide sequence of SEQ ID NO: 33 (BCMA scFv). In some embodiments, the polynucleotide encoding the BCMA-binding domain comprises the nucleotide sequence of SEQ ID NO: 33 (BCMA scFv).

In some embodiments, the BCMA-binding domain comprises an amino acid sequence having at least 70% sequence identity with the amino acid sequence of SEQ ID NO: 34 (BCMA scFv). In some embodiments, the BCMA-binding domain comprises an amino acid sequence having at least 75% sequence identity with the amino acid sequence of SEQ ID NO: 34 (BCMA scFv). In some embodiments, the BCMA-binding domain comprises an amino acid sequence having at least 80% sequence identity with the amino acid sequence of SEQ ID NO: 34 (BCMA scFv). In some embodiments, the BCMA-binding domain comprises an amino acid sequence having at least 85% sequence identity with the amino acid sequence of SEQ ID NO: 34 (BCMA scFv). In some embodiments, the BCMA-binding domain comprises an amino acid sequence having at least 90% sequence identity with the amino acid sequence of SEQ ID NO: 34 (BCMA scFv). In some embodiments, the BCMA-binding domain comprises an amino acid sequence having at least 95% sequence identity with the amino acid sequence of SEQ ID NO: 34 (BCMA scFv). In some embodiments, the BCMA-binding domain comprises an amino acid sequence having at least 96% sequence identity with the amino acid sequence of SEQ ID NO: 34 (BCMA scFv). In some embodiments, the BCMA-binding domain comprises an amino acid sequence having at least 97% sequence identity with the amino acid sequence of SEQ ID NO: 34 (BCMA scFv). In some embodiments, the BCMA-binding domain comprises an amino acid sequence having at least 98% sequence identity with the amino acid sequence of SEQ ID NO: 34 (BCMA scFv). In some embodiments, the BCMA-binding domain comprises an amino acid sequence having at least 99% sequence identity with the amino acid sequence of SEQ ID NO: 34 (BCMA scFv). In some embodiments, the BCMA-binding domain comprises the amino acid sequence of SEQ ID NO: 34 (BCMA scFv).

In some embodiments, the CDR sequences of the BCMA-binding domain have 100% identity with the CDR sequences of the amino acid sequence of SEQ ID NO: 34 (BCMA scFv) (i.e., the BCMA-binding domain comprises an amino acid sequence comprising a CDRH1, CDRH2, CDRH3, CDRL1, CDRL2, and CDRL3, each having 100% identity to the corresponding CDR in the amino acid sequence of SEQ ID NO: 34 (BCMA scFv)). In some embodiments, the CDR sequences of the BCMA-binding domain have 100% identity with the CDR sequences of the amino acid sequence of SEQ ID NO: 34 (BCMA scFv), and the non-CDR (e.g., framework) sequences of the BCMA-binding domain have at least 80% sequence identity with the non-CDR (e.g., framework) sequences of the amino acid sequence of SEQ ID NO: 34 (BCMA scFv). In some embodiments, the CDR sequences of the BCMA-binding domain have 100% identity with the CDR sequences of the amino acid sequence of SEQ ID NO: 34 (BCMA scFv), and the non-CDR (e.g., framework) sequences of the BCMA-binding domain have at least 85% sequence identity with the non-CDR (e.g., framework) sequences of the amino acid sequence of SEQ ID NO: 34 (BCMA scFv). In some embodiments, the CDR sequences of the BCMA-binding domain have 100% identity with the CDR sequences of the amino acid sequence of SEQ ID NO: 34 (BCMA scFv), and the non-CDR (e.g., framework) sequences of the BCMA-binding domain have at least 90% sequence identity with the non-CDR (e.g., framework) sequences of the amino acid sequence of SEQ ID NO: 34 (BCMA scFv). In some embodiments, the CDR sequences of the BCMA-binding domain have 100% identity with the CDR sequences of the amino acid sequence of SEQ ID NO: 34 (BCMA scFv), and the non-CDR (e.g., framework) sequences of the BCMA-binding domain have at least 95% sequence identity with the non-CDR (e.g., framework) sequences of the amino acid sequence of SEQ ID NO: 34 (BCMA scFv). In some embodiments, the CDR sequences of the BCMA-binding domain have 100% identity with the CDR sequences of the amino acid sequence of SEQ ID NO: 34 (BCMA scFv), and the non-CDR (e.g., framework) sequences of the BCMA-binding domain have at least 96% sequence identity with the non-CDR (e.g., framework) sequences of the amino acid sequence of SEQ ID NO: 34 (BCMA scFv). In some embodiments, the CDR sequences of the BCMA-binding domain have 100% identity with the CDR sequences of the amino acid sequence of SEQ ID NO: 34 (BCMA scFv), and the non-CDR (e.g., framework) sequences of the BCMA-binding domain have at least 97% sequence identity with the non-CDR (e.g., framework) sequences of the amino acid sequence of SEQ ID NO: 34 (BCMA scFv). In some embodiments, the CDR sequences of the BCMA-binding domain have 100% identity with the CDR sequences of the amino acid sequence of SEQ ID NO: 34 (BCMA scFv), and the non-CDR (e.g., framework) sequences of the BCMA-binding domain have at least 98% sequence identity with the non-CDR (e.g., framework) sequences of the amino acid sequence of SEQ ID NO: 34 (BCMA scFv). In some embodiments, the CDR sequences of the BCMA-binding domain have 100% identity with the CDR sequences of the amino acid sequence of SEQ ID NO: 34 (BCMA scFv), and the non-CDR (e.g., framework) sequences of the BCMA-binding domain have at least 99% sequence identity with the non-CDR (e.g., framework) sequences of the amino acid sequence of SEQ ID NO: 34 (BCMA scFv).

In some embodiments, the polynucleotide encoding the BCMA-binding domain comprises a nucleotide sequence having at least 70% sequence identity with the nucleotide sequence of SEQ ID NO: 51 (3625 BCMA scFv, Vh-Vl). In some embodiments, the polynucleotide encoding the BCMA-binding domain comprises a nucleotide sequence having at least 75% sequence identity with the nucleotide sequence of SEQ ID NO: 51 (3625 BCMA scFv, Vh-Vl). In some embodiments, the polynucleotide encoding the BCMA-binding domain comprises a nucleotide sequence having at least 80% sequence identity with the nucleotide sequence of SEQ ID NO: 51 (3625 BCMA scFv, Vh-Vl). In some embodiments, the polynucleotide encoding the BCMA-binding domain comprises a nucleotide sequence having at least 85% sequence identity with the nucleotide sequence of SEQ ID NO: 51 (3625 BCMA scFv, Vh-Vl). In some embodiments, the polynucleotide encoding the BCMA-binding domain comprises a nucleotide sequence having at least 90% sequence identity with the nucleotide sequence of SEQ ID NO: 51 (3625 BCMA scFv, Vh-Vl). In some embodiments, the polynucleotide encoding the BCMA-binding domain comprises a nucleotide sequence having at least 95% sequence identity with the nucleotide sequence of SEQ ID NO: 51 (3625 BCMA scFv, Vh-Vl). In some embodiments, the polynucleotide encoding the BCMA-binding domain comprises a nucleotide sequence having at least 96% sequence identity with the nucleotide sequence of SEQ ID NO: 51 (3625 BCMA scFv, Vh-Vl). In some embodiments, the polynucleotide encoding the BCMA-binding domain comprises a nucleotide sequence having at least 97% sequence identity with the nucleotide sequence of SEQ ID NO: 51 (3625 BCMA scFv, Vh-Vl). In some embodiments, the polynucleotide encoding the BCMA-binding domain comprises a nucleotide sequence having at least 98% sequence identity with the nucleotide sequence of SEQ ID NO: 51 (3625 BCMA scFv, Vh-Vl). In some embodiments, the polynucleotide encoding the BCMA-binding domain comprises a nucleotide sequence having at least 99% sequence identity with the nucleotide sequence of SEQ ID NO: 51 (3625 BCMA scFv, Vh-Vl). In some embodiments, the polynucleotide encoding the BCMA-binding domain comprises the nucleotide sequence of SEQ ID NO: 51 (3625 BCMA scFv, Vh-Vl).

In some embodiments, the BCMA-binding domain comprises an amino acid sequence having at least 70% sequence identity with the amino acid sequence of SEQ ID NO: 52 (3625 BCMA scFv, Vh-Vl). In some embodiments, the BCMA-binding domain comprises an amino acid sequence having at least 75% sequence identity with the amino acid sequence of SEQ ID NO: 52 (3625 BCMA scFv, Vh-Vl). In some embodiments, the BCMA-binding domain comprises an amino acid sequence having at least 80% sequence identity with the amino acid sequence of SEQ ID NO: 52 (3625 BCMA scFv, Vh-Vl). In some embodiments, the BCMA-binding domain comprises an amino acid sequence having at least 85% sequence identity with the amino acid sequence of SEQ ID NO: 52 (3625 BCMA scFv, Vh-Vl). In some embodiments, the BCMA-binding domain comprises an amino acid sequence having at least 90% sequence identity with the amino acid sequence of SEQ ID NO: 52 (3625 BCMA scFv, Vh-Vl). In some embodiments, the BCMA-binding domain comprises an amino acid sequence having at least 95% sequence identity with the amino acid sequence of SEQ ID NO: 52 (3625 BCMA scFv, Vh-Vl). In some embodiments, the BCMA-binding domain comprises an amino acid sequence having at least 96% sequence identity with the amino acid sequence of SEQ ID NO: 52 (3625 BCMA scFv, Vh-Vl). In some embodiments, the BCMA-binding domain comprises an amino acid sequence having at least 97% sequence identity with the amino acid sequence of SEQ ID NO: 52 (3625 BCMA scFv, Vh-Vl). In some embodiments, the BCMA-binding domain comprises an amino acid sequence having at least 98% sequence identity with the amino acid sequence of SEQ ID NO: 52 (3625 BCMA scFv, Vh-Vl). In some embodiments, the BCMA-binding domain comprises an amino acid sequence having at least 99% sequence identity with the amino acid sequence of SEQ ID NO: 52 (3625 BCMA scFv, Vh-Vl). In some embodiments, the BCMA-binding domain comprises the amino acid sequence of SEQ ID NO: 52 (3625 BCMA scFv, Vh-Vl).

In some embodiments, the CDR sequences of the BCMA-binding domain have 100% identity with the CDR sequences of the amino acid sequence of SEQ ID NO: 52 (3625 BCMA scFv, Vh-Vl) (i.e., the BCMA-binding domain comprises an amino acid sequence comprising a CDRH1, CDRH2, CDRH3, CDRL1, CDRL2, and CDRL3, each having 100% identity to the corresponding CDR in the amino acid sequence of SEQ ID NO: 52 (3625 BCMA scFv, Vh-Vl)). In some embodiments, the CDR sequences of the BCMA-binding domain have 100% identity with the CDR sequences of the amino acid sequence of SEQ ID NO: 52 (3625 BCMA scFv, Vh-Vl), and the non-CDR (e.g., framework) sequences of the BCMA-binding domain have at least 80% sequence identity with the non-CDR (e.g., framework) sequences of the amino acid sequence of SEQ ID NO: 52 (3625 BCMA scFv, Vh-Vl). In some embodiments, the CDR sequences of the BCMA-binding domain have 100% identity with the CDR sequences of the amino acid sequence of SEQ ID NO: 52 (3625 BCMA scFv, Vh-Vl), and the non-CDR (e.g., framework) sequences of the BCMA-binding domain have at least 85% sequence identity with the non-CDR (e.g., framework) sequences of the amino acid sequence of SEQ ID NO: 52 (3625 BCMA scFv, Vh-Vl). In some embodiments, the CDR sequences of the BCMA-binding domain have 100% identity with the CDR sequences of the amino acid sequence of SEQ ID NO: 52 (3625 BCMA scFv, Vh-Vl), and the non-CDR (e.g., framework) sequences of the BCMA-binding domain have at least 90% sequence identity with the non-CDR (e.g., framework) sequences of the amino acid sequence of SEQ ID NO: 52 (3625 BCMA scFv, Vh-Vl). In some embodiments, the CDR sequences of the BCMA-binding domain have 100% identity with the CDR sequences of the amino acid sequence of SEQ ID NO: 52 (3625 BCMA scFv, Vh-Vl), and the non-CDR (e.g., framework) sequences of the BCMA-binding domain have at least 95% sequence identity with the non-CDR (e.g., framework) sequences of the amino acid sequence of SEQ ID NO: 52 (3625 BCMA scFv, Vh-Vl). In some embodiments, the CDR sequences of the BCMA-binding domain have 100% identity with the CDR sequences of the amino acid sequence of SEQ ID NO: 52 (3625 BCMA scFv, Vh-Vl), and the non-CDR (e.g., framework) sequences of the BCMA-binding domain have at least 96% sequence identity with the non-CDR (e.g., framework) sequences of the amino acid sequence of SEQ ID NO: 52 (3625 BCMA scFv, Vh-Vl). In some embodiments, the CDR sequences of the BCMA-binding domain have 100% identity with the CDR sequences of the amino acid sequence of SEQ ID NO: 52 (3625 BCMA scFv, Vh-Vl), and the non-CDR (e.g., framework) sequences of the BCMA-binding domain have at least 97% sequence identity with the non-CDR (e.g., framework) sequences of the amino acid sequence of SEQ ID NO: 52 (3625 BCMA scFv, Vh-Vl). In some embodiments, the CDR sequences of the BCMA-binding domain have 100% identity with the CDR sequences of the amino acid sequence of SEQ ID NO: 52 (3625 BCMA scFv, Vh-Vl), and the non-CDR (e.g., framework) sequences of the BCMA-binding domain have at least 98% sequence identity with the non-CDR (e.g., framework) sequences of the amino acid sequence of SEQ ID NO: 52 (3625 BCMA scFv, Vh-Vl). In some embodiments, the CDR sequences of the BCMA-binding domain have 100% identity with the CDR sequences of the amino acid sequence of SEQ ID NO: 52 (3625 BCMA scFv, Vh-Vl), and the non-CDR (e.g., framework) sequences of the BCMA-binding domain have at least 99% sequence identity with the non-CDR (e.g., framework) sequences of the amino acid sequence of SEQ ID NO: 52 (3625 BCMA scFv, Vh-Vl).

In some embodiments, the polynucleotide encoding the BCMA-binding domain comprises a nucleotide sequence having at least 70% sequence identity with the nucleotide sequence of SEQ ID NO: 53 (3625 BCMA scFv, Vl-Vh). In some embodiments, the polynucleotide encoding the BCMA-binding domain comprises a nucleotide sequence having at least 75% sequence identity with the nucleotide sequence of SEQ ID NO: 53 (3625 BCMA scFv, Vl-Vh). In some embodiments, the polynucleotide encoding the BCMA-binding domain comprises a nucleotide sequence having at least 80% sequence identity with the nucleotide sequence of SEQ ID NO: 53 (3625 BCMA scFv, Vl-Vh). In some embodiments, the polynucleotide encoding the BCMA-binding domain comprises a nucleotide sequence having at least 85% sequence identity with the nucleotide sequence of SEQ ID NO: 53 (3625 BCMA scFv, Vl-Vh). In some embodiments, the polynucleotide encoding the BCMA-binding domain comprises a nucleotide sequence having at least 90% sequence identity with the nucleotide sequence of SEQ ID NO: 53 (3625 BCMA scFv, Vl-Vh). In some embodiments, the polynucleotide encoding the BCMA-binding domain comprises a nucleotide sequence having at least 95% sequence identity with the nucleotide sequence of SEQ ID NO: 53 (3625 BCMA scFv, Vl-Vh). In some embodiments, the polynucleotide encoding the BCMA-binding domain comprises a nucleotide sequence having at least 96% sequence identity with the nucleotide sequence of SEQ ID NO: 53 (3625 BCMA scFv, Vl-Vh). In some embodiments, the polynucleotide encoding the BCMA-binding domain comprises a nucleotide sequence having at least 97% sequence identity with the nucleotide sequence of SEQ ID NO: 53 (3625 BCMA scFv, Vl-Vh). In some embodiments, the polynucleotide encoding the BCMA-binding domain comprises a nucleotide sequence having at least 98% sequence identity with the nucleotide sequence of SEQ ID NO: 53 (3625 BCMA scFv, Vl-Vh). In some embodiments, the polynucleotide encoding the BCMA-binding domain comprises a nucleotide sequence having at least 99% sequence identity with the nucleotide sequence of SEQ ID NO: 53 (3625 BCMA scFv, Vl-Vh). In some embodiments, the polynucleotide encoding the BCMA-binding domain comprises the nucleotide sequence of SEQ ID NO: 53 (3625 BCMA scFv, Vl-Vh).

In some embodiments, the BCMA-binding domain comprises an amino acid sequence having at least 70% sequence identity with the amino acid sequence of SEQ ID NO: 54 (3625 BCMA scFv, Vl-Vh). In some embodiments, the BCMA-binding domain comprises an amino acid sequence having at least 75% sequence identity with the amino acid sequence of SEQ ID NO: 54 (3625 BCMA scFv, Vl-Vh). In some embodiments, the BCMA-binding domain comprises an amino acid sequence having at least 80% sequence identity with the amino acid sequence of SEQ ID NO: 54 (3625 BCMA scFv, Vl-Vh). In some embodiments, the BCMA-binding domain comprises an amino acid sequence having at least 85% sequence identity with the amino acid sequence of SEQ ID NO: 54 (3625 BCMA scFv, Vl-Vh). In some embodiments, the BCMA-binding domain comprises an amino acid sequence having at least 90% sequence identity with the amino acid sequence of SEQ ID NO: 54 (3625 BCMA scFv, Vl-Vh). In some embodiments, the BCMA-binding domain comprises an amino acid sequence having at least 95% sequence identity with the amino acid sequence of SEQ ID NO: 54 (3625 BCMA scFv, Vl-Vh). In some embodiments, the BCMA-binding domain comprises an amino acid sequence having at least 96% sequence identity with the amino acid sequence of SEQ ID NO: 54 (3625 BCMA scFv, Vl-Vh). In some embodiments, the BCMA-binding domain comprises an amino acid sequence having at least 97% sequence identity with the amino acid sequence of SEQ ID NO: 54 (3625 BCMA scFv, Vl-Vh). In some embodiments, the BCMA-binding domain comprises an amino acid sequence having at least 98% sequence identity with the amino acid sequence of SEQ ID NO: 54 (3625 BCMA scFv, Vl-Vh). In some embodiments, the BCMA-binding domain comprises an amino acid sequence having at least 99% sequence identity with the amino acid sequence of SEQ ID NO: 54 (3625 BCMA scFv, Vl-Vh). In some embodiments, the BCMA-binding domain comprises the amino acid sequence of SEQ ID NO: 54 (3625 BCMA scFv, Vl-Vh).

In some embodiments, the CDR sequences of the BCMA-binding domain have 100% identity with the CDR sequences of the amino acid sequence of SEQ ID NO: 54 (3625 BCMA scFv, Vl-Vh) (i.e., the BCMA-binding domain comprises an amino acid sequence comprising a CDRH1, CDRH2, CDRH3, CDRL1, CDRL2, and CDRL3, each having 100% identity to the corresponding CDR in the amino acid sequence of SEQ ID NO: 54 (3625 BCMA scFv, Vl-Vh)). In some embodiments, the CDR sequences of the BCMA-binding domain have 100% identity with the CDR sequences of the amino acid sequence of SEQ ID NO: 54 (3625 BCMA scFv, Vl-Vh), and the non-CDR (e.g., framework) sequences of the BCMA-binding domain have at least 80% sequence identity with the non-CDR (e.g., framework) sequences of the amino acid sequence of SEQ ID NO: 54 (3625 BCMA scFv, Vl-Vh). In some embodiments, the CDR sequences of the BCMA-binding domain have 100% identity with the CDR sequences of the amino acid sequence of SEQ ID NO: 54 (3625 BCMA scFv, Vl-Vh), and the non-CDR (e.g., framework) sequences of the BCMA-binding domain have at least 85% sequence identity with the non-CDR (e.g., framework) sequences of the amino acid sequence of SEQ ID NO: 54 (3625 BCMA scFv, Vl-Vh). In some embodiments, the CDR sequences of the BCMA-binding domain have 100% identity with the CDR sequences of the amino acid sequence of SEQ ID NO: 54 (3625 BCMA scFv, Vl-Vh), and the non-CDR (e.g., framework) sequences of the BCMA-binding domain have at least 90% sequence identity with the non-CDR (e.g., framework) sequences of the amino acid sequence of SEQ ID NO: 54 (3625 BCMA scFv, Vl-Vh). In some embodiments, the CDR sequences of the BCMA-binding domain have 100% identity with the CDR sequences of the amino acid sequence of SEQ ID NO: 54 (3625 BCMA scFv, Vl-Vh), and the non-CDR (e.g., framework) sequences of the BCMA-binding domain have at least 95% sequence identity with the non-CDR (e.g., framework) sequences of the amino acid sequence of SEQ ID NO: 54 (3625 BCMA scFv, Vl-Vh). In some embodiments, the CDR sequences of the BCMA-binding domain have 100% identity with the CDR sequences of the amino acid sequence of SEQ ID NO: 54 (3625 BCMA scFv, Vl-Vh), and the non-CDR (e.g., framework) sequences of the BCMA-binding domain have at least 96% sequence identity with the non-CDR (e.g., framework) sequences of the amino acid sequence of SEQ ID NO: 54 (3625 BCMA scFv, Vl-Vh). In some embodiments, the CDR sequences of the BCMA-binding domain have 100% identity with the CDR sequences of the amino acid sequence of SEQ ID NO: 54 (3625 BCMA scFv, Vl-Vh), and the non-CDR (e.g., framework) sequences of the BCMA-binding domain have at least 97% sequence identity with the non-CDR (e.g., framework) sequences of the amino acid sequence of SEQ ID NO: 54 (3625 BCMA scFv, Vl-Vh). In some embodiments, the CDR sequences of the BCMA-binding domain have 100% identity with the CDR sequences of the amino acid sequence of SEQ ID NO: 54 (3625 BCMA scFv, Vl-Vh), and the non-CDR (e.g., framework) sequences of the BCMA-binding domain have at least 98% sequence identity with the non-CDR (e.g., framework) sequences of the amino acid sequence of SEQ ID NO: 54 (3625 BCMA scFv, Vl-Vh). In some embodiments, the CDR sequences of the BCMA-binding domain have 100% identity with the CDR sequences of the amino acid sequence of SEQ ID NO: 54 (3625 BCMA scFv, Vl-Vh), and the non-CDR (e.g., framework) sequences of the BCMA-binding domain have at least 99% sequence identity with the non-CDR (e.g., framework) sequences of the amino acid sequence of SEQ ID NO: 54 (3625 BCMA scFv, Vl-Vh).

In some embodiments, the tumor antigen is a CD19 antigen. In some embodiments, the target-specific antigen-binding domain is a scFv that binds CD19. In some embodiments, the target-specific antigen-binding domain is a scFv that specifically binds CD19. In some embodiments, the scFv that binds CD19 is encoded by the nucleotide sequence of SEQ ID NO: 35 or comprises the amino acid sequence of SEQ ID NO: 36.

In some embodiments, the polynucleotide encoding the CD19-binding domain comprises a nucleotide sequence having at least 70% sequence identity with the nucleotide sequence of SEQ ID NO: 35 (CD19 scFv). In some embodiments, the polynucleotide encoding the CD19-binding domain comprises a nucleotide sequence having at least 75% sequence identity with the nucleotide sequence of SEQ ID NO: 35 (CD19 scFv). In some embodiments, the polynucleotide encoding the CD19-binding domain comprises a nucleotide sequence having at least 80% sequence identity with the nucleotide sequence of SEQ ID NO: 35 (CD19 scFv). In some embodiments, the polynucleotide encoding the CD19-binding domain comprises a nucleotide sequence having at least 85% sequence identity with the nucleotide sequence of SEQ ID NO: 35 (CD19 scFv). In some embodiments, the polynucleotide encoding the CD19-binding domain comprises a nucleotide sequence having at least 90% sequence identity with the nucleotide sequence of SEQ ID NO: 35 (CD19 scFv). In some embodiments, the polynucleotide encoding the CD19-binding domain comprises a nucleotide sequence having at least 95% sequence identity with the nucleotide sequence of SEQ ID NO: 35 (CD19 scFv). In some embodiments, the polynucleotide encoding the CD19-binding domain comprises a nucleotide sequence having at least 96% sequence identity with the nucleotide sequence of SEQ ID NO: 35 (CD19 scFv). In some embodiments, the polynucleotide encoding the CD19-binding domain comprises a nucleotide sequence having at least 97% sequence identity with the nucleotide sequence of SEQ ID NO: 35 (CD19 scFv). In some embodiments, the polynucleotide encoding the CD19-binding domain comprises a nucleotide sequence having at least 98% sequence identity with the nucleotide sequence of SEQ ID NO: 35 (CD19 scFv). In some embodiments, the polynucleotide encoding the CD19-binding domain comprises a nucleotide sequence having at least 99% sequence identity with the nucleotide sequence of SEQ ID NO: 35 (CD19 scFv). In some embodiments, the polynucleotide encoding the CD19-binding domain comprises the nucleotide sequence of SEQ ID NO: 35 (CD19 scFv).

In some embodiments, the CD19-binding domain comprises an amino acid sequence having at least 70% sequence identity with the amino acid sequence of SEQ ID NO: 36 (CD19 scFv). In some embodiments, the CD19-binding domain comprises an amino acid sequence having at least 75% sequence identity with the amino acid sequence of SEQ ID NO: 36 (CD19 scFv). In some embodiments, the CD19-binding domain comprises an amino acid sequence having at least 80% sequence identity with the amino acid sequence of SEQ ID NO: 36 (CD19 scFv). In some embodiments, the CD19-binding domain comprises an amino acid sequence having at least 85% sequence identity with the amino acid sequence of SEQ ID NO: 36 (CD19 scFv). In some embodiments, the CD19-binding domain comprises an amino acid sequence having at least 90% sequence identity with the amino acid sequence of SEQ ID NO: 36 (CD19 scFv). In some embodiments, the CD19-binding domain comprises an amino acid sequence having at least 95% sequence identity with the amino acid sequence of SEQ ID NO: 36 (CD19 scFv). In some embodiments, the CD19-binding domain comprises an amino acid sequence having at least 96% sequence identity with the amino acid sequence of SEQ ID NO: 36 (CD19 scFv). In some embodiments, the CD19-binding domain comprises an amino acid sequence having at least 97% sequence identity with the amino acid sequence of SEQ ID NO: 36 (CD19 scFv). In some embodiments, the CD19-binding domain comprises an amino acid sequence having at least 98% sequence identity with the amino acid sequence of SEQ ID NO: 36 (CD19 scFv). In some embodiments, the CD19-binding domain comprises an amino acid sequence having at least 99% sequence identity with the amino acid sequence of SEQ ID NO: 36 (CD19 scFv). In some embodiments, the CD19-binding domain comprises the amino acid sequence of SEQ ID NO: 36 (CD19 scFv).

In some embodiments, the CDR sequences of the CD19-binding domain have 100% identity with the CDR sequences of the amino acid sequence of SEQ ID NO: 36 (CD19 scFv) (i.e., the CD19-binding domain comprises an amino acid sequence comprising a CDRH1, CDRH2, CDRH3, CDRL1, CDRL2, and CDRL3, each having 100% identity to the corresponding CDR in the amino acid sequence of SEQ ID NO: 36 (CD19 scFv)). In some embodiments, the CDR sequences of the CD19-binding domain have 100% identity with the CDR sequences of the amino acid sequence of SEQ ID NO: 36 (CD19 scFv), and the non-CDR (e.g., framework) sequences of the CD19-binding domain have at least 80% sequence identity with the non-CDR (e.g., framework) sequences of the amino acid sequence of SEQ ID NO: 36 (CD19 scFv). In some embodiments, the CDR sequences of the CD19-binding domain have 100% identity with the CDR sequences of the amino acid sequence of SEQ ID NO: 36 (CD19 scFv), and the non-CDR (e.g., framework) sequences of the CD19-binding domain have at least 85% sequence identity with the non-CDR (e.g., framework) sequences of the amino acid sequence of SEQ ID NO: 36 (CD19 scFv). In some embodiments, the CDR sequences of the CD19-binding domain have 100% identity with the CDR sequences of the amino acid sequence of SEQ ID NO: 36 (CD19 scFv), and the non-CDR (e.g., framework) sequences of the CD19-binding domain have at least 90% sequence identity with the non-CDR (e.g., framework) sequences of the amino acid sequence of SEQ ID NO: 36 (CD19 scFv). In some embodiments, the CDR sequences of the CD19-binding domain have 100% identity with the CDR sequences of the amino acid sequence of SEQ ID NO: 36 (CD19 scFv), and the non-CDR (e.g., framework) sequences of the CD19-binding domain have at least 95% sequence identity with the non-CDR (e.g., framework) sequences of the amino acid sequence of SEQ ID NO: 36 (CD19 scFv). In some embodiments, the CDR sequences of the CD19-binding domain have 100% identity with the CDR sequences of the amino acid sequence of SEQ ID NO: 36 (CD19 scFv), and the non-CDR (e.g., framework) sequences of the CD19-binding domain have at least 96% sequence identity with the non-CDR (e.g., framework) sequences of the amino acid sequence of SEQ ID NO: 36 (CD19 scFv). In some embodiments, the CDR sequences of the CD19-binding domain have 100% identity with the CDR sequences of the amino acid sequence of SEQ ID NO: 36 (CD19 scFv), and the non-CDR (e.g., framework) sequences of the CD19-binding domain have at least 97% sequence identity with the non-CDR (e.g., framework) sequences of the amino acid sequence of SEQ ID NO: 36 (CD19 scFv). In some embodiments, the CDR sequences of the CD19-binding domain have 100% identity with the CDR sequences of the amino acid sequence of SEQ ID NO: 36 (CD19 scFv), and the non-CDR (e.g., framework) sequences of the CD19-binding domain have at least 98% sequence identity with the non-CDR (e.g., framework) sequences of the amino acid sequence of SEQ ID NO: 36 (CD19 scFv). In some embodiments, the CDR sequences of the CD19-binding domain have 100% identity with the CDR sequences of the amino acid sequence of SEQ ID NO: 36 (CD19 scFv), and the non-CDR (e.g., framework) sequences of the CD19-binding domain have at least 99% sequence identity with the non-CDR (e.g., framework) sequences of the amino acid sequence of SEQ ID NO: 36 (CD19 scFv).

Amino acid and nucleotide sequences of exemplary antigen-binding domains that bind a target are provided in Table 1.

TABLE 1
Table of Sequences

| | | Nucleotide/ |
|---|---|---|
| SEQ ID NO | Description | Amino Acid |
| SEQ ID NO: 7 | DARPin specific for Her2 antigen | Nucleotide |
| SEQ ID NO: 8 | DARPin specific for Her2 antigen | Amino Acid |
| SEQ ID NO: 33 | ScFv specific for BCMA antigen | Nucleotide |
| SEQ ID NO: 34 | ScFv specific for BCMA antigen | Amino Acid |
| SEQ ID NO: 35 | ScFv specific for CD19 antigen | Nucleotide |
| SEQ ID NO: 36 | ScFv specific for CD19 antigen | Amino Acid |
| SEQ ID NO: 51 | 3625 scFv BCMA Vh-Vl | Nucleotide |
| SEQ ID NO: 52 | 3625 scFv BCMA Vh-Vl | Amino Acid |
| SEQ ID NO: 53 | 3625 scFv BCMA Vl-Vh | Nucleotide |
| SEQ ID NO: 54 | 3625 scFv BCMA Vl-Vh | Amino Acid |

Antigen-Binding Domain that Binds a TCR Complex

In certain embodiments, the TAC comprises an antigen-binding domain that binds a protein associated with the TCR complex. A “TCR complex protein antigen-binding domain,” also referred to as a “TCR complex antigen-binding domain,” “antigen-binding domain that binds the TCR complex,” or “antigen-binding domain that binds a protein associated with the TCR complex,” refers to any substance or molecule that binds, directly or indirectly, to a protein associated with a TCR complex. In some embodiments, the antigen-binding domain that binds a protein associated with a TCR complex binds to a protein of the TCR. In some embodiments, the antigen-binding domain that binds a protein associated with a TCR complex comprises a substance that specifically binds to a protein of the TCR.

In some embodiments, the TCR complex protein antigen-binding domain is selected from antibodies or fragments thereof, for example, single chain antibodies (e.g., single-chain fragment variable antibodies (scFvs)), single domain antibodies (e.g., heavy-chain-only antibodies (VHH), shark heavy-chain-only antibodies (VNAR)), nanobodies, diabodies, minibodies, Fab fragments, Fab′ fragments, F(ab′)₂ fragments, or Fv fragments that bind to a protein of the TCR. In some embodiments, the TCR complex protein antigen-binding domain is selected from ankyrin repeat proteins (DARPins), affibodies, adnectins, affilins, phylomers; fynomers, affimers, peptide aptamers, lectins, knottins, centyrins, anticalins, peptides, peptidomimetics, proteins, glycoproteins, or proteoglycans that bind to a protein of the TCR, or naturally occurring ligands for a protein of the TCR. In some embodiments, the TCR complex protein antigen-binding domain is a non-protein compound that binds to a protein of the TCR, including but not limited to carbohydrates, lipids, nucleic acids, or small molecules. In some embodiments, the TCR complex protein antigen-binding domain is a designed ankyrin repeat (DARPin) targeted to a protein of the TCR. In some embodiments, the TCR complex protein antigen-binding domain is a single-chain variable fragment (ScFv) targeted to a protein of the TCR. In some embodiments, the TCR complex protein antigen-binding domain is a nanobody targeted to a protein of the TCR.

Proteins associated with the TCR include, but are not limited, to the TCR alpha (α) chain, TCR beta (β) chain, TCR gamma (γ) chain, TCR delta (δ) chain, CD3γ chain, CD3δ chain and CD3ε chains. In some embodiments, an antigen-binding domain that binds a protein associated with the TCR complex is an antibody to the TCR alpha (α) chain, TCR beta (β) chain, TCR gamma (γ) chain, TCR delta (δ) chain, CD3γ chain, CD3δ chain and/or CD3ε chain. In some embodiments, the protein associated with a TCR complex is CD3. In some embodiments, the protein associated with a TCR complex is CD3ε. In some embodiments, the antigen-binding domain that binds CD3 is an antibody, for example, a single chain antibody, for example a single-chain variable fragment (scFv). Examples of CD3 antibodies, include, but are not limited to, UCHT1, OKT3, F6A, L2K, muromonab, otelixizumab, teplizumab, visilizumab, CD3-12, MEM-57, 4D10A6, CD3D, or TR66.

In some embodiments, the antigen-binding domain that binds the TCR complex is UCHT1, or a variant thereof. In some embodiments, the UCHT1 antigen-binding domain is encoded by SEQ ID NO: 13. In some embodiments, the UCHT1 antigen-binding domain comprises SEQ ID NO: 14. In some embodiments, the UCHT1 antigen-binding domain is mutated. In some embodiments, the UCHT1 antigen-binding domain comprises a Y to T mutation at a position corresponding to amino acid 182 of SEQ ID NO: 14 (Y182T). In some embodiments, the UCHT1 (Y182T) antigen-binding domain is encoded by SEQ ID NO: 71. In some embodiments, the UCHT1 (Y182T) antigen-binding domain comprises SEQ ID NO: 72. In some embodiments, the antigen-binding domain that binds the TCR complex is a humanized UCHT1 (huUCHT1). In some embodiments, the huUCHT1 antigen-binding domain is encoded by SEQ ID NO: 43. In some embodiments, the huUCHT1 antigen-binding domain comprises SEQ ID NO: 44. In some embodiments, the huUCHT1 has a Y to T mutation at a position corresponding to amino acid 177 of SEQ ID NO: 44 (Y177T). In some embodiments, the huUCHT1 (Y177T) antigen-binding domain is encoded by SEQ ID NO: 45. In some embodiments, the huUCHT1 antigen-binding domain comprises SEQ ID NO: 46.

In some embodiments, the polynucleotide encoding the antigen-binding domain that binds the protein associated with the TCR complex comprises a nucleotide sequence having at least 70% sequence identity with the nucleotide sequence of SEQ ID NO: 13 (UCHT1). In some embodiments, the polynucleotide encoding the antigen-binding domain that binds the protein associated with the TCR complex comprises a nucleotide sequence having at least 75% sequence identity with the nucleotide sequence of SEQ ID NO: 13 (UCHT1). In some embodiments, the polynucleotide encoding the antigen-binding domain that binds the protein associated with the TCR complex comprises a nucleotide sequence having at least 80% sequence identity with the nucleotide sequence of SEQ ID NO: 13 (UCHT1). In some embodiments, the polynucleotide encoding the antigen-binding domain that binds the protein associated with the TCR complex comprises a nucleotide sequence having at least 85% sequence identity with the nucleotide sequence of SEQ ID NO: 13 (UCHT1). In some embodiments, the polynucleotide encoding the antigen-binding domain that binds the protein associated with the TCR complex comprises a nucleotide sequence having at least 90% sequence identity with the nucleotide sequence of SEQ ID NO: 13 (UCHT1). In some embodiments, the polynucleotide encoding the antigen-binding domain that binds the protein associated with the TCR complex comprises a nucleotide sequence having at least 95% sequence identity with the nucleotide sequence of SEQ ID NO: 13 (UCHT1). In some embodiments, the polynucleotide encoding the antigen-binding domain that binds the protein associated with the TCR complex comprises a nucleotide sequence having at least 96% sequence identity with the nucleotide sequence of SEQ ID NO: 13 (UCHT1). In some embodiments, the polynucleotide encoding the antigen-binding domain that binds the protein associated with the TCR complex comprises a nucleotide sequence having at least 97% sequence identity with the nucleotide sequence of SEQ ID NO: 13 (UCHT1). In some embodiments, the polynucleotide encoding the antigen-binding domain that binds the protein associated with the TCR complex comprises a nucleotide sequence having at least 98% sequence identity with the nucleotide sequence of SEQ ID NO: 13 (UCHT1). In some embodiments, the polynucleotide encoding the antigen-binding domain that binds the protein associated with the TCR complex comprises a nucleotide sequence having at least 99% sequence identity with the nucleotide sequence of SEQ ID NO: 13 (UCHT1). In some embodiments, the polynucleotide encoding the antigen-binding domain that binds the protein associated with the TCR complex comprises the nucleotide sequence of SEQ ID NO: 13 (UCHT1).

In some embodiments, the antigen-binding domain that binds the protein associated with the TCR complex comprises an amino acid sequence having at least 70% sequence identity with the amino acid sequence of SEQ ID NO: 14 (UCHT1). In some embodiments, the antigen-binding domain that binds the protein associated with the TCR complex comprises an amino acid sequence having at least 75% sequence identity with the amino acid sequence of SEQ ID NO: 14 (UCHT1). In some embodiments, the antigen-binding domain that binds the protein associated with the TCR complex comprises an amino acid sequence having at least 80% sequence identity with the amino acid sequence of SEQ ID NO: 14 (UCHT1). In some embodiments, the antigen-binding domain that binds the protein associated with the TCR complex comprises an amino acid sequence having at least 85% sequence identity with the amino acid sequence of SEQ ID NO: 14 (UCHT1). In some embodiments, the antigen-binding domain that binds the protein associated with the TCR complex comprises an amino acid sequence having at least 90% sequence identity with the amino acid sequence of SEQ ID NO: 14 (UCHT1). In some embodiments, the antigen-binding domain that binds the protein associated with the TCR complex comprises an amino acid sequence having at least 95% sequence identity with the amino acid sequence of SEQ ID NO: 14 (UCHT1). In some embodiments, the antigen-binding domain that binds the protein associated with the TCR complex comprises an amino acid sequence having at least 96% sequence identity with the amino acid sequence of SEQ ID NO: 14 (UCHT1). In some embodiments, the antigen-binding domain that binds the protein associated with the TCR complex comprises an amino acid sequence having at least 97% sequence identity with the amino acid sequence of SEQ ID NO: 14 (UCHT1). In some embodiments, the antigen-binding domain that binds the protein associated with the TCR complex comprises an amino acid sequence having at least 98% sequence identity with the amino acid sequence of SEQ ID NO: 14 (UCHT1). In some embodiments, the antigen-binding domain that binds the protein associated with the TCR complex comprises an amino acid sequence having at least 99% sequence identity with the amino acid sequence of SEQ ID NO: 14 (UCHT1). In some embodiments, the antigen-binding domain that binds the protein associated with the TCR complex comprises the amino acid sequence of SEQ ID NO: 14 (UCHT1). In some embodiments, the CDR sequences of the antigen-binding domain that binds the protein associated with the TCR complex have 100% identity with the CDR sequences of the amino acid sequence of SEQ ID NO: 14 (UCHT1) (i.e., the antigen-binding domain that binds the protein associated with the TCR complex comprises an amino acid sequence comprising a CDRH1, CDRH2, CDRH3, CDRL1, CDRL2, and CDRL3, each having 100% identity to the corresponding CDR in the amino acid sequence of SEQ ID NO: 14 (UCHT1). In some embodiments, the CDR sequences of the antigen-binding domain that binds the protein associated with the TCR complex have 100% identity with the CDR sequences of the amino acid sequence of SEQ ID NO: 14 (UCHT1), and the non-CDR (e.g., framework) sequences of the antigen-binding domain that binds the protein associated with the TCR complex have at least 80% sequence identity with the non-CDR (e.g., framework) sequences of the amino acid sequence of SEQ ID NO: 14 (UCHT1). In some embodiments, the CDR sequences of the antigen-binding domain that binds the protein associated with the TCR complex have 100% identity with the CDR sequences of the amino acid sequence of SEQ ID NO: 14 (UCHT1), and the non-CDR (e.g., framework) sequences of the antigen-binding domain that binds the protein associated with the TCR complex have at least 85% sequence identity with the non-CDR (e.g., framework) sequences of the amino acid sequence of SEQ ID NO: 14 (UCHT1). In some embodiments, the CDR sequences of the antigen-binding domain that binds the protein associated with the TCR complex have 100% identity with the CDR sequences of the amino acid sequence of SEQ ID NO: 14 (UCHT1), and the non-CDR (e.g., framework) sequences of the antigen-binding domain that binds the protein associated with the TCR complex have at least 90% sequence identity with the non-CDR (e.g., framework) sequences of the amino acid sequence of SEQ ID NO: 14 (UCHT1). In some embodiments, the CDR sequences of the antigen-binding domain that binds the protein associated with the TCR complex have 100% identity with the CDR sequences of the amino acid sequence of SEQ ID NO: 14 (UCHT1), and the non-CDR (e.g., framework) sequences of the antigen-binding domain that binds the protein associated with the TCR complex have at least 95% sequence identity with the non-CDR (e.g., framework) sequences of the amino acid sequence of SEQ ID NO: 14 (UCHT1). In some embodiments, the CDR sequences of the antigen-binding domain that binds the protein associated with the TCR complex have 100% identity with the CDR sequences of the amino acid sequence of SEQ ID NO: 14 (UCHT1), and the non-CDR (e.g., framework) sequences of the antigen-binding domain that binds the protein associated with the TCR complex have at least 96% sequence identity with the non-CDR (e.g., framework) sequences of the amino acid sequence of SEQ ID NO: 14 (UCHT1). In some embodiments, the CDR sequences of the antigen-binding domain that binds the protein associated with the TCR complex have 100% identity with the CDR sequences of the amino acid sequence of SEQ ID NO: 14 (UCHT1), and the non-CDR (e.g., framework) sequences of the antigen-binding domain that binds the protein associated with the TCR complex have at least 97% sequence identity with the non-CDR (e.g., framework) sequences of the amino acid sequence of SEQ ID NO: 14 (UCHT1). In some embodiments, the CDR sequences of the antigen-binding domain that binds the protein associated with the TCR complex have 100% identity with the CDR sequences of the amino acid sequence of SEQ ID NO: 14 (UCHT1), and the non-CDR (e.g., framework) sequences of the antigen-binding domain that binds the protein associated with the TCR complex have at least 98% sequence identity with the non-CDR (e.g., framework) sequences of the amino acid sequence of SEQ ID NO: 14 (UCHT1). In some embodiments, the CDR sequences of the antigen-binding domain that binds the protein associated with the TCR complex have 100% identity with the CDR sequences of the amino acid sequence of SEQ ID NO: 14 (UCHT1), and the non-CDR (e.g., framework) sequences of the antigen-binding domain that binds the protein associated with the TCR complex have at least 99% sequence identity with the non-CDR (e.g., framework) sequences of the amino acid sequence of SEQ ID NO: 14 (UCHT1).

In some embodiments, the polynucleotide encoding the antigen-binding domain that binds the protein associated with the TCR complex comprises a nucleotide sequence having at least 70% sequence identity with the nucleotide sequence of SEQ ID NO: 71 (UCHT1 (Y182T)). In some embodiments, the polynucleotide encoding the antigen-binding domain that binds the protein associated with the TCR complex comprises a nucleotide sequence having at least 75% sequence identity with the nucleotide sequence of SEQ ID NO: 71 (UCHT1 (Y182T)). In some embodiments, the polynucleotide encoding the antigen-binding domain that binds the protein associated with the TCR complex comprises a nucleotide sequence having at least 80% sequence identity with the nucleotide sequence of SEQ ID NO: 71 (UCHT1 (Y182T)). In some embodiments, the polynucleotide encoding the antigen-binding domain that binds the protein associated with the TCR complex comprises a nucleotide sequence having at least 85% sequence identity with the nucleotide sequence of SEQ ID NO: 71 (UCHT1 (Y182T)). In some embodiments, the polynucleotide encoding the antigen-binding domain that binds the protein associated with the TCR complex comprises a nucleotide sequence having at least 90% sequence identity with the nucleotide sequence of SEQ ID NO: 71 (UCHT1 (Y182T)). In some embodiments, the polynucleotide encoding the antigen-binding domain that binds the protein associated with the TCR complex comprises a nucleotide sequence having at least 95% sequence identity with the nucleotide sequence of SEQ ID NO: 71 (UCHT1 (Y182T)). In some embodiments, the polynucleotide encoding the antigen-binding domain that binds the protein associated with the TCR complex comprises a nucleotide sequence having at least 96% sequence identity with the nucleotide sequence of SEQ ID NO: 71 (UCHT1 (Y182T)). In some embodiments, the polynucleotide encoding the antigen-binding domain that binds the protein associated with the TCR complex comprises a nucleotide sequence having at least 97% sequence identity with the nucleotide sequence of SEQ ID NO: 71 (UCHT1 (Y182T)). In some embodiments, the polynucleotide encoding the antigen-binding domain that binds the protein associated with the TCR complex comprises a nucleotide sequence having at least 98% sequence identity with the nucleotide sequence of SEQ ID NO: 71 (UCHT1 (Y182T)). In some embodiments, the polynucleotide encoding the antigen-binding domain that binds the protein associated with the TCR complex comprises a nucleotide sequence having at least 99% sequence identity with the nucleotide sequence of SEQ ID NO: 71 (UCHT1 (Y182T)). In some embodiments, the polynucleotide encoding the antigen-binding domain that binds the protein associated with the TCR complex comprises the nucleotide sequence of SEQ ID NO: 71 (UCHT1 (Y182T)).

In some embodiments, the antigen-binding domain that binds the protein associated with the TCR complex comprises an amino acid sequence having at least 70% sequence identity with the amino acid sequence of SEQ ID NO: 72 (UCHT1 (Y182T)). In some embodiments, the antigen-binding domain that binds the protein associated with the TCR complex comprises an amino acid sequence having at least 75% sequence identity with the amino acid sequence of SEQ ID NO: 72 (UCHT1 (Y182T)). In some embodiments, the antigen-binding domain that binds the protein associated with the TCR complex comprises an amino acid sequence having at least 80% sequence identity with the amino acid sequence of SEQ ID NO: 72 (UCHT1 (Y182T)). In some embodiments, the antigen-binding domain that binds the protein associated with the TCR complex comprises an amino acid sequence having at least 85% sequence identity with the amino acid sequence of SEQ ID NO: 72 (UCHT1 (Y182T)). In some embodiments, the antigen-binding domain that binds the protein associated with the TCR complex comprises an amino acid sequence having at least 90% sequence identity with the amino acid sequence of SEQ ID NO: 72 (UCHT1 (Y182T)). In some embodiments, the antigen-binding domain that binds the protein associated with the TCR complex comprises an amino acid sequence having at least 95% sequence identity with the amino acid sequence of SEQ ID NO: 72 (UCHT1 (Y182T)). In some embodiments, the antigen-binding domain that binds the protein associated with the TCR complex comprises an amino acid sequence having at least 96% sequence identity with the amino acid sequence of SEQ ID NO: 72 (UCHT1 (Y182T)). In some embodiments, the antigen-binding domain that binds the protein associated with the TCR complex comprises an amino acid sequence having at least 97% sequence identity with the amino acid sequence of SEQ ID NO: 72 (UCHT1 (Y182T)). In some embodiments, the antigen-binding domain that binds the protein associated with the TCR complex comprises an amino acid sequence having at least 98% sequence identity with the amino acid sequence of SEQ ID NO: 72 (UCHT1 (Y182T)). In some embodiments, the antigen-binding domain that binds the protein associated with the TCR complex comprises an amino acid sequence having at least 99% sequence identity with the amino acid sequence of SEQ ID NO: 72 (UCHT1 (Y182T)). In some embodiments, the antigen-binding domain that binds the protein associated with the TCR complex comprises the amino acid sequence of SEQ ID NO: 72 (UCHT1 (Y182T)). In some embodiments, the CDR sequences of the antigen-binding domain that binds the protein associated with the TCR complex have 100% identity with the CDR sequences of the amino acid sequence of SEQ ID NO: 72 (UCHT1 (Y182T)) (i.e., the antigen-binding domain that binds the protein associated with the TCR complex comprises an amino acid sequence comprising a CDRH1, CDRH2, CDRH3, CDRL1, CDRL2, and CDRL3, each having 100% identity to the corresponding CDR in the amino acid sequence of SEQ ID NO: 72 (UCHT1 (Y182T)). In some embodiments, the CDR sequences of the antigen-binding domain that binds the protein associated with the TCR complex have 100% identity with the CDR sequences of the amino acid sequence of SEQ ID NO: 72 (UCHT1 (Y182T)), and the non-CDR (e.g., framework) sequences of the antigen-binding domain that binds the protein associated with the TCR complex have at least 80% sequence identity with the non-CDR (e.g., framework) sequences of the amino acid sequence of SEQ ID NO: 72 (UCHT1 (Y182T)). In some embodiments, the CDR sequences of the antigen-binding domain that binds the protein associated with the TCR complex have 100% identity with the CDR sequences of the amino acid sequence of SEQ ID NO: 72 (UCHT1 (Y182T)), and the non-CDR (e.g., framework) sequences of the antigen-binding domain that binds the protein associated with the TCR complex have at least 85% sequence identity with the non-CDR (e.g., framework) sequences of the amino acid sequence of SEQ ID NO: 72 (UCHT1 (Y182T)). In some embodiments, the CDR sequences of the antigen-binding domain that binds the protein associated with the TCR complex have 100% identity with the CDR sequences of the amino acid sequence of SEQ ID NO: 72 (UCHT1 (Y182T)), and the non-CDR (e.g., framework) sequences of the antigen-binding domain that binds the protein associated with the TCR complex have at least 90% sequence identity with the non-CDR (e.g., framework) sequences of the amino acid sequence of SEQ ID NO: 72 (UCHT1 (Y182T)). In some embodiments, the CDR sequences of the antigen-binding domain that binds the protein associated with the TCR complex have 100% identity with the CDR sequences of the amino acid sequence of SEQ ID NO: 72 (UCHT1 (Y182T)), and the non-CDR (e.g., framework) sequences of the antigen-binding domain that binds the protein associated with the TCR complex have at least 95% sequence identity with the non-CDR (e.g., framework) sequences of the amino acid sequence of SEQ ID NO: 72 (UCHT1 (Y182T)). In some embodiments, the CDR sequences of the antigen-binding domain that binds the protein associated with the TCR complex have 100% identity with the CDR sequences of the amino acid sequence of SEQ ID NO: 72 (UCHT1 (Y182T)), and the non-CDR (e.g., framework) sequences of the antigen-binding domain that binds the protein associated with the TCR complex have at least 96% sequence identity with the non-CDR (e.g., framework) sequences of the amino acid sequence of SEQ ID NO: 72 (UCHT1 (Y182T)). In some embodiments, the CDR sequences of the antigen-binding domain that binds the protein associated with the TCR complex have 100% identity with the CDR sequences of the amino acid sequence of SEQ ID NO: 72 (UCHT1 (Y182T)), and the non-CDR (e.g., framework) sequences of the antigen-binding domain that binds the protein associated with the TCR complex have at least 97% sequence identity with the non-CDR (e.g., framework) sequences of the amino acid sequence of SEQ ID NO: 72 (UCHT1 (Y182T)). In some embodiments, the CDR sequences of the antigen-binding domain that binds the protein associated with the TCR complex have 100% identity with the CDR sequences of the amino acid sequence of SEQ ID NO: 72 (UCHT1 (Y182T)), and the non-CDR (e.g., framework) sequences of the antigen-binding domain that binds the protein associated with the TCR complex have at least 98% sequence identity with the non-CDR (e.g., framework) sequences of the amino acid sequence of SEQ ID NO: 72 (UCHT1 (Y182T)). In some embodiments, the CDR sequences of the antigen-binding domain that binds the protein associated with the TCR complex have 100% identity with the CDR sequences of the amino acid sequence of SEQ ID NO: 72 (UCHT1 (Y182T)), and the non-CDR (e.g., framework) sequences of the antigen-binding domain that binds the protein associated with the TCR complex have at least 99% sequence identity with the non-CDR (e.g., framework) sequences of the amino acid sequence of SEQ ID NO: 72 (UCHT1 (Y182T)).

In some embodiments, the polynucleotide encoding the antigen-binding domain that binds the protein associated with the TCR complex comprises a nucleotide sequence having at least 70% sequence identity with the nucleotide sequence of SEQ ID NO: 43 (huUCHT1). In some embodiments, the polynucleotide encoding the antigen-binding domain that binds the protein associated with the TCR complex comprises a nucleotide sequence having at least 75% sequence identity with the nucleotide sequence of SEQ ID NO: 43 (huUCHT1). In some embodiments, the polynucleotide encoding the antigen-binding domain that binds the protein associated with the TCR complex comprises a nucleotide sequence having at least 80% sequence identity with the nucleotide sequence of SEQ ID NO: 43 (huUCHT1). In some embodiments, the polynucleotide encoding the antigen-binding domain that binds the protein associated with the TCR complex comprises a nucleotide sequence having at least 85% sequence identity with the nucleotide sequence of SEQ ID NO: 43 (huUCHT1). In some embodiments, the polynucleotide encoding the antigen-binding domain that binds the protein associated with the TCR complex comprises a nucleotide sequence having at least 90% sequence identity with the nucleotide sequence of SEQ ID NO: 43 (huUCHT1). In some embodiments, the polynucleotide encoding the antigen-binding domain that binds the protein associated with the TCR complex comprises a nucleotide sequence having at least 95% sequence identity with the nucleotide sequence of SEQ ID NO: 43 (huUCHT1). In some embodiments, the polynucleotide encoding the antigen-binding domain that binds the protein associated with the TCR complex comprises a nucleotide sequence having at least 96% sequence identity with the nucleotide sequence of SEQ ID NO: 43 (huUCHT1). In some embodiments, the polynucleotide encoding the antigen-binding domain that binds the protein associated with the TCR complex comprises a nucleotide sequence having at least 97% sequence identity with the nucleotide sequence of SEQ ID NO: 43 (huUCHT1). In some embodiments, the polynucleotide encoding the antigen-binding domain that binds the protein associated with the TCR complex comprises a nucleotide sequence having at least 98% sequence identity with the nucleotide sequence of SEQ ID NO: 43 (huUCHT1). In some embodiments, the polynucleotide encoding the antigen-binding domain that binds the protein associated with the TCR complex comprises a nucleotide sequence having at least 99% sequence identity with the nucleotide sequence of SEQ ID NO: 43 (huUCHT1). In some embodiments, the polynucleotide encoding the antigen-binding domain that binds the protein associated with the TCR complex comprises the nucleotide sequence of SEQ ID NO: 43 (huUCHT1).

In some embodiments, the antigen-binding domain that binds the protein associated with the TCR complex comprises an amino acid sequence having at least 70% sequence identity with the amino acid sequence of SEQ ID NO: 44 (huUCHT1). In some embodiments, the antigen-binding domain that binds the protein associated with the TCR complex comprises an amino acid sequence having at least 75% sequence identity with the amino acid sequence of SEQ ID NO: 44 (huUCHT1). In some embodiments, the antigen-binding domain that binds the protein associated with the TCR complex comprises an amino acid sequence having at least 80% sequence identity with the amino acid sequence of SEQ ID NO: 44 (huUCHT1). In some embodiments, the antigen-binding domain that binds the protein associated with the TCR complex comprises an amino acid sequence having at least 85% sequence identity with the amino acid sequence of SEQ ID NO: 44 (huUCHT1). In some embodiments, the antigen-binding domain that binds the protein associated with the TCR complex comprises an amino acid sequence having at least 90% sequence identity with the amino acid sequence of SEQ ID NO: 44 (huUCHT1). In some embodiments, the antigen-binding domain that binds the protein associated with the TCR complex comprises an amino acid sequence having at least 95% sequence identity with the amino acid sequence of SEQ ID NO: 44 (huUCHT1). In some embodiments, the antigen-binding domain that binds the protein associated with the TCR complex comprises an amino acid sequence having at least 96% sequence identity with the amino acid sequence of SEQ ID NO: 44 (huUCHT1). In some embodiments, the antigen-binding domain that binds the protein associated with the TCR complex comprises an amino acid sequence having at least 97% sequence identity with the amino acid sequence of SEQ ID NO: 44 (huUCHT1). In some embodiments, the antigen-binding domain that binds the protein associated with the TCR complex comprises an amino acid sequence having at least 98% sequence identity with the amino acid sequence of SEQ ID NO: 44 (huUCHT1). In some embodiments, the antigen-binding domain that binds the protein associated with the TCR complex comprises an amino acid sequence having at least 99% sequence identity with the amino acid sequence of SEQ ID NO: 44 (huUCHT1). In some embodiments, the antigen-binding domain that binds the protein associated with the TCR complex comprises the amino acid sequence of SEQ ID NO: 44 (huUCHT1). In some embodiments, the CDR sequences of the antigen-binding domain that binds the protein associated with the TCR complex have 100% identity with the CDR sequences of the amino acid sequence of SEQ ID NO: 44 (huUCHT1) (i.e., the antigen-binding domain that binds the protein associated with the TCR complex comprises an amino acid sequence comprising a CDRH1, CDRH2, CDRH3, CDRL1, CDRL2, and CDRL3, each having 100% identity to the corresponding CDR in the amino acid sequence of SEQ ID NO: 44 (huUCHT1). In some embodiments, the CDR sequences of the antigen-binding domain that binds the protein associated with the TCR complex have 100% identity with the CDR sequences of the amino acid sequence of SEQ ID NO: 44 (huUCHT1), and the non-CDR (e.g., framework) sequences of the antigen-binding domain that binds the protein associated with the TCR complex have at least 80% sequence identity with the non-CDR (e.g., framework) sequences of the amino acid sequence of SEQ ID NO: 44 (huUCHT1). In some embodiments, the CDR sequences of the antigen-binding domain that binds the protein associated with the TCR complex have 100% identity with the CDR sequences of the amino acid sequence of SEQ ID NO: 44 (huUCHT1), and the non-CDR (e.g., framework) sequences of the antigen-binding domain that binds the protein associated with the TCR complex have at least 85% sequence identity with the non-CDR (e.g., framework) sequences of the amino acid sequence of SEQ ID NO: 44 (huUCHT1). In some embodiments, the CDR sequences of the antigen-binding domain that binds the protein associated with the TCR complex have 100% identity with the CDR sequences of the amino acid sequence of SEQ ID NO: 44 (huUCHT1), and the non-CDR (e.g., framework) sequences of the antigen-binding domain that binds the protein associated with the TCR complex have at least 90% sequence identity with the non-CDR (e.g., framework) sequences of the amino acid sequence of SEQ ID NO: 44 (huUCHT1). In some embodiments, the CDR sequences of the antigen-binding domain that binds the protein associated with the TCR complex have 100% identity with the CDR sequences of the amino acid sequence of SEQ ID NO: 44 (huUCHT1), and the non-CDR (e.g., framework) sequences of the antigen-binding domain that binds the protein associated with the TCR complex have at least 95% sequence identity with the non-CDR (e.g., framework) sequences of the amino acid sequence of SEQ ID NO: 44 (huUCHT1). In some embodiments, the CDR sequences of the antigen-binding domain that binds the protein associated with the TCR complex have 100% identity with the CDR sequences of the amino acid sequence of SEQ ID NO: 44 (huUCHT1), and the non-CDR (e.g., framework) sequences of the antigen-binding domain that binds the protein associated with the TCR complex have at least 96% sequence identity with the non-CDR (e.g., framework) sequences of the amino acid sequence of SEQ ID NO: 44 (huUCHT1). In some embodiments, the CDR sequences of the antigen-binding domain that binds the protein associated with the TCR complex have 100% identity with the CDR sequences of the amino acid sequence of SEQ ID NO: 44 (huUCHT1), and the non-CDR (e.g., framework) sequences of the antigen-binding domain that binds the protein associated with the TCR complex have at least 97% sequence identity with the non-CDR (e.g., framework) sequences of the amino acid sequence of SEQ ID NO: 44 (huUCHT1). In some embodiments, the CDR sequences of the antigen-binding domain that binds the protein associated with the TCR complex have 100% identity with the CDR sequences of the amino acid sequence of SEQ ID NO: 44 (huUCHT1), and the non-CDR (e.g., framework) sequences of the antigen-binding domain that binds the protein associated with the TCR complex have at least 98% sequence identity with the non-CDR (e.g., framework) sequences of the amino acid sequence of SEQ ID NO: 44 (huUCHT1). In some embodiments, the CDR sequences of the antigen-binding domain that binds the protein associated with the TCR complex have 100% identity with the CDR sequences of the amino acid sequence of SEQ ID NO: 44 (huUCHT1), and the non-CDR (e.g., framework) sequences of the antigen-binding domain that binds the protein associated with the TCR complex have at least 99% sequence identity with the non-CDR (e.g., framework) sequences of the amino acid sequence of SEQ ID NO: 44 (huUCHT1).

In some embodiments, the polynucleotide encoding the antigen-binding domain that binds the protein associated with the TCR complex comprises a nucleotide sequence having at least 70% sequence identity with the nucleotide sequence of SEQ ID NO: 45 (huUCHT1 (Y177T)). In some embodiments, the polynucleotide encoding the antigen-binding domain that binds the protein associated with the TCR complex comprises a nucleotide sequence having at least 75% sequence identity with the nucleotide sequence of SEQ ID NO: 45 (huUCHT1 (Y177T)). In some embodiments, the polynucleotide encoding the antigen-binding domain that binds the protein associated with the TCR complex comprises a nucleotide sequence having at least 80% sequence identity with the nucleotide sequence of SEQ ID NO: 45 (huUCHT1 (Y177T)). In some embodiments, the polynucleotide encoding the antigen-binding domain that binds the protein associated with the TCR complex comprises a nucleotide sequence having at least 85% sequence identity with the nucleotide sequence of SEQ ID NO: 45 (huUCHT1 (Y177T)). In some embodiments, the polynucleotide encoding the antigen-binding domain that binds the protein associated with the TCR complex comprises a nucleotide sequence having at least 90% sequence identity with the nucleotide sequence of SEQ ID NO: 45 (huUCHT1 (Y177T)). In some embodiments, the polynucleotide encoding the antigen-binding domain that binds the protein associated with the TCR complex comprises a nucleotide sequence having at least 95% sequence identity with the nucleotide sequence of SEQ ID NO: 45 (huUCHT1 (Y177T)). In some embodiments, the polynucleotide encoding the antigen-binding domain that binds the protein associated with the TCR complex comprises a nucleotide sequence having at least 96% sequence identity with the nucleotide sequence of SEQ ID NO: 45 (huUCHT1 (Y177T)). In some embodiments, the polynucleotide encoding the antigen-binding domain that binds the protein associated with the TCR complex comprises a nucleotide sequence having at least 97% sequence identity with the nucleotide sequence of SEQ ID NO: 45 (huUCHT1 (Y177T)). In some embodiments, the polynucleotide encoding the antigen-binding domain that binds the protein associated with the TCR complex comprises a nucleotide sequence having at least 98% sequence identity with the nucleotide sequence of SEQ ID NO: 45 (huUCHT1 (Y177T)). In some embodiments, the polynucleotide encoding the antigen-binding domain that binds the protein associated with the TCR complex comprises a nucleotide sequence having at least 99% sequence identity with the nucleotide sequence of SEQ ID NO: 45 (huUCHT1 (Y177T)). In some embodiments, the polynucleotide encoding the antigen-binding domain that binds the protein associated with the TCR complex comprises the nucleotide sequence of SEQ ID NO: 45 (huUCHT1 (Y177T)).

In some embodiments, the antigen-binding domain that binds the protein associated with the TCR complex comprises an amino acid sequence having at least 70% sequence identity with the amino acid sequence of SEQ ID NO: 46 (huUCHT1 (Y177T)). In some embodiments, the antigen-binding domain that binds the protein associated with the TCR complex comprises an amino acid sequence having at least 75% sequence identity with the amino acid sequence of SEQ ID NO: 46 (huUCHT1 (Y177T)). In some embodiments, the antigen-binding domain that binds the protein associated with the TCR complex comprises an amino acid sequence having at least 80% sequence identity with the amino acid sequence of SEQ ID NO: 46 (huUCHT1 (Y177T)). In some embodiments, the antigen-binding domain that binds the protein associated with the TCR complex comprises an amino acid sequence having at least 85% sequence identity with the amino acid sequence of SEQ ID NO: 46 (huUCHT1 (Y177T)). In some embodiments, the antigen-binding domain that binds the protein associated with the TCR complex comprises an amino acid sequence having at least 90% sequence identity with the amino acid sequence of SEQ ID NO: 46 (huUCHT1 (Y177T)). In some embodiments, the antigen-binding domain that binds the protein associated with the TCR complex comprises an amino acid sequence having at least 95% sequence identity with the amino acid sequence of SEQ ID NO: 46 (huUCHT1 (Y177T)). In some embodiments, the antigen-binding domain that binds the protein associated with the TCR complex comprises an amino acid sequence having at least 96% sequence identity with the amino acid sequence of SEQ ID NO: 46 (huUCHT1 (Y177T)). In some embodiments, the antigen-binding domain that binds the protein associated with the TCR complex comprises an amino acid sequence having at least 97% sequence identity with the amino acid sequence of SEQ ID NO: 46 (huUCHT1 (Y177T)). In some embodiments, the antigen-binding domain that binds the protein associated with the TCR complex comprises an amino acid sequence having at least 98% sequence identity with the amino acid sequence of SEQ ID NO: 46 (huUCHT1 (Y177T)). In some embodiments, the antigen-binding domain that binds the protein associated with the TCR complex comprises an amino acid sequence having at least 99% sequence identity with the amino acid sequence of SEQ ID NO: 46 (huUCHT1 (Y177T)). In some embodiments, the antigen-binding domain that binds the protein associated with the TCR complex comprises the amino acid sequence of SEQ ID NO: 46 (huUCHT1 (Y177T)). In some embodiments, the CDR sequences of the antigen-binding domain that binds the protein associated with the TCR complex have 100% identity with the CDR sequences of the amino acid sequence of SEQ ID NO: 46 (huUCHT1 (Y177T)) (i.e., the antigen-binding domain that binds the protein associated with the TCR complex comprises an amino acid sequence comprising a CDRH1, CDRH2, CDRH3, CDRL1, CDRL2, and CDRL3, each having 100% identity to the corresponding CDR in the amino acid sequence of SEQ ID NO: 46 (huUCHT1 (Y177T)). In some embodiments, the CDR sequences of the antigen-binding domain that binds the protein associated with the TCR complex have 100% identity with the CDR sequences of the amino acid sequence of SEQ ID NO: 46 (huUCHT1 (Y177T)), and the non-CDR (e.g., framework) sequences of the antigen-binding domain that binds the protein associated with the TCR complex have at least 80% sequence identity with the non-CDR (e.g., framework) sequences of the amino acid sequence of SEQ ID NO: 46 (huUCHT1 (Y177T)). In some embodiments, the CDR sequences of the antigen-binding domain that binds the protein associated with the TCR complex have 100% identity with the CDR sequences of the amino acid sequence of SEQ ID NO: 46 (huUCHT1 (Y177T)), and the non-CDR (e.g., framework) sequences of the antigen-binding domain that binds the protein associated with the TCR complex have at least 85% sequence identity with the non-CDR (e.g., framework) sequences of the amino acid sequence of SEQ ID NO: 46 (huUCHT1 (Y177T)). In some embodiments, the CDR sequences of the antigen-binding domain that binds the protein associated with the TCR complex have 100% identity with the CDR sequences of the amino acid sequence of SEQ ID NO: 46 (huUCHT1 (Y177T)), and the non-CDR (e.g., framework) sequences of the antigen-binding domain that binds the protein associated with the TCR complex have at least 90% sequence identity with the non-CDR (e.g., framework) sequences of the amino acid sequence of SEQ ID NO: 46 (huUCHT1 (Y177T)). In some embodiments, the CDR sequences of the antigen-binding domain that binds the protein associated with the TCR complex have 100% identity with the CDR sequences of the amino acid sequence of SEQ ID NO: 46 (huUCHT1 (Y177T)), and the non-CDR (e.g., framework) sequences of the antigen-binding domain that binds the protein associated with the TCR complex have at least 95% sequence identity with the non-CDR (e.g., framework) sequences of the amino acid sequence of SEQ ID NO: 46 (huUCHT1 (Y177T)). In some embodiments, the CDR sequences of the antigen-binding domain that binds the protein associated with the TCR complex have 100% identity with the CDR sequences of the amino acid sequence of SEQ ID NO: 46 (huUCHT1 (Y177T)), and the non-CDR (e.g., framework) sequences of the antigen-binding domain that binds the protein associated with the TCR complex have at least 96% sequence identity with the non-CDR (e.g., framework) sequences of the amino acid sequence of SEQ ID NO: 46 (huUCHT1 (Y177T)). In some embodiments, the CDR sequences of the antigen-binding domain that binds the protein associated with the TCR complex have 100% identity with the CDR sequences of the amino acid sequence of SEQ ID NO: 46 (huUCHT1 (Y177T)), and the non-CDR (e.g., framework) sequences of the antigen-binding domain that binds the protein associated with the TCR complex have at least 97% sequence identity with the non-CDR (e.g., framework) sequences of the amino acid sequence of SEQ ID NO: 46 (huUCHT1 (Y177T)). In some embodiments, the CDR sequences of the antigen-binding domain that binds the protein associated with the TCR complex have 100% identity with the CDR sequences of the amino acid sequence of SEQ ID NO: 46 (huUCHT1 (Y177T)), and the non-CDR (e.g., framework) sequences of the antigen-binding domain that binds the protein associated with the TCR complex have at least 98% sequence identity with the non-CDR (e.g., framework) sequences of the amino acid sequence of SEQ ID NO: 46 (huUCHT1 (Y177T)). In some embodiments, the CDR sequences of the antigen-binding domain that binds the protein associated with the TCR complex have 100% identity with the CDR sequences of the amino acid sequence of SEQ ID NO: 46 (huUCHT1 (Y177T)), and the non-CDR (e.g., framework) sequences of the antigen-binding domain that binds the protein associated with the TCR complex have at least 99% sequence identity with the non-CDR (e.g., framework) sequences of the amino acid sequence of SEQ ID NO: 46 (huUCHT1 (Y177T)).

In some embodiments, the antigen-binding domain that binds to the protein associated with the TCR complex is OKT3. In some embodiments, the murine OKT3 antigen-binding domain is encoded by SEQ ID NO: 21. In some embodiments, the OKT3 antigen-binding domain comprises SEQ ID NO: 22.

In some embodiments, the polynucleotide encoding the antigen-binding domain that binds the protein associated with the TCR complex comprises a nucleotide sequence having at least 70% sequence identity with the nucleotide sequence of SEQ ID NO: 21 (OKT3). In some embodiments, the polynucleotide encoding the antigen-binding domain that binds the protein associated with the TCR complex comprises a nucleotide sequence having at least 75% sequence identity with the nucleotide sequence of SEQ ID NO: 21 (OKT3). In some embodiments, the polynucleotide encoding the antigen-binding domain that binds the protein associated with the TCR complex comprises a nucleotide sequence having at least 80% sequence identity with the nucleotide sequence of SEQ ID NO: 21 (OKT3). In some embodiments, the polynucleotide encoding the antigen-binding domain that binds the protein associated with the TCR complex comprises a nucleotide sequence having at least 85% sequence identity with the nucleotide sequence of SEQ ID NO: 21 (OKT3). In some embodiments, the polynucleotide encoding the antigen-binding domain that binds the protein associated with the TCR complex comprises a nucleotide sequence having at least 90% sequence identity with the nucleotide sequence of SEQ ID NO: 21 (OKT3). In some embodiments, the polynucleotide encoding the antigen-binding domain that binds the protein associated with the TCR complex comprises a nucleotide sequence having at least 95% sequence identity with the nucleotide sequence of SEQ ID NO: 21 (OKT3). In some embodiments, the polynucleotide encoding the antigen-binding domain that binds the protein associated with the TCR complex comprises a nucleotide sequence having at least 96% sequence identity with the nucleotide sequence of SEQ ID NO: 21 (OKT3). In some embodiments, the polynucleotide encoding the antigen-binding domain that binds the protein associated with the TCR complex comprises a nucleotide sequence having at least 97% sequence identity with the nucleotide sequence of SEQ ID NO: 21 (OKT3). In some embodiments, the polynucleotide encoding the antigen-binding domain that binds the protein associated with the TCR complex comprises a nucleotide sequence having at least 98% sequence identity with the nucleotide sequence of SEQ ID NO: 21 (OKT3). In some embodiments, the polynucleotide encoding the antigen-binding domain that binds the protein associated with the TCR complex comprises a nucleotide sequence having at least 99% sequence identity with the nucleotide sequence of SEQ ID NO: 21 (OKT3). In some embodiments, the polynucleotide encoding the antigen-binding domain that binds the protein associated with the TCR complex comprises the nucleotide sequence of SEQ ID NO: 21 (OKT3).

In some embodiments, the antigen-binding domain that binds the protein associated with the TCR complex comprises an amino acid sequence having at least 70% sequence identity with the amino acid sequence of SEQ ID NO: 22 (OKT3). In some embodiments, the antigen-binding domain that binds the protein associated with the TCR complex comprises an amino acid sequence having at least 75% sequence identity with the amino acid sequence of SEQ ID NO: 22 (OKT3). In some embodiments, the antigen-binding domain that binds the protein associated with the TCR complex comprises an amino acid sequence having at least 80% sequence identity with the amino acid sequence of SEQ ID NO: 22 (OKT3). In some embodiments, the antigen-binding domain that binds the protein associated with the TCR complex comprises an amino acid sequence having at least 85% sequence identity with the amino acid sequence of SEQ ID NO: 22 (OKT3). In some embodiments, the antigen-binding domain that binds the protein associated with the TCR complex comprises an amino acid sequence having at least 90% sequence identity with the amino acid sequence of SEQ ID NO: 22 (OKT3). In some embodiments, the antigen-binding domain that binds the protein associated with the TCR complex comprises an amino acid sequence having at least 95% sequence identity with the amino acid sequence of SEQ ID NO: 22 (OKT3). In some embodiments, the antigen-binding domain that binds the protein associated with the TCR complex comprises an amino acid sequence having at least 96% sequence identity with the amino acid sequence of SEQ ID NO: 22 (OKT3). In some embodiments, the antigen-binding domain that binds the protein associated with the TCR complex comprises an amino acid sequence having at least 97% sequence identity with the amino acid sequence of SEQ ID NO: 22 (OKT3). In some embodiments, the antigen-binding domain that binds the protein associated with the TCR complex comprises an amino acid sequence having at least 98% sequence identity with the amino acid sequence of SEQ ID NO: 22 (OKT3). In some embodiments, the antigen-binding domain that binds the protein associated with the TCR complex comprises an amino acid sequence having at least 99% sequence identity with the amino acid sequence of SEQ ID NO: 22 (OKT3). In some embodiments, the antigen-binding domain that binds the protein associated with the TCR complex comprises the amino acid sequence of SEQ ID NO: 22 (OKT3). In some embodiments, the CDR sequences of the antigen-binding domain that binds the protein associated with the TCR complex have 100% identity with the CDR sequences of the amino acid sequence of SEQ ID NO: 22 (OKT3) (i.e., the antigen-binding domain that binds the protein associated with the TCR complex comprises an amino acid sequence comprising a CDRH1, CDRH2, CDRH3, CDRL1, CDRL2, and CDRL3, each having 100% identity to the corresponding CDR in the amino acid sequence of SEQ ID NO: 22 (OKT3). In some embodiments, the CDR sequences of the antigen-binding domain that binds the protein associated with the TCR complex have 100% identity with the CDR sequences of the amino acid sequence of SEQ ID NO: 22 (OKT3), and the non-CDR (e.g., framework) sequences of the antigen-binding domain that binds the protein associated with the TCR complex have at least 80% sequence identity with the non-CDR (e.g., framework) sequences of the amino acid sequence of SEQ ID NO: 22 (OKT3). In some embodiments, the CDR sequences of the antigen-binding domain that binds the protein associated with the TCR complex have 100% identity with the CDR sequences of the amino acid sequence of SEQ ID NO: 22 (OKT3), and the non-CDR (e.g., framework) sequences of the antigen-binding domain that binds the protein associated with the TCR complex have at least 85% sequence identity with the non-CDR (e.g., framework) sequences of the amino acid sequence of SEQ ID NO: 22 (OKT3). In some embodiments, the CDR sequences of the antigen-binding domain that binds the protein associated with the TCR complex have 100% identity with the CDR sequences of the amino acid sequence of SEQ ID NO: 22 (OKT3), and the non-CDR (e.g., framework) sequences of the antigen-binding domain that binds the protein associated with the TCR complex have at least 90% sequence identity with the non-CDR (e.g., framework) sequences of the amino acid sequence of SEQ ID NO: 22 (OKT3). In some embodiments, the CDR sequences of the antigen-binding domain that binds the protein associated with the TCR complex have 100% identity with the CDR sequences of the amino acid sequence of SEQ ID NO: 22 (OKT3), and the non-CDR (e.g., framework) sequences of the antigen-binding domain that binds the protein associated with the TCR complex have at least 95% sequence identity with the non-CDR (e.g., framework) sequences of the amino acid sequence of SEQ ID NO: 22 (OKT3). In some embodiments, the CDR sequences of the antigen-binding domain that binds the protein associated with the TCR complex have 100% identity with the CDR sequences of the amino acid sequence of SEQ ID NO: 22 (OKT3), and the non-CDR (e.g., framework) sequences of the antigen-binding domain that binds the protein associated with the TCR complex have at least 96% sequence identity with the non-CDR (e.g., framework) sequences of the amino acid sequence of SEQ ID NO: 22 (OKT3). In some embodiments, the CDR sequences of the antigen-binding domain that binds the protein associated with the TCR complex have 100% identity with the CDR sequences of the amino acid sequence of SEQ ID NO: 22 (OKT3), and the non-CDR (e.g., framework) sequences of the antigen-binding domain that binds the protein associated with the TCR complex have at least 97% sequence identity with the non-CDR (e.g., framework) sequences of the amino acid sequence of SEQ ID NO: 22 (OKT3). In some embodiments, the CDR sequences of the antigen-binding domain that binds the protein associated with the TCR complex have 100% identity with the CDR sequences of the amino acid sequence of SEQ ID NO: 22 (OKT3), and the non-CDR (e.g., framework) sequences of the antigen-binding domain that binds the protein associated with the TCR complex have at least 98% sequence identity with the non-CDR (e.g., framework) sequences of the amino acid sequence of SEQ ID NO: 22 (OKT3). In some embodiments, the CDR sequences of the antigen-binding domain that binds the protein associated with the TCR complex have 100% identity with the CDR sequences of the amino acid sequence of SEQ ID NO: 22 (OKT3), and the non-CDR (e.g., framework) sequences of the antigen-binding domain that binds the protein associated with the TCR complex have at least 99% sequence identity with the non-CDR (e.g., framework) sequences of the amino acid sequence of SEQ ID NO: 22 (OKT3).

In some embodiments, the antigen-binding domain that binds to the protein associated with the TCR complex is F6A. In some embodiments, the murine F6A antigen-binding domain is encoded by SEQ ID NO: 23. In some embodiments, the F6A antigen-binding domain comprises SEQ ID NO: 24.

In some embodiments, the polynucleotide encoding the antigen-binding domain that binds the protein associated with the TCR complex comprises a nucleotide sequence having at least 70% sequence identity with the nucleotide sequence of SEQ ID NO: 23 (F6A). In some embodiments, the polynucleotide encoding the antigen-binding domain that binds the protein associated with the TCR complex comprises a nucleotide sequence having at least 75% sequence identity with the nucleotide sequence of SEQ ID NO: 23 (F6A). In some embodiments, the polynucleotide encoding the antigen-binding domain that binds the protein associated with the TCR complex comprises a nucleotide sequence having at least 80% sequence identity with the nucleotide sequence of SEQ ID NO: 23 (F6A). In some embodiments, the polynucleotide encoding the antigen-binding domain that binds the protein associated with the TCR complex comprises a nucleotide sequence having at least 85% sequence identity with the nucleotide sequence of SEQ ID NO: 23 (F6A). In some embodiments, the polynucleotide encoding the antigen-binding domain that binds the protein associated with the TCR complex comprises a nucleotide sequence having at least 90% sequence identity with the nucleotide sequence of SEQ ID NO: 23 (F6A). In some embodiments, the polynucleotide encoding the antigen-binding domain that binds the protein associated with the TCR complex comprises a nucleotide sequence having at least 95% sequence identity with the nucleotide sequence of SEQ ID NO: 23 (F6A). In some embodiments, the polynucleotide encoding the antigen-binding domain that binds the protein associated with the TCR complex comprises a nucleotide sequence having at least 96% sequence identity with the nucleotide sequence of SEQ ID NO: 23 (F6A). In some embodiments, the polynucleotide encoding the antigen-binding domain that binds the protein associated with the TCR complex comprises a nucleotide sequence having at least 97% sequence identity with the nucleotide sequence of SEQ ID NO: 23 (F6A). In some embodiments, the polynucleotide encoding the antigen-binding domain that binds the protein associated with the TCR complex comprises a nucleotide sequence having at least 98% sequence identity with the nucleotide sequence of SEQ ID NO: 23 (F6A). In some embodiments, the polynucleotide encoding the antigen-binding domain that binds the protein associated with the TCR complex comprises a nucleotide sequence having at least 99% sequence identity with the nucleotide sequence of SEQ ID NO: 23 (F6A). In some embodiments, the polynucleotide encoding the antigen-binding domain that binds the protein associated with the TCR complex comprises the nucleotide sequence of SEQ ID NO: 23 (F6A).

In some embodiments, the antigen-binding domain that binds the protein associated with the TCR complex comprises an amino acid sequence having at least 70% sequence identity with the amino acid sequence of SEQ ID NO: 24 (F6A). In some embodiments, the antigen-binding domain that binds the protein associated with the TCR complex comprises an amino acid sequence having at least 75% sequence identity with the amino acid sequence of SEQ ID NO: 24 (F6A). In some embodiments, the antigen-binding domain that binds the protein associated with the TCR complex comprises an amino acid sequence having at least 80% sequence identity with the amino acid sequence of SEQ ID NO: 24 (F6A). In some embodiments, the antigen-binding domain that binds the protein associated with the TCR complex comprises an amino acid sequence having at least 85% sequence identity with the amino acid sequence of SEQ ID NO: 24 (F6A). In some embodiments, the antigen-binding domain that binds the protein associated with the TCR complex comprises an amino acid sequence having at least 90% sequence identity with the amino acid sequence of SEQ ID NO: 24 (F6A). In some embodiments, the antigen-binding domain that binds the protein associated with the TCR complex comprises an amino acid sequence having at least 95% sequence identity with the amino acid sequence of SEQ ID NO: 24 (F6A). In some embodiments, the antigen-binding domain that binds the protein associated with the TCR complex comprises an amino acid sequence having at least 96% sequence identity with the amino acid sequence of SEQ ID NO: 24 (F6A). In some embodiments, the antigen-binding domain that binds the protein associated with the TCR complex comprises an amino acid sequence having at least 97% sequence identity with the amino acid sequence of SEQ ID NO: 24 (F6A). In some embodiments, the antigen-binding domain that binds the protein associated with the TCR complex comprises an amino acid sequence having at least 98% sequence identity with the amino acid sequence of SEQ ID NO: 24 (F6A). In some embodiments, the antigen-binding domain that binds the protein associated with the TCR complex comprises an amino acid sequence having at least 99% sequence identity with the amino acid sequence of SEQ ID NO: 24 (F6A). In some embodiments, the antigen-binding domain that binds the protein associated with the TCR complex comprises the amino acid sequence of SEQ ID NO: 24 (F6A). In some embodiments, the CDR sequences of the antigen-binding domain that binds the protein associated with the TCR complex have 100% identity with the CDR sequences of the amino acid sequence of SEQ ID NO: 24 (F6A) (i.e., the antigen-binding domain that binds the protein associated with the TCR complex comprises an amino acid sequence comprising a CDRH1, CDRH2, CDRH3, CDRL1, CDRL2, and CDRL3, each having 100% identity to the corresponding CDR in the amino acid sequence of SEQ ID NO: 24 (F6A). In some embodiments, the CDR sequences of the antigen-binding domain that binds the protein associated with the TCR complex have 100% identity with the CDR sequences of the amino acid sequence of SEQ ID NO: 24 (F6A), and the non-CDR (e.g., framework) sequences of the antigen-binding domain that binds the protein associated with the TCR complex have at least 80% sequence identity with the non-CDR (e.g., framework) sequences of the amino acid sequence of SEQ ID NO: 24 (F6A). In some embodiments, the CDR sequences of the antigen-binding domain that binds the protein associated with the TCR complex have 100% identity with the CDR sequences of the amino acid sequence of SEQ ID NO: 24 (F6A), and the non-CDR (e.g., framework) sequences of the antigen-binding domain that binds the protein associated with the TCR complex have at least 85% sequence identity with the non-CDR (e.g., framework) sequences of the amino acid sequence of SEQ ID NO: 24 (F6A). In some embodiments, the CDR sequences of the antigen-binding domain that binds the protein associated with the TCR complex have 100% identity with the CDR sequences of the amino acid sequence of SEQ ID NO: 24 (F6A), and the non-CDR (e.g., framework) sequences of the antigen-binding domain that binds the protein associated with the TCR complex have at least 90% sequence identity with the non-CDR (e.g., framework) sequences of the amino acid sequence of SEQ ID NO: 24 (F6A). In some embodiments, the CDR sequences of the antigen-binding domain that binds the protein associated with the TCR complex have 100% identity with the CDR sequences of the amino acid sequence of SEQ ID NO: 24 (F6A), and the non-CDR (e.g., framework) sequences of the antigen-binding domain that binds the protein associated with the TCR complex have at least 95% sequence identity with the non-CDR (e.g., framework) sequences of the amino acid sequence of SEQ ID NO: 24 (F6A). In some embodiments, the CDR sequences of the antigen-binding domain that binds the protein associated with the TCR complex have 100% identity with the CDR sequences of the amino acid sequence of SEQ ID NO: 24 (F6A), and the non-CDR (e.g., framework) sequences of the antigen-binding domain that binds the protein associated with the TCR complex have at least 96% sequence identity with the non-CDR (e.g., framework) sequences of the amino acid sequence of SEQ ID NO: 24 (F6A). In some embodiments, the CDR sequences of the antigen-binding domain that binds the protein associated with the TCR complex have 100% identity with the CDR sequences of the amino acid sequence of SEQ ID NO: 24 (F6A), and the non-CDR (e.g., framework) sequences of the antigen-binding domain that binds the protein associated with the TCR complex have at least 97% sequence identity with the non-CDR (e.g., framework) sequences of the amino acid sequence of SEQ ID NO: 24 (F6A). In some embodiments, the CDR sequences of the antigen-binding domain that binds the protein associated with the TCR complex have 100% identity with the CDR sequences of the amino acid sequence of SEQ ID NO: 24 (F6A), and the non-CDR (e.g., framework) sequences of the antigen-binding domain that binds the protein associated with the TCR complex have at least 98% sequence identity with the non-CDR (e.g., framework) sequences of the amino acid sequence of SEQ ID NO: 24 (F6A). In some embodiments, the CDR sequences of the antigen-binding domain that binds the protein associated with the TCR complex have 100% identity with the CDR sequences of the amino acid sequence of SEQ ID NO: 24 (F6A), and the non-CDR (e.g., framework) sequences of the antigen-binding domain that binds the protein associated with the TCR complex have at least 99% sequence identity with the non-CDR (e.g., framework) sequences of the amino acid sequence of SEQ ID NO: 24 (F6A).

In some embodiments, the antigen-binding domain that binds to the protein associated with the TCR complex is L2K. In some embodiments, the murine L2K antigen-binding domain is encoded by SEQ ID NO: 25. In some embodiments, the L2K antigen-binding domain comprises SEQ ID NO: 26.

In some embodiments, the polynucleotide encoding the antigen-binding domain that binds the protein associated with the TCR complex comprises a nucleotide sequence having at least 70% sequence identity with the nucleotide sequence of SEQ ID NO: 25 (L2K). In some embodiments, the polynucleotide encoding the antigen-binding domain that binds the protein associated with the TCR complex comprises a nucleotide sequence having at least 75% sequence identity with the nucleotide sequence of SEQ ID NO: 25 (L2K). In some embodiments, the polynucleotide encoding the antigen-binding domain that binds the protein associated with the TCR complex comprises a nucleotide sequence having at least 80% sequence identity with the nucleotide sequence of SEQ ID NO: 25 (L2K). In some embodiments, the polynucleotide encoding the antigen-binding domain that binds the protein associated with the TCR complex comprises a nucleotide sequence having at least 85% sequence identity with the nucleotide sequence of SEQ ID NO: 25 (L2K). In some embodiments, the polynucleotide encoding the antigen-binding domain that binds the protein associated with the TCR complex comprises a nucleotide sequence having at least 90% sequence identity with the nucleotide sequence of SEQ ID NO: 25 (L2K). In some embodiments, the polynucleotide encoding the antigen-binding domain that binds the protein associated with the TCR complex comprises a nucleotide sequence having at least 95% sequence identity with the nucleotide sequence of SEQ ID NO: 25 (L2K). In some embodiments, the polynucleotide encoding the antigen-binding domain that binds the protein associated with the TCR complex comprises a nucleotide sequence having at least 96% sequence identity with the nucleotide sequence of SEQ ID NO: 25 (L2K). In some embodiments, the polynucleotide encoding the antigen-binding domain that binds the protein associated with the TCR complex comprises a nucleotide sequence having at least 97% sequence identity with the nucleotide sequence of SEQ ID NO: 25 (L2K). In some embodiments, the polynucleotide encoding the antigen-binding domain that binds the protein associated with the TCR complex comprises a nucleotide sequence having at least 98% sequence identity with the nucleotide sequence of SEQ ID NO: 25 (L2K). In some embodiments, the polynucleotide encoding the antigen-binding domain that binds the protein associated with the TCR complex comprises a nucleotide sequence having at least 99% sequence identity with the nucleotide sequence of SEQ ID NO: 25 (L2K). In some embodiments, the polynucleotide encoding the antigen-binding domain that binds the protein associated with the TCR complex comprises the nucleotide sequence of SEQ ID NO: 25 (L2K).

In some embodiments, the antigen-binding domain that binds the protein associated with the TCR complex comprises an amino acid sequence having at least 70% sequence identity with the amino acid sequence of SEQ ID NO: 26 (L2K). In some embodiments, the antigen-binding domain that binds the protein associated with the TCR complex comprises an amino acid sequence having at least 75% sequence identity with the amino acid sequence of SEQ ID NO: 26 (L2K). In some embodiments, the antigen-binding domain that binds the protein associated with the TCR complex comprises an amino acid sequence having at least 80% sequence identity with the amino acid sequence of SEQ ID NO: 26 (L2K). In some embodiments, the antigen-binding domain that binds the protein associated with the TCR complex comprises an amino acid sequence having at least 85% sequence identity with the amino acid sequence of SEQ ID NO: 26 (L2K). In some embodiments, the antigen-binding domain that binds the protein associated with the TCR complex comprises an amino acid sequence having at least 90% sequence identity with the amino acid sequence of SEQ ID NO: 26 (L2K). In some embodiments, the antigen-binding domain that binds the protein associated with the TCR complex comprises an amino acid sequence having at least 95% sequence identity with the amino acid sequence of SEQ ID NO: 26 (L2K). In some embodiments, the antigen-binding domain that binds the protein associated with the TCR complex comprises an amino acid sequence having at least 96% sequence identity with the amino acid sequence of SEQ ID NO: 26 (L2K). In some embodiments, the antigen-binding domain that binds the protein associated with the TCR complex comprises an amino acid sequence having at least 97% sequence identity with the amino acid sequence of SEQ ID NO: 26 (L2K). In some embodiments, the antigen-binding domain that binds the protein associated with the TCR complex comprises an amino acid sequence having at least 98% sequence identity with the amino acid sequence of SEQ ID NO: 26 (L2K). In some embodiments, the antigen-binding domain that binds the protein associated with the TCR complex comprises an amino acid sequence having at least 99% sequence identity with the amino acid sequence of SEQ ID NO: 26 (L2K). In some embodiments, the antigen-binding domain that binds the protein associated with the TCR complex comprises the amino acid sequence of SEQ ID NO: 26 (L2K). In some embodiments, the CDR sequences of the antigen-binding domain that binds the protein associated with the TCR complex have 100% identity with the CDR sequences of the amino acid sequence of SEQ ID NO: 26 (L2K) (i.e., the antigen-binding domain that binds the protein associated with the TCR complex comprises an amino acid sequence comprising a CDRH1, CDRH2, CDRH3, CDRL1, CDRL2, and CDRL3, each having 100% identity to the corresponding CDR in the amino acid sequence of SEQ ID NO: 26 (L2K). In some embodiments, the CDR sequences of the antigen-binding domain that binds the protein associated with the TCR complex have 100% identity with the CDR sequences of the amino acid sequence of SEQ ID NO: 26 (L2K), and the non-CDR (e.g., framework) sequences of the antigen-binding domain that binds the protein associated with the TCR complex have at least 80% sequence identity with the non-CDR (e.g., framework) sequences of the amino acid sequence of SEQ ID NO: 26 (L2K). In some embodiments, the CDR sequences of the antigen-binding domain that binds the protein associated with the TCR complex have 100% identity with the CDR sequences of the amino acid sequence of SEQ ID NO: 26 (L2K), and the non-CDR (e.g., framework) sequences of the antigen-binding domain that binds the protein associated with the TCR complex have at least 85% sequence identity with the non-CDR (e.g., framework) sequences of the amino acid sequence of SEQ ID NO: 26 (L2K). In some embodiments, the CDR sequences of the antigen-binding domain that binds the protein associated with the TCR complex have 100% identity with the CDR sequences of the amino acid sequence of SEQ ID NO: 26 (L2K), and the non-CDR (e.g., framework) sequences of the antigen-binding domain that binds the protein associated with the TCR complex have at least 90% sequence identity with the non-CDR (e.g., framework) sequences of the amino acid sequence of SEQ ID NO: 26 (L2K). In some embodiments, the CDR sequences of the antigen-binding domain that binds the protein associated with the TCR complex have 100% identity with the CDR sequences of the amino acid sequence of SEQ ID NO: 26 (L2K), and the non-CDR (e.g., framework) sequences of the antigen-binding domain that binds the protein associated with the TCR complex have at least 95% sequence identity with the non-CDR (e.g., framework) sequences of the amino acid sequence of SEQ ID NO: 26 (L2K). In some embodiments, the CDR sequences of the antigen-binding domain that binds the protein associated with the TCR complex have 100% identity with the CDR sequences of the amino acid sequence of SEQ ID NO: 26 (L2K), and the non-CDR (e.g., framework) sequences of the antigen-binding domain that binds the protein associated with the TCR complex have at least 96% sequence identity with the non-CDR (e.g., framework) sequences of the amino acid sequence of SEQ ID NO: 26 (L2K). In some embodiments, the CDR sequences of the antigen-binding domain that binds the protein associated with the TCR complex have 100% identity with the CDR sequences of the amino acid sequence of SEQ ID NO: 26 (L2K), and the non-CDR (e.g., framework) sequences of the antigen-binding domain that binds the protein associated with the TCR complex have at least 97% sequence identity with the non-CDR (e.g., framework) sequences of the amino acid sequence of SEQ ID NO: 26 (L2K). In some embodiments, the CDR sequences of the antigen-binding domain that binds the protein associated with the TCR complex have 100% identity with the CDR sequences of the amino acid sequence of SEQ ID NO: 26 (L2K), and the non-CDR (e.g., framework) sequences of the antigen-binding domain that binds the protein associated with the TCR complex have at least 98% sequence identity with the non-CDR (e.g., framework) sequences of the amino acid sequence of SEQ ID NO: 26 (L2K). In some embodiments, the CDR sequences of the antigen-binding domain that binds the protein associated with the TCR complex have 100% identity with the CDR sequences of the amino acid sequence of SEQ ID NO: 26 (L2K), and the non-CDR (e.g., framework) sequences of the antigen-binding domain that binds the protein associated with the TCR complex have at least 99% sequence identity with the non-CDR (e.g., framework) sequences of the amino acid sequence of SEQ ID NO: 26 (L2K).

Amino acid and nucleotide sequences of exemplary antigen-binding domains that bind a protein associated with the TCR complex are provided in Table 2.

TABLE 2
Table of Sequences

| SEQ ID NO | Description | Nucleotide/Amino Acid |
|---|---|---|
| SEQ ID NO: 13 | UCHT1¹ | Nucleotide |
| SEQ ID NO: 14 | UCHT1² | Amino Acid |
| SEQ ID NO: 21 | OKT3 | Nucleotide |
| SEQ ID NO: 22 | OKT3 | Amino Acid |
| SEQ ID NO: 23 | F6A | Nucleotide |
| SEQ ID NO: 24 | F6A | Amino Acid |
| SEQ ID NO: 25 | L2K | Nucleotide |
| SEQ ID NO: 26 | L2K | Amino Acid |
| SEQ ID NO: 43 | huUCHT1 | Nucleotide |
| SEQ ID NO: 44 | huUCHT1 | Amino Acid |
| SEQ ID NO: 45 | huUCHT1 (Y177T) | Nucleotide |
| SEQ ID NO: 46 | huUCHT1 (Y177T) | Amino Acid |
| SEQ ID NO: 71 | UCHT1 (Y182T) | Nucleotide |
| SEQ ID NO: 72 | UCHT1 (Y182T) | Amino Acid |

¹Light chain, nucleotides 1-324; Linker, nucleotides 325-387; Heavy chain, nucleotides 388-750
²Light chain, amino acids 1-108; Linker, amino acids 109-128; Heavy chain, amino acids 129-250

### Transmembrane Domain and Cytosolic Domain

In some embodiments, a T cell antigen coupler (TAC) comprises a T cell receptor (TCR) signaling domain polypeptide. In some embodiments, a TAC comprises a transmembrane domain of a TCR signaling domain polypeptide. In some embodiments, a TAC comprises a cytosolic domain of a TCR signaling domain polypeptide. In some embodiments, a TAC comprises a transmembrane domain and a cytosolic domain of a TCR signaling domain polypeptide.

In some embodiments, the T cell receptor (TCR) signaling domain polypeptide comprises a TCR co-receptor polypeptide. In some embodiments, the TCR signaling domain polypeptide comprises a transmembrane domain and/or a cytosolic domain of a TCR co-receptor polypeptide. In some embodiments, the TCR co-receptor is CD4, CD8, LAG3, or a chimeric variation thereof.

In some embodiments, the TCR co-receptor is CD4. In some embodiments, the TAC comprises a transmembrane domain and a cytosolic domain of a CD4 co-receptor. In some embodiments, the polynucleotide encoding the cytosolic and transmembrane domain comprises a nucleotide sequence having at least 70% sequence identity with the nucleotide sequence of SEQ ID NO: 17 (CD4 transmembrane and cytosolic domain). In some embodiments, the polynucleotide encoding the cytosolic and transmembrane domain comprises a nucleotide sequence having at least 75% sequence identity with the nucleotide sequence of SEQ ID NO: 17 (CD4 transmembrane and cytosolic domain). In some embodiments, the polynucleotide encoding the cytosolic and transmembrane domain comprises a nucleotide sequence having at least 80% sequence identity with the nucleotide sequence of SEQ ID NO: 17 (CD4 transmembrane and cytosolic domain). In some embodiments, the polynucleotide encoding the cytosolic and transmembrane domain comprises a nucleotide sequence having at least 85% sequence identity with the nucleotide sequence of SEQ ID NO: 17 (CD4 transmembrane and cytosolic domain). In some embodiments, the polynucleotide encoding the cytosolic and transmembrane domain comprises a nucleotide sequence having at least 90% sequence identity with the nucleotide sequence of SEQ ID NO: 17 (CD4 transmembrane and cytosolic domain). In some embodiments, the polynucleotide encoding the cytosolic and transmembrane domain comprises a nucleotide sequence having at least 95% sequence identity with the nucleotide sequence of SEQ ID NO: 17 (CD4 transmembrane and cytosolic domain). In some embodiments, the polynucleotide encoding the cytosolic and transmembrane domain comprises a nucleotide sequence having at least 96% sequence identity with the nucleotide sequence of SEQ ID NO: 17 (CD4 transmembrane and cytosolic domain). In some embodiments, the polynucleotide encoding the cytosolic and transmembrane domain comprises a nucleotide sequence having at least 97% sequence identity with the nucleotide sequence of SEQ ID NO: 17 (CD4 transmembrane and cytosolic domain). In some embodiments, the polynucleotide encoding the cytosolic and transmembrane domain comprises a nucleotide sequence having at least 98% sequence identity with the nucleotide sequence of SEQ ID NO: 17 (CD4 transmembrane and cytosolic domain). In some embodiments, the polynucleotide encoding the cytosolic and transmembrane domain comprises a nucleotide sequence having at least 99% sequence identity with the nucleotide sequence of SEQ ID NO: 17 (CD4 transmembrane and cytosolic domain). In some embodiments, the polynucleotide encoding the cytosolic and transmembrane domain comprises the nucleotide sequence of SEQ ID NO: 17 (CD4 transmembrane and cytosolic domain). In some embodiments, the cytosolic and transmembrane domain comprise an amino acid sequence having at least 70% sequence identity with the amino acid sequence of SEQ ID NO: 18 (CD4 transmembrane and cytosolic domain). In some embodiments, the cytosolic and transmembrane domain comprise an amino acid sequence having at least 75% sequence identity with the amino acid sequence of SEQ ID NO: 18 (CD4 transmembrane and cytosolic domain). In some embodiments, the cytosolic and transmembrane domain comprise an amino acid sequence having at least 80% sequence identity with the amino acid sequence of SEQ ID NO: 18 (CD4 transmembrane and cytosolic domain). In some embodiments, the cytosolic and transmembrane domain comprise an amino acid sequence having at least 85% sequence identity with the amino acid sequence of SEQ ID NO: 18 (CD4 transmembrane and cytosolic domain). In some embodiments, the cytosolic and transmembrane domain comprise an amino acid sequence having at least 90% sequence identity with the amino acid sequence of SEQ ID NO: 18 (CD4 transmembrane and cytosolic domain). In some embodiments, the cytosolic and transmembrane domain comprise an amino acid sequence having at least 95% sequence identity with the amino acid sequence of SEQ ID NO: 18 (CD4 transmembrane and cytosolic domain). In some embodiments, the cytosolic and transmembrane domain comprise an amino acid sequence having at least 96% sequence identity with the amino acid sequence of SEQ ID NO: 18 (CD4 transmembrane and cytosolic domain). In some embodiments, the cytosolic and transmembrane domain comprise an amino acid sequence having at least 97% sequence identity with the amino acid sequence of SEQ ID NO: 18 (CD4 transmembrane and cytosolic domain). In some embodiments, the cytosolic and transmembrane domain comprise an amino acid sequence having at least 98% sequence identity with the amino acid sequence of SEQ ID NO: 18 (CD4 transmembrane and cytosolic domain). In some embodiments, the cytosolic and transmembrane domain comprise an amino acid sequence having at least 99% sequence identity with the amino acid sequence of SEQ ID NO: 18 (CD4 transmembrane and cytosolic domain). In some embodiments, the cytosolic and transmembrane domain comprise the amino acid sequence of SEQ ID NO: 18 (CD4 transmembrane and cytosolic domain).

In some embodiments, the TCR co-receptor is CD8. In some embodiments, the TCR co-receptor is CD8α. In some embodiments, the polynucleotide encoding the cytosolic and transmembrane domain comprises a nucleotide sequence having at least 70% sequence identity with the nucleotide sequence of SEQ ID NO: 37 (CD8 transmembrane and cytosolic domain). In some embodiments, the polynucleotide encoding the cytosolic and transmembrane domain comprises a nucleotide sequence having at least 75% sequence identity with the nucleotide sequence of SEQ ID NO: 37 (CD8 transmembrane and cytosolic domain). In some embodiments, the polynucleotide encoding the cytosolic and transmembrane domain comprises a nucleotide sequence having at least 80% sequence identity with the nucleotide sequence of SEQ ID NO: 37 (CD8 transmembrane and cytosolic domain). In some embodiments, the polynucleotide encoding the cytosolic and transmembrane domain comprises a nucleotide sequence having at least 85% sequence identity with the nucleotide sequence of SEQ ID NO: 37 (CD8 transmembrane and cytosolic domain). In some embodiments, the polynucleotide encoding the cytosolic and transmembrane domain comprises a nucleotide sequence having at least 90% sequence identity with the nucleotide sequence of SEQ ID NO: 37 (CD8 transmembrane and cytosolic domain). In some embodiments, the polynucleotide encoding the cytosolic and transmembrane domain comprises a nucleotide sequence having at least 95% sequence identity with the nucleotide sequence of SEQ ID NO: 37 (CD8 transmembrane and cytosolic domain). In some embodiments, the polynucleotide encoding the cytosolic and transmembrane domain comprises a nucleotide sequence having at least 96% sequence identity with the nucleotide sequence of SEQ ID NO: 37 (CD8 transmembrane and cytosolic domain). In some embodiments, the polynucleotide encoding the cytosolic and transmembrane domain comprises a nucleotide sequence having at least 97% sequence identity with the nucleotide sequence of SEQ ID NO: 37 (CD8 transmembrane and cytosolic domain). In some embodiments, the polynucleotide encoding the cytosolic and transmembrane domain comprises a nucleotide sequence having at least 98% sequence identity with the nucleotide sequence of SEQ ID NO: 37 (CD8 transmembrane and cytosolic domain). In some embodiments, the polynucleotide encoding the cytosolic and transmembrane domain comprises a nucleotide sequence having at least 99% sequence identity with the nucleotide sequence of SEQ ID NO: 37 (CD8 transmembrane and cytosolic domain). In some embodiments, the polynucleotide encoding the cytosolic and transmembrane domain comprises the nucleotide sequence of SEQ ID NO: 37 (CD8 transmembrane and cytosolic domain). In some embodiments, the cytosolic and transmembrane domain comprise an amino acid sequence having at least 70% sequence identity with the amino acid sequence of SEQ ID NO: 38 (CD8 transmembrane and cytosolic domain). In some embodiments, the cytosolic and transmembrane domain comprise an amino acid sequence having at least 75% sequence identity with the amino acid sequence of SEQ ID NO: 38 (CD8 transmembrane and cytosolic domain). In some embodiments, the cytosolic and transmembrane domain comprise an amino acid sequence having at least 80% sequence identity with the amino acid sequence of SEQ ID NO: 38 (CD8 transmembrane and cytosolic domain). In some embodiments, the cytosolic and transmembrane domain comprise an amino acid sequence having at least 85% sequence identity with the amino acid sequence of SEQ ID NO: 38 (CD8 transmembrane and cytosolic domain). In some embodiments, the cytosolic and transmembrane domain comprise an amino acid sequence having at least 90% sequence identity with the amino acid sequence of SEQ ID NO: 38 (CD8 transmembrane and cytosolic domain). In some embodiments, the cytosolic and transmembrane domain comprise an amino acid sequence having at least 95% sequence identity with the amino acid sequence of SEQ ID NO: 38 (CD8 transmembrane and cytosolic domain). In some embodiments, the cytosolic and transmembrane domain comprise an amino acid sequence having at least 96% sequence identity with the amino acid sequence of SEQ ID NO: 38 (CD8 transmembrane and cytosolic domain). In some embodiments, the cytosolic and transmembrane domain comprise an amino acid sequence having at least 97% sequence identity with the amino acid sequence of SEQ ID NO: 38 (CD8 transmembrane and cytosolic domain). In some embodiments, the cytosolic and transmembrane domain comprise an amino acid sequence having at least 98% sequence identity with the amino acid sequence of SEQ ID NO: 38 (CD8 transmembrane and cytosolic domain). In some embodiments, the cytosolic and transmembrane domain comprise an amino acid sequence having at least 99% sequence identity with the amino acid sequence of SEQ ID NO: 38 (CD8 transmembrane and cytosolic domain). In some embodiments, the cytosolic and transmembrane domain comprise the amino acid sequence of SEQ ID NO: 38 (CD8 transmembrane and cytosolic domain).

In some embodiments, the TCR signaling domain polypeptide comprises a chimera of sequences or domains from co-receptors. In some embodiments, the TCR signaling domain polypeptide comprises a chimera of CD8α and CD8β, wherein the CD8α arginine rich region is replaced with the CD8β arginine rich region (CD8α+R(β) chimera). In some embodiments, the polynucleotide encoding the cytosolic and transmembrane domain comprises a nucleotide sequence having at least 70% sequence identity with the nucleotide sequence of SEQ ID NO: 39 (CD8α+R(β) chimera). In some embodiments, the polynucleotide encoding the cytosolic and transmembrane domain comprises a nucleotide sequence having at least 75% sequence identity with the nucleotide sequence of SEQ ID NO: 39 (CD8α+R(β) chimera). In some embodiments, the polynucleotide encoding the cytosolic and transmembrane domain comprises a nucleotide sequence having at least 80% sequence identity with the nucleotide sequence of SEQ ID NO: 39 (CD8α+R(β) chimera). In some embodiments, the polynucleotide encoding the cytosolic and transmembrane domain comprises a nucleotide sequence having at least 85% sequence identity with the nucleotide sequence of SEQ ID NO: 39 (CD8α+R(β) chimera). In some embodiments, the polynucleotide encoding the cytosolic and transmembrane domain comprises a nucleotide sequence having at least 90% sequence identity with the nucleotide sequence of SEQ ID NO: 39 (CD8α+R(β) chimera). In some embodiments, the polynucleotide encoding the cytosolic and transmembrane domain comprises a nucleotide sequence having at least 95% sequence identity with the nucleotide sequence of SEQ ID NO: 39 (CD8α+R(β) chimera). In some embodiments, the polynucleotide encoding the cytosolic and transmembrane domain comprises a nucleotide sequence having at least 96% sequence identity with the nucleotide sequence of SEQ ID NO: 39 (CD8α+R(β) chimera). In some embodiments, the polynucleotide encoding the cytosolic and transmembrane domain comprises a nucleotide sequence having at least 97% sequence identity with the nucleotide sequence of SEQ ID NO: 39 (CD8α+R(β) chimera). In some embodiments, the polynucleotide encoding the cytosolic and transmembrane domain comprises a nucleotide sequence having at least 98% sequence identity with the nucleotide sequence of SEQ ID NO: 39 (CD8α+R(β) chimera). In some embodiments, the polynucleotide encoding the cytosolic and transmembrane domain comprises a nucleotide sequence having at least 99% sequence identity with the nucleotide sequence of SEQ ID NO: 39 (CD8α+R(β) chimera). In some embodiments, the polynucleotide encoding the cytosolic and transmembrane domain comprises the nucleotide sequence of SEQ ID NO: 39 (CD8α+R(β) chimera). In some embodiments, the cytosolic and transmembrane domain comprise an amino acid sequence having at least 70% sequence identity with the amino acid sequence of SEQ ID NO: 40 (CD8α+R(β) chimera). In some embodiments, the cytosolic and transmembrane domain comprise an amino acid sequence having at least 75% sequence identity with the amino acid sequence of SEQ ID NO: 40 (CD8α+R(β) chimera). In some embodiments, the cytosolic and transmembrane domain comprise an amino acid sequence having at least 80% sequence identity with the amino acid sequence of SEQ ID NO: 40 (CD8α+R(β) chimera). In some embodiments, the cytosolic and transmembrane domain comprise an amino acid sequence having at least 85% sequence identity with the amino acid sequence of SEQ ID NO: 40 (CD8α+R(β) chimera). In some embodiments, the cytosolic and transmembrane domain comprise an amino acid sequence having at least 90% sequence identity with the amino acid sequence of SEQ ID NO: 40 (CD8α+R(β) chimera). In some embodiments, the cytosolic and transmembrane domain comprise an amino acid sequence having at least 95% sequence identity with the amino acid sequence of SEQ ID NO: 40 (CD8α+R(β) chimera). In some embodiments, the cytosolic and transmembrane domain comprise an amino acid sequence having at least 96% sequence identity with the amino acid sequence of SEQ ID NO: 40 (CD8α+R(β) chimera). In some embodiments, the cytosolic and transmembrane domain comprise an amino acid sequence having at least 97% sequence identity with the amino acid sequence of SEQ ID NO: 40 (CD8α+R(β) chimera). In some embodiments, the cytosolic and transmembrane domain comprise an amino acid sequence having at least 98% sequence identity with the amino acid sequence of SEQ ID NO: 40 (CD8α+R(β) chimera). In some embodiments, the cytosolic and transmembrane domain comprise an amino acid sequence having at least 99% sequence identity with the amino acid sequence of SEQ ID NO: 40 (CD8α+R(β) chimera). In some embodiments, the cytosolic and transmembrane domain comprise the amino acid sequence of SEQ ID NO: 40 (CD8α+R(β) chimera).

In some embodiments, the TCR signaling domain polypeptide comprises a chimera of CD8α and CD8β, where the CD8α CXCP domain, which contains an Lck binding motif, is appended to the C-terminus of the CD8β cytosolic domain (CD8β+Lck chimera). In some embodiments, the polynucleotide encoding the cytosolic and transmembrane domain comprises a nucleotide sequence having at least 70% sequence identity with the nucleotide sequence of SEQ ID NO: 41 (CD8β+Lck chimera). In some embodiments, the polynucleotide encoding the cytosolic and transmembrane domain comprises a nucleotide sequence having at least 75% sequence identity with the nucleotide sequence of SEQ ID NO: 41 (CD8β+Lck chimera). In some embodiments, the polynucleotide encoding the cytosolic and transmembrane domain comprises a nucleotide sequence having at least 80% sequence identity with the nucleotide sequence of SEQ ID NO: 41 (CD8β+Lck chimera). In some embodiments, the polynucleotide encoding the cytosolic and transmembrane domain comprises a nucleotide sequence having at least 85% sequence identity with the nucleotide sequence of SEQ ID NO: 41 (CD8β+Lck chimera). In some embodiments, the polynucleotide encoding the cytosolic and transmembrane domain comprises a nucleotide sequence having at least 90% sequence identity with the nucleotide sequence of SEQ ID NO: 41 (CD8β+Lck chimera). In some embodiments, the polynucleotide encoding the cytosolic and transmembrane domain comprises a nucleotide sequence having at least 95% sequence identity with the nucleotide sequence of SEQ ID NO: 41 (CD8β+Lck chimera). In some embodiments, the polynucleotide encoding the cytosolic and transmembrane domain comprises a nucleotide sequence having at least 96% sequence identity with the nucleotide sequence of SEQ ID NO: 41 (CD8β+Lck chimera). In some embodiments, the polynucleotide encoding the cytosolic and transmembrane domain comprises a nucleotide sequence having at least 97% sequence identity with the nucleotide sequence of SEQ ID NO: 41 (CD8β+Lck chimera). In some embodiments, the polynucleotide encoding the cytosolic and transmembrane domain comprises a nucleotide sequence having at least 98% sequence identity with the nucleotide sequence of SEQ ID NO: 41 (CD8β+Lck chimera). In some embodiments, the polynucleotide encoding the cytosolic and transmembrane domain comprises a nucleotide sequence having at least 99% sequence identity with the nucleotide sequence of SEQ ID NO: 41 (CD8β+Lck chimera). In some embodiments, the polynucleotide encoding the cytosolic and transmembrane domain comprises the nucleotide sequence of SEQ ID NO: 41 (CD8β+Lck chimera). In some embodiments, the cytosolic and transmembrane domain comprise an amino acid sequence having at least 70% sequence identity with the amino acid sequence of SEQ ID NO: 42 (CD8β+Lck chimera). In some embodiments, the cytosolic and transmembrane domain comprise an amino acid sequence having at least 75% sequence identity with the amino acid sequence of SEQ ID NO: 42 (CD8β+Lck chimera). In some embodiments, the cytosolic and transmembrane domain comprise an amino acid sequence having at least 80% sequence identity with the amino acid sequence of SEQ ID NO: 42 (CD8β+Lck chimera). In some embodiments, the cytosolic and transmembrane domain comprise an amino acid sequence having at least 85% sequence identity with the amino acid sequence of SEQ ID NO: 42 (CD8β+Lck chimera). In some embodiments, the cytosolic and transmembrane domain comprise an amino acid sequence having at least 90% sequence identity with the amino acid sequence of SEQ ID NO: 42 (CD8β+Lck chimera). In some embodiments, the cytosolic and transmembrane domain comprise an amino acid sequence having at least 95% sequence identity with the amino acid sequence of SEQ ID NO: 42 (CD8β+Lck chimera). In some embodiments, the cytosolic and transmembrane domain comprise an amino acid sequence having at least 96% sequence identity with the amino acid sequence of SEQ ID NO: 42 (CD8β+Lck chimera). In some embodiments, the cytosolic and transmembrane domain comprise an amino acid sequence having at least 97% sequence identity with the amino acid sequence of SEQ ID NO: 42 (CD8β+Lck chimera). In some embodiments, the cytosolic and transmembrane domain comprise an amino acid sequence having at least 98% sequence identity with the amino acid sequence of SEQ ID NO: 42 (CD8β+Lck chimera). In some embodiments, the cytosolic and transmembrane domain comprise an amino acid sequence having at least 99% sequence identity with the amino acid sequence of SEQ ID NO: 42 (CD8β+Lck chimera). In some embodiments, the cytosolic and transmembrane domain comprise the amino acid sequence of SEQ ID NO: 42 (CD8β+Lck chimera).

In some embodiments, the TCR signaling domain polypeptide includes both a cytosolic domain and a transmembrane domain of a TCR co-receptor protein. In some embodiments, the cytosolic domain and transmembrane domain are from the same co-receptor or from different co-receptors.

Amino acid and nucleotide sequences of exemplary transmembrane and cytosolic domains are provided in Table 3.

TABLE 3
Table of Sequences

| SEQ ID NO | Description | Nucleotide/Amino Acid |
|---|---|---|
| SEQ ID NO: 17 | CD4 Domain¹ | Nucleotide |
| SEQ ID NO: 18 | CD4 Domain² | Amino Acid |
| SEQ ID NO: 37 | CD8α Domain | Nucleotide |
| SEQ ID NO: 38 | CD8α Domain | Amino Acid |
| SEQ ID NO: 39 | CD8α + R(β) Domain | Nucleotide |
| SEQ ID NO: 40 | CD8α + R(β) Domain | Amino Acid |
| SEQ ID NO: 41 | CD8 α + Lck Domain | Nucleotide |
| SEQ ID NO: 42 | CD8 α + Lck Domain | Amino Acid |

¹Extracellular linker, nucleotides 1-66; Transmembrane domain, nucleotides 67-132; Cytosolic domain, nucleotides 133-254
²Extracellular linker, amino acids 1-22; Transmembrane domain, amino acids 23-44; Cytosolic domain, amino acids 45-84

### Linkers, Connectors, and Configurations

In some embodiments, a nucleic acid disclosed herein is in an order of (1) a first polynucleotide encoding an antigen-binding domain that binds a target (e.g., a CD19 antigen, a HER2 antigen, or a BCMA antigen); (2) a second polynucleotide encoding an antigen-binding domain that binds a TCR complex; (3) a third polynucleotide encoding a transmembrane domain and a cytosolic domain. In some embodiments, a nucleic acid disclosed herein is in an order of (1) a first polynucleotide encoding an antigen-binding domain that binds a target (e.g., a CD19 antigen, a HER2 antigen, or a BCMA antigen); (2) a second polynucleotide encoding an antigen-binding domain that binds a TCR complex; (3) a third polynucleotide encoding a transmembrane domain and a cytosolic domain, wherein the order is 5′ end to 3′ end. In some embodiments, a nucleic acid disclosed herein is in an order of (1) a first polynucleotide encoding an antigen-binding domain that binds a target (e.g., a CD19 antigen, a HER2 antigen, or a BCMA antigen); (2) a second polynucleotide encoding an antigen-binding domain that binds a TCR complex; (3) a third polynucleotide encoding a transmembrane domain and a cytosolic domain, wherein the order is 3′ end to 5′ end. In some embodiments, a nucleic acid described herein is in an order of (1) a first polynucleotide encoding an antigen-binding domain that binds a TCR complex; (2) a second polynucleotide encoding an antigen-binding domain that binds a target (e.g., a CD19 antigen, a HER2 antigen, or a BCMA antigen); (3) a third polynucleotide encoding a transmembrane domain and a cytosolic domain. In some embodiments, a nucleic acid described herein is in an order of (1) a first polynucleotide encoding an antigen-binding domain that binds a TCR complex; (2) a second polynucleotide encoding an antigen-binding domain that binds a target (e.g., a CD19 antigen, a HER2 antigen, or a BCMA antigen); (3) a third polynucleotide encoding a transmembrane domain and a cytosolic domain, wherein the order is 5′ end to 3′ end. In some embodiments, a nucleic acid described herein is in an order of (1) a first polynucleotide encoding an antigen-binding domain that binds a TCR complex; (2) a second polynucleotide encoding an antigen-binding domain that binds a target (e.g., a CD19 antigen, a HER2 antigen, or a BCMA antigen); (3) a third polynucleotide encoding a transmembrane domain and a cytosolic domain, wherein the order is 3′ end to 5′ end.

In some embodiments, a target (e.g., a CD19 antigen, a HER2 antigen, or a BCMA antigen) TAC polypeptide disclosed herein is in an order of (1) an antigen-binding domain that binds a target (e.g., a CD19 antigen, a HER2 antigen, or a BCMA antigen); (2) an antigen-binding domain that binds a TCR complex; (3) a transmembrane domain and a cytosolic domain, wherein the order is N-terminus to C-terminus. In some embodiments, a target (e.g., a CD19 antigen, a HER2 antigen, or a BCMA antigen) TAC polypeptide disclosed herein is in an order of (1) an antigen-binding domain that binds a target (e.g., a CD19 antigen, a HER2 antigen, or a BCMA antigen); (2) an antigen-binding domain that binds a TCR complex; (3) a transmembrane domain and a cytosolic domain, wherein the order is C-terminus to N-terminus. In some embodiments, a target (e.g., a CD19 antigen, a HER2 antigen, or a BCMA antigen) TAC polypeptide described herein is in an order of (1) an antigen-binding domain that binds a TCR complex; (2) an antigen-binding domain that binds a target (e.g., a CD19 antigen, a HER2 antigen, or a BCMA antigen); (3) a transmembrane domain and a cytosolic domain, wherein the order is N-terminus to C-terminus. In some embodiments, a target (e.g., a CD19 antigen, a HER2 antigen, or a BCMA antigen) TAC polypeptide described herein is in an order of (1) an antigen-binding domain that binds a TCR complex; (2) an antigen-binding domain that binds a target (e.g., a CD19 antigen, a HER2 antigen, or a BCMA antigen); (3) a transmembrane domain and a cytosolic domain, wherein the order is C-terminus to N-terminus.

In some embodiments, the antigen-binding domain that binds a target (e.g., a CD19 antigen, a HER2 antigen, or a BCMA antigen), the antigen-binding domain that binds the TCR complex, and/or the transmembrane domain and cytosolic domain are directly fused. For example, the antigen-binding domain that binds a target (e.g., a CD19 antigen, a HER2 antigen, or a BCMA antigen) and the transmembrane domain and cytosolic domain are both fused to the antigen-binding domain that binds the TCR complex. In some embodiments, the antigen-binding domain that binds a target (e.g., a CD19 antigen, a HER2 antigen, or a BCMA antigen), the antigen-binding domain that binds the TCR complex, and/or the transmembrane domain and cytosolic domain are joined by at least one linker. In some embodiments, the antigen-binding domain that binds a target (e.g., a CD19 antigen, a HER2 antigen, or a BCMA antigen) and the antigen-binding domain that binds the TCR complex are directly fused, and joined to the transmembrane domain and cytosolic domain by a linker. In some embodiments, the antigen-binding domain that binds the TCR complex and the transmembrane domain and cytosolic domain are directly fused, and joined to the antigen-binding domain that binds a target (e.g., a CD19 antigen, a HER2 antigen, or a BCMA antigen) by a linker.

In some embodiments, the linker is a peptide linker. In some embodiments, the peptide linker comprises 1 to 40 amino acids. In some embodiments, the peptide linker comprises 1 to 30 amino acids. In some embodiments, the peptide linker comprises 1 to 15 amino acids. In some embodiments, the peptide linker comprises 1 to 10 amino acids. In some embodiments, the peptide linker comprises 1 to 6 amino acids. In some embodiments, the peptide linker comprises 30 to 40 amino acids. In some embodiments, the peptide linker comprises 32 to 36 amino acids. In some embodiments, the peptide linker comprises 5 to 30 amino acids. In some embodiments, the peptide linker comprises 5 amino acids. In some embodiments, the peptide linker comprises 10 amino acids. In some embodiments, the peptide linker comprises 15 amino acids. In some embodiments, the peptide linker comprises 20 amino acids. In some embodiments, the peptide linker comprises 25 amino acids. In some embodiments, the peptide linker comprises 30 amino acids. In some embodiments, the peptide linker comprises a glycine and/or serine-rich linker.

In some embodiments, the at least one linker comprises an amino acid sequence having at least 80% identity with the amino acid sequence of SEQ ID NO: 12 ((G4S)4-based linker), SEQ ID NO: 16 (G4S-based linker), SEQ ID NO: 20 (CD4 based linker), SEQ ID NO: 28 (short helix connector), SEQ ID NO: 30 (long helix connector), SEQ ID NO: 32 (large domain connector), SEQ ID NO: 69 (flexible connector), SEQ ID NO: 73 (G4S flexible linker), or SEQ ID NO: 74 (G4S3 flexible linker). In some embodiments, the at least one linker comprises an amino acid sequence having at least 85% identity with the amino acid sequence of SEQ ID NO: 12 ((G4S)4-based linker), SEQ ID NO: 16 (G4S-based linker), SEQ ID NO: 20 (CD4 based linker), SEQ ID NO: 28 (short helix connector), SEQ ID NO: 30 (long helix connector), SEQ ID NO: 32 (large domain connector), SEQ ID NO: 69 (flexible connector), SEQ ID NO: 73 (G4S flexible linker), or SEQ ID NO: 74 (G4S3 flexible linker). In some embodiments, the at least one linker comprises an amino acid sequence having at least 90% identity with the amino acid sequence of SEQ ID NO: 12 ((G4S)4-based linker), SEQ ID NO: 16 (G4S-based linker), SEQ ID NO: 20 (CD4 based linker), SEQ ID NO: 28 (short helix connector), SEQ ID NO: 30 (long helix connector), SEQ ID NO: 32 (large domain connector), SEQ ID NO: 69 (flexible connector), SEQ ID NO: 73 (G4S flexible linker), or SEQ ID NO: 74 (G4S3 flexible linker). In some embodiments, the at least one linker comprises an amino acid sequence having at least 95% identity with the amino acid sequence of SEQ ID NO: 12 ((G4S)4-based linker), SEQ ID NO: 16 (G4S-based linker), SEQ ID NO: 20 (CD4 based linker), SEQ ID NO: 28 (short helix connector), SEQ ID NO: 30 (long helix connector), SEQ ID NO: 32 (large domain connector), SEQ ID NO: 69 (flexible connector), SEQ ID NO: 73 (G4S flexible linker), or SEQ ID NO: 74 (G4S3 flexible linker). In some embodiments, the at least one linker comprises an amino acid sequence having at least 96% identity with the amino acid sequence of SEQ ID NO: 12 ((G4S)4-based linker), SEQ ID NO: 16 (G4S-based linker), SEQ ID NO: 20 (CD4 based linker), SEQ ID NO: 28 (short helix connector), SEQ ID NO: 30 (long helix connector), SEQ ID NO: 32 (large domain connector), SEQ ID NO: 69 (flexible connector), SEQ ID NO: 73 (G4S flexible linker), or SEQ ID NO: 74 (G4S3 flexible linker). In some embodiments, the at least one linker comprises an amino acid sequence having at least 97% identity with the amino acid sequence of SEQ ID NO: 12 ((G4S)4-based linker), SEQ ID NO: 16 (G4S-based linker), SEQ ID NO: 20 (CD4 based linker), SEQ ID NO: 28 (short helix connector), SEQ ID NO: 30 (long helix connector), SEQ ID NO: 32 (large domain connector), SEQ ID NO: 69 (flexible connector), SEQ ID NO: 73 (G4S flexible linker), or SEQ ID NO: 74 (G4S3 flexible linker). In some embodiments, the at least one linker comprises an amino acid sequence having at least 98% identity with the amino acid sequence of SEQ ID NO: 12 ((G4S)4-based linker), SEQ ID NO: 16 (G4S-based linker), SEQ ID NO: 20 (CD4 based linker), SEQ ID NO: 28 (short helix connector), SEQ ID NO: 30 (long helix connector), SEQ ID NO: 32 (large domain connector), SEQ ID NO: 69 (flexible connector), SEQ ID NO: 73 (G4S flexible linker), or SEQ ID NO: 74 (G4S3 flexible linker). In some embodiments, the at least one linker comprises an amino acid sequence having at least 80% identity with the amino acid sequence of SEQ ID NO: 12 ((G4S)4-based linker), SEQ ID NO: 16 (G4S-based linker), SEQ ID NO: 20 (CD4 based linker), SEQ ID NO: 28 (short helix connector), SEQ ID NO: 30 (long helix connector), SEQ ID NO: 32 (large domain connector), SEQ ID NO: 69 (flexible connector), SEQ ID NO: 73 (G4S flexible linker), or SEQ ID NO: 74 (G4S3 flexible linker). In some embodiments, the at least one linker comprises the amino acid sequence of SEQ ID NO: 12 ((G4S)4-based linker), SEQ ID NO: 16 (G4S-based linker), SEQ ID NO: 20 (CD4 based linker), SEQ ID NO: 28 (short helix connector), SEQ ID NO: 30 (long helix connector), SEQ ID NO: 32 (large domain connector), SEQ ID NO: 69 (flexible connector), SEQ ID NO: 73 (G4S flexible linker), or SEQ ID NO: 74 (G4S3 flexible linker).

In some embodiments, the peptide linker that joins the antigen-binding domain that binds a target (e.g., a CD19 antigen, a HER2 antigen, or a BCMA antigen) to the antigen-binding domain that binds a TCR complex (e.g., UCHT1) is known as the connector to distinguish this protein domain from other linkers in the TAC. The connector may be of any size. In some embodiments, the connector between the antigen-binding domain that binds a TCR complex and the antigen-binding domain that binds a target (e.g., a CD19 antigen, a HER2 antigen, or a BCMA antigen) is a short helix comprising SEQ ID NO: 28. In some embodiments, the connector between the antigen-binding domain that binds a TCR complex and the antigen-binding domain that binds a target (e.g., a CD19 antigen, a HER2 antigen, or a BCMA antigen) is a short helix encoded by SEQ ID NO: 27. In some embodiments, the connector between the antigen-binding domain that binds a TCR complex and the antigen-binding domain that binds a target (e.g., a CD19 antigen, a HER2 antigen, or a BCMA antigen) is a long helix comprising SEQ ID NO: 30. In some embodiments, the connector between the antigen-binding domain that binds a TCR complex and the antigen-binding domain that binds a target (e.g., a CD19 antigen, a HER2 antigen, or a BCMA antigen) is a long helix encoded by SEQ ID NO: 29. In some embodiments, the connector between the antigen-binding domain that binds a TCR complex and the antigen-binding domain that binds a target (e.g., a CD19 antigen, a HER2 antigen, or a BCMA antigen) is a large domain comprising SEQ ID NO: 32. In some embodiments, the connector between the antigen-binding domain that binds a TCR complex and the antigen-binding domain that binds a target (e.g., a CD19 antigen, a HER2 antigen, or a BCMA antigen) is a large domain encoded by SEQ ID NO: 31.

In some embodiments, a nucleic acid or TAC disclosed herein comprises a leader sequence. In some embodiments, the leader sequence is encoded by a nucleotide sequence having at least 80% sequence identity with the nucleotide sequence of SEQ ID NO: 5 (muIgG leader), SEQ ID NO: 47 (huIgG leader), or SEQ ID NO: 49 (huCD8a leader). In some embodiments, the leader sequence is encoded by a nucleotide sequence having at least 85% sequence identity with the nucleotide sequence of SEQ ID NO: 5 (muIgG leader), SEQ ID NO: 47 (huIgG leader), or SEQ ID NO: 49 (huCD8a leader). In some embodiments, the leader sequence is encoded by a nucleotide sequence having at least 90% sequence identity with the nucleotide sequence of SEQ ID NO: 5 (muIgG leader), SEQ ID NO: 47 (huIgG leader), or SEQ ID NO: 49 (huCD8a leader). In some embodiments, the leader sequence is encoded by a nucleotide sequence having at least 95% sequence identity with the nucleotide sequence of SEQ ID NO: 5 (muIgG leader), SEQ ID NO: 47 (huIgG leader), or SEQ ID NO: 49 (huCD8a leader). In some embodiments, the leader sequence is encoded by a nucleotide sequence having at least 96% sequence identity with the nucleotide sequence of SEQ ID NO: 5 (muIgG leader), SEQ ID NO: 47 (huIgG leader), or SEQ ID NO: 49 (huCD8a leader). In some embodiments, the leader sequence is encoded by a nucleotide sequence having at least 97% sequence identity with the nucleotide sequence of SEQ ID NO: 5 (muIgG leader), SEQ ID NO: 47 (huIgG leader), or SEQ ID NO: 49 (huCD8a leader). In some embodiments, the leader sequence is encoded by a nucleotide sequence having at least 98% sequence identity with the nucleotide sequence of SEQ ID NO: 5 (muIgG leader), SEQ ID NO: 47 (huIgG leader), or SEQ ID NO: 49 (huCD8a leader). In some embodiments, the leader sequence is encoded by a nucleotide sequence having at least 99% sequence identity with the nucleotide sequence of SEQ ID NO: 5 (muIgG leader), SEQ ID NO: 47 (huIgG leader), or SEQ ID NO: 49 (huCD8a leader). In some embodiments, the leader sequence comprises the nucleotide sequence of SEQ ID NO: 5 (muIgG leader), SEQ ID NO: 47 (huIgG leader), or SEQ ID NO: 49 (huCD8a leader).

In some embodiments, a nucleic acid or TAC disclosed herein comprises a leader sequence. In some embodiments, the leader sequence comprises an amino acid sequence having at least 80% sequence identity with the amino acid sequence of SEQ ID NO: 6 (muIgG leader), SEQ ID NO: 48 (huIgG leader), or SEQ ID NO: 50 (huCD8a leader). In some embodiments, the leader sequence comprises an amino acid sequence having at least 85% sequence identity with the amino acid sequence of SEQ ID NO: 6 (muIgG leader), SEQ ID NO: 48 (huIgG leader), or SEQ ID NO: 50 (huCD8a leader). In some embodiments, the leader sequence comprises an amino acid sequence having at least 90% sequence identity with the amino acid sequence of SEQ ID NO: 6 (muIgG leader), SEQ ID NO: 48 (huIgG leader), or SEQ ID NO: 50 (huCD8a leader). In some embodiments, the leader sequence comprises an amino acid sequence having at least 95% sequence identity with the amino acid sequence of SEQ ID NO: 6 (muIgG leader), SEQ ID NO: 48 (huIgG leader), or SEQ ID NO: 50 (huCD8a leader). In some embodiments, the leader sequence comprises an amino acid sequence having at least 96% sequence identity with the amino acid sequence of SEQ ID NO: 6 (muIgG leader), SEQ ID NO: 48 (huIgG leader), or SEQ ID NO: 50 (huCD8a leader). In some embodiments, the leader sequence comprises an amino acid sequence having at least 97% sequence identity with the amino acid sequence of SEQ ID NO: 6 (muIgG leader), SEQ ID NO: 48 (huIgG leader), or SEQ ID NO: 50 (huCD8a leader). In some embodiments, the leader sequence comprises an amino acid sequence having at least 98% sequence identity with the amino acid sequence of SEQ ID NO: 6 (muIgG leader), SEQ ID NO: 48 (huIgG leader), or SEQ ID NO: 50 (huCD8a leader). In some embodiments, the leader sequence comprises an amino acid sequence having at least 99% sequence identity with the amino acid sequence of SEQ ID NO: 6 (muIgG leader), SEQ ID NO: 48 (huIgG leader), or SEQ ID NO: 50 (huCD8a leader). In some embodiments, the leader sequence comprises the amino acid sequence of SEQ ID NO: 6 (muIgG leader), SEQ ID NO: 48 (huIgG leader), or SEQ ID NO: 50 (huCD8a leader).

Amino acid and nucleotide sequences of exemplary linkers, connectors, and leader sequences are provided in Table 4.

TABLE 4
Table of Sequences

| | | Nucleotide/ |
|---|---|---|
| SEQ ID NO | Description | Amino Acid |
| SEQ ID NO: 5 | muIgG leader (secretion signal) | Nucleotide |
| SEQ ID NO: 6 | muIgG leader (secretion signal) | Amino Acid |
| SEQ ID NO: 9 | Myc Tag | Nucleotide |
| SEQ ID NO: 10 | Myc Tag | Amino Acid |
| SEQ ID NO: 11 | (G4S)4-based linker | Nucleotide |
| SEQ ID NO: 12 | (G4S)4-based linker | Amino Acid |
| SEQ ID NO: 15 | G4S-based linker | Nucleotide |
| SEQ ID NO: 16 | G4S-based linker | Amino Acid |
| SEQ ID NO: 19 | CD4-based linker | Nucleotide |
| SEQ ID NO: 20 | CD4-based linker | Amino Acid |
| SEQ ID NO: 27 | Short Helix connector | Nucleotide |
| SEQ ID NO: 28 | Short Helix connector | Amino Acid |
| SEQ ID NO: 29 | Long Helix connector | Nucleotide |
| SEQ ID NO: 30 | Long Helix connector | Amino Acid |
| SEQ ID NO: 31 | Large domain connector | Nucleotide |
| SEQ ID NO: 32 | Large domain connector | Amino Acid |
| SEQ ID NO: 47 | huIgG leader | Nucleotide |
| SEQ ID NO: 48 | huIgG leader | Amino Acid |
| SEQ ID NO: 49 | huCD8a leader | Nucleotide |
| SEQ ID NO: 50 | huCD8a leader | Amino Acid |
| SEQ ID NO: 69 | Flexible Connector | Amino Acid |
| SEQ ID NO: 70 | Flexible Connector | Nucleotide |
| SEQ ID NO: 73 | G4S flexible linker | Amino Acid |
| SEQ ID NO: 74 | G4S3 linker | Amino Acid |
| SEQ ID NO: 77 | G4S3 linker | Nucleotide |

### Gamma Delta T Cells Comprising Specific TACs

Disclosed herein, in certain embodiments, are gamma delta T cells comprising a TAC comprising (a) a target-specific antigen-binding domain, (b) a single-chain antibody (scFv) that binds CD3ε, and (c) a transmembrane and cytosolic domain of the CD4 co-receptor. In some embodiments, the TAC comprises (a) a target-specific antigen-binding domain, (b) UCHT1, and (c) a transmembrane and cytosolic domain of the CD4 co-receptor. In some embodiments, the TAC comprises (a) a target-specific antigen-binding domain, (b) UCHT1 (Y182T), and (c) a transmembrane and cytosolic domain of the CD4 co-receptor. In some embodiments, the TAC comprises (a) a target-specific antigen-binding domain, (b) huUCHT1, and (c) a transmembrane and cytosolic domain of the CD4 co-receptor. In some embodiments, the TAC comprises (a) a target-specific antigen-binding domain, (b) huUCHT1 (Y177T), and (c) a transmembrane and cytosolic domain of the CD4 co-receptor. In some embodiments, the TAC comprises (a) a target-specific antigen-binding domain, (b) OKT3, and (c) a transmembrane and cytosolic domain of the CD4 co-receptor. In some embodiments, the TAC comprises (a) a target-specific antigen-binding domain, (b) F6A, and (c) a transmembrane and cytosolic domain of the CD4 co-receptor. In some embodiments, the TAC comprises (a) a target-specific antigen-binding domain, (b) L2K, and (c) a transmembrane and cytosolic domain of the CD4 co-receptor.

Disclosed herein, in certain embodiments, are gamma delta T cells comprising a TAC comprising (a) a DARPin, (b) UCHT1, and (c) a transmembrane and cytosolic domain of the CD4 co-receptor. In some embodiments, the TAC comprises (a) a DARPin, (b) UCHT1 (Y182T), and (c) a transmembrane and cytosolic domain of the CD4 co-receptor. In some embodiments, the TAC comprises (a) a DARPin, (b) huUCHT1, and (c) a transmembrane and cytosolic domain of the CD4 co-receptor. In some embodiments, the TAC comprises (a) a DARPin, (b) huUCHT1 (Y177T), and (c) a transmembrane and cytosolic domain of the CD4 co-receptor. In some embodiments, the TAC comprises (a) a DARPin, (b) OKT3, and (c) a transmembrane and cytosolic domain of the CD4 co-receptor. In some embodiments, the TAC comprises (a) a DARPin, (b) F6A, and (c) a transmembrane and cytosolic domain of the CD4 co-receptor. In some embodiments, the TAC comprises (a) a DARPin, (b) L2K, and (c) a transmembrane and cytosolic domain of the CD4 co-receptor.

Disclosed herein, in certain embodiments, are gamma delta T cells comprising a TAC comprising (a) a scFv, (b) UCHT1, and (c) a transmembrane and cytosolic domain of the CD4 co-receptor. In some embodiments, the TAC comprises (a) a scFv, (b) UCHT1 (Y182T), and (c) a transmembrane and cytosolic domain of the CD4 co-receptor. In some embodiments, the TAC comprises (a) a scFv, (b) huUCHT1, and (c) a transmembrane and cytosolic domain of the CD4 co-receptor. In some embodiments, the TAC comprises (a) a scFv, (b) huUCHT1 (Y177T), and (c) a transmembrane and cytosolic domain of the CD4 co-receptor. In some embodiments, the TAC comprises (a) a scFv, (b) OKT3, and (c) a transmembrane and cytosolic domain of the CD4 co-receptor. In some embodiments, the TAC comprises (a) a scFv, (b) F6A, and (c) a transmembrane and cytosolic domain of the CD4 co-receptor. In some embodiments, the TAC comprises (a) a scFv, (b) L2K, and (c) a transmembrane and cytosolic domain of the CD4 co-receptor.

Disclosed herein, in certain embodiments, are gamma delta T cells comprising a TAC comprising (a) a HER2-specific DARPin, (b) UCHT1, and (c) a transmembrane and cytosolic domain of the CD4 co-receptor. In some embodiments, the TAC comprises (a) a HER2-specific DARPin, (b) UCHT1 (Y182T), and (c) a transmembrane and cytosolic domain of the CD4 co-receptor. In some embodiments, the TAC comprises (a) a HER2-specific DARPin, (b) huUCHT1, and (c) a transmembrane and cytosolic domain of the CD4 co-receptor. In some embodiments, the TAC comprises (a) a target-specific antigen-binding domain, (b) huUCHT1 (Y177T), and (c) a transmembrane and cytosolic domain of the CD4 co-receptor. In some embodiments, the TAC comprises (a) a HER2-specific DARPin, (b) OKT3, and (c) a transmembrane and cytosolic domain of the CD4 co-receptor. In some embodiments, the TAC comprises (a) a HER2-specific DARPin, (b) F6A, and (c) a transmembrane and cytosolic domain of the CD4 co-receptor. In some embodiments, the TAC comprises (a) a HER2-specific DARPin, (b) L2K, and (c) a transmembrane and cytosolic domain of the CD4 co-receptor.

Disclosed herein, in certain embodiments, are gamma delta T cells comprising a TAC comprising (a) a BCMA-specific ScFv, (b) UCHT1, and (c) a transmembrane and cytosolic domain of the CD4 co-receptor. In some embodiments, the TAC comprises (a) a BCMA-specific ScFv, (b) UCHT1 (Y182T), and (c) a transmembrane and cytosolic domain of the CD4 co-receptor. In some embodiments, the TAC comprises (a) a BCMA-specific ScFv, (b) huUCHT1, and (c) a transmembrane and cytosolic domain of the CD4 co-receptor. In some embodiments, the TAC comprises (a) a BCMA-specific ScFv, (b) huUCHT1 (Y177T), and (c) a transmembrane and cytosolic domain of the CD4 co-receptor. In some embodiments, the TAC comprises (a) a BCMA-specific ScFv, (b) OKT3, and (c) a transmembrane and cytosolic domain of the CD4 co-receptor. In some embodiments, the TAC comprises (a) a BCMA-specific ScFv, (b) F6A, and (c) a transmembrane and cytosolic domain of the CD4 co-receptor. In some embodiments, the TAC comprises (a) a BCMA-specific ScFv, (b) L2K, and (c) a transmembrane and cytosolic domain of the CD4 co-receptor.

Disclosed herein, in certain embodiments, are gamma delta T cells comprising a TAC comprising (a) a CD19-specific ScFv, (b) UCHT1, and (c) a transmembrane and cytosolic domain of the CD4 co-receptor. In some embodiments, the TAC comprises (a) a CD19-specific ScFv, (b) UCHT1 (Y182T), and (c) a transmembrane and cytosolic domain of the CD4 co-receptor. In some embodiments, the TAC comprises (a) a CD19-specific ScFv, (b) huUCHT1, and (c) a transmembrane and cytosolic domain of the CD4 co-receptor. In some embodiments, the TAC comprises (a) a CD19-specific ScFv, (b) huUCHT1 (Y177T), and (c) a transmembrane and cytosolic domain of the CD4 co-receptor. In some embodiments, the TAC comprises (a) a CD19-specific ScFv, (b) OKT3, and (c) a transmembrane and cytosolic domain of the CD4 co-receptor. In some embodiments, the TAC comprises (a) a CD19-specific ScFv, (b) F6A, and (c) a transmembrane and cytosolic domain of the CD4 co-receptor. In some embodiments, the TAC comprises (a) a CD19-specific ScFv, (b) L2K, and (c) a transmembrane and cytosolic domain of the CD4 co-receptor.

Disclosed herein, in certain embodiments, are gamma delta T cells comprising a TAC comprising (a) a target-specific antigen-binding domain, (b) a single-chain antibody (scFv) that binds CD3ε, and (c) a transmembrane and cytosolic domain of the CD8 co-receptor. In some embodiments, the TAC comprises (a) a target-specific antigen-binding domain, (b) UCHT1, and (c) a transmembrane and cytosolic domain of the CD8 co-receptor. In some embodiments, the TAC comprises (a) a target-specific antigen-binding domain, (b) UCHT1 (Y182T), and (c) a transmembrane and cytosolic domain of the CD8 co-receptor. In some embodiments, the TAC comprises (a) a target-specific antigen-binding domain, (b) huUCHT1, and (c) a transmembrane and cytosolic domain of the CD8 co-receptor. In some embodiments, the TAC comprises (a) a target-specific antigen-binding domain, (b) huUCHT1 (Y177T), and (c) a transmembrane and cytosolic domain of the CD8 co-receptor. In some embodiments, the TAC comprises (a) a target-specific antigen-binding domain, (b) OKT3, and (c) a transmembrane and cytosolic domain of the CD8 co-receptor. In some embodiments, the TAC comprises (a) a target-specific antigen-binding domain, (b) F6A, and (c) a transmembrane and cytosolic domain of the CD8 co-receptor. In some embodiments, the TAC comprises (a) a target-specific antigen-binding domain, (b) L2K, and (c) a transmembrane and cytosolic domain of the CD8 co-receptor.

Disclosed herein, in certain embodiments, are gamma delta T cells comprising a TAC comprising (a) a DARPin, (b) UCHT1, and (c) a transmembrane and cytosolic domain of the CD4 co-receptor. In some embodiments, the TAC comprises (a) a DARPin, (b) UCHT1 (Y182T), and (c) a transmembrane and cytosolic domain of the CD8 co-receptor. In some embodiments, the TAC comprises (a) a DARPin, (b) huUCHT1, and (c) a transmembrane and cytosolic domain of the CD8 co-receptor. In some embodiments, the TAC comprises (a) a DARPin, (b) huUCHT1 (Y177T), and (c) a transmembrane and cytosolic domain of the CD8 co-receptor. In some embodiments, the TAC comprises (a) a DARPin, (b) OKT3, and (c) a transmembrane and cytosolic domain of the CD8 co-receptor. In some embodiments, the TAC comprises (a) a DARPin, (b) F6A, and (c) a transmembrane and cytosolic domain of the CD8 co-receptor. In some embodiments, the TAC comprises (a) a DARPin, (b) L2K, and (c) a transmembrane and cytosolic domain of the CD8 co-receptor.

Disclosed herein, in certain embodiments, are gamma delta T cells comprising a TAC comprising (a) a scFv, (b) UCHT1, and (c) a transmembrane and cytosolic domain of the CD8 co-receptor. In some embodiments, the TAC comprises (a) a scFv, (b) UCHT1 (Y182T), and (c) a transmembrane and cytosolic domain of the CD8 co-receptor. In some embodiments, the TAC comprises (a) a scFv, (b) huUCHT1, and (c) a transmembrane and cytosolic domain of the CD8 co-receptor. In some embodiments, the TAC comprises (a) a scFv, (b) huUCHT1 (Y177T), and (c) a transmembrane and cytosolic domain of the CD8 co-receptor. In some embodiments, the TAC comprises (a) a scFv, (b) OKT3, and (c) a transmembrane and cytosolic domain of the CD8 co-receptor. In some embodiments, the TAC comprises (a) a scFv, (b) F6A, and (c) a transmembrane and cytosolic domain of the CD8 co-receptor. In some embodiments, the TAC comprises (a) a scFv, (b) L2K, and (c) a transmembrane and cytosolic domain of the CD8 co-receptor.

Disclosed herein, in certain embodiments, are gamma delta T cells comprising a TAC comprising (a) a HER2-specific DARPin, (b) UCHT1, and (c) a transmembrane and cytosolic domain of the CD8 co-receptor. In some embodiments, the TAC comprises (a) a HER2-specific DARPin, (b) UCHT1 (Y182T), and (c) a transmembrane and cytosolic domain of the CD8 co-receptor. In some embodiments, the TAC comprises (a) a HER2-specific DARPin, (b) huUCHT1, and (c) a transmembrane and cytosolic domain of the CD8 co-receptor. In some embodiments, the TAC comprises (a) a target-specific antigen-binding domain, (b) huUCHT1 (Y177T), and (c) a transmembrane and cytosolic domain of the CD8 co-receptor. In some embodiments, the TAC comprises (a) a HER2-specific DARPin, (b) OKT3, and (c) a transmembrane and cytosolic domain of the CD8 co-receptor. In some embodiments, the TAC comprises (a) a HER2-specific DARPin, (b) F6A, and (c) a transmembrane and cytosolic domain of the CD8 co-receptor. In some embodiments, the TAC comprises (a) a HER2-specific DARPin, (b) L2K, and (c) a transmembrane and cytosolic domain of the CD8 co-receptor.

Disclosed herein, in certain embodiments, are gamma delta T cells comprising a TAC comprising (a) a BCMA-specific ScFv, (b) UCHT1, and (c) a transmembrane and cytosolic domain of the CD8 co-receptor. In some embodiments, the TAC comprises (a) a BCMA-specific ScFv, (b) UCHT1 (Y182T), and (c) a transmembrane and cytosolic domain of the CD8 co-receptor. In some embodiments, the TAC comprises (a) a BCMA-specific ScFv, (b) huUCHT1, and (c) a transmembrane and cytosolic domain of the CD8 co-receptor. In some embodiments, the TAC comprises (a) a BCMA-specific ScFv, (b) huUCHT1 (Y177T), and (c) a transmembrane and cytosolic domain of the CD8 co-receptor. In some embodiments, the TAC comprises (a) a BCMA-specific ScFv, (b) OKT3, and (c) a transmembrane and cytosolic domain of the CD8 co-receptor. In some embodiments, the TAC comprises (a) a BCMA-specific ScFv, (b) F6A, and (c) a transmembrane and cytosolic domain of the CD8 co-receptor. In some embodiments, the TAC comprises (a) a BCMA-specific ScFv, (b) L2K, and (c) a transmembrane and cytosolic domain of the CD8 co-receptor.

Disclosed herein, in certain embodiments, are gamma delta T cells comprising a TAC comprising (a) a CD19-specific ScFv, (b) UCHT1, and (c) a transmembrane and cytosolic domain of the CD8 co-receptor. In some embodiments, the TAC comprises (a) a CD19-specific ScFv, (b) UCHT1 (Y182T), and (c) a transmembrane and cytosolic domain of the CD8 co-receptor. In some embodiments, the TAC comprises (a) a CD19-specific ScFv, (b) huUCHT1, and (c) a transmembrane and cytosolic domain of the CD8 co-receptor. In some embodiments, the TAC comprises (a) a CD19-specific ScFv, (b) huUCHT1 (Y177T), and (c) a transmembrane and cytosolic domain of the CD8 co-receptor. In some embodiments, the TAC comprises (a) a CD19-specific ScFv, (b) OKT3, and (c) a transmembrane and cytosolic domain of the CD8 co-receptor. In some embodiments, the TAC comprises (a) a CD19-specific ScFv, (b) F6A, and (c) a transmembrane and cytosolic domain of the CD8 co-receptor. In some embodiments, the TAC comprises (a) a CD19-specific ScFv, (b) L2K, and (c) a transmembrane and cytosolic domain of the CD8 co-receptor.

In certain instances, the TAC draws CD3 and TCR into lipid raft regions of the membrane, and brings Lck into the proximity of the TCR, similar to natural MHC binding.

In some embodiments, the TAC disclosed herein is the anti-HER2 DARPin TAC (also referred to as configuration 1; SEQ ID NO: 1 and 2) includes, in order:
i) the anti-HER2 TAC leader sequence (secretion signal) (SEQ ID NO: 5 and 6)
ii) DARPin specific for HER2 antigen (SEQ ID NO: 7 and 8)
iii) Myc tag (SEQ ID NO: 9 and 10)
iv) Connector (SEQ ID NO: 11 and 12)
v) UCHT1 (SEQ ID NO: 13 and 14)
vi) Linker (SEQ ID NO: 15 and 16)
vii) CD4 (SEQ ID NO: 17 and 18).

Disclosed herein, in certain embodiments, are gamma delta T cells comprising a HER2-TAC encoded by a nucleotide sequence having at least 70% sequence identity with SEQ ID NO: 65 (HER2 TAC: huIgG leader; huUCHT1 CD3-binding domain). In some embodiments, the HER2-TAC is encoded by a nucleotide sequence having at least 75% sequence identity with SEQ ID NO: 65 (HER2 TAC: huIgG leader; huUCHT1 CD3-binding domain). In some embodiments, the HER2-TAC is encoded by a nucleotide sequence having at least 80% sequence identity with SEQ ID NO: 65 (HER2 TAC: huIgG leader; huUCHT1 CD3-binding domain). In some embodiments, the HER2-TAC is encoded by a nucleotide sequence having at least 85% sequence identity with SEQ ID NO: 65 (HER2 TAC: huIgG leader; huUCHT1 CD3-binding domain). In some embodiments, the HER2-TAC is encoded by a nucleotide sequence having at least 90% sequence identity with SEQ ID NO: 65 (HER2 TAC: huIgG leader; huUCHT1 CD3-binding domain). In some embodiments, the HER2-TAC is encoded by a nucleotide sequence having at least 95% sequence identity with SEQ ID NO: 65 (HER2 TAC: huIgG leader; huUCHT1 CD3-binding domain). In some embodiments, the HER2-TAC is encoded by a nucleotide sequence having at least 96% sequence identity with SEQ ID NO: 65 (HER2 TAC: huIgG leader; huUCHT1 CD3-binding domain). In some embodiments, the HER2-TAC is encoded by a nucleotide sequence having at least 97% sequence identity with SEQ ID NO: 65 (HER2 TAC: huIgG leader; huUCHT1 CD3-binding domain). In some embodiments, the HER2-TAC is encoded by a nucleotide sequence having at least 98% sequence identity with SEQ ID NO: 65 (HER2 TAC: huIgG leader; huUCHT1 CD3-binding domain). In some embodiments, the HER2-TAC is encoded by a nucleotide sequence having at least 99% sequence identity with SEQ ID NO: 65 (HER2 TAC: huIgG leader; huUCHT1 CD3-binding domain). In some embodiments, the HER2-TAC is encoded by a nucleotide sequence of SEQ ID NO: 65 (HER2 TAC: huIgG leader; huUCHT1 CD3-binding domain).

Disclosed herein, in certain embodiments, are gamma delta T cells comprising a HER2-TAC comprising an amino acid sequence having at least 70% sequence identity with SEQ ID NO: 66 (HER2 TAC: huIgG leader; huUCHT1 CD3-binding domain). In some embodiments, the HER2-TAC comprises an amino acid sequence having at least 75% sequence identity with SEQ ID NO: 66 (HER2 TAC: huIgG leader; huUCHT1 CD3-binding domain). In some embodiments, the HER2-TAC comprises an amino acid sequence having at least 80% sequence identity with SEQ ID NO: 66 (HER2 TAC: huIgG leader; huUCHT1 CD3-binding domain). In some embodiments, the HER2-TAC comprises an amino acid sequence having at least 85% sequence identity with SEQ ID NO: 66 (HER2 TAC: huIgG leader; huUCHT1 CD3-binding domain). In some embodiments, the HER2-TAC an amino acid sequence having at least 90% sequence identity with SEQ ID NO: 66 (HER2 TAC: huIgG leader; huUCHT1 CD3-binding domain). In some embodiments, the HER2-TAC comprises an amino acid sequence having at least 95% sequence identity with SEQ ID NO: 66 (HER2 TAC: huIgG leader; huUCHT1 CD3-binding domain). In some embodiments, the HER2-TAC comprises an amino acid sequence having at least 96% sequence identity with SEQ ID NO: 66 (HER2 TAC: huIgG leader; huUCHT1 CD3-binding domain). In some embodiments, the HER2-TAC comprises an amino acid sequence having at least 97% sequence identity with SEQ ID NO: 66 (HER2 TAC: huIgG leader; huUCHT1 CD3-binding domain). In some embodiments, the HER2-TAC comprises an amino acid sequence having at least 98% sequence identity with SEQ ID NO: 66 (HER2 TAC: huIgG leader; huUCHT1 CD3-binding domain). In some embodiments, the HER2-TAC comprises an amino acid sequence having at least 99% sequence identity with SEQ ID NO: 66 (HER2 TAC: huIgG leader; huUCHT1 CD3-binding domain). In some embodiments, the HER2-TAC comprises an amino acid sequence of SEQ ID NO: 66 (HER2 TAC: huIgG leader; huUCHT1 CD3-binding domain).

Disclosed herein, in certain embodiments, are gamma delta T cells comprising a HER2-TAC encoded by a nucleotide sequence having at least 70% sequence identity with SEQ ID NO: 67 (HER2 TAC: CD8a leader; huUCHT1 CD3-binding domain). In some embodiments, the HER2-TAC is encoded by a nucleotide sequence having at least 75% sequence identity with SEQ ID NO: 67 (HER2 TAC: CD8a leader; huUCHT1 CD3-binding domain). In some embodiments, the HER2-TAC is encoded by a nucleotide sequence having at least 80% sequence identity with SEQ ID NO: 67 (HER2 TAC: CD8a leader; huUCHT1 CD3-binding domain). In some embodiments, the HER2-TAC is encoded by a nucleotide sequence having at least 85% sequence identity with SEQ ID NO: 67 (HER2 TAC: CD8a leader; huUCHT1 CD3-binding domain). In some embodiments, the HER2-TAC is encoded by a nucleotide sequence having at least 90% sequence identity with SEQ ID NO: 67 (HER2 TAC: CD8a leader; huUCHT1 CD3-binding domain). In some embodiments, the HER2-TAC is encoded by a nucleotide sequence having at least 95% sequence identity with SEQ ID NO: 67 (HER2 TAC: CD8a leader; huUCHT1 CD3-binding domain). In some embodiments, the HER2-TAC is encoded by a nucleotide sequence having at least 96% sequence identity with SEQ ID NO: 67 (HER2 TAC: CD8a leader; huUCHT1 CD3-binding domain). In some embodiments, the HER2-TAC is encoded by a nucleotide sequence having at least 97% sequence identity with SEQ ID NO: 67 (HER2 TAC: CD8a leader; huUCHT1 CD3-binding domain). In some embodiments, the HER2-TAC is encoded by a nucleotide sequence having at least 98% sequence identity with SEQ ID NO: 67 (HER2 TAC: CD8a leader; huUCHT1 CD3-binding domain). In some embodiments, the HER2-TAC is encoded by a nucleotide sequence having at least 99% sequence identity with SEQ ID NO: 67 (HER2 TAC: CD8a leader; huUCHT1 CD3-binding domain). In some embodiments, the HER2-TAC is encoded by a nucleotide sequence of SEQ ID NO: 67 (HER2 TAC: CD8a leader; huUCHT1 CD3-binding domain).

Disclosed herein, in certain embodiments, are gamma delta T cells comprising a HER2-TAC comprising an amino acid sequence having at least 70% sequence identity with SEQ ID NO: 68 (HER2 TAC: CD8a leader; huUCHT1 CD3-binding domain). In some embodiments, the HER2-TAC comprises an amino acid sequence having at least 75% sequence identity with SEQ ID NO: 68 (HER2 TAC: CD8a leader; huUCHT1 CD3-binding domain). In some embodiments, the HER2-TAC comprises an amino acid sequence having at least 80% sequence identity with SEQ ID NO: 68 (HER2 TAC: CD8a leader; huUCHT1 CD3-binding domain). In some embodiments, the HER2-TAC comprises an amino acid sequence having at least 85% sequence identity with SEQ ID NO: 68 (HER2 TAC: CD8a leader; huUCHT1 CD3-binding domain). In some embodiments, the HER2-TAC an amino acid sequence having at least 90% sequence identity with SEQ ID NO: 68 (HER2 TAC: CD8a leader; huUCHT1 CD3-binding domain). In some embodiments, the HER2-TAC comprises an amino acid sequence having at least 95% sequence identity with SEQ ID NO: 68 (HER2 TAC: CD8a leader; huUCHT1 CD3-binding domain). In some embodiments, the HER2-TAC comprises an amino acid sequence having at least 96% sequence identity with SEQ ID NO: 68 (HER2 TAC: CD8a leader; huUCHT1 CD3-binding domain). In some embodiments, the HER2-TAC comprises an amino acid sequence having at least 97% sequence identity with SEQ ID NO: 68 (HER2 TAC: CD8a leader; huUCHT1 CD3-binding domain). In some embodiments, the HER2-TAC comprises an amino acid sequence having at least 98% sequence identity with SEQ ID NO: 68 (HER2 TAC: CD8a leader; huUCHT1 CD3-binding domain). In some embodiments, the HER2-TAC comprises an amino acid sequence having at least 99% sequence identity with SEQ ID NO: 68 (HER2 TAC: CD8a leader; huUCHT1 CD3-binding domain). In some embodiments, the HER2-TAC comprises an amino acid sequence of SEQ ID NO: 68 (HER2 TAC: CD8a leader; huUCHT1 CD3-binding domain).

Disclosed herein, in certain embodiments, are gamma delta T cells comprising a HER2-TAC encoded by a nucleotide sequence having at least 70% sequence identity with SEQ ID NO: 75 (HER2 TAC: muIgG leader; huUCHT1 CD3-binding domain). In some embodiments, the HER2-TAC is encoded by a nucleotide sequence having at least 75% sequence identity with SEQ ID NO: 75 (HER2 TAC: muIgG leader; huUCHT1 CD3-binding domain). In some embodiments, the HER2-TAC is encoded by a nucleotide sequence having at least 80% sequence identity with SEQ ID NO: 75 (HER2 TAC: muIgG leader; huUCHT1 CD3-binding domain). In some embodiments, the HER2-TAC is encoded by a nucleotide sequence having at least 85% sequence identity with SEQ ID NO: 75 (HER2 TAC: muIgG leader; huUCHT1 CD3-binding domain). In some embodiments, the HER2-TAC is encoded by a nucleotide sequence having at least 90% sequence identity with SEQ ID NO: 75 (HER2 TAC: muIgG leader; huUCHT1 CD3-binding domain). In some embodiments, the HER2-TAC is encoded by a nucleotide sequence having at least 95% sequence identity with SEQ ID NO: 75 (HER2 TAC: muIgG leader; huUCHT1 CD3-binding domain). In some embodiments, the HER2-TAC is encoded by a nucleotide sequence having at least 96% sequence identity with SEQ ID NO: 75 (HER2 TAC: muIgG leader; huUCHT1 CD3-binding domain). In some embodiments, the HER2-TAC is encoded by a nucleotide sequence having at least 97% sequence identity with SEQ ID NO: 75 (HER2 TAC: muIgG leader; huUCHT1 CD3-binding domain). In some embodiments, the HER2-TAC is encoded by a nucleotide sequence having at least 98% sequence identity with SEQ ID NO: 75 (HER2 TAC: muIgG leader; huUCHT1 CD3-binding domain). In some embodiments, the HER2-TAC is encoded by a nucleotide sequence having at least 99% sequence identity with SEQ ID NO: 75 (HER2 TAC: muIgG leader; huUCHT1 CD3-binding domain). In some embodiments, the HER2-TAC is encoded by a nucleotide sequence of SEQ ID NO: 75 (HER2 TAC: muIgG leader; huUCHT1 CD3-binding domain).

Disclosed herein, in certain embodiments, are gamma delta T cells comprising a HER2-TAC comprising an amino acid sequence having at least 70% sequence identity with SEQ ID NO: 76 (HER2 TAC: muIgG leader; huUCHT1 CD3-binding domain). In some embodiments, the HER2-TAC comprises an amino acid sequence having at least 75% sequence identity with SEQ ID NO: 76 (HER2 TAC: muIgG leader; huUCHT1 CD3-binding domain). In some embodiments, the HER2-TAC comprises an amino acid sequence having at least 80% sequence identity with SEQ ID NO: 76 (HER2 TAC: muIgG leader; huUCHT1 CD3-binding domain). In some embodiments, the HER2-TAC comprises an amino acid sequence having at least 85% sequence identity with SEQ ID NO: 76 (HER2 TAC: muIgG leader; huUCHT1 CD3-binding domain). In some embodiments, the HER2-TAC an amino acid sequence having at least 90% sequence identity with SEQ ID NO: 76 (HER2 TAC: muIgG leader; huUCHT1 CD3-binding domain). In some embodiments, the HER2-TAC comprises an amino acid sequence having at least 95% sequence identity with SEQ ID NO: 76 (HER2 TAC: muIgG leader; huUCHT1 CD3-binding domain). In some embodiments, the HER2-TAC comprises an amino acid sequence having at least 96% sequence identity with SEQ ID NO: 76 (HER2 TAC: muIgG leader; huUCHT1 CD3-binding domain). In some embodiments, the HER2-TAC comprises an amino acid sequence having at least 97% sequence identity with SEQ ID NO: 76 (HER2 TAC: muIgG leader; huUCHT1 CD3-binding domain). In some embodiments, the HER2-TAC comprises an amino acid sequence having at least 98% sequence identity with SEQ ID NO: 76 (HER2 TAC: muIgG leader; huUCHT1 CD3-binding domain). In some embodiments, the HER2-TAC comprises an amino acid sequence having at least 99% sequence identity with SEQ ID NO: 76 (HER2 TAC: muIgG leader; huUCHT1 CD3-binding domain). In some embodiments, the HER2-TAC comprises an amino acid sequence of SEQ ID NO: 76 (HER2 TAC: muIgG leader; huUCHT1 CD3-binding domain).

Disclosed herein, in certain embodiments, are gamma delta T cells comprising a BCMA-TAC encoded by a nucleotide sequence having at least 70% sequence identity with SEQ ID NO: 55 (BCMA TAC: 3625 BCMA scFv, Vh-Vl, helix linker; huUCHT1 CD3-binding domain). In some embodiments, the BCMA-TAC is encoded by a nucleotide sequence having at least 75% sequence identity with SEQ ID NO: 55 (BCMA TAC: 3625 BCMA scFv, Vh-Vl, helix linker; huUCHT1 CD3-binding domain). In some embodiments, the BCMA-TAC is encoded by a nucleotide sequence having at least 80% sequence identity with SEQ ID NO: 55 (BCMA TAC: 3625 BCMA scFv, Vh-Vl, helix linker; huUCHT1 CD3-binding domain). In some embodiments, the BCMA-TAC is encoded by a nucleotide sequence having at least 85% sequence identity with SEQ ID NO: 55 (BCMA TAC: 3625 BCMA scFv, Vh-Vl, helix linker; huUCHT1 CD3-binding domain). In some embodiments, the BCMA-TAC is encoded by a nucleotide sequence having at least 90% sequence identity with SEQ ID NO: 55 (BCMA TAC: 3625 BCMA scFv, Vh-Vl, helix linker; huUCHT1 CD3-binding domain). In some embodiments, the BCMA-TAC is encoded by a nucleotide sequence having at least 95% sequence identity with SEQ ID NO: 55 (BCMA TAC: 3625 BCMA scFv, Vh-Vl, helix linker; huUCHT1 CD3-binding domain). In some embodiments, the BCMA-TAC is encoded by a nucleotide sequence having at least 96% sequence identity with SEQ ID NO: 55 (BCMA TAC: 3625 BCMA scFv, Vh-Vl, helix linker; huUCHT1 CD3-binding domain). In some embodiments, the BCMA-TAC is encoded by a nucleotide sequence having at least 97% sequence identity with SEQ ID NO: 55 (BCMA TAC: 3625 BCMA scFv, Vh-Vl, helix linker; huUCHT1 CD3-binding domain). In some embodiments, the BCMA-TAC is encoded by a nucleotide sequence having at least 98% sequence identity with SEQ ID NO: 55 (BCMA TAC: 3625 BCMA scFv, Vh-Vl, helix linker; huUCHT1 CD3-binding domain). In some embodiments, the BCMA-TAC is encoded by a nucleotide sequence having at least 99% sequence identity with SEQ ID NO: 55 (BCMA TAC: 3625 BCMA scFv, Vh-Vl, helix linker; huUCHT1 CD3-binding domain). In some embodiments, the BCMA-TAC is encoded by a nucleotide sequence of SEQ ID NO: 55 (BCMA TAC: 3625 BCMA scFv, Vh-Vl, helix linker; huUCHT1 CD3-binding domain).

Disclosed herein, in certain embodiments, are gamma delta T cells comprising a BCMA-TAC comprising an amino acid sequence having at least 70% sequence identity with SEQ ID NO: 56 (BCMA TAC: 3625 BCMA scFv, Vh-Vl, helix linker; huUCHT1 CD3-binding domain). In some embodiments, the BCMA-TAC comprises an amino acid sequence having at least 75% sequence identity with SEQ ID NO: 56 (BCMA TAC: 3625 BCMA scFv, Vh-Vl, helix linker; huUCHT1 CD3-binding domain). In some embodiments, the BCMA-TAC comprises an amino acid sequence having at least 80% sequence identity with SEQ ID NO: 56 (BCMA TAC: 3625 BCMA scFv, Vh-Vl, helix linker; huUCHT1 CD3-binding domain). In some embodiments, the BCMA-TAC comprises an amino acid sequence having at least 85% sequence identity with SEQ ID NO: 56 (BCMA TAC: 3625 BCMA scFv, Vh-Vl, helix linker; huUCHT1 CD3-binding domain). In some embodiments, the BCMA-TAC an amino acid sequence having at least 90% sequence identity with SEQ ID NO: 56 (BCMA TAC: 3625 BCMA scFv, Vh-Vl, helix linker; huUCHT1 CD3-binding domain). In some embodiments, the BCMA-TAC comprises an amino acid sequence having at least 95% sequence identity with SEQ ID NO: 56 (BCMA TAC: 3625 BCMA scFv, Vh-Vl, helix linker; huUCHT1 CD3-binding domain). In some embodiments, the BCMA-TAC comprises an amino acid sequence having at least 96% sequence identity with SEQ ID NO: 56 (BCMA TAC: 3625 BCMA scFv, Vh-Vl, helix linker; huUCHT1 CD3-binding domain). In some embodiments, the BCMA-TAC comprises an amino acid sequence having at least 97% sequence identity with SEQ ID NO: 56 (BCMA TAC: 3625 BCMA scFv, Vh-Vl, helix linker; huUCHT1 CD3-binding domain). In some embodiments, the BCMA-TAC comprises an amino acid sequence having at least 98% sequence identity with SEQ ID NO: 56 (BCMA TAC: 3625 BCMA scFv, Vh-Vl, helix linker; huUCHT1 CD3-binding domain). In some embodiments, the BCMA-TAC comprises an amino acid sequence having at least 99% sequence identity with SEQ ID NO: 56 (BCMA TAC: 3625 BCMA scFv, Vh-Vl, helix linker; huUCHT1 CD3-binding domain). In some embodiments, the BCMA-TAC comprises an amino acid sequence of SEQ ID NO: 56 (BCMA TAC: 3625 BCMA scFv, Vh-Vl, helix linker; huUCHT1 CD3-binding domain).

Disclosed herein, in certain embodiments, are gamma delta T cells comprising a BCMA-TAC encoded by a nucleotide sequence having at least 70% sequence identity with SEQ ID NO: 57 (BCMA TAC: 3625 BCMA scFv, Vl-Vh, helix linker; huUCHT1 CD3-binding domain). In some embodiments, the BCMA-TAC is encoded by a nucleotide sequence having at least 75% sequence identity with SEQ ID NO: 57 (BCMA TAC: 3625 BCMA scFv, Vl-Vh, helix linker; huUCHT1 CD3-binding domain). In some embodiments, the BCMA-TAC is encoded by a nucleotide sequence having at least 80% sequence identity with SEQ ID NO: 57 (BCMA TAC: 3625 BCMA scFv, Vl-Vh, helix linker; huUCHT1 CD3-binding domain). In some embodiments, the BCMA-TAC is encoded by a nucleotide sequence having at least 85% sequence identity with SEQ ID NO: 57 (BCMA TAC: 3625 BCMA scFv, Vl-Vh, helix linker; huUCHT1 CD3-binding domain). In some embodiments, the BCMA-TAC is encoded by a nucleotide sequence having at least 90% sequence identity with SEQ ID NO: 57 (BCMA TAC: 3625 BCMA scFv, Vl-Vh, helix linker; huUCHT1 CD3-binding domain). In some embodiments, the BCMA-TAC is encoded by a nucleotide sequence having at least 95% sequence identity with SEQ ID NO: 57 (BCMA TAC: 3625 BCMA scFv, Vl-Vh, helix linker; huUCHT1 CD3-binding domain). In some embodiments, the BCMA-TAC is encoded by a nucleotide sequence having at least 96% sequence identity with SEQ ID NO: 57 (BCMA TAC: 3625 BCMA scFv, Vl-Vh, helix linker; huUCHT1 CD3-binding domain). In some embodiments, the BCMA-TAC is encoded by a nucleotide sequence having at least 97% sequence identity with SEQ ID NO: 57 (BCMA TAC: 3625 BCMA scFv, Vl-Vh, helix linker; huUCHT1 CD3-binding domain). In some embodiments, the BCMA-TAC is encoded by a nucleotide sequence having at least 98% sequence identity with SEQ ID NO: 57 (BCMA TAC: 3625 BCMA scFv, Vl-Vh, helix linker; huUCHT1 CD3-binding domain). In some embodiments, the BCMA-TAC is encoded by a nucleotide sequence having at least 99% sequence identity with SEQ ID NO: 57 (BCMA TAC: 3625 BCMA scFv, Vl-Vh, helix linker; huUCHT1 CD3-binding domain). In some embodiments, the BCMA-TAC is encoded by a nucleotide sequence of SEQ ID NO: 57 (BCMA TAC: 3625 BCMA scFv, Vl-Vh, helix linker; huUCHT1 CD3-binding domain).

Disclosed herein, in certain embodiments, are gamma delta T cells comprising a BCMA-TAC comprising an amino acid sequence having at least 70% sequence identity with SEQ ID NO: 58 (BCMA TAC: 3625 BCMA scFv, Vl-Vh, helix linker; huUCHT1 CD3-binding domain). In some embodiments, the BCMA-TAC comprises an amino acid sequence having at least 75% sequence identity with SEQ ID NO: 58 (BCMA TAC: 3625 BCMA scFv, Vl-Vh, helix linker; huUCHT1 CD3-binding domain). In some embodiments, the BCMA-TAC comprises an amino acid sequence having at least 80% sequence identity with SEQ ID NO: 58 (BCMA TAC: 3625 BCMA scFv, Vl-Vh, helix linker; huUCHT1 CD3-binding domain). In some embodiments, the BCMA-TAC comprises an amino acid sequence having at least 85% sequence identity with SEQ ID NO: 58 (BCMA TAC: 3625 BCMA scFv, Vl-Vh, helix linker; huUCHT1 CD3-binding domain). In some embodiments, the BCMA-TAC an amino acid sequence having at least 90% sequence identity with SEQ ID NO: 58 (BCMA TAC: 3625 BCMA scFv, Vl-Vh, helix linker; huUCHT1 CD3-binding domain). In some embodiments, the BCMA-TAC comprises an amino acid sequence having at least 95% sequence identity with SEQ ID NO: 58 (BCMA TAC: 3625 BCMA scFv, Vl-Vh, helix linker; huUCHT1 CD3-binding domain). In some embodiments, the BCMA-TAC comprises an amino acid sequence having at least 96% sequence identity with SEQ ID NO: 58 (BCMA TAC: 3625 BCMA scFv, Vl-Vh, helix linker; huUCHT1 CD3-binding domain). In some embodiments, the BCMA-TAC comprises an amino acid sequence having at least 97% sequence identity with SEQ ID NO: 58 (BCMA TAC: 3625 BCMA scFv, Vl-Vh, helix linker; huUCHT1 CD3-binding domain). In some embodiments, the BCMA-TAC comprises an amino acid sequence having at least 98% sequence identity with SEQ ID NO: 58 (BCMA TAC: 3625 BCMA scFv, Vl-Vh, helix linker; huUCHT1 CD3-binding domain). In some embodiments, the BCMA-TAC comprises an amino acid sequence having at least 99% sequence identity with SEQ ID NO: 58 (BCMA TAC: 3625 BCMA scFv, Vl-Vh, helix linker; huUCHT1 CD3-binding domain). In some embodiments, the BCMA-TAC comprises an amino acid sequence of SEQ ID NO: 58 (BCMA TAC: 3625 BCMA scFv, Vl-Vh, helix linker; huUCHT1 CD3-binding domain).

Disclosed herein, in certain embodiments, are gamma delta T cells comprising a BCMA-TAC encoded by a nucleotide sequence having at least 70% sequence identity with SEQ ID NO: 59 (BCMA TAC: 3625 BCMA scFv, Vh-Vl, G4S linker; huUCHT1 CD3-binding domain). In some embodiments, the BCMA-TAC is encoded by a nucleotide sequence having at least 75% sequence identity with SEQ ID NO: 59 (BCMA TAC: 3625 BCMA scFv, Vh-Vl, G4S linker; huUCHT1 CD3-binding domain). In some embodiments, the BCMA-TAC is encoded by a nucleotide sequence having at least 80% sequence identity with SEQ ID NO: 59 (BCMA TAC: 3625 BCMA scFv, Vh-Vl, G4S linker; huUCHT1 CD3-binding domain). In some embodiments, the BCMA-TAC is encoded by a nucleotide sequence having at least 85% sequence identity with SEQ ID NO: 59 (BCMA TAC: 3625 BCMA scFv, Vh-Vl, G4S linker; huUCHT1 CD3-binding domain). In some embodiments, the BCMA-TAC is encoded by a nucleotide sequence having at least 90% sequence identity with SEQ ID NO: 59 (BCMA TAC: 3625 BCMA scFv, Vh-Vl, G4S linker; huUCHT1 CD3-binding domain). In some embodiments, the BCMA-TAC is encoded by a nucleotide sequence having at least 95% sequence identity with SEQ ID NO: 59 (BCMA TAC: 3625 BCMA scFv, Vh-Vl, G4S linker; huUCHT1 CD3-binding domain). In some embodiments, the BCMA-TAC is encoded by a nucleotide sequence having at least 96% sequence identity with SEQ ID NO: 59 (BCMA TAC: 3625 BCMA scFv, Vh-Vl, G4S linker; huUCHT1 CD3-binding domain). In some embodiments, the BCMA-TAC is encoded by a nucleotide sequence having at least 97% sequence identity with SEQ ID NO: 59 (BCMA TAC: 3625 BCMA scFv, Vh-Vl, G4S linker; huUCHT1 CD3-binding domain). In some embodiments, the BCMA-TAC is encoded by a nucleotide sequence having at least 98% sequence identity with SEQ ID NO: 59 (BCMA TAC: 3625 BCMA scFv, Vh-Vl, G4S linker; huUCHT1 CD3-binding domain). In some embodiments, the BCMA-TAC is encoded by a nucleotide sequence having at least 99% sequence identity with SEQ ID NO: 59 (BCMA TAC: 3625 BCMA scFv, Vh-Vl, G4S linker; huUCHT1 CD3-binding domain). In some embodiments, the BCMA-TAC is encoded by a nucleotide sequence of SEQ ID NO: 59 (BCMA TAC: 3625 BCMA scFv, Vh-Vl, G4S linker; huUCHT1 CD3-binding domain).

Disclosed herein, in certain embodiments, are gamma delta T cells comprising a BCMA-TAC comprising an amino acid sequence having at least 70% sequence identity with SEQ ID NO: 60 (BCMA TAC: 3625 BCMA scFv, Vh-Vl, G4S linker; huUCHT1 CD3-binding domain). In some embodiments, the BCMA-TAC comprises an amino acid sequence having at least 75% sequence identity with SEQ ID NO: 60 (BCMA TAC: 3625 BCMA scFv, Vh-Vl, G4S linker; huUCHT1 CD3-binding domain). In some embodiments, the BCMA-TAC comprises an amino acid sequence having at least 80% sequence identity with SEQ ID NO: 60 (BCMA TAC: 3625 BCMA scFv, Vh-Vl, G4S linker; huUCHT1 CD3-binding domain). In some embodiments, the BCMA-TAC comprises an amino acid sequence having at least 85% sequence identity with SEQ ID NO: 60 (BCMA TAC: 3625 BCMA scFv, Vh-Vl, G4S linker; huUCHT1 CD3-binding domain). In some embodiments, the BCMA-TAC an amino acid sequence having at least 90% sequence identity with SEQ ID NO: 60 (BCMA TAC: 3625 BCMA scFv, Vh-Vl, G4S linker; huUCHT1 CD3-binding domain). In some embodiments, the BCMA-TAC comprises an amino acid sequence having at least 95% sequence identity with SEQ ID NO: 60 (BCMA TAC: 3625 BCMA scFv, Vh-Vl, G4S linker; huUCHT1 CD3-binding domain). In some embodiments, the BCMA-TAC comprises an amino acid sequence having at least 96% sequence identity with SEQ ID NO: 60 (BCMA TAC: 3625 BCMA scFv, Vh-Vl, G4S linker; huUCHT1 CD3-binding domain). In some embodiments, the BCMA-TAC comprises an amino acid sequence having at least 97% sequence identity with SEQ ID NO: 60 (BCMA TAC: 3625 BCMA scFv, Vh-Vl, G4S linker; huUCHT1 CD3-binding domain). In some embodiments, the BCMA-TAC comprises an amino acid sequence having at least 98% sequence identity with SEQ ID NO: 60 (BCMA TAC: 3625 BCMA scFv, Vh-Vl, G4S linker; huUCHT1 CD3-binding domain). In some embodiments, the BCMA-TAC comprises an amino acid sequence having at least 99% sequence identity with SEQ ID NO: 60 (BCMA TAC: 3625 BCMA scFv, Vh-Vl, G4S linker; huUCHT1 CD3-binding domain). In some embodiments, the BCMA-TAC comprises an amino acid sequence of SEQ ID NO: 60 (BCMA TAC: 3625 BCMA scFv, Vh-Vl, G4S linker; huUCHT1 CD3-binding domain).

Disclosed herein, in certain embodiments, are gamma delta T cells comprising a BCMA-TAC encoded by a nucleotide sequence having at least 70% sequence identity with SEQ ID NO: 61 (BCMA TAC: 3625 BCMA scFv, Vl-Vh, G4S linker; huUCHT1 CD3-binding domain). In some embodiments, the BCMA-TAC is encoded by a nucleotide sequence having at least 75% sequence identity with SEQ ID NO: 61 (BCMA TAC: 3625 BCMA scFv, Vl-Vh, G4S linker; huUCHT1 CD3-binding domain). In some embodiments, the BCMA-TAC is encoded by a nucleotide sequence having at least 80% sequence identity with SEQ ID NO: 61 (BCMA TAC: 3625 BCMA scFv, Vl-Vh, G4S linker; huUCHT1 CD3-binding domain). In some embodiments, the BCMA-TAC is encoded by a nucleotide sequence having at least 85% sequence identity with SEQ ID NO: 61 (BCMA TAC: 3625 BCMA scFv, Vl-Vh, G4S linker; huUCHT1 CD3-binding domain). In some embodiments, the BCMA-TAC is encoded by a nucleotide sequence having at least 90% sequence identity with SEQ ID NO: 61 (BCMA TAC: 3625 BCMA scFv, Vl-Vh, G4S linker; huUCHT1 CD3-binding domain). In some embodiments, the BCMA-TAC is encoded by a nucleotide sequence having at least 95% sequence identity with SEQ ID NO: 61 (BCMA TAC: 3625 BCMA scFv, Vl-Vh, G4S linker; huUCHT1 CD3-binding domain). In some embodiments, the BCMA-TAC is encoded by a nucleotide sequence having at least 96% sequence identity with SEQ ID NO: 61 (BCMA TAC: 3625 BCMA scFv, Vl-Vh, G4S linker; huUCHT1 CD3-binding domain). In some embodiments, the BCMA-TAC is encoded by a nucleotide sequence having at least 97% sequence identity with SEQ ID NO: 61 (BCMA TAC: 3625 BCMA scFv, Vl-Vh, G4S linker; huUCHT1 CD3-binding domain). In some embodiments, the BCMA-TAC is encoded by a nucleotide sequence having at least 98% sequence identity with SEQ ID NO: 61 (BCMA TAC: 3625 BCMA scFv, Vl-Vh, G4S linker; huUCHT1 CD3-binding domain). In some embodiments, the BCMA-TAC is encoded by a nucleotide sequence having at least 99% sequence identity with SEQ ID NO: 61 (BCMA TAC: 3625 BCMA scFv, Vl-Vh, G4S linker; huUCHT1 CD3-binding domain). In some embodiments, the BCMA-TAC is encoded by a nucleotide sequence of SEQ ID NO: 61 (BCMA TAC: 3625 BCMA scFv, Vl-Vh, G4S linker; huUCHT1 CD3-binding domain).

Disclosed herein, in certain embodiments, are gamma delta T cells comprising a BCMA-TAC comprising an amino acid sequence having at least 70% sequence identity with SEQ ID NO: 62 (BCMA TAC: 3625 BCMA scFv, Vl-Vh, G4S linker; huUCHT1 CD3-binding domain). In some embodiments, the BCMA-TAC comprises an amino acid sequence having at least 75% sequence identity with SEQ ID NO: 62 (BCMA TAC: 3625 BCMA scFv, Vl-Vh, G4S linker; huUCHT1 CD3-binding domain). In some embodiments, the BCMA-TAC comprises an amino acid sequence having at least 80% sequence identity with SEQ ID NO: 62 (BCMA TAC: 3625 BCMA scFv, Vl-Vh, G4S linker; huUCHT1 CD3-binding domain). In some embodiments, the BCMA-TAC comprises an amino acid sequence having at least 85% sequence identity with SEQ ID NO: 62 (BCMA TAC: 3625 BCMA scFv, Vl-Vh, G4S linker; huUCHT1 CD3-binding domain). In some embodiments, the BCMA-TAC an amino acid sequence having at least 90% sequence identity with SEQ ID NO: 62 (BCMA TAC: 3625 BCMA scFv, Vl-Vh, G4S linker; huUCHT1 CD3-binding domain). In some embodiments, the BCMA-TAC comprises an amino acid sequence having at least 95% sequence identity with SEQ ID NO: 62 (BCMA TAC: 3625 BCMA scFv, Vl-Vh, G4S linker; huUCHT1 CD3-binding domain). In some embodiments, the BCMA-TAC comprises an amino acid sequence having at least 96% sequence identity with SEQ ID NO: 62 (BCMA TAC: 3625 BCMA scFv, Vl-Vh, G4S linker; huUCHT1 CD3-binding domain). In some embodiments, the BCMA-TAC comprises an amino acid sequence having at least 97% sequence identity with SEQ ID NO: 62 (BCMA TAC: 3625 BCMA scFv, Vl-Vh, G4S linker; huUCHT1 CD3-binding domain). In some embodiments, the BCMA-TAC comprises an amino acid sequence having at least 98% sequence identity with SEQ ID NO: 62 (BCMA TAC: 3625 BCMA scFv, Vl-Vh, G4S linker; huUCHT1 CD3-binding domain). In some embodiments, the BCMA-TAC comprises an amino acid sequence having at least 99% sequence identity with SEQ ID NO: 62 (BCMA TAC: 3625 BCMA scFv, Vl-Vh, G4S linker; huUCHT1 CD3-binding domain). In some embodiments, the BCMA-TAC comprises an amino acid sequence of SEQ ID NO: 62 (BCMA TAC: 3625 BCMA scFv, Vl-Vh, G4S linker; huUCHT1 CD3-binding domain).

Disclosed herein, in certain embodiments, are gamma delta T cells comprising a CD19-TAC encoded by a nucleotide sequence having at least 70% sequence identity with SEQ ID NO: 63 (CD19-TAC). In some embodiments, the CD19-TAC is encoded by a nucleotide sequence having at least 75% sequence identity with SEQ ID NO: 63 (CD19-TAC). In some embodiments, the CD19-TAC is encoded by a nucleotide sequence having at least 80% sequence identity with SEQ ID NO: 63 (CD19-TAC). In some embodiments, the CD19-TAC is encoded by a nucleotide sequence having at least 85% sequence identity with SEQ ID NO: 63 (CD19-TAC). In some embodiments, the CD19-TAC is encoded by a nucleotide sequence having at least 90% sequence identity with SEQ ID NO: 63 (CD19-TAC). In some embodiments, the CD19-TAC is encoded by a nucleotide sequence having at least 95% sequence identity with SEQ ID NO: 63 (CD19-TAC). In some embodiments, the CD19-TAC is encoded by a nucleotide sequence having at least 96% sequence identity with SEQ ID NO: 63 (CD19-TAC). In some embodiments, the CD19-TAC is encoded by a nucleotide sequence having at least 97% sequence identity with SEQ ID NO: 63 (CD19-TAC). In some embodiments, the CD19-TAC is encoded by a nucleotide sequence having at least 98% sequence identity with SEQ ID NO: 63 (CD19-TAC). In some embodiments, the CD19-TAC is encoded by a nucleotide sequence having at least 99% sequence identity with SEQ ID NO: 63 (CD19-TAC). In some embodiments, the CD19-TAC is encoded by a nucleotide sequence of SEQ ID NO: 63 (CD19-TAC).

Disclosed herein, in certain embodiments, are gamma delta T cells comprising a CD19-TAC comprising an amino acid sequence having at least 70% sequence identity with SEQ ID NO: 64 (CD19-TAC). In some embodiments, the CD19-TAC comprises an amino acid sequence having at least 75% sequence identity with SEQ ID NO: 64 (CD19-TAC). In some embodiments, the CD19-TAC comprises an amino acid sequence having at least 80% sequence identity with SEQ ID NO: 64 (CD19-TAC). In some embodiments, the CD19-TAC comprises an amino acid sequence having at least 85% sequence identity with SEQ ID NO: 64 (CD19-TAC). In some embodiments, the CD19-TAC an amino acid sequence having at least 90% sequence identity with SEQ ID NO: 64 (CD19-TAC). In some embodiments, the CD19-TAC comprises an amino acid sequence having at least 95% sequence identity with SEQ ID NO: 64 (CD19-TAC). In some embodiments, the CD19-TAC comprises an amino acid sequence having at least 96% sequence identity with SEQ ID NO: 64 (CD19-TAC). In some embodiments, the CD19-TAC comprises an amino acid sequence having at least 97% sequence identity with SEQ ID NO: 64 (CD19-TAC). In some embodiments, the CD19-TAC comprises an amino acid sequence having at least 98% sequence identity with SEQ ID NO: 64 (CD19-TAC). In some embodiments, the CD19-TAC comprises an amino acid sequence having at least 99% sequence identity with SEQ ID NO: 64 (CD19-TAC). In some embodiments, the CD19-TAC comprises an amino acid sequence of SEQ ID NO: 64 (CD19-TAC).

Amino acid and nucleotide sequences corresponding to TACs, or fragments, of TACs, disclosed herein are identified in Table 5.

TABLE 5
Table of Sequences

| | | Nucleotide/ |
|---|---|---|
| SEQ ID NO | Description | Amino Acid |
| SEQ ID NO: 1 | Tri TAC Configuration 1 | Nucleotide |
| SEQ ID NO: 2 | Tri TAC Configuration 1 | Amino Acid |
| SEQ ID NO: 3 | Tri TAC Configuration 2 | Nucleotide |
| SEQ ID NO: 4 | Tri TAC Configuration 2 | Amino Acid |
| SEQ ID NO: 55 | 3625 TAC Helix Vh-Vl huUCHT1 | Nucleotide |
| SEQ ID NO: 56 | 3625 TAC Helix Vh-Vl huUCHT1 | Amino Acid |
| SEQ ID NO: 57 | 3625 TAC Helix Vl-Vh huUCHT1 | Nucleotide |
| SEQ ID NO: 58 | 3625 TAC Helix Vl-Vh huUCHT1 | Amino Acid |
| SEQ ID NO: 59 | 3625 TAC G4S Vh-Vl huUCHT1 | Nucleotide |
| SEQ ID NO: 60 | 3625 TAC G4S Vh-Vl huUCHT1 | Amino Acid |
| SEQ ID NO: 61 | 3625 TAC G4S VL-VH huUCHT1 | Nucleotide |
| SEQ ID NO: 62 | 3625 TAC G4S VL-VH huUCHT1 | Amino Acid |
| SEQ ID NO: 63 | CD19-TAC | Nucleotide |
| SEQ ID NO: 64 | CD19-TAC | Amino Acid |
| SEQ ID NO: 65 | huIgG Her2 TAC huUCHT1 | Nucleotide |
| SEQ ID NO: 66 | huIgG Her2 TAC huUCHT1 | Amino Acid |
| SEQ ID NO: 67 | CD8a Her2 TAC huUCHT1 | Nucleotide |
| SEQ ID NO: 68 | CD8a Her2 TAC huUCHT1 | Amino Acid |
| SEQ ID NO: 75 | muIgG Her2 TAC huUCHT1 | Nucleotide |
| SEQ ID NO: 76 | muIgG Her2 TAC huUCHT1 | Amino Acid |

### Polypeptides and Vector Constructs

Disclosed herein, in certain embodiments, are gamma delta T cells comprising a vector comprising a TAC nucleic acid sequence as disclosed herein. In some embodiments, the vectors further comprise a promoter. In some embodiments, the promoter is functional in a mammalian cell. Promoters, regions of DNA that initiate transcription of a particular nucleic acid sequence, are well known in the art. A “promoter functional in a mammalian cell” refers to a promoter that drives expression of the associated nucleic acid sequence in a mammalian cell. A promoter that drives expression of a nucleic acid sequence is referred to as being “operably connected” to the nucleic acid sequence.

A variety of delivery vectors and expression vehicles are employed to introduce nucleic acids described herein into a cell.

Disclosed herein, in certain embodiments, are gamma delta T cells comprising a vector comprising:
a. a first polynucleotide encoding a target-specific antigen-binding domain;
b. a second polynucleotide encoding an ligand that binds a protein associated with a TCR complex;
c. a third polynucleotide encoding a T cell receptor signaling domain polypeptide; and
d. a promoter that is functional in a mammalian cell.

In some embodiments, the first polynucleotide and third polynucleotide are fused to the second polynucleotide and the coding sequence is operably connected to the promoter. In some embodiments, the second polynucleotide and third polynucleotide are fused to the first polynucleotide and the coding sequence is operably connected to the promoter. In some embodiments, the vector is designed for expression in mammalian cells such as γδ T cells. In some embodiments, the vector is a viral vector. In some embodiments, the viral vector is a retroviral vector.

In some embodiments, vectors that are useful comprise vectors derived from retroviruses, lentiviruses, Murine Stem Cell Viruses (MSCV), pox viruses, adenoviruses, and adeno-associated viruses. Other delivery vectors that are useful comprise vectors derived from herpes simplex viruses, transposons, vaccinia viruses, human papilloma virus, Simian immunodeficiency viruses, HTLV, human foamy virus and variants thereof. Further vectors that are useful comprise vectors derived from spumaviruses, mammalian type B retroviruses, mammalian type C retroviruses, avian type C retroviruses, mammalian type D retroviruses and HTLV/BLV type retroviruses. One example of a lentiviral vector useful in the disclosed compositions and methods is the pCCL4 vector.

### Methods of Manufacturing Gamma Delta T Cells

Disclosed herein, in certain embodiments, are methods of making γδ T cells, for example, γδ T cells comprising or expressing a TAC polypeptide. In some embodiments, the method comprises one or more of the following steps: (a) contacting γδ T cells isolated from an individual with zoledronate, a cytokine (e.g., IL-2 and/or IL-15), and/or a CD16 agonist, (b) contacting the γδ T cells with an expression vector comprising a nucleic acid encoding the TAC polypeptide, (c) culturing and/or expanding the cells (for example, for 10-14 days), and (d) removing αβ T cells from the culture.

In some embodiments, the method comprises contacting the γδ T cells with zoledronate. In some embodiments, the method comprises contacting the γδ T cells with IL-2. In some embodiments, the method comprises contacting the γδ T cells with zoledronate and IL-2. In some embodiments, the method comprises contacting the γδ T cells with a CD16 agonist. In some embodiments, the method comprises contacting the γδ T cells with zoledronate, IL-2, and a CD16 agonist.

In some embodiments, the γδ T cells are cultured and/or expanded for 10, 11, 12, 13, or 14 days, or for at least 10, 11, 12, 13, or 14 days.

In some embodiments, αβ T cells are removed by negative selection of cells including CD4 and/or CD8. In some embodiments, the method results in a culture or composition that is substantially free of αβ T cells (e.g., at least 90%, 95%, 96%, 97%, 98%, or 99%, or 100% of the T cells present in the resulting culture or composition are γδ T cells). In some embodiments, the method results in a culture or composition that is substantially free of cells other than γδ T cells (e.g., at least 90%, 95%, 96%, 97%, 98%, or 99%, or 100% of the cells present in the resulting culture or composition are γδ T cells).

In some embodiments, once obtained, the γδ T cells are optionally enriched in vitro. In some embodiments, a population of cells is enriched by positive or negative selection. Further, the γδ T cells are optionally frozen or cryopreserved and then thawed at a later date.

In some embodiments, γδ T cells are activated and/or expanded before or after introducing the TAC to the γδ T cells. In some embodiments, the γδ T cells are expanded by contact with a surface having attached thereto an agent that stimulates a CD3/TCR complex associated signal and an antigen-binding domain that stimulates a co-stimulator molecule on the surface of the γδ T cells. In some embodiments, the γδ T cells are expanded by contact with one or more soluble agents that stimulate CD3/TCR complex signaling and co-stimulator molecule signaling.

In some embodiments, the γδ T cells are transduced or transfected with nucleic acid sequences. The transduced or transfected γδ T cells express proteins coded for by the transfected or transduced nucleic acid sequences. A nucleic acid may be introduced into a cell by physical, chemical, or biological means. Physical means include, but are not limited to, microinjection, electroporation, particle bombardment, lipofection and calcium phosphate precipitation. Biological means include the use of DNA and RNA vectors.

Viral vectors, including, e.g., retroviral vectors, are used to introduce and express a nucleic acid into a γδ T cell. Viral vectors include vectors derived from lentivirus, retrovirus, Murine Stem Cell Viruses (MSCV), pox viruses, herpes simplex virus I, adenovirus and adeno-associated viruses. The vector optionally includes a promoter that drives expression of the transduced nucleic acid molecule in a γδ T cell (e.g., a CMV promoter, eF1a promoter, or MSCV promoter). In some embodiments, the expression vector is a lentiviral vector, for example, a VSV-G pseudotyped lentiviral vector. In some embodiments, the expression vector is a γ retroviral vector, for example, a GALV pseudotyped γ-retroviral vector.

Any suitable assay is used to confirm the presence and/or expression of the transduced nucleic acid sequence and/or the polypeptide encoded by the nucleic acid in the γδ T cell. Assays include, but are not limited to, Southern and Northern blotting, RT-PCR and PCR, ELISA, Western blotting, and flow cytometry.

In some embodiments, a γδ T cell expressing a TAC has increased T cell activation in the presence of an antigen compared to a T cell not expressing a TAC and/or as compared to a γδ T cell expressing a traditional CAR. Increased γδ T cell activation may be ascertained by numerous methods, including but not limited to, increased tumor cell line killing, increased cytokine production, increased cytolysis, increased degranulation and/or increased expression of activation markers such as CD107α, IFNγ, IL2 or TNFα. In some embodiments, increases are measured in an individual cell or in a population of cells.

The terms “increased” or “increasing” as used herein refer to at least a 1%, 2%, 5%, 10%, 25%, 50%, 100% or 200% increase in a γδ T cell or population of γδ T cells expressing a TAC compared to a γδ T cell or population of γδ T cells not expressing a TAC and/or as compared to a γδ T cell or population of γδ T cells expressing a traditional CAR.

### Pharmaceutical Compositions

Disclosed herein, in certain embodiments, are pharmaceutical compositions comprising an engineered gamma delta T cell disclosed herein (transduced with and/or expressing a TAC), and a pharmaceutically acceptable carrier. Pharmaceutically acceptable carriers include, but are not limited to, buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); preservatives (e.g., cryopreservatives); or DMSO. In some embodiments, the engineered γδ T cells are formulated for intravenous administration.

Pharmaceutical compositions are administered in a manner appropriate to the disease to be treated (or prevented). The quantity and frequency of administration is determined by such factors as the condition of the patient, and the type and severity of the patient's disease, although appropriate dosages are determined by clinical trials. When “an immunologically effective amount,” “an anti-tumor effective amount,” “a tumor-inhibiting effective amount,” or “therapeutic amount” is indicated, the precise amount of the compositions of the present invention to be administered is determined by a physician with consideration of individual differences in age, weight, tumor size, extent of infection or metastasis, and condition of the patient (subject).

In some embodiments, the engineered gamma delta T cells and/or pharmaceutical compositions described herein are administered at a dosage of 10¹ to 10¹⁵ cells per kg body weight, 10⁴ to 10⁹ cells per kg body weight, optionally 10⁵ to 10⁸ cells per kg body weight, 10⁶ to 10⁷ cells per kg body weight or 10⁵ to 10⁶ cells per kg body weight, including all integer values within those ranges. In some embodiments, the modified γδ T cells and/or pharmaceutical compositions described herein are administered at a dosage of greater than 10¹ cells per kg body weight. In some embodiments, the modified γδ T cells and/or pharmaceutical compositions described herein are administered at a dosage of less than 10¹⁵ cells per kg body weight.

In some embodiments, the engineered gamma delta and/or pharmaceutical compositions described herein are administered at a dosage of 0.5×10⁶ cells, 2×10⁶ cells, 4×10⁶ cells, 5×10⁶ cells, 1.2×10⁷ cells, 2×10⁷ cells, 5×10⁷ cells, 2×10⁸ cells, 5×10⁸ cells, 2×10⁹ cells, 0.5-2000×10⁶ cells, 0.5-2×10⁶ cells, 0.5-2×10⁷ cells, 0.5-2×10⁸ cells, or 0.5-2×10⁹ cells, including all integer values within those ranges.

In some embodiments, γδ T cell compositions are administered multiple times at these dosages. In some embodiments, the dosage is administered a single time or multiple times, for example daily, weekly, biweekly, or monthly, hourly, or is administered upon recurrence, relapse or progression of the cancer being treated. The cells, in some embodiments, are administered by using infusion techniques that are commonly known in immunotherapy (see, e.g., Rosenberg et al., New Eng. J. of Med. 319:1676, 1988).

In some embodiments, the pharmaceutical composition is substantially free of, e.g., there are no detectable levels of a contaminant, e.g., selected from the group consisting of endotoxin, mycoplasma, replication competent lentivirus (RCL), p24, VSV-G nucleic acid, HIV gag, residual anti-CD3/anti-CD28 coated beads, mouse antibodies, pooled human serum, bovine serum albumin, bovine serum, culture media components, vector packaging cell or plasmid components, a bacterium a fungus, mycoplasma, IL-2, and IL-7. In some embodiments, there may be a therapeutically acceptable level of a contaminant, e.g., one of the foregoing contaminants.

The modified/engineered γδ T cells and/or pharmaceutical compositions are administered by methods including, but not limited to, aerosol inhalation, injection, infusion, ingestion, transfusion, implantation or transplantation. The modified γδ T cells and/or pharmaceutical compositions are administered to a subject transarterially, subcutaneously, intradermally, intratumorally, intranodally, intramedullary, intramuscularly, by intravenous (i.v.) injection, by intravenous (i.v.) infusion, or intraperitoneally. The modified/engineered γδ T cells and/or pharmaceutical compositions thereof are administered to a patient by intradermal or subcutaneous injection. The modified/engineered γδ T cells and/or pharmaceutical compositions thereof are administered by i.v. injection. The modified/engineered γδ T cells and/or pharmaceutical compositions thereof are injected directly into a tumor, lymph node, or site of infection.

A pharmaceutical composition is prepared by known methods for the preparation of pharmaceutically acceptable compositions that are administered to subjects, such that an effective quantity of the γδ T cells is combined in a mixture with a pharmaceutically acceptable carrier. Suitable carriers are described, for example, in Remington's Pharmaceutical Sciences (Remington's Pharmaceutical Sciences, 20^{th} ed., Mack Publishing Company, Easton, Pa., USA, 2000). On this basis, the compositions include, albeit not exclusively, solutions of the substances in association with one or more pharmaceutically acceptable carriers or diluents, and contained in buffered solutions with a suitable pH and iso-osmotic with the physiological fluids.

Suitable pharmaceutically acceptable carriers include essentially chemically inert and nontoxic compositions that do not interfere with the effectiveness of the biological activity of the pharmaceutical composition. Examples of suitable pharmaceutical carriers include, but are not limited to, water, saline solutions, glycerol solutions, N-(1(2,3-dioleyloxy)propyl)N,N,N-trimethylammonium chloride (DOTMA), diolesylphosphotidyl-ethanolamine (DOPE), and liposomes. In some embodiments, such compositions contain a therapeutically effective amount of the compound, together with a suitable amount of carrier so as to provide the form for direct administration to the patient.

Pharmaceutical compositions include, without limitation, lyophilized powders or aqueous or non-aqueous sterile injectable solutions or suspensions, which may further contain antioxidants, buffers, bacteriostats and solutes that render the compositions substantially compatible with the tissues or the blood of an intended recipient. Other components that may be present in such compositions include water, surfactants (such as Tween), alcohols, polyols, glycerin and vegetable oils, for example. Extemporaneous injection solutions and suspensions may be prepared from sterile powders, granules, tablets, or concentrated solutions or suspensions.

A pharmaceutical composition disclosed herein is formulated into a variety of forms and administered by a number of different means. A pharmaceutical formulation is administered orally, rectally, or parenterally, in formulations containing conventionally acceptable carriers, adjuvants, and vehicles as desired. The term “parenteral” as used herein includes subcutaneous, intravenous, intramuscular, or intrasternal injection and infusion techniques. Administration includes injection or infusion, including intra-arterial, intracardiac, intracerebroventricular, intradermal, intraduodenal, intramedullary, intramuscular, intraosseous, intraperitoneal, intrathecal, intravascular, intravenous, intravitreal, epidural and subcutaneous), inhalational, transdermal, transmucosal, sublingual, buccal and topical (including epicutaneous, dermal, enema, eye drops, ear drops, intranasal, vaginal) administration. In some exemplary embodiments, a route of administration is via an injection such as an intramuscular, intravenous, subcutaneous, or intraperitoneal injection.

Liquid formulations include an oral formulation, an intravenous formulation, an intranasal formulation, an ocular formulation, an otic formulation, an aerosol, and the like. In certain embodiments, a combination of various formulations is administered. In certain embodiments a composition is formulated for an extended release profile.

### Methods of Treatment and Use

Disclosed herein, in certain embodiments, are methods of using engineered gamma delta T cells disclosed herein in the treatment of cancer in an individual in need thereof.

In some embodiments, a target-specific antigen-binding domain of the TACs disclosed herein bind to a tumor antigen or tumor associated antigen on a tumor cell. In some embodiments, a target-specific antigen-binding domain of the TACs disclosed herein selectively bind to a tumor antigen or tumor associated antigen on a tumor cell. In some embodiments, a target-specific antigen-binding domain of the TACs disclosed herein specifically bind to a tumor antigen or tumor associated antigen on a tumor cell. In some embodiments, the target antigen is a tumor antigen. Examples of tumor antigens include, but are not limited to, CD19, HER2 (erbB-2), B-cell maturation antigen (BCMA), alphafetoprotein (AFP), carcinoembryonic antigen (CEA), CA-125, MUC-1, epithelial tumor antigen (ETA), tyrosinase, melanoma-associated antigen (MAGE), prostate-specific antigen (PSA), glioma-associated antigen, β-human chorionic gonadotropin, thyroglobulin, RAGE-1, MN-CA IX, human telomerase reverse transcriptase, RU1, RU2 (AS), intestinal carboxyl esterase, mut hsp70-2, M-CSF, prostase, PAP, NY-ESO-1, LAGE-1a, p53, prostein, PSMA, survivin and telomerase, prostate-carcinoma tumor antigen-1 (PCTA-1), ELF2M, neutrophil elastase, CD22, insulin growth factor (IGF)-I, IGF-II, IGF-I receptor and mesothelin.

Disclosed herein, in certain embodiments, are methods of treating a cancer expressing a target antigen in an individual in need thereof, comprising administering to the individual engineered gamma delta T cells disclosed herein. In some embodiments, the target antigen is CD19. In some embodiments, the method of treating a cancer expressing CD19 in an individual in need thereof comprises administering to the individual engineered gamma delta T cells comprising a TAC comprising a CD19-targeting antigen-binding domain. In some embodiments, examples of cancers that are treated by a gamma delta T cell comprising a CD19-targeting TAC include, but are not limited to B cell malignancies. In some embodiments, examples of cancers that are treated by a gamma delta T cell comprising a CD19-targeting TAC include, but are not limited to B cell lymphomas, acute lymphoblastic leukemia (ALL), and chronic lymphocytic leukemia (CLL). In some embodiments, examples of cancers that are treated by a gamma delta T cell comprising a CD19-targeting TAC include, but are not limited to Non-Hodgkin's lymphoma (NHL).

In some embodiments, the target antigen is HER2. In some embodiments, the method of treating a cancer wherein a cancer cell expresses HER2 in an individual in need thereof comprises administering to the individual engineered gamma delta T cells comprising a TAC comprising a HER2-targeting antigen-binding domain. In some embodiments, examples of cancers that are treated by a gamma delta T cell comprising a HER2-targeting TAC include, but are not limited to breast cancer, bladder cancer, pancreatic cancer, ovarian cancer, and stomach cancer.

In some embodiments, the target antigen is BCMA. In some embodiments, the method of treating a cancer wherein a cancer cell expresses BCMA in an individual in need thereof comprises administering to the individual engineered gamma delta T cells comprising a TAC comprising a BCMA-targeting antigen-binding domain. In some embodiments, examples of cancers that are treated by a gamma delta T cell comprising a BCMA-targeting TAC include, but are not limited to leukemia, lymphomas, and multiple myeloma.

Further disclosed herein is use of an engineered gamma delta T cell disclosed herein in the preparation of a medicament to treat cancer in an individual in need thereof. Also disclosed herein is the use of a mixture of gamma delta T cells comprising engineered/modified and unmodified gamma delta T cells, or comprising different populations of engineered/modified gamma delta T cells with or without unmodified gamma delta T cells. One of ordinary skill in the art would understand that a therapeutic quantity of engineered/modified gamma delta T cells need not be homogenous in nature.

The engineered gamma delta T cells described herein can be used alone or in combination with other therapeutic agents and/or modalities. The term administered “in combination,” as used herein, is understood to mean that two (or more) different treatments are delivered to the subject during the course of the subject's affliction with the disorder, such that the effects of the treatments on the patient overlap at a point in time. In certain embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In certain embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In certain embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

In some embodiments, a gamma delta T cell or pharmaceutical composition is administered in combination with zoledronate.

In some embodiments, a gamma delta T cell or pharmaceutical composition is administered in combination with a cytokine, e.g., IL-2, IL-15, and/or an IL-15 IL-15Rα fusion protein. It is understood, that with regard to a combination therapy with a peptide, polypeptide, or protein (e.g., IL-2, IL-15, and/or an IL-15 IL-15Rα fusion protein), a gamma delta T cell or pharmaceutical composition described herein may be administered in combination with (i) the peptide, polypeptide, or protein itself or (ii) a nucleic acid or expression vector encoding the peptide, polypeptide, or protein. Alternatively, a gamma delta T cell may be engineered to express the peptide, polypeptide, or protein. When a gamma delta T cell is engineered to express a peptide, polypeptide, or protein, the peptide, polypeptide, or protein may be expressed in an intracellular, secreted, or membrane bound form. For example, IL-15 may be expressed as a membrane-bound IL-15 and IL-15Rα fusion protein.

In some embodiments, a gamma delta T cell or pharmaceutical composition is administered in combination with a CD16 agonist (e.g., an anti-CD16 antibody).

In some embodiments, effectiveness of a therapy disclosure herein is assessed multiple times. In some embodiments, patients are stratified based on a response to a treatment disclosed herein. In some embodiments, an effectiveness of treatment determines entrance into a trial.

In some embodiments, cancers that are treated engineered gamma delta T cells comprising any one of the TACs disclosed herein include any form of neoplastic disease. In some embodiments, examples of cancers that are treated include, but are not limited to breast cancer, lung cancer and leukemia, for example mixed lineage leukemia (MLL), chronic lymphocytic leukemia (CLL) acute lymphoblastic leukemia (ALL). In some embodiments, examples of cancers that are treated include, but are not limited to large B-cell lymphoma, diffuse large B-cell lymphoma, primary mediastinal B cell lymphoma, high grade B-cell lymphoma, or large B cell lymphoma arising from follicular lymphoma. Other cancers include carcinomas, blastomas, melanomas, sarcomas, hematological cancers, lymphoid malignancies, benign and malignant tumors, and malignancies. In some embodiments, the cancer comprises non-solid tumors or solid tumors. In some embodiments, cancers that are treated include tumors that are not vascularized, or not yet substantially vascularized, as well as vascularized tumors. In some embodiments, the cancer is a solid cancer or comprises a solid tumor. In some embodiments, the cancer is a liquid cancer or comprises a liquid tumor. In some embodiments, the cancer is a lung cancer, a breast cancer, a colon cancer, multiple myeloma, glioblastoma, gastric cancer, ovarian cancer, stomach cancer, colorectal cancer, urothelial cancer, endometrial cancer, or a melanoma. In some embodiments, the cancer is a lung cancer. In some embodiments, the cancer is a breast cancer. In some embodiments, the cancer is a colon cancer. In some embodiments, the cancer is multiple myeloma. In some embodiments, the cancer is a glioblastoma. In some embodiments, the cancer is a gastric cancer. In some embodiments, the cancer is an ovarian cancer. In some embodiments, the cancer is a stomach cancer. In some embodiments, the cancer is a colorectal cancer. In some embodiments, the cancer is urothelial cancer. In some embodiments, the cancer is an endometrial cancer. In some embodiments, the cancer is a melanoma.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1. Materials and Methods

### Isolation of PBMCs

Human peripheral blood mononuclear cells (PBMCs) were obtained from volunteer healthy donors at the McMaster Immunology Research Centre; in some cases, the PBMC were isolated from leukapheresis products purchased from HemaCare Corporation (Van Nuys, CA). PBMCs were isolated from heparinized whole blood by density gradient centrifugation over Ficoll-Paque® Plus (Biosciences, Piscataway, NJ). After the purification process, PBMCs were frozen down in 90% human AB serum/10% DMSO.

This study was approved by the Research Ethics Board of McMaster University, Hamilton, Canada. Written informed consent was obtained from all healthy donors who provided peripheral blood samples.

### Viruses and Chimeric Receptors.

All lentiviruses were manufactured at Lentigen. The CD19-TAC virus included the nucleotide sequence of SEQ ID NO: 63, which encoded a TAC receptor having the amino acid sequence of SEQ ID NO: 64, which included a CD8a leader sequence, FMC63 single-chain antibody that recognizes CD19, humanized UCHT1 single-chain antibody bearing the Y177T mutation and CD4 transmembrane/cytoplasmic domains. The HER2-TAC virus included the nucleotide sequence of SEQ ID NO: 65, which encoded a TAC receptor having the amino acid sequence of SEQ ID NO: 66, which included a IgGκ leader, the H10-2-G3 DARPin that recognizes HER2, humanized UCHT1 single-chain antibody and CD4 transmembrane/cytoplasmic domains.

GALV-pseudotyped γ-retroviruses were manufactured at McMaster University. The BCMA-TAC virus is a γ-retrovirus that encodes a TAC receptor containing a single-chain antibody that recognizes BCMA.

### γδ T Cell Manufacturing

PBMC from healthy donors were isolated from leukapheresis products purchased from Hemacare and Stem Cell Technologies. PBMC were seeded into 96-well round bottom plates at a density of 2×10⁵ cells/well in RPMI 1640 containing 10% heat-inactivated fetal bovine serum 10 mM HEPES, 2 mM L-glutamine, 1000 U/ml Penicillin and Streptomycin, 55 uM 2-mercaptoethanol. 1 mM sodium pyruvate and non-essential amino acids (hereafter referred to as cRPMI). IL-2 and zoledronate were included in the starting cultures at concentrations of 10 ng/ml and 1 ug/ml, respectively. After approximately 18-24 hours from the start of culture, 100 ul of culture medium was removed from each well and lentivirus was added in a volume of 10-50 ul; the volume of lentivirus is dependent upon the multiplicity of infection (MOI), which is determined for each individual virus and donor to achieve transduction of 30%-40% γδ T cells. Alternatively, the cells are transduced with γ-retrovirus 72 hours after activation. After another 16-24 hours of culture, each well received 100 ul of cRPMI supplemented with 10 ng/ml IL-2. Cultures were monitored regularly and scaled as needed to keep cell density around 1×10⁶ cells/ml. The cultures were scaled by the addition of fresh cRPMI supplemented with 10 ng/ml IL-2 and transferred to larger culture vessels as needed. After 14 days of culture, the γδ T cells were enriched by removal of CD4-positive and CD8-positive T cells yielding a culture of >98% pure γδ T cells. After the enrichment step, the γδ T cells were resuspended in CryoStor10™ and cryopreserved in liquid nitrogen.

### Phenotypic Analysis of Cell Surface Markers by Flow Cytometry

CAR and transduction marker tNGFR expression was evaluated through immunostaining and analysis by flow cytometry. To measure surface expression of the HER2-CAR, T cells were incubated with recombinant HER2-Fc chimera protein (R&D Systems) followed by phycoerythrin-conjugated anti-human IgG Fc secondary conjugated antibody (Jackson ImmunoResearch). To measure surface expression of the BCMA-CAR, T cells were incubated with recombinant BCMA-Fc chimera protein (R&D Systems) followed by phycoerythrin-conjugated anti-human IgG Fc secondary conjugated antibody (Jackson ImmunoResearch). To measure CD19-TAC expression, T cells were incubated with biotinylated-Protein L (Thermo Fisher Scientific) followed by phycoerythrin-conjugated streptavidin (BD Pharmingen). The expression of T cell phenotypic markers (CD4, CD8 and tNGFR) was detected by direct staining with conjugated antibodies (BD Biosciences). Flow cytometry was conducted on BD LSRII or BD LSRFortessa™ cytometers (BD Bioscience) and analyzed using FlowJo vX software.

### Tumor Cell Lines

All tumor lines were cultured in RPMI 1640 supplemented with 10% heat-inactivated FBS, 2 mM L-glutamine, 10 mM HEPES, 100 U/mL penicillin, 100 μg/mL streptomycin, and 55 nM β-mercaptoethanol (Cell culture medium and additives were purchased from Thermo Fisher Scientific). Cell lines were routinely tested for presence of mycoplasma using a commercial kit from InvivoGen. The cell lines were engineered with a lentivirus that encodes enhanced firefly luciferase (as described in Rabinovich et al. (2008) Proc. Natl. Acad. Sci. USA 105:14342-6) to permit use in the luciferase-based cytotoxicity assay and to enable in vivo monitoring.

### In Vitro Cytotoxicity Luminescence Assay

To evaluate cytotoxicity, 5×10⁴ luciferase engineered tumor cells were co-cultured with T cells in a white flat bottom 96-well plate (Corning) at indicated effector:target for 18 h at 37° C. A549 and OVCAR-3 were used as targets for HER2-CAR T cells. JeKo-1, Raji and NALM-6 was used as a target for CD19-TAC T cells. After co-culture, 0.15 mg/mL D-Luciferin (Perkin Elmer, Waltham, MA) was added per well and luminescence was measured using a SpectraMax® i3 (Molecular Devices, Sunnyvale, CA) across all wavelengths. The % Target Killing was determined as: 1−[((Emission of Test Well−Background)/(Emission of Well with Tumor Cells Alone−Background))]×100%. Each condition was tested in triplicate.

### Adoptive Transfer and In Vivo Monitoring

Five-week-old female NOD.Cg-Rag1tm1MomIl2rgtm1Wjl/SzJ (NRG) mice were purchased from The Jackson Laboratory (Bar Harbor, ME) or bred in-house. Seven to eleven-week-old male NRG mice were injected with 5×10⁵ JeKo-1-effLuc cells intravenously. A single dose of engineered T cells was administered after 7 days of tumor growth. Tumor burden was monitored through bioluminescent imaging as described previously (Helsen et al. (2018) Nat. Commun. 9:3049, Hammill et al. (2020) Mol. Ther Oncolytics. 17:278-292). Mice were injected intraperitoneally with D-Luciferin solution (15 mg/ml; Perkin Elmer; Waltham, MA) at a dose of 10 uL D-Luciferin solution/gram of body weight 14 min prior to dorsal and ventral imaging using an IVIS Spectrum (Caliper Life Sciences; Waltham, MA). Images were analyzed using Living Image Software v4.2 for MacOSX (Perkin Elmer) and dorsal and ventral radiance was summed. Termination criteria included moribundity or hind limb paralysis. The McMaster Animal Research Ethics Board approved all murine experiments and in all cases animal treatment strictly adhered to McMaster Animal Research Ethics Board instructions and guidelines.

### Example 2. Engineering TAC-Expressing γδ T Cells

This Example describes a process for engineering γδ T cells with TAC receptors.

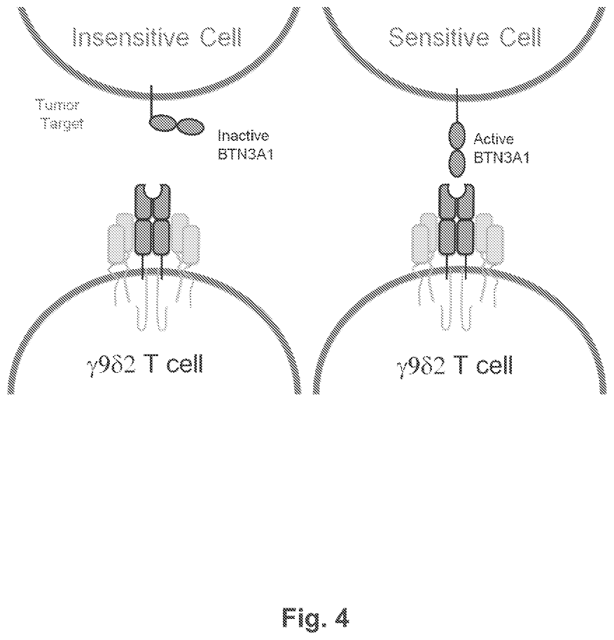

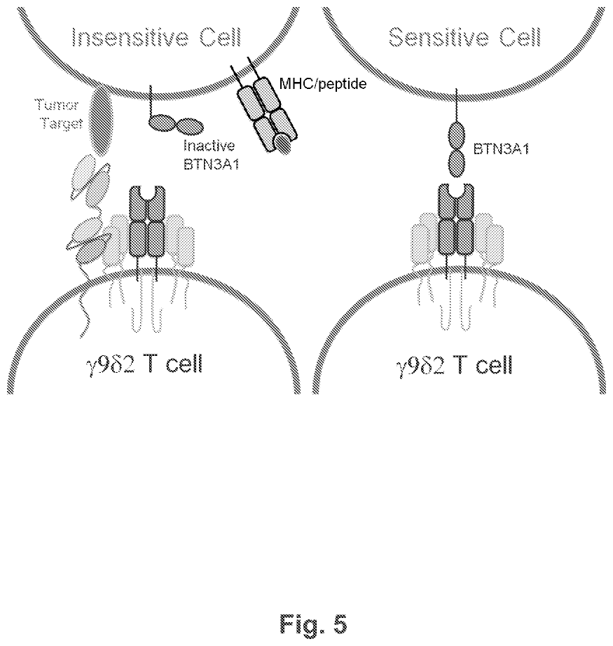

As shown in FIG. 4, cells that display an inactive form of the ligand BTN3A1 cannot bind the γ9δ2 TCR and are therefore insensitive to γ9δ2 T cells. However, as shown in FIG. 5, a TAC receptor can co-opt the γ9δ2 TCR, similar to a conventional αβ TCR, and direct γ9δ2 T cells to attack otherwise insensitive targets.

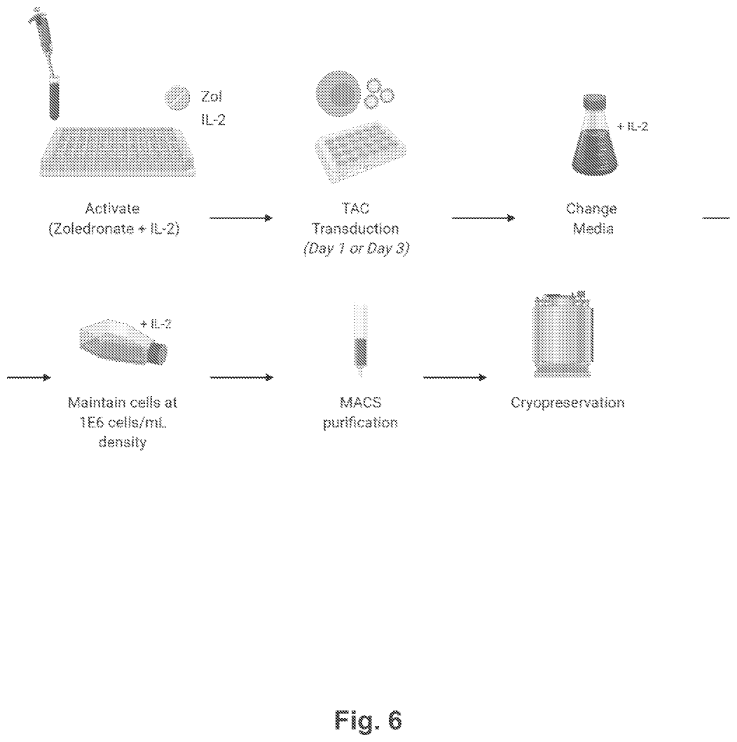

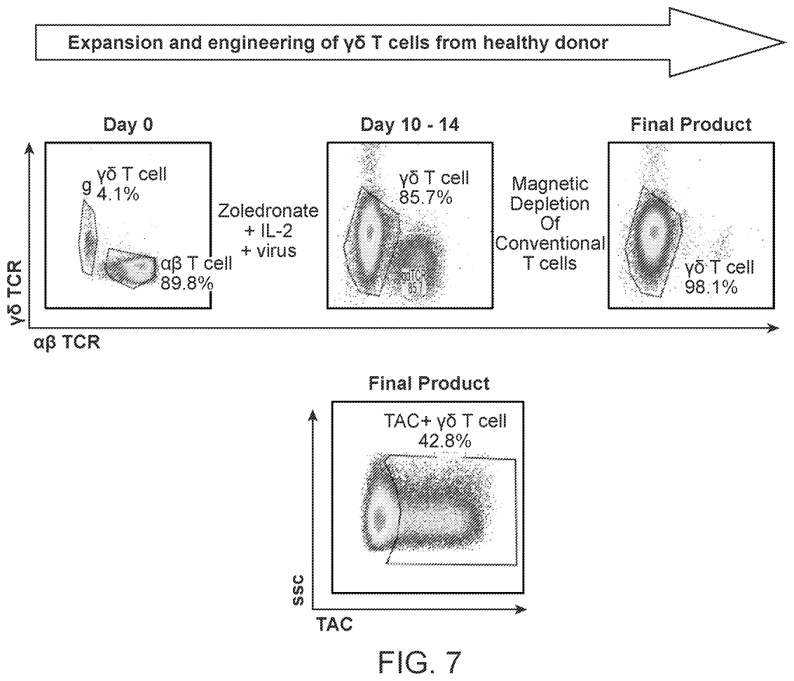

A schematic depiction of a method for generating such TAC-expressing γ9δ2 T cells is depicted in FIG. 6. γ9δ2 T cells were activated with zoledronate and cultured in the presence of IL-2. Following activation, the γ9δ2 T cells were infected with lentivirus or γ-retrovirus encoding the TAC receptor and allowed to expand for 10-14 days at which point they became the dominant population in the culture. Contaminating αβ T cells were then removed from the cell product by negative selection of CD4 and CD8 receptors yielding a final product that was >98% γ9δ2 T cells. The process routinely yielded transduction efficiencies of 40%-50%, which is comparable to the transduction efficiency in conventional αβ T cells. The frequency of γδ T cells at various points in the process is shown in FIG. 7.

### Example 3. In Vitro Cytotoxicity of TAC-Expressing γδ T Cells

This Example describes pre-clinical data demonstrating that TAC receptors successfully direct γδ T cells towards discrete targets in vitro and enable robust killing of tumor cells that are otherwise resistant to γδ T cells.

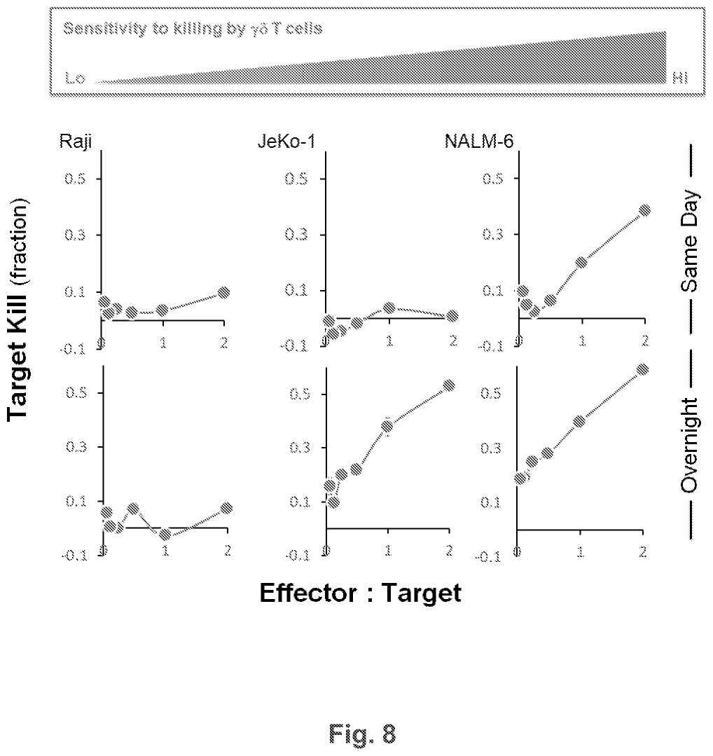

A culture of γ9δ2 T cells was generated using the zoledronate-based manufacturing process described in Example 2, except that the T cells were not engineered to express a TAC receptor. The non-engineered γ9δ2 T cells were co-cultured with CD19-positive targets (Raji, JeKo-1 and NALM-6) at varying effector:target ratios, and the cells were tested for in vitro cytotoxicity using the luminescence-based assay described above in Example 1. Results are shown in FIG. 8. Killing of the targets was ascertained after 6 hours (Same Day) or 18 hours (Overnight). NALM-6 cells were found to be most sensitive to killing by the non-engineered γ9δ2 T cells. JeKo-1 cells were found to display intermediate sensitivity to γ9δ2 T cells. Raji cells were found to be resistant to killing by non-engineered γ9δ2 T cells.

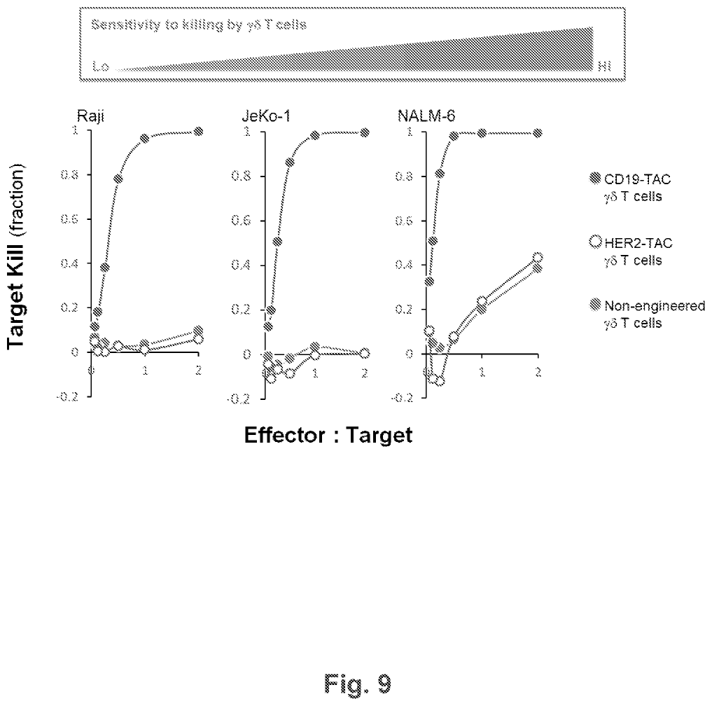

Three cultures of γ9δ2 T cells were produced using the zoledronate-based manufacturing process described in Example 2: (i) a culture engineered with a TAC specific for CD19 (CD19-TAC γδ T cells), (ii) a culture engineered with a TAC specific for HER2 (HER2-TAC γδ T cells), and (iii) a culture of non-engineered γδ T cells. The three γ9δ2 T cell products were co-cultured with CD19-positive/HER2-negative targets (Raji, JeKo-1 and NALM-6) at varying effector:target ratios, and the cells were tested for in vitro cytotoxicity using the luminescence-based assay described above in Example 1. Results are shown in FIG. 9. The CD19-TAC γδ T cells displayed lytic activity against all targets, including the Raji targets which were otherwise insensitive to non-engineered γδ T cells. In contrast, the HER2-TAC γδ T cells displayed no increase in lytic activity compared to non-engineered γδ T cells confirming that the enhancement in killing by engineering γδ T cells with the CD19-TAC is due to antigen-specific targeting. The benefit of the antigen-specific TAC engineering was seen with all tumors, regardless of their inherent sensitivity to non-engineered γδ T cells.

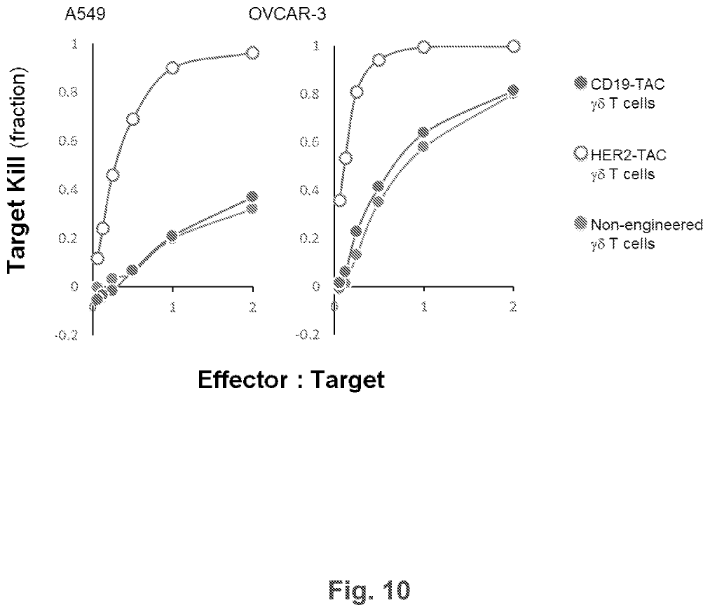

Three cultures of γ9δ2 T cells were produced using the zoledronate-based manufacturing process described in Example 2: (i) a culture engineered with a TAC specific for CD19 (CD19-TAC γδ T cells), (ii) a culture engineered with a TAC specific for HER2 (HER2-TAC γδ T cells), and (iii) a culture of non-engineered γδ T cells. The three γ9δ2 T cell products were co-cultured with HER2-positive/CD19-negative targets (A549 and OVCAR-3) at varying effector:target ratios, and the cells were tested for in vitro cytotoxicity using the luminescence-based assay described above in Example 1. Results are shown in FIG. 10. While both tumor lines displayed some sensitivity to lysis by non-engineered γδ T cells, engineering with the HER2-TAC enhanced the ability of the γδ T cells to lyse both targets. In contrast, the CD19-TAC γδ T cells displayed no increase in lytic activity compared to non-engineered γδ T cells confirming that the enhancement in killing by engineering γδ T cells with the HER2-TAC is due to antigen-specific targeting. Again, the benefit of the antigen-specific TAC engineering was seen with all tumors, regardless of their inherent sensitivity to non-engineered γδ T cells.

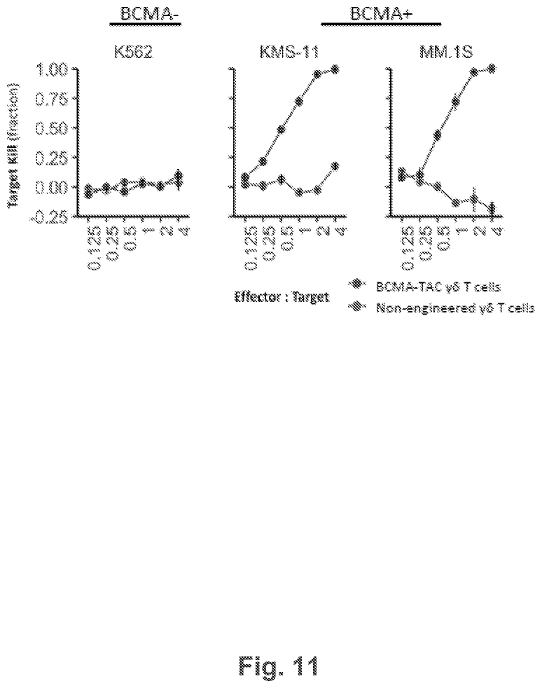

The following cultures of γ9δ2 T cells were produced: (i) a culture engineered with a GALV-pseudotyped γ-retrovirus encoding a TAC specific for BCMA (BCMA-TAC γδ T cells), and (ii) a culture of non-engineered γδ T cells. The γ9δ2 T cell products were co-cultured with BCMA+ (KMS-11 and MM.1S) or BCMA− (K562) cells at varying effector:target ratios, and the cells were tested for in vitro cytotoxicity using a luminescence-based assay. Results are shown in FIG. 11, and demonstrate that engineering with the BCMA-TAC dramatically enhanced the ability of the γδ T cells to lyse the BCMA+ targets.

### Example 4. In Vivo Cytotoxicity of TAC-Expressing γδ T Cells

This Example describes pre-clinical data demonstrating that TAC-receptors enhance therapeutic activity of γ9δ2 T cells in vivo, including against tumors that are otherwise insensitive to γ9δ2 T cells.

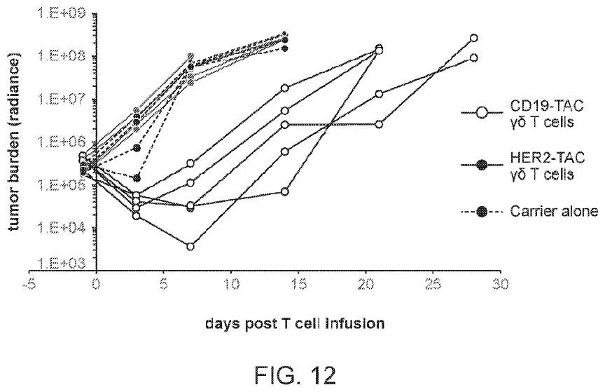

Mice bearing CD19-positive/HER2-negative JeKo-1 xenografts were treated with: (i) γ9δ2 T cells engineered with a TAC specific for CD19 (CD19-TAC γδ T cells), (ii) γ9δ2 T cells engineered with a TAC specific for HER2 (HER2-TAC γδ T cells), or (iii) carrier medium (Cryostor10™) alone. Tumor growth was monitored weekly by bioluminescent imaging. Results are shown in FIG. 12. Tumors in mice treated with HER2-TAC γδ T cells grew at the same pace as tumor treated with carrier alone, demonstrating that this tumor is insensitive to γδ T cells and that engineering with the TAC receptor does not lead to non-antigen specific anti-tumor activity. In contrast, treatment with CD19-TAC γδ T cells caused regression of all tumors demonstrating clear antigen-specific anti-tumor activity mediated by the TAC receptor.

### Example 5. Cytotoxicity of TAC-Expressing γδ T Cells

γδ T cells were engineered to express a TAC specific for HER2 (HER2-TAC γδ T cells), generally using the zoledronate-based manufacturing process described in Example 2. Non-engineered γδ T cells (NTD γδ T cells) were also used in experiments. HT1080 cells (naturally HER2-positive) were engineered to express enhanced luciferase (eLuc) to permit use in luciferase-based cytotoxicity assays (HT1080^{eLuc} cells). NCI-N87 (naturally HER2-positive) cells were also engineered to express eLuc (NCI-N87^{eLuc} cells).

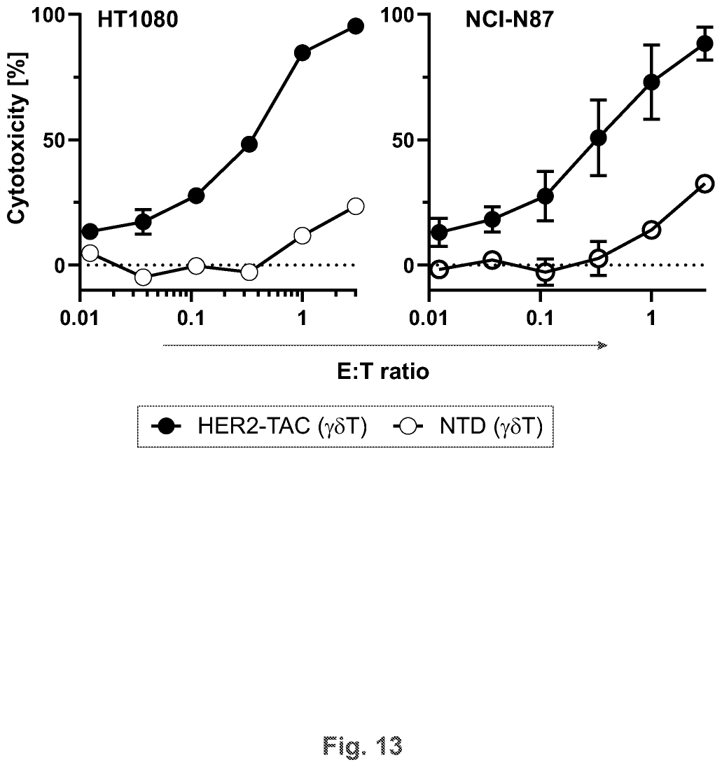

HER2-TAC or NTD γδ T cells were co-cultured with HT1080^{eLuc} or NCI-N87^{eLuc} cells at various effector to target (E:T) ratios for 14 hours. At the end of the co-culture the viability of the tumor cells was assessed by measuring luminescence relative to an untreated control. Results are shown in FIG. 13. HER2-TAC γδ T cells induced a more potent cytotoxic response relative to NTD γδ T cells. NTD γδ T cells still displayed some cytotoxicity, potentially due their innate ability to recognize certain stress ligands expressed by tumor cells.

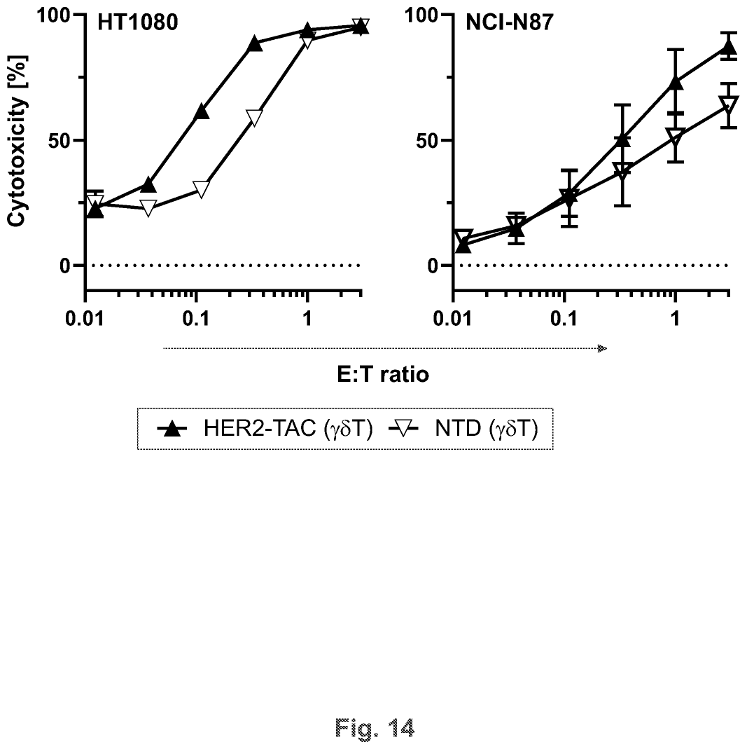

In an additional experiment, target cells were pretreated with 5 μM zoledronate overnight prior to co-culture. Following pretreatment, HER2-TAC or NTD γδ T cells were co-cultured with HT1080^{eLuc} or NCI-N87^{eLuc} Cells at various effector to target (E:T) ratios for 14 hours. At the end of the co-culture the viability of the tumor cells was assessed by measuring luminescence relative to an untreated control. Results are shown in FIG. 14. Zoledronate treatment enhanced cytotoxicity induced by NTD γδ T cells. However, HER2-TAC γδ T cells still showed superior cytotoxicity relative to NTD γδ T cells in all conditions tested.

While activated HER2-TAC γδ T cells displayed cytotoxicity towards HT1080^{eLuc} cells (FIG. 13), zoledronate pretreatment further enhanced cytotoxicity induced by activated HER2-TAC γδ T cells (FIG. 14). This enhanced cytotoxicity was unexpected because zoledronate and TAC are expected to activate γδ T cells via a TCR-dependent mechanism. While zoledronate-mediated activation can vary depending on the availability of phosphoantigens on tumor cells, the HER2 antigen alone was expected to be sufficient for full activation of HER2-TAC γδ T cells. Furthermore, zoledronate had already been used during manufacturing of the HER2-TAC γδ T cells. Therefore, it was unexpected that in HER2-expressing tumor cells, the addition of zoledronate would further enhance the activity of HER2-TAC γδ T cells.

### Example 6. Cytokine Production by TAC-Expressing γδ T Cells

γδ T cells were engineered to express a TAC specific for HER2 (HER2-TAC γδ T cells), generally using the zoledronate-based manufacturing process described in Example 2. Non-engineered γδ T cells (NTD γδ T cells) and NCI-N87 cells were also used in experiments.

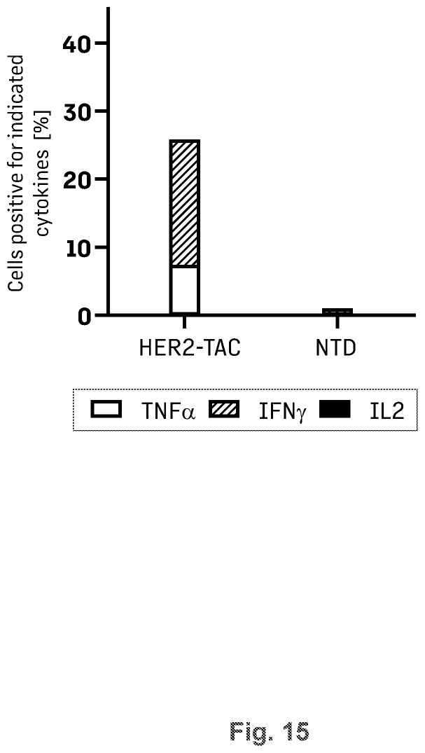

HER2-TAC or NTD γδ T cells were co-cultured with NCI-N87 cells at an effector to target (E:T) ratio of 1:1. Cells were co-cultured for 4 hours, and stained for either TNFα, IFNγ or IL2. The percentage of cells positive for the indicated cytokine in shown in FIG. 15. As depicted, only HER2-TAC but not NTD γδ T cells showed cytokine production when co-cultured with HER2-positive target cells

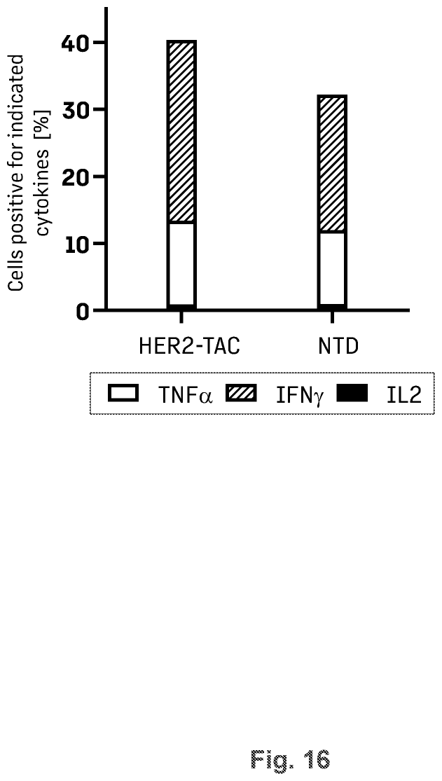

In an additional experiment, target cells were pretreated with 5 μM zoledronate overnight prior to co-culture. Following pretreatment, HER2-TAC or NTD γδ T cells were co-cultured with NCI-N87 cells at an effector to target (E:T) ratio of 1:1 for 4 hours, and stained for either TNFα, IFNγ or IL2, as described above. The percentage of cells positive for the indicated cytokine in shown in FIG. 16. Both HER2-TAC and NTD γδ T cells showed cytokine production when co-cultured with HER2-positive target cells, although cytokine production was greater in HER2-TAC-engineered cells.

While activated HER2-TAC γδ T cells displayed cytokine production when co-cultured with target cells (FIG. 15), zoledronate pretreatment further enhanced cytokine production in activated HER2-TAC γδ T cells (FIG. 16). This enhanced cytokine production was unexpected because zoledronate and TAC are expected to activate γδ T cells via a TCR-dependent mechanism. While zoledronate-mediated activation can vary depending on the availability of phosphoantigens on tumor cells, the HER2 antigen alone was expected to be sufficient for full activation of HER2-TAC γδ T cells. Furthermore, zoledronate had already been used during manufacturing of the HER2-TAC γδ T cells. Therefore, it was unexpected that in HER2-expressing tumor cells, the addition of zoledronate would further enhance the activity of HER2-TAC γδ T cells.

### Example 7. Cytotoxicity of TAC-Expressing γδ and αβ T Cells

γδ and αβ T cells were engineered to express a TAC specific for HER2 (HER2-TAC γδ T cells and HER2-TAC αβ T cells, respectively). γδ T cells were manufactured generally using the zoledronate-based manufacturing process described in Example 2. Non-engineered γδ and αβ T cells (NTD γδ T cells and NTD αβ T cells, respectively) and NCI-N87 cells were also used in experiments.

HER2-TAC γδ, NTD γδ, HER2-TAC αβ, or NTD αβ T cells were co-cultured with NCI-N87 cells at varying effector to target (E:T) ratios in the presence of 50 IU/ml IL2. Cells were co-cultured for 5 days, and monitored in real time by a fluorescence microscope (Cytation 5). T cells were stained with Vybrant® DiD cell-labeling solution (DiD). A DNA binding death dye (Cell Tox Green) was included. Cell death was measured as an increase in area of the cell death dye (the area of T cells that stained positive for the death dye.

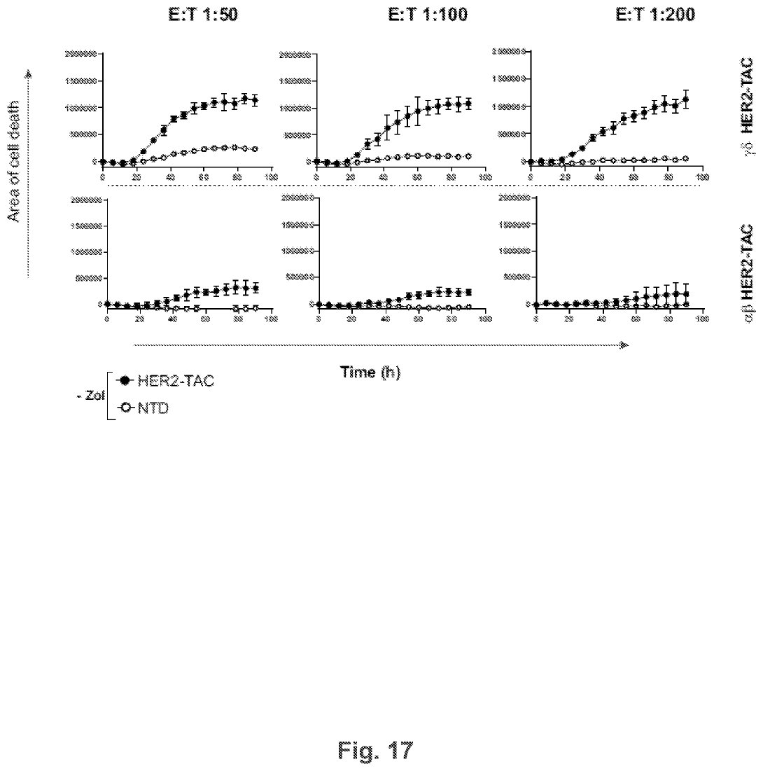

Results are shown in FIG. 17. HER2-TAC αβ T cells showed a lower level of cytotoxicity relative to HER2-TAC γδ T cells, suggesting that HER2-TAC γδ T cells are more potent than corresponding HER2-TAC αβ T cells. NTD αβ T cells showed no meaningful cytotoxicity, while some low level of non-specific cytotoxicity was observed for γδ NTD cells. Cytotoxicity was dose dependent in all cases with most efficacy observed at an E:T ratio of 1:50 and least efficacy observed at an E:T ratio of 1:200.

In an additional experiment, target cells were pretreated with zoledronate overnight prior to co-culture. Following pretreatment, HER2-TAC γδ, NTD γδ, HER2-TAC ac or NTD αβ T cells were co-cultured with NCI-N87 cells at varying effector to target (E:T) ratios in the presence of 50 IU/ml IL2 and cytotoxicity was assayed as described above.

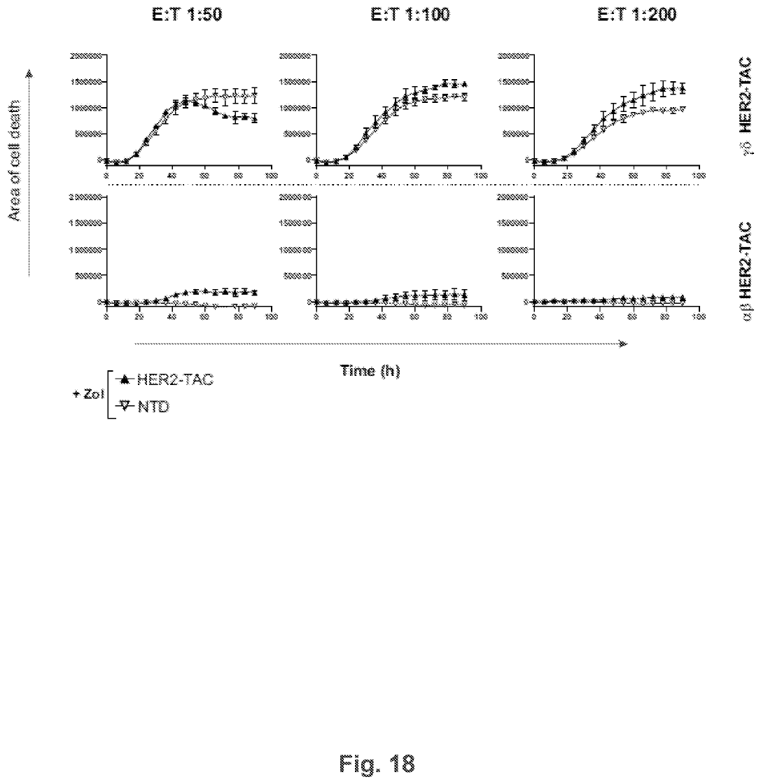

Results are shown in FIG. 18. Activated HER2-TAC αβ T cells showed a low level of cytotoxicity which was not further enhanced by the addition of zoledronate. In contrast, activated HER2-TAC γδ T cells showed an enhanced level of cytotoxicity after target cells had been pretreated with zoledronate. HER2-TAC γδ T induced cytotoxicity was greater than NTD γδ T cell induced cytotoxicity. NTD αβ T cells showed no meaningful cytotoxicity. Cytotoxicity was dose dependent in all cases with most efficacy observed at an E:T ratio of 1:50 and least efficacy observed at an E:T ratio of 1:200.

While activated HER2-TAC γδ T cells displayed cytotoxicity (FIG. 17), zoledronate pretreatment further enhanced cytotoxicity induced by activated HER2-TAC γδ T cells (FIG. 18). This enhanced cytotoxicity was unexpected because zoledronate and TAC are expected to activate γδ T cells via a TCR-dependent mechanism. While zoledronate-mediated activation can vary depending on the availability of phosphoantigens on tumor cells, the HER2 antigen alone was expected to be sufficient for full activation of HER2-TAC γδ T cells. Furthermore, zoledronate had already been used during manufacturing of the HER2-TAC γδ T cells. Therefore, it was unexpected that in HER2-expressing tumor cells, the addition of zoledronate would further enhance the activity of HER2-TAC γδ T cells.

### Example 8. CD16 Stimulation of TAC-Expressing γδ T Cells

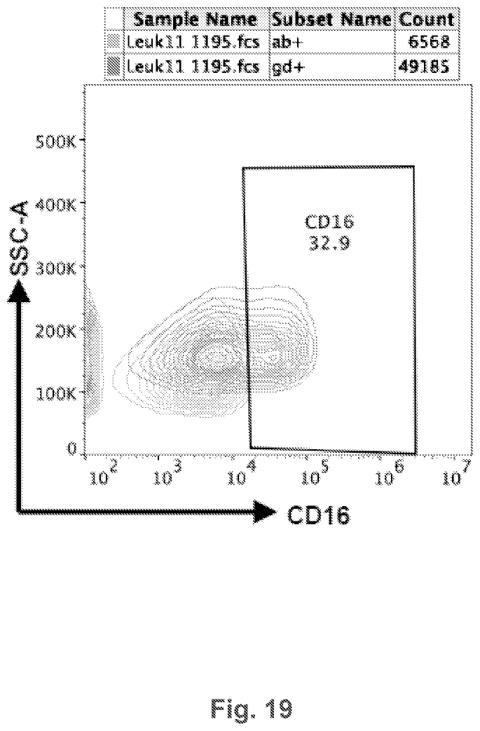

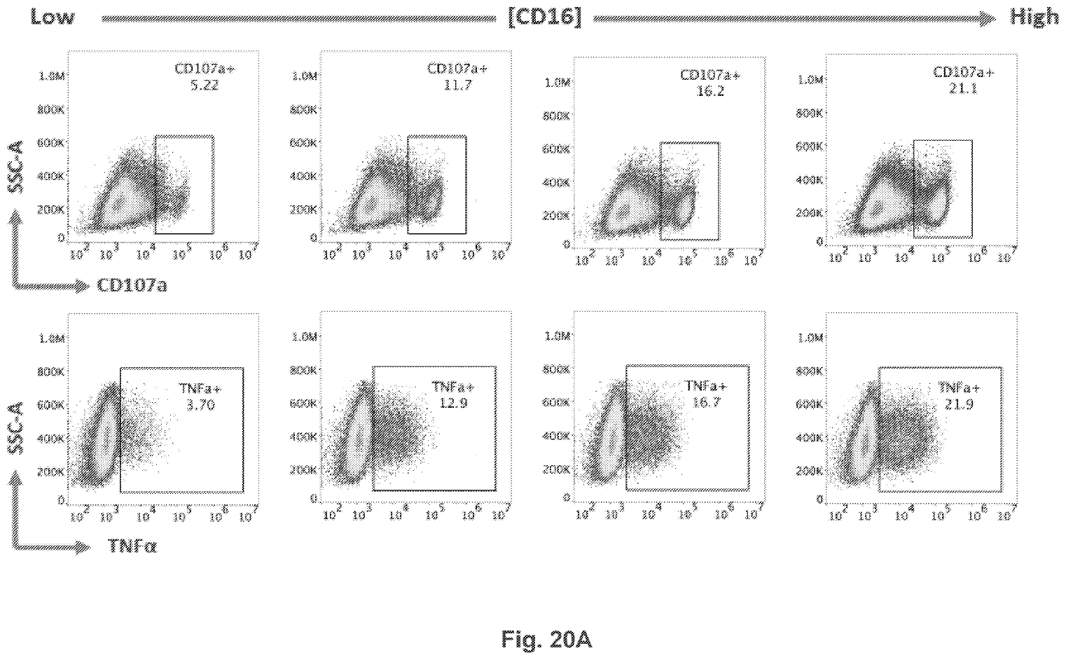

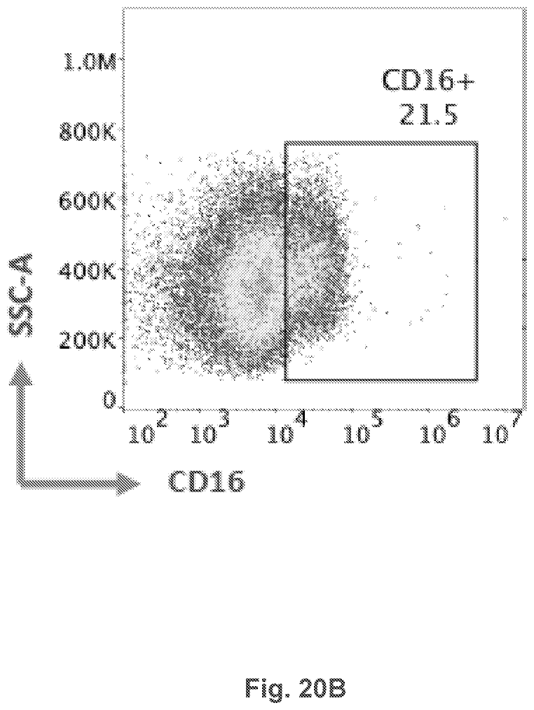

We assessed CD16 (FcγRIII) expression on γδ T cells. γδ T cells were manufactured generally using the zoledronate-based manufacturing process described in Example 2. A manufactured γδ T cell culture demonstrated expression of CD16, both when non-transduced (data not shown) and when expressing a BCMA-TAC (FIG. 19). CD16 expression was higher in γδ T cells than αβ T cells.

To determine whether CD16 expressed on manufactured γδ T cells was functional, we performed a 4-hour plate-bound stimulation with an agonistic CD16 monoclonal antibody (bound at 10, 25, 50, or 100 ng/μL concentrations) and assessed CD107a mobilization and TNFα expression.

Results are shown in FIGS. 20A and 20B. A dose-dependent response to CD16 stimulation was observed for both CD107a mobilization and TNFα expression (FIG. 20A). Stimulation at the highest CD16 concentration (100 ng/μL) resulted in proportions of CD107a+ and TNFα+ γδ T cells that were similar to the proportions of CD16+ γδ T cells in the non-stimulated culture (FIG. 20B), suggesting that this CD16 concentration stimulated all CD16+ γδ T cells in the culture.

Next, we tested whether effector functions from TAC stimulation could be boosted by CD16 stimulation. To minimize the impact of donor variability, we screened 10 donors for CD16+ γδ T cell population and selected 7 CD16+ donors for large-scale γδ T cell manufacturing. We first assessed whether BCMA-TAC stimulation by BCMA antigen (as measured by CD107a and TNFα expression in Vδ2+ NGFR+ cells) could be enhanced by direct stimulation of CD16 using an agonistic CD16 mAb in a plate-bound assay repeated across 6 donors. Cells were stimulated for 4 hours with plate-bound BCMA-Fc bound at 0 or 2 ng/μL concentrations, and/or CD16 bound at 0, 5, 25, or 100 ng/μL concentrations.

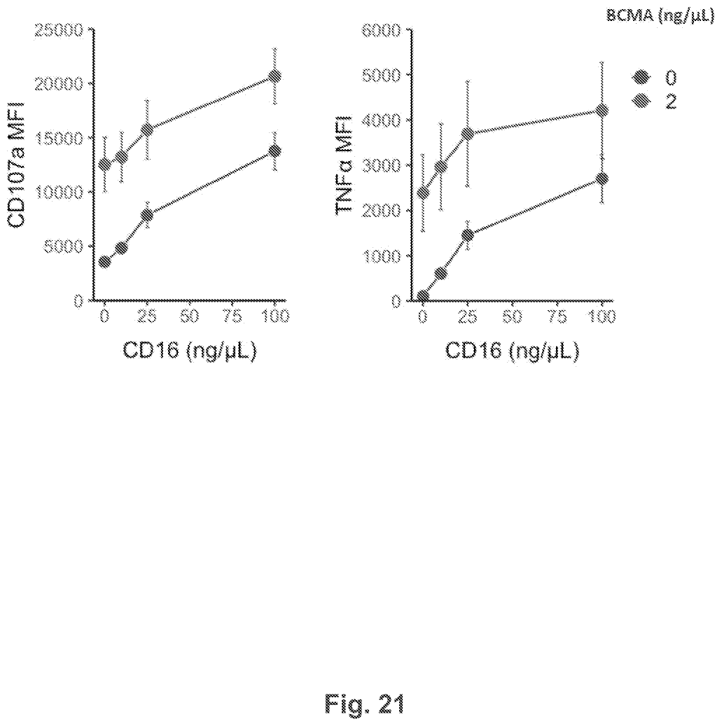

Results are shown in FIG. 21. Generally, there appeared to be a dose-dependent response to CD16 in BCMA-TAC γδ T cells, whether the cells were dual stimulated with BCMA and CD16 or stimulated with CD16 alone.

The effect of CD16 on degranulation of γδ T cells (as measured by CD107a expression) was more pronounced in BCMA-TAC γδ T cells stimulated with CD16 alone, as demonstrated by significant differences between all CD16 only stimulation conditions. While BCMA-TAC γδ T cells stimulated with BCMA and CD16 exhibited a similar trend, there was only a significant difference in degranulation between BCMA-TAC γδ T cells stimulated with BCMA alone and dual stimulated with the highest CD16 concentration (100 ng/μL). Together, these results indicate that CD16 can enhance BCMA-TAC γδ T cell degranulation.

Similarly, CD16 stimulation (alone) of BCMA-TAC γδ T cells resulted in a dose-dependent response for TNFα expression. Dual stimulation of BCMA-TAC γδ T cells with BCMA and CD16 followed a similar trend, although there was a higher variance in response. Furthermore, CD16 boosting of TNFα expression appeared to plateau between 25 ng/μL and 100 ng/μL in dual stimulated cells. Overall, these results demonstrate that BCMA-TAC γδ T cells can have increased TNFα expression when stimulated via CD16 and/or the TAC receptor.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of treating a cancer in an individual in need thereof, comprising administering to the individual a therapeutically effective amount of:
(i) a γδ T cell comprising a T cell-antigen coupler (TAC) polypeptide comprising:
(a) an antigen-binding domain that binds a CD19 antigen, a HER2 antigen, or a BCMA antigen;
(b) an antigen-binding domain that binds a protein associated with a TCR complex; and
(c) a TCR co-receptor cytosolic domain and transmembrane domain;
wherein (a), (b), and (c) are fused directly to each other, or joined by at least one linker; and
(ii) zoledronate.

2. The method of claim 1, wherein the zoledronate is administered before, after, or simultaneously with the γδ T cell

3. The method of claim 1 or 2, wherein the antigen-binding domain that binds a CD19 antigen, a HER2 antigen, or a BCMA antigen is a designed ankyrin repeat (DARPin) polypeptide, or a single chain variable fragment (scFv).

4. The method of any one of claims 1-3, wherein the protein associated with the TCR complex is a CD3 protein.

5. The method of claim 4, wherein the CD3 protein is of a TCR complex on the γδ T cell.

6. The method of claim 4 or 5, wherein binding of the CD3 protein induces activation of the γδ T cell.

7. The method of any one of claims 1-6, wherein the antigen-binding domain that binds the protein associated with the TCR complex is selected from UCHT1, OKT3, F6A, L2K, or any variants thereof.

8. The method of claim 7, wherein the antigen-binding domain that binds the protein associated with the TCR complex is a UCHT1 antigen-binding domain.

9. The method of claim 8, wherein the UCHT1 antigen-binding domain is a single chain antibody.

10. The method of claim 8 or 9, wherein the UCHT1 antigen-binding domain comprises a Y to T mutation at a position corresponding to amino acid 182 of SEQ ID NO: 14 (Y182T).

11. The method of any one of claims 8-10, wherein the UCHT1 antigen-binding domain is a humanized variant of UCHT1 (huUCHT1).

12. The method of claim 11, wherein the UCHT1 antigen-binding domain is a humanized variant of UCHT1 comprising a Y to T mutation at a position corresponding to amino acid 177 of SEQ ID NO: 44 (huUCHT1 (Y177T)).

13. The method of any one of claims 8-12, wherein the UCHT1 antigen-binding domain comprises an amino acid sequence having at least 80%, at least 85%, at least 90%, at least 95%, at least 96%, at least 97%, at least 98%, at least 99%, or 100% sequence identity with SEQ ID NO: 14 (UCHT1), SEQ ID NO: 72 (UCHT1 (Y182T)), SEQ ID NO: 44 (huUCHT1), or SEQ ID NO: 46 (huUCHT1 (Y177T)).

14. The method of claim 7, wherein the antigen-binding domain that binds the protein associated with the TCR complex is OKT3.

15. The method of claim 14, wherein the antigen-binding domain that binds a protein associated with the TCR complex comprises an amino acid sequence having at least 80%, at least 85%, at least 90%, at least 95%, at least 96%, at least 97%, at least 98%, at least 99%, or 100% sequence identity with SEQ ID NO: 22.

16. The method of claim 7, wherein the antigen-binding domain that binds the protein associated with the TCR complex is F6A.

17. The method of claim 16, wherein the antigen-binding domain that binds the protein associated with the TCR complex comprises an amino acid sequence having at least 80%, at least 85%, at least 90%, at least 95%, at least 96%, at least 97%, at least 98%, at least 99%, or 100% sequence identity with SEQ ID NO: 24.

18. The method of claim 7, wherein the antigen-binding domain that binds the protein associated with the TCR complex is L2K.

19. The method of claim 18, wherein the antigen-binding domain that binds the protein associated with the TCR complex comprises an amino acid sequence having at least 80%, at least 85%, at least 90%, at least 95%, at least 96%, at least 97%, at least 98%, at least 99%, or 100% sequence identity with SEQ ID NO: 26.

20. The method of any one of claims 1-19, wherein the cytosolic domain is a CD4 cytosolic domain and the transmembrane domain is a CD4 transmembrane domain.

21. The method of claim 20, wherein the TCR co-receptor cytosolic domain and transmembrane domain comprises an amino acid sequence having at least 80%, at least 85%, at least 90%, at least 95%, at least 96%, at least 97%, at least 98%, at least 99%, or 100% sequence identity with SEQ ID NO: 18.

22. The method of any one of claims 1-19, wherein the cytosolic domain is a CD8 cytosolic domain and the transmembrane domain is a CD8 transmembrane domain.

23. The method of any one of claims 1-22, wherein (a) and (c) are fused to (b).

24. The method of any one of claims 1-22, wherein (b) and (c) are fused to (a).

25. The method of any one of claims 1-24, wherein at least one linker joins (a) to (b).

26. The method of claim 25, wherein the at least one linker is a G₄S flexible linker, a large protein domain, a long helix structure, or a short helix structure.

27. The method of claim 26, wherein the at least one linker comprises an amino acid sequence having at least 80%, at least 85%, at least 90%, at least 95%, at least 96%, at least 97%, at least 98%, at least 99%, or 100% sequence identity with SEQ ID NO: 12 ((G4S)4-based linker), SEQ ID NO: 16 (G4S-based linker), SEQ ID NO: 20 (CD4 based linker), SEQ ID NO: 28 (short helix connector), SEQ ID NO: 30 (long helix connector), SEQ ID NO: 32 (large domain connector), SEQ ID NO: 69 (flexible connector), SEQ ID NO: 73 (G4S flexible linker), or SEQ ID NO: 74 (G4S3 flexible linker).

28. The method of any one of claims 1-27, wherein the TAC polypeptide does not comprise a co-stimulatory domain.

29. The method of any one of claims 1-28, wherein the TAC polypeptide does not comprise an activation domain.

30. The method of any one of claims 1-28, wherein the TAC polypeptide further comprises a leader sequence.

31. The method of claim 30, wherein the leader sequence comprises an amino acid sequence having at least 80%, at least 85%, at least 90%, at least 95%, at least 96%, at least 97%, at least 98%, at least 99%, or 100% sequence identity with SEQ ID NO: 6 (muIgG leader), SEQ ID NO: 48 (huIgG leader), or SEQ ID NO: 50 (huCD8a leader).

32. The method of any one of claims 1-31, wherein the TAC polypeptide comprises an antigen-binding domain that binds a CD19 antigen.

33. The method of claim 32, wherein the antigen-binding domain that binds a CD19 antigen comprises an amino acid sequence having at least 80%, at least 85%, at least 90%, at least 95%, at least 96%, at least 97%, at least 98%, at least 99%, or 100% sequence identity with SEQ ID NO: 36 (CD19 scFv).

34. The method of claim 32 or 33, wherein the TAC polypeptide comprises an amino acid sequence having at least 80%, at least 85%, at least 90%, at least 95%, at least 96%, at least 97%, at least 98%, at least 99%, or 100% sequence identity with SEQ ID NO: 64 (CD19 TAC).

35. The method of any one of claims 32-34, wherein the TAC polypeptide is encoded by a nucleic acid comprising a nucleotide sequence having at least 80%, at least 85%, at least 90%, at least 95%, at least 96%, at least 97%, at least 98%, at least 99%, or 100% sequence identity with SEQ ID NO: 63 (CD19 TAC).

36. The method of any one of claims 1-31, wherein the TAC polypeptide comprises an antigen-binding domain that binds a HER2 antigen.

37. The method of claim 36, wherein the antigen-binding domain that binds a HER2 antigen comprises an antigen binding domain of an antibody selected from Trastuzumab, Pertuzumab, Lapatinib, Neratinib, Ado-trastuzumab Emtansine, Gancotamab, Margetuximab, Timigutuzumab, and Ertumaxomab.

38. The method of claim 36 or 37, wherein the antigen-binding domain that binds a HER2 antigen comprises an amino acid sequence having at least 80%, at least 85%, at least 90%, at least 95%, at least 96%, at least 97%, at least 98%, at least 99%, or 100% sequence identity with SEQ ID NO: 8 (HER2 DARPin).

39. The method of any one of claims 36-38, wherein the TAC polypeptide comprises an amino acid sequence having at least 80%, at least 85%, at least 90%, at least 95%, at least 96%, at least 97%, at least 98%, at least 99%, or 100% sequence identity with SEQ ID NO: 66 (HER2 TAC: huIgG leader sequence; huUCHT1 CD3-binding domain), SEQ ID NO: 68 (HER2 TAC: CD8a leader; huUCHT1 CD3-binding domain), or SEQ ID NO: 76 (HER2 TAC: muIgG leader; huUCHT1 CD3-binding domain).

40. The method of any one of claims 36-39, wherein the TAC polypeptide is encoded by a nucleic acid comprising a nucleotide sequence having at least 80%, at least 85%, at least 90%, at least 95%, at least 96%, at least 97%, at least 98%, at least 99%, or 100% sequence identity with SEQ ID NO: 65 (HER2 TAC: huIgG leader; huUCHT1 CD3-binding domain), SEQ ID NO: 67 (HER2 TAC: CD8a leader; huUCHT1 CD3-binding domain), or SEQ ID NO: 75 (HER2 TAC: muIgG leader; huUCHT1 CD3-binding domain).

41. The method of any one of claims 1-31, wherein the TAC polypeptide comprises an antigen-binding domain that binds a BCMA antigen.

42. The method of claim 41, wherein the antigen-binding domain that binds a BCMA antigen comprises an antigen binding domain of Belantamab mafodotin.

43. The method of claim 41 or 42, wherein the antigen-binding domain that binds a BCMA antigen comprises an amino acid sequence having at least 80%, at least 85%, at least 90%, at least 95%, at least 96%, at least 97%, at least 98%, at least 99%, or 100% sequence identity with SEQ ID NO: 34 (BCMA scFv), SEQ ID NO: 52 (3625 BCMA scFv, Vh-Vl), or SEQ ID NO: 54 (3625 BCMA scFv, Vl-Vh).

44. The method of any one of claims 41-43, wherein the TAC polypeptide comprises an amino acid sequence having at least 80%, at least 85%, at least 90%, at least 95%, at least 96%, at least 97%, at least 98%, at least 99%, or 100% sequence identity with SEQ ID NO: 56 (BCMA TAC: 3625 BCMA scFv, Vh-Vl, helix linker; huUCHT1 CD3-binding domain), SEQ ID NO: 58 (BCMA TAC: 3625 BCMA scFv, Vl-Vh, helix linker; huUCHT1 CD3-binding domain), SEQ ID NO: 60 (BCMA TAC: 3625 BCMA scFv, Vh-Vl, G4S linker; huUCHT1 CD3-binding domain), or SEQ ID NO: 62 (BCMA TAC: 3625 BCMA scFv, Vl-Vh, G4S linker; huUCHT1 CD3-binding domain).

45. The method of any one of claims 41-44, wherein the TAC polypeptide is encoded by a nucleic acid comprising a nucleotide sequence having at least 80%, at least 85%, at least 90%, at least 95%, at least 96%, at least 97%, at least 98%, at least 99%, or 100% sequence identity with SEQ ID NO: 55 (BCMA TAC: 3625 BCMA scFv, Vh-Vl, helix linker; huUCHT1 CD3-binding domain), SEQ ID NO: 57 (BCMA TAC: 3625 BCMA scFv, Vl-Vh, helix linker; huUCHT1 CD3-binding domain), SEQ ID NO: 59 (BCMA TAC: 3625 BCMA scFv, Vh-Vl, G4S linker; huUCHT1 CD3-binding domain), or SEQ ID NO: 61 (BCMA TAC: 3625 BCMA scFv, Vl-Vh, G4S linker; huUCHT1 CD3-binding domain).

46. A method of making γδ T cells comprising a TAC polypeptide, comprising:
(a) contacting γδ T cells isolated from an individual with zoledronate and IL-2;
(b) contacting the γδ T cells with an expression vector comprising a nucleic acid encoding the TAC polypeptide;
(c) culturing the cells; and
(d) removing αβ T cells from the culture.

47. A γδ T cell made by the method of claim 46.

48. A pharmaceutical composition comprising the γδ T cell of claim 47.

49. A method of treating a cancer in an individual in need thereof, comprising administering to the individual a therapeutically effective amount of the pharmaceutical composition of claim 48.

50. The method of claim 49, further comprising administering zoledronate to the individual.

51. The method of claim 50, wherein the zoledronate is administered before, after, or simultaneously with the γδ T cell

52. A γδ T cell comprising a T cell-antigen coupler (TAC) polypeptide comprising:
(a) an antigen-binding domain that binds a CD19 antigen, a HER2 antigen, or a BCMA antigen;
(b) an antigen-binding domain that binds a protein associated with a TCR complex; and
(c) a TCR co-receptor cytosolic domain and transmembrane domain;
wherein (a), (b), and (c) are fused directly to each other, or joined by at least one linker.

53. The γδ T cell of claim 52, wherein the antigen-binding domain that binds a CD19 antigen, a HER2 antigen, or a BCMA antigen is a designed ankyrin repeat (DARPin) polypeptide, or a single chain variable fragment (scFv).

54. The γδ T cell of claim 52 or 53, wherein the protein associated with the TCR complex is a CD3 protein.

55. The γδ T cell of claim 54, wherein the CD3 protein is of a TCR complex on the γδ T cell.

56. The γδ T cell of claim 54 or 55, wherein binding of the CD3 protein induces activation of the γδ T cell.

57. The γδ T cell of any one of claims 52-56, wherein the antigen-binding domain that binds the protein associated with the TCR complex is selected from UCHT1, OKT3, F6A, L2K, or any variants thereof.

58. The γδ T cell of claim 57, wherein the antigen-binding domain that binds the protein associated with the TCR complex is a UCHT1 antigen-binding domain.

59. The γδ T cell of claim 58, wherein the UCHT1 antigen-binding domain is a single chain antibody.

60. The γδ T cell of claim 58 or 59, wherein the UCHT1 antigen-binding domain comprises a Y to T mutation at a position corresponding to amino acid 182 of SEQ ID NO: 14 (Y182T).

61. The γδ T cell of any one of claims 58-60, wherein the UCHT1 antigen-binding domain is a humanized variant of UCHT1 (huUCHT1).

62. The γδ T cell of claim 61, wherein the UCHT1 antigen-binding domain is a humanized variant of UCHT1 comprising a Y to T mutation at a position corresponding to amino acid 177 of SEQ ID NO: 44 (huUCHT1 (Y177T)).

63. The γδ T cell of any one of claims 58-62, wherein the UCHT1 antigen-binding domain comprises an amino acid sequence having at least 80%, at least 85%, at least 90%, at least 95%, at least 96%, at least 97%, at least 98%, at least 99%, or 100% sequence identity with SEQ ID NO: 14 (UCHT1), SEQ ID NO: 72 (UCHT1 (Y182T)), SEQ ID NO: 44 (huUCHT1), or SEQ ID NO: 46 (huUCHT1 (Y177T)).

64. The γδ T cell of claim 57, wherein the antigen-binding domain that binds the protein associated with the TCR complex is OKT3.

65. The γδ T cell of claim 64, wherein the antigen-binding domain that binds a protein associated with the TCR complex comprises an amino acid sequence having at least 80%, at least 85%, at least 90%, at least 95%, at least 96%, at least 97%, at least 98%, at least 99%, or 100% sequence identity with SEQ ID NO: 22.

66. The γδ T cell of claim 57, wherein the antigen-binding domain that binds the protein associated with the TCR complex is F6A.

67. The γδ T cell of claim 66, wherein the antigen-binding domain that binds the protein associated with the TCR complex comprises an amino acid sequence having at least 80%, at least 85%, at least 90%, at least 95%, at least 96%, at least 97%, at least 98%, at least 99%, or 100% sequence identity with SEQ ID NO: 24.

68. The γδ T cell of claim 57, wherein the antigen-binding domain that binds the protein associated with the TCR complex is L2K.

69. The γδ T cell of claim 68, wherein the antigen-binding domain that binds the protein associated with the TCR complex comprises an amino acid sequence having at least 80%, at least 85%, at least 90%, at least 95%, at least 96%, at least 97%, at least 98%, at least 99%, or 100% sequence identity with SEQ ID NO: 26.

70. The γδ T cell of any one of claims 52-69, wherein the cytosolic domain is a CD4 cytosolic domain and the transmembrane domain is a CD4 transmembrane domain.

71. The γδ T cell of claim 70, wherein the TCR co-receptor cytosolic domain and transmembrane domain comprise an amino acid sequence having at least 80%, at least 85%, at least 90%, at least 95%, at least 96%, at least 97%, at least 98%, at least 99%, or 100% sequence identity with SEQ ID NO: 18.

72. The γδ T cell of any one of claims 52-69, wherein the cytosolic domain is a CD8 cytosolic domain and the transmembrane domain is a CD8 transmembrane domain.

73. The γδ T cell of any one of claims 52-72, wherein (a) and (c) are fused to (b).

74. The γδ T cell of any one of claims 52-72, wherein (b) and (c) are fused to (a).

75. The γδ T cell of any one of claims 52-74, wherein at least one linker joins (a) to (b).

76. The γδ T cell of claim 75, wherein the at least one linker is a G₄S flexible linker, a large protein domain, a long helix structure, or a short helix structure.

77. The γδ T cell of claim 76, wherein the at least one linker comprises an amino acid sequence having at least 80%, at least 85%, at least 90%, at least 95%, at least 96%, at least 97%, at least 98%, at least 99%, or 100% sequence identity with SEQ ID NO: 12 ((G4S)4-based linker), SEQ ID NO: 16 (G4S-based linker), SEQ ID NO: 20 (CD4 based linker), SEQ ID NO: 28 (short helix connector), SEQ ID NO: 30 (long helix connector), SEQ ID NO: 32 (large domain connector), SEQ ID NO: 69 (flexible connector), SEQ ID NO: 73 (G4S flexible linker), or SEQ ID NO: 74 (G4S3 flexible linker).

78. The γδ T cell of any one of claims 52-77, wherein the TAC polypeptide does not comprise a co-stimulatory domain.

79. The γδ T cell of any one of claims 52-78, wherein the TAC polypeptide does not comprise an activation domain.

80. The γδ T cell of any one of claims 52-79, wherein the TAC polypeptide further comprises a leader sequence.

81. The γδ T cell of claim 80, wherein the leader sequence comprises an amino acid sequence having at least 80%, at least 85%, at least 90%, at least 95%, at least 96%, at least 97%, at least 98%, at least 99%, or 100% sequence identity with SEQ ID NO: 6 (muIgG leader), SEQ ID NO: 48 (huIgG leader), or SEQ ID NO: 50 (huCD8a leader).

82. The γδ T cell of any one of claims 52-81, wherein the TAC polypeptide comprises an antigen-binding domain that binds a CD19 antigen.

83. The γδ T cell of claim 82, wherein the antigen-binding domain that binds a CD19 antigen comprises an amino acid sequence having at least 80%, at least 85%, at least 90%, at least 95%, at least 96%, at least 97%, at least 98%, at least 99%, or 100% sequence identity with SEQ ID NO: 36 (CD19 scFv).

84. The γδ T cell of claim 82 or 83, wherein the TAC polypeptide comprises an amino acid sequence having at least 80%, at least 85%, at least 90%, at least 95%, at least 96%, at least 97%, at least 98%, at least 99%, or 100% sequence identity with SEQ ID NO: 64 (CD19 TAC).

85. The γδ T cell of any one of claims 82-84, wherein the TAC polypeptide is encoded by a nucleic acid comprising a nucleotide sequence having at least 80%, at least 85%, at least 90%, at least 95%, at least 96%, at least 97%, at least 98%, at least 99%, or 100% sequence identity with SEQ ID NO: 63 (CD19 TAC).

86. The γδ T cell of any one of claims 52-81, wherein the TAC polypeptide comprises an antigen-binding domain that binds a HER2 antigen.

87. The γδ T cell of claim 86, wherein the antigen-binding domain that binds a HER2 antigen comprises an antigen binding domain of an antibody selected from Trastuzumab, Pertuzumab, Lapatinib, Neratinib, Ado-trastuzumab Emtansine, Gancotamab, Margetuximab, Timigutuzumab, and Ertumaxomab.

88. The γδ T cell of claim 86 or 87, wherein the antigen-binding domain that binds a HER2 antigen comprises an amino acid sequence having at least 80%, at least 85%, at least 90%, at least 95%, at least 96%, at least 97%, at least 98%, at least 99%, or 100% sequence identity with SEQ ID NO: 8 (HER2 DARPin).

89. The γδ T cell of any one of claims 86-88, wherein the TAC polypeptide comprises an amino acid sequence having at least 80%, at least 85%, at least 90%, at least 95%, at least 96%, at least 97%, at least 98%, at least 99%, or 100% sequence identity with SEQ ID NO: 66 (HER2 TAC: huIgG leader sequence; huUCHT1 CD3-binding domain), SEQ ID NO: 68 (HER2 TAC: CD8a leader; huUCHT1 CD3-binding domain), or SEQ ID NO: 76 (HER2 TAC: muIgG leader; huUCHT1 CD3-binding domain).

90. The γδ T cell of any one of claims 86-89, wherein the TAC polypeptide is encoded by a nucleic acid comprising a nucleotide sequence having at least 80%, at least 85%, at least 90%, at least 95%, at least 96%, at least 97%, at least 98%, at least 99%, or 100% sequence identity with SEQ ID NO: 65 (HER2 TAC: huIgG leader; huUCHT1 CD3-binding domain), SEQ ID NO: 67 (HER2 TAC: CD8a leader; huUCHT1 CD3-binding domain), or SEQ ID NO: 75 (HER2 TAC: muIgG leader; huUCHT1 CD3-binding domain).

91. The γδ T cell of any one of claims 52-81, wherein the TAC polypeptide comprises an antigen-binding domain that binds a BCMA antigen.

92. The γδ T cell of claim 91, wherein the antigen-binding domain that binds a BCMA antigen comprises an antigen binding domain of Belantamab mafodotin.

93. The γδ T cell of claim 91 or 92, wherein the antigen-binding domain that binds a BCMA antigen comprises an amino acid sequence having at least 80%, at least 85%, at least 90%, at least 95%, at least 96%, at least 97%, at least 98%, at least 99%, or 100% sequence identity with SEQ ID NO: 34 (BCMA scFv), SEQ ID NO: 52 (3625 BCMA scFv, Vh-Vl), or SEQ ID NO: 54 (3625 BCMA scFv, Vl-Vh).

94. The γδ T cell of any one of claims 91-93, wherein the TAC polypeptide comprises an amino acid sequence having at least 80%, at least 85%, at least 90%, at least 95%, at least 96%, at least 97%, at least 98%, at least 99%, or 100% sequence identity with SEQ ID NO: 56 (BCMA TAC: 3625 BCMA scFv, Vh-Vl, helix linker; huUCHT1 CD3-binding domain), SEQ ID NO: 58 (BCMA TAC: 3625 BCMA scFv, Vl-Vh, helix linker; huUCHT1 CD3-binding domain), SEQ ID NO: 60 (BCMA TAC: 3625 BCMA scFv, Vh-Vl, G4S linker; huUCHT1 CD3-binding domain), or SEQ ID NO: 62 (BCMA TAC: 3625 BCMA scFv, Vl-Vh, G4S linker; huUCHT1 CD3-binding domain).

95. The γδ T cell of any one of claims 91-94, wherein the TAC polypeptide is encoded by a nucleic acid comprising a nucleotide sequence having at least 80%, at least 85%, at least 90%, at least 95%, at least 96%, at least 97%, at least 98%, at least 99%, or 100% sequence identity with SEQ ID NO: 55 (BCMA TAC: 3625 BCMA scFv, Vh-Vl, helix linker; huUCHT1 CD3-binding domain), SEQ ID NO: 57 (BCMA TAC: 3625 BCMA scFv, Vl-Vh, helix linker; huUCHT1 CD3-binding domain), SEQ ID NO: 59 (BCMA TAC: 3625 BCMA scFv, Vh-Vl, G4S linker; huUCHT1 CD3-binding domain), or SEQ ID NO: 61 (BCMA TAC: 3625 BCMA scFv, Vl-Vh, G4S linker; huUCHT1 CD3-binding domain).

96. A γδ T cell comprising a nucleic acid encoding a T cell-antigen coupler (TAC) polypeptide, the nucleic acid comprising:
(a) a first polynucleotide encoding an antigen-binding domain that binds a CD19 antigen, a HER2 antigen, or a BCMA antigen;
(b) a second polynucleotide encoding an antigen-binding domain that binds a protein associated with a TCR complex; and
(c) a third polynucleotide encoding a TCR co-receptor cytosolic domain and transmembrane domain;
wherein components encoded by (a), components encoded by (b), and components encoded by (c) are fused directly to each other, or joined by at least one linker.

97. A γδ T cell comprising an expression vector comprising a nucleic acid encoding a T cell-antigen coupler (TAC) polypeptide, the nucleic acid comprising:
(a) a first polynucleotide encoding an antigen-binding domain that binds a CD19 antigen, a HER2 antigen, or a BCMA antigen;
(b) a second polynucleotide encoding an antigen-binding domain that binds a protein associated with a TCR complex; and
(c) a third polynucleotide encoding a TCR co-receptor cytosolic domain and transmembrane domain;
wherein components encoded by (a), components encoded by (b), and components encoded by (c) are fused directly to each other, or joined by at least one linker.

98. The γδ T cell of claim 97, wherein the expression vector is a lentiviral vector.

99. The γδ T cell of claim 98, wherein the lentiviral vector is a VSV-G pseudotyped lentivirus.

100. The γδ T cell of claim 97, wherein the expression vector is a γ retroviral vector.

101. The γδ T cell of claim 100, wherein the γ retroviral vector is a GALV pseudotyped γ-retrovirus.

102. The γδ T cell of any one of claims 52-101, wherein the γδ T cell is a δ2 T cell.

103. The γδ T cell of any one of claims 52-102, wherein the γδ T cell is a γ9δ2 T cell.

104. A pharmaceutical composition comprising the γδ T cell of any one of claims 52-103, and a pharmaceutically acceptable excipient.

105. A pharmaceutical composition comprising the γδ T cell of any one of claims 82-85, and a pharmaceutically acceptable excipient.

106. A pharmaceutical composition comprising the γδ T cell of any one of claims 86-90, and a pharmaceutically acceptable excipient.

107. A pharmaceutical composition comprising the γδ T cell of any one of claims 91-95, and a pharmaceutically acceptable excipient.

108. A method of treating a cancer in an individual in need thereof, comprising administering to the individual a therapeutically effective amount of the pharmaceutical composition of claim 104.

109. The method of claim 108, wherein the individual is a mammal.

110. The method of claim 108 or 109, wherein the cancer is a solid cancer or a liquid cancer.

111. The method of any one of claims 108-110, wherein the cancer is a lung cancer, a breast cancer, multiple myeloma, glioblastoma, gastric cancer, ovarian cancer, stomach cancer, colorectal cancer, urothelial cancer, endometrial cancer, or a colon cancer.

112. A method of treating a cancer comprising a CD19-expressing cancer cell in an individual in need thereof, comprising administering to the individual a therapeutically effective amount of the pharmaceutical composition of claim 105.

113. The method of claim 112, wherein the cancer is a B cell malignancy.

114. The method of claim 112 or 113, wherein the cancer is B cell lymphoma, acute lymphoblastic leukemia (ALL), chronic lymphocytic leukemia (CLL), or Non-Hodgkins Lymphoma.

115. A method of treating a cancer comprising a HER2-expressing cancer cell in an individual in need thereof, comprising administering to the individual a therapeutically effective amount of the pharmaceutical composition of claim 106.

116. The method of claim 115, wherein the cancer is breast cancer, bladder cancer, pancreatic cancer, ovarian cancer, or stomach cancer.

117. A method of treating a cancer comprising a BCMA-expressing cancer cell in an individual in need thereof, comprising administering to the individual a therapeutically effective amount of the pharmaceutical composition of claim 107.

118. The method of claim 117, wherein the cancer is leukemia, lymphoma, or multiple myeloma.

119. The method of any one of claims 108-118, wherein the pharmaceutical composition is administered in combination with zoledronate.

120. The method of any one of claims 108-119, wherein the pharmaceutical composition is administered in combination with IL-2.

121. The method of any one of claims 108-120, wherein the pharmaceutical composition is administered in combination with a CD16 agonist.
